SEQUENCE LISTING (70 sequences)

SEQ ID NO: 1 - DNA, 620 bp, Homo sapiens
tctgaaagttgatagagagttgatagaacaatcttttcataaggtataaattctatctatttttccctaaaaacaaacaaacagcaaccattcttgcttctaattgggcagtacaatctgataggttggctagagacttgcagtggggtgtccctggtacctattcaaagactgtagctttcttctatctcattctcattttctattctttgcattgtagagttttggagcaaagaaggtcatcaaacttatacagtgarcctaacagtttccttttaagatgaggaaactgagccccagccagccatgtgattcatcacagttccttggtggctgagttgggaggagaacacacatcttctcagctcctcccactgctctttccattaagacagacagcctctcattcaaagtaagagaatttccatcatatgagcaagggacaatgagagaactgcttctcagtactccccgcttcttcctcacctacttcctcttcactggatttgtcaactcacctgtctttacgcaatagttacaatgccagcatttctctacattacatacttcagcgattctcttactggctttgcaaagtcacccaaacacgaatggaaata

SEQ ID NO: 2 - DNA, 400 bp, Homo sapiens
ggcaatcttgagtgagctctattaattattattctctttggctcagttgctaagctattttatgcatgttatgccctttgacaattagtctttagctgtaatcccccagccatcctcagaaatgtggtgagtagccatagtgttcccaagattagaaaaaatgtaatggcagagccaagaggaaggtaaatggtccacatyttatgaagcatcatctaaatggccctattggttagagtgaggagatgcaagtagttcaatttgcttgcctagaaggcagggtactggaaaagttgttgcaattcttaattttaaactttatatatcagtaagccatatataaatatgattgggggtgtttattttaaaatctattatggaaattgagagactgacctaa

SEQ ID NO: 3 - DNA, 1308 bp, Homo sapiens
tgtggtatttctttccactagcattttgttggctttcgcttttccagttagcagctctttgaattatctttctaagatacagatttaattatgtcactattcaattcagaggttctgctatggaatgtagtttaaactgcttagcttggcacacagagatttatttctagccccttctccaccttcctatttcctccttcrtttcagaatcttcctctccctcatccaatgctggcaaacaccagtgggggtggagtagtgggtgtaagctctagggagaaggcttggattggaatccaagttattccattacaagtagtgtgacctttaatacattatgtatattgtctaagtttcagctttattgtctgaaaaagaaaaataattgtgtgttcctcataatattgtggtacgaattgattctttcactcaagaaatatttactggagtacctactacatgcctggtgctgttgtagaccttgagataccttactcaagcaaaacagccaaggatccctgcccctggggaatttgaaattaagcaagggacagataaacaatgaacaaaatacataatatgtaagtctattgcatggcattctctaaggtgattggtgtcatggaaaaatagttaaaggagagcaggacagggaaattaggagtcctatgtatggtggagtgggagggctagaggtttaaaagggtaattatatgtggccttattgaggagatgccatttgaggaagcgctttaagaagtaagagaggtagctatttgaattccaggcaaaaggtatatccttgcaaaggctctgaagagattttcctggagtggtagaagaaccagcagaccagtgtgctgggcccagaagacggaagagaaaatcagccacacttgagaggaattcaggggaagcaatgtccttaggggagggccagtttatcttttgagaaggaggaagttgaggatatgatggatttggttagttctgggctgtaaattccagaagacccagtgagacaaagtaagagaggttgtcataaaagggaacgtgcatagggatgtgttgtgagtctgagacttcttatgattaccgacataaacaagataatggatatagtgagattagttctaccagctgtggaacgtgtagtggtggcaagatcatgaatgtcaaggatagagagggttagacatctggggcttccttctcaacaatttcacataaacctccaacagcaacagtaggattatgtgaaatagatcacacaaaggatcatttgagtcattgacaataatcaggggt

SEQ ID NO: 4 - DNA, 620 bp, Homo sapiens
tctgaaagttgatagagagttgatagaacaatcttttcataaggtataaattctatctatttttccctaaaaacaaacaaacagcaaccattcttgcttctaattgggcagtacaatctgataggttggctagagacttgcagtggggtgtccctggtacctattcaaagactgtagctttcttctatctcattctcattttctattctttgcattgtagagttttggagcaaagaaggtcatcaaacttatacagtgagcctaacagtttccttttaagatgaggaaactgagccccagccagccatgtgattcatcacagttccttggtggctgagttgggaggagaacacacatcttctcagctcctcccactgctctttccattaagacagacagcctctcattcaaagtaagagaatttccatcatatgagcaagggacaatgagagaactgcttctcagtactcccygcttcttcctcacctacttcctcttcactggatttgtcaactcacctgtctttacgcaatagttacaatgccagcatttctctacattacatacttcagcgattctcttactggctttgcaaagtcacccaaacacgaatggaaata

SEQ ID NO: 5 - DNA, 909 bp, Homo sapiens
acttgtatagagaatctaagattaattttaaggaggataattttggaaaaactcagggagatggtaatttttaagccgggcttggatggatggctactactctcaggggcacaaatgaggggaaaaagaactcaagaccaaagaaacagcatgagcaaaggtccagggtactttttttttttttttttaaagaaatgactaggccgggtgcggtggctcacgcctgtaatcccagcactttgggaggccaaggcgggcggatcacgaggtcaggagatcgagaccatcctgattaacacagtgaaaccccgtctgtactaaaaatagcacaaaaaaaaaaaaaaaaaaaaaaattagccgggcgtggcgagtgcctgtagtcccagctactcgggaggctgaggcgggagaatggcgtgaatccgggaggcagagcttgcagtgagccgagattgcgccactgcactccagcctgggtgacagagcaagactccgtctcaaaaaaaaaaaaaaaaaaaaagaaatgactagtcatccaatgtgccaaaataataataaacttttattagtgattactatatgccaggaaaaattcctagcactttatgaggattacctgatttaattttcaactgaagcatggaagaagatactattatcaagccagttttacaggtaaggagactgagtcatagaagatttaagaagytaactcacaatcatatagctagatagtagaggagtcaggaatcaagtttgccccataactgcaatactgttatgtacacagtacaggtagaaatgcaaagtgggtttgaaccaaagagtggagggctttttgtgccatcccaaagtgttgtacttcataaataaattacaaaggaggagaaagaatcctatttttttttg

SEQ ID NO: 6 - DNA, 801 bp, Homo sapiens
  misc_feature (401)..(401): N = absent or G
gagagacatggcaacaggtctcctttggttataaactagacactcagcacttgtttctaatccagtggtgcccctggcttactgttcagtcctggataagtctcttagtttcttggtgatgatttgaacattggaaagtaaaatctgtcacttgcaaacacacagcttgtcgaaaattttttctactctgcaggaactgggccttaaaaaaatgaaaaaaaatctgtggtttcttccttctggaagctacaaacctcctgtttcttgatgggcaatcttgagtgagctctattaattattattctctttggctcagttgctaagctattttatgcatgttatgccctttgacaattagtctttagctgtaatcccccagccatcctcagaaatgtggtgangtagccatagtgttcccaagattagaaaaaatgtaatggcagagccaagaggaaggtaaatggtccacattttatgaagcatcatctaaatggccctattggttagagtgaggagatgcaagtagttcaatttgcttgcctagaaggcagggtactggaaaagttgttgcaattcttaattttaaactttatatatcagtaagccatatataaatatgattgggggtgtttattttaaaatctattatggaaattgagagactgacctaatctgggagaaattaaaaattacagttttcactcgttttggatttggtgttttctagggtacctaacctagatcagtggttctcaaacttaggtggatgtcagaatcacctggggagcttagtgaatgcac

SEQ ID NO: 7 - DNA, 2734 bp, Homo sapiens
gaccagactttgcctaggttgaggaccactgggagccaattgattttcacagctctaagaaaagccacagttagaacagggttgatttcaattctacagtgggcatacctcagagatactgtgggttcagttccaaatcaccacaataaagcaaatatcacaataaagtgagtcacacaaattttttggtttcccagtgcatatagaagttatgtttacactatactatagtctattaagtatgcaataatattatgtctaaaaaacaatgtacatatcttaatttaaaaatactttacaggctagcgttggtggctcatgcctataatcctagcactttgagaggcagtcgtgggagaatcacttgaagccaggagttcaagaccagactgggcaacatagcaagacccagtctctaccaaaaaaatttaaacattagctgggcatgatggcatgcgcctctagtcctagatagtcaggagaatgaggcaaggggatctcttgagcccaggagttcgaaattacagtgaactctgatcattccactgtactctagtccaggtgacagagtgagaccatgtctcgaaaacataaaagatattttattgctaaatatcgaaaatgattatctgagcctttggcaagttgtaatagtttttgctgctggagggtcttgcctagatgttgatggctactagctgatcaggatggtggttgtggaaggttggggtggytatggcaatttgttgaaataagacaacaatgtgctttgctgtattgattgactcttcctttcataaaagatttctctgtggcatgcaacactgcttgatagcatattacccacggtagaacttctttcaaaattggagccaatcctctcaaatcctgccactgctctatcaactaagtttatgtaatattctaaatcctttgccatcatttcaacagtgttcacagcatcttcaccaagagtagatttcatctcaagaaatcaccttctctgttcatctctaagaagcaactcctcacatactcaaattttatcaggaggttgcagcaattcacttgcagcttcaggctccacttctgcttcttttgctatttccaccacatgtgcagttactttctccactaaagtcttgaatcccttaaagtcatccacgagggttggaattaacttcttccaaactcctattaatgtttatattttaaactcctctcatgaatcatgaaagttcttaatagcagccagaattgtgaattttttccgggtgattctcagttcacttttcccagatctattcatggaatcactatctatggcagctataggcttttaaaatttatttcttaaataatacaacctgaaagttgaaattactccttgatccatgggctgcagaataaagcctaacacagaaggcatgagctcttgggtgactaggtgcattgtcaatgagaagtgacattttgaaagaaatatttttttctgagctgtaggtctccacagttggcttaaaatattcagtaaaccatgctgtaaacagatgtgctgttatccaggctttgttgttccattaacagagcacaggcagagtagatttaactgatgttaaattcttaaggactttaagattttgggaaggatatataagcatgggtttccacttaaagtcacagccacattagcccccctaacaagagagtcaatctgtcctttaaagctttgaaaccaggacttgacttctcctctctggctatgaaactcctagatggcatattcttccaatataaggctatttcatctgcatttaaaatccattgtttagtgtagccaccttcaacattgaacttagctacatcttttgcataacttgctgcaacctctccatcagtacatgcagcttcaccttgcacatttgtgttatagagacagcttctttccttaaatttcatgaaccgacttctgcttccttcaaacatttcttctgtagcttcttcacctctcttagccttcacagaattgaagagatttaggattttgctctggtttaggctttagcttaagagaatgttgtggctggtttggtcttctatccaggctactgaaactttcttcatagcagcaataagatagttttactttcttgtcactaatgtgttcattgatgtcacacttttaatttccttcaagaacttttcctttgcattcaccacttggctaactgtttggtgcaagaggactggctttcagaccatctcggctttggacatgcctttctcactaagcttaatcatttctagcttttgatttaaagtgagaaaacatgtgactcttcctttcacttgaacacttacgggacattgtagggtgattaattgtcctgctttcaatattgttgtgtcccagagaatagggaggctcaagaagagggagaaaaacagggaacacctggttggtggagcagttagaacacacacaacatttatcgattaagatctctgtcttacaggggcacagatctcggcgccccaaaacaattacaatagtgacatcaaagatcactgatcacagatcaccacaacacatataataataatgaaaaggtttgaaacattatgagaattatcaaaatgtggcacagagatacaaagtgagcatatgctgttggaaaaacagagccaatagaccaggtggatataaggggttaccacaaaccttcaattagta

SEQ ID NO: 8 - DNA, 823 bp, Homo sapiens
tgtgattgaagattacctataaatacatgctgagctttctctatgtacctgattttgtggaaactatttacggttctgctgttttattctgatatagctttccaagtgtttcctcaaattttactacattgtgtattttactcatttagccaacaaagatttatttgttttacttattaagtgtcaggctctgtcctaaaygttaaacaggtgaacataccattcttgatagggggacacagaaataaacaaaggagtaaacataaaggatgtcagaataacaagaacaaacaagcaggagtgggggggtttcagggactggggaagggcggggactggtttgctcttaaaaaaaaggctgatcagagctgggcacagtggctcattcctgtaatcccagcactttgggaggctgaggtgggtggatcacgaggtcaggagattgagaccatcctggctaacactgtgaaaccctgtctctactaaaaatacaaaaaaattagccgggtgtggtggcaggcacctgtagtcccagctacttgggaggctgaggcaggagaatggcgtgaatctgggaggcggagcttgcagtgagctgagatcacaccactgcactacagcctgggtgatagaggagactctgtctccgaaaaaaaaaaaaaaaagggctggtcaggaaaaagctcaccaatgaggtgacatttttgcacagacctgaaggatccttacaatgactaaggagtagagagtaaaaagattattgattttggttttgtaatttatgtggatgtagaaacaggcttggggatgttaaatattttta

SEQ ID NO: 9 - DNA, 1743 bp, Homo sapiens
caagaagaattcagagaaggaatctcatttgactagggatgggagtgagaatatgagaggtggcaaaaatgaacagatgggtagggtcacaggtaatatgcacaagacctctcttctcatgaagcttacattttagtagagtcaaagaaaggaagataataaacaaggcaatcaacaaagaaacaagataatttcaaagcatgaggataatatgaaggaaataacaaaggtgatttggaattactaggagtggatggagatccttcctcagctgggtygggaacgtcatgtcaaaggaagagacccttgagctgacacgtaaatgaaaggaacggactgtgggaaggcctggggaagggtactccagggagaggagctagcatctacaaatgcccaagacagagctgaacttgcacttttcagaagcagaaaggtcagctaagagacaacacaggccaggagacaaggtcagagagaaaggctaggcaattaatgtaggtctttcttggccagataataaggtttattctcagtgcaagggaagccattgaaaggcatcaaacaggaagggatatgctttgatttacacttcttaagttctctctagaagctcaatgaagctggattcaggggcaaggtatgagtggaaacaatgagaccagttagaaggaggactcttccagtgtccaggtgagacatggcagtgacctgggccagggtatactaatggggataggagaagcggaaggatttgagatatattggggcggtagaactgcaagaatgtgctgatgaatttggtttgggatatgagggaaaagaagaaataaaaaatccctgtaattgcaaaaatggccctagcaattgagtaggtgacaatttatcatataataataacaacttatgcgtataaagtttttattatatagcagtcatggctctaacctctttacatatattacctcacatgaaccccacaacaaccctacaagataggtactattctcatccctattgtacagacaagggaagagagggacggacagattaacctcactttgttgttaaattacagcctctatgtgaagctttatcggcttcagagtctgtgtgcttaaccatgatatctttacgttttgtattaccaggttgtggaatactagagaatgaactgattttagaaggagaaacaaattttccggttttgacatattgtttttgagatgtcttacatggaaatatcgagtacataattgaatgtgtgagcatggaattcagggactaggtcaaccctggagacattagcacactgatagtatttaaagccatggggttgaattagctgtatagagagcaatagagtacatggagattacaagaagccacaactagccctgagtcctccaatctgtagtgttctgatagagaagaaactcacttgcaagatcaagaagcagcatctaagtgaggcagaaagaatcccagaggagagtgtggattttcagaactgagtgattaacatgttggcttgattctcagccagtctctgtcctcatggtggcaagatggctgcagcaattccaaccaatactcttccaagcttatagttcatagaaaagagaaagactcattttccagaactcatttataaatcctggaatccactctgattgggccttgttgggtcataggcccattcctgaatcttcaccaatcattgtgactagaggaccctagagtagg

SEQ ID NO: 10 - DNA, 835 bp, Homo sapiens
ggagcaagactgagtttgagtccaggctccatcttttaccagctgtgtaaactgtgtgaatctaggcaagctccttaaagtctctggactctacttcacaggttttttgtgggattcaaatgagttatatgtgcagctcttggaataatacttggcatatagcaagcacaatgtgtgctcatcatttttatttccattttatgggtttttttcccttgtaacctgatttagaagttgtatttgtacatttcttcatgtttaacgtatttgttcaggttaaattgaaatattttacatatagaaactgaggttgggttacctcagaaacagagcttgagacaaggattttttttttttttttttttttttggtggtgattctaggaagcaccagtagaaaagaggcaaagagattcagggaagggaaggaagtcagttcagggtggttcccaaagggagctactgtagtcaactgagactcagcccactatagacctctggrtgatggtgtagcccataccccaaagttatcctgcccaagggacgaagaagttggggtatctatcctgcgactatctttagcactgtctgagcactgctcccagggcattaaacccctagctcttccagtcttcctcatgtgaaaatagaaagaagcccttaggccaagaatagtgaactgttacagtcacaggcagagggtaagaagagagagggaggctgctgagaggatgttggcaaggcaggtagtatctgctatgagaagttattaattattccctcatattttttttcagtttttattacatcctttatttttcggcattagtgtcagt

SEQ ID NO: 11 - DNA, 603 bp, Homo sapiens
atctcaaaagctgctgttcatagtcattcactgttggacattaatgacaaatcactttcatgaactgacatccatttaaagggattttttaaaaatgtgtttatctcataactgctcctgtttatatgaggatcctgtcttctttgagattataatgacaacaaatgttattcgttttctgcactattcatataaacaacrtaactgggcataatactttcatgatatcatgtcattactaataaatcacctttttaaaacatctctatgatagtatcatggttaacaaacagcacagacaaaggagcaagactgagtttgagtccaggctccatcttttaccagctgtgtaaactgtgtgaatctaggcaagctccttaaagtctctggactctacttcacaggttttttgtgggattcaaatgagttatatgtgcagctcttggaataatacttggcatatagcaagcacaatgtgtgctcatcatttttatttccattttatgggtttttttcccttgtaacctgatttagaagttgtatttgtacatttcttcatgtttaacgtatttgttcaggttaaattgaaatattttacatata

SEQ ID NO: 12 - DNA, 1701 bp, Homo sapiens
ctcccacagagcagcattcaccagctggaaggtaagttagccattaaggcatttaattgaaacactgcactaattcatcaaatacttgctgagctacatatttatatcatcagggaaatgcaaattaaaacaacaagatacccacacacccattatgaaatggcaaaaatctggaacactgacaacaccaaatgctggctgagacgtggagcatcaggaactctgactgaaggtacagccactttggaagacagttttgcagtttcttataaaactaaccttactctcactataccagccaccaatcacaacattcctttgtatttacccaaaggagttgaagtcttatgtccacacaaaaatctgcacacagatgtttatagtagttttattcatagttacaaaaacttggaagtaaccatgatatccttcagcagatgaatggtttcataactgtggtgtatccatacagtggaatgttattcagcctaaaaagaagtgagctgtcaagcaataaaaagacatggaggaaccttcaatacatatcactaagtgagagaaaccagtctgaagagactacacactgcatgattcaaccatatgacagtctgaaaaagaaagatcagtgattgccagaggttggcaagaggaatgaaaaggtgaacacagagcattcttaggacatgcaaacactttgtgtgggactcagaatgagagatacacattctgcctttgttcaaacccatagaagtttcaacactgagagtgcaaaccatggactttggatgatgatgatgccattgtaggttcatcagtggtaacgagcgtacctctctcatgggagatgttgattatggggagaggctgtatatgtgtaggggacagagggcatacggaaaatctctgtaccctccttttaattttgctgtgaacctaaaagtgctcaaaaaaaataaagcctattaaaaaatacttgttgatgtgcaagacattcttctaggcactgaagaaacagcaagaactaacaaaaaagggacaaaactcctgtccccatgggccttacattgtagtggagaagattaacataaacaaacatgtaattgtgtaatacaatgtcaggttgtgattatgatttgaaaaaggaaagcaggagaatggaatagtgctattttagataggggggttggggaagacttttctgaggaaagaacatttgagcagagacctgactgaaggtggtgagggagtcatggacacgactgggaaccatgtcccaggcagagaagagccaaatggaaaaagycaagacagacgccccttcagcgagggctgagtcatagcaggggtcatgtgtctggacctgaggagcaggcagtggggttggaaagataaccaggggccagatcatgcccccagaaagcattttgggttttattctagaggaaatggggtactctctactgggttttgaacaagagagtgacatgatctgagatatattttaatgggatcactgtggtcagcaaatggaaatttggctctaatgggacaagggcagaaactgagaggccaatttaggaggcttctgtactcatccaggaaaatccaactgtggggctccaacagttcaaatgaattcccacccaaagagtcagaaaaaatatggcaacacgccccctcacaaatcatgtgtaccatata

SEQ ID NO: 13 - DNA, 804 bp, Homo sapiens
tggagcgtaaactccacgtcagtttatgtggctacacataaagataactccaataaaccaccttcagggagcctgctcgaagtacttggcatgctggctccttacacggtttcacttaacataatgttgttacataagtacatctacctatcttttttttcttttctttttgtttgattcatgcccttttttttcttttctttttgtttgattcatgcccttttttttttttttttttttttcttgagtgaatcctaagtcaggaggcagtaggggttagcaatttaaagcccagacaaaaaaytctggttcaaatcctagctccatcttcactaattgtgtgacaatgggcaagttacttagctttttaggatcttactttcctcaattaaaagtagggaagaaaatagcacctatcccatagagttgctgtgaagaataagtgttgttgggtggctcatgcctgtaatcctagcactttgggaggccaaggcaggtggatcacttgagctcaggagtttgagaccagcctaagcaacaaggcaaaactctgtctcaacaaaaaatgcaaaaattagcctggtgtggtgccttgcacctgtagtcccagctacttggggggctgaggtgggaagatcacttgagcccaggaagtcggggctgcagtgagctgagatggtgaggctgcactccagcctgggtaacagagtgagaccctgtctcaaaaaatcaattaatcaataaagtgttgttgatgtttatgaaacccttagagctctaccaggcatacagtgaactacgatg

SEQ ID NO: 14 - DNA, 2158 bp, Homo sapiens
gttcttggaagttcttttttgtgtgtttttttctattctgtttgtttgcttgttcttcattttctctctctgcatttcagtttgggaagtttctatttacctatcttcaagctcactgattctcttcaagctcactgatacgttgtgtttactggagcctatcgaagacaatcttcatttctgtcacagtattttttatttatagtatttctatttgattcttttcttagaatttccatctctctactgacattacccatctgttcttgcatgttgtctactttctcccttaacatattaattttagttattttaaatttcttacctggtaatcccaaactctatgtcatatccgagtctggttttgatgtttgctgtatcgcttcaggctgtgttttctctcacctttccgtgtgccagaggcttcaagttctctggcattcttgcctttgtctcccatctttaccttgtgcttccgtaactactcctacttagacagagtctgtgccttgcagctctttcacctgtgatccactgttattactggagccctgtggtatgtagtaaagtatggggaaagggaagtgttttataatctttaaatctcagcattttagtgggcctgtgtctcaggactgtgatcttcacaagtgtttcttcttgtatagctttaggtgtaacaggacaactagaagggactcaagttagagaaacatccttcccccacagccctctcacaggagtctggtaaagcctttcccctggagagcagacctttgtttctggacatacttcagaaggttactcgtcccctccccctgccagagccacaggggtatctttgtcagaacttcaccaggagaacttggtgggattcctgtaggtatgctcacgaaaacaaggaggacccatcacagttcggcccccaggtgtttctcactcccatgctagtccacactcagcctccagcaagtcatcaaaattaccatttaagtgttttaacaagttaattactccagtggattcaggtccaagtaagcagatcttggctgtgaatttctggatttgcctactctccagattttattgtggcagtttgtcctgcaaattccgttctatgatggaactaaaaaactcgctggttttatttgtccagcttttccttgttttaaaggctggagtaacaacttccatgctctgtatatgttggagctaaaattggaagtctgtcacgatggttttttttcttttttttctttttttttttttttcctgagatagagtctcactctgtcatccaggctagcgtgcagtggcatgatctcagctcaccacaacctccacctcccgggttcaagcgattctcctgcctcagcctcctgagtagctggaactacaggcatgtgccaccatgtccagctaatttttgtatttttagtagagatgaggttttaccatgttggtcagaatggtctcaatctcttcacctcaggtgatccgcccgcctcggcctcccacagtgctgggattacaggtgtaagccaccacacccagcccatgatggtttttttcattgaggcctcagttggaaaattcaaatgcttggagctacaatcatctaagagcttgctcacacacatctgatgatttgtgctgatgctgagtggaagccttactggaactcttggccagaatatgcacacatggtttccccatgcagcctgaacatctcaacatgatgttgggttctgagggcaaaagtcttgagatggagagaagccaggtagagactgcaccctagacttcaaaggatgtgacttcatttccatttcacttcactggtaagcaaagtcacaagcccccgcccagtatttaggggaggaccataccctcatctttaagttgggggagtgtcagtcacattacaagaagagcatggggatggggtgaatatatakgtgtgattacttttggaaatttcacctgttgcaagttaaatatggggaattctgagtcatcaagaattttagacctcaccagtctgtgactctgaaataatctcagagtgactttttcgtatttatattttgaaaaaatattgcaggctgggcgccttcaaatcccagcactttgggaggccaaggtgggtgaatcactg

SEQ ID NO: 15 - DNA, 772 bp, Homo sapiens
gtaagaagagagagggaggctgctgagaggatgttggcaaggcaggtagtatctgctatgagaagttattaattattccctcatattttttttcagtttttattacatcctttatttttcggcattagtgtcagtataccaacaagttgcatttgccaggacttttgtggtgacaagtgacgaaaattccagtcacactattttgatcaaagaaaggatctcagagacaggtactcaagtgttgacaggatttgtctctctagctgtcactkctgcttctctttgtgagacaatgtcaatcctgcctcccacagagcagcattcaccagctggaaggtaagttagccattaaggcatttaattgaaacactgcactaattcatcaaatacttgctgagctacatatttatatcatcagggaaatgcaaattaaaacaacaagatacccacacacccattatgaaatggcaaaaatctggaacactgacaacaccaaatgctggctgagacgtggagcatcaggaactctgactgaaggtacagccactttggaagacagttttgcagtttcttataaaactaaccttactctcactataccagccaccaatcacaacattcctttgtatttacccaaaggagttgaagtcttatgtccacacaaaaatctgcacacagatgtttatagtagttttattcatagttacaaaaacttggaagtaaccatgatatccttcagcagatgaatggtttcataactgtggtgtatcca

SEQ ID NO: 16 - DNA, 701 bp, Homo sapiens
ctgtttgtcctcccaaacacagcaggcagaagagtcactccacccagggcaaagtgaaggagagggtggagggagattgggaatgctgtgctcatagatctctcttgacaagaatggggagaaaagttccacaccaaaggagggcaaagccagagaaatagggaagaggtctcgggatctgcacagtgagtttgtggagcrtaaactccacgtcagtttatgtggctacacataaagataactccaataaaccaccttcagggagcctgctcgaagtacttggcatgctggctccttacacggtttcacttaacataatgttgttacataagtacatctacctatcttttttttcttttctttttgtttgattcatgcccttttttttcttttctttttgtttgattcatgcccttttttttttttttttttttttcttgagtgaatcctaagtcaggaggcagtaggggttagcaatttaaagcccagacaaaaaattctggttcaaatcctagctccatcttcactaattgtgtgacaatgggcaagttacttagctttttaggatcttactttcctcaattaaaagtagggaagaaaatagcacctatcccatagagttgctgtgaagaataagtgttgttgggtggctcatgcctgtaatcctagcactttgggaggccaaggcaggtggatcactt

SEQ ID NO: 17 - DNA, 1001 bp, Homo sapiens
taagtcaggaggcagtaggggttagcaatttaaagcccagacaaaaaattctggttcaaatcctagctccatcttcactaattgtgtgacaatgggcaagttacttagctttttaggatcttactttcctcaattaaaagtagggaagaaaatagcacctatcccatagagttgctgtgaagaataagtgttgttgggtggctcatgcctgtaatcctagcactttgggaggccaaggcaggtggatcacttgagctcaggagtttgagaccagcctaagcaacaaggcaaaactctgtctcaacaaaaaatgcaaaaattagcctggtgtggtgccttgcacctgtagtcccagctacttggggggctgaggtgggaagatcacttgagcccaggaagtcggggctgcagtgagctgagatggtgaggctgcactccagcctgggtaacagagtgagaccctgtctcaaaaaatcaattaatcaataaagtgttgttgaygtttatgaaacccttagagctctaccaggcatacagtgaactacgatgttgttgatgatgataatcatctttattggcacatgccaggacttgataaccttagtttgtaatgtgaatcctatttaaaagtatttaaaagtatttccactacaacttaagaaactgtcatccagtgcaaagctcagggtagacagcagagagttggatttagccatgattgattggagtttttccaggaaaatacgatgaaggaagacaagaacaaatgacagaccatggaattgaggctcgataatgagagaagtaaagacataaagtggagaggaaccgtgaaaagatgctaggaataatgttttttttcaattccattggaatttaatgacagctagagtgggttatagaaagggcaagctgaaaagtcatagagtaggagtcatgtcattgagataatgtggggaatgggggctgttgctattacaatgcaacttctaggatcctcc

SEQ ID NO: 18 - DNA, 601 bp, Homo sapiens
gagcgatggtcctatttcccagaggaataagagctctgggctccttcaggaaacctggggaagaggatgtccaagtctgcatgaataccaacagatgaggccatcggaagaagggctcctaagaaagagaaaccacacacagaaaggaagaagtgaatatgacccatgctcacacaccaacatgcctatagccaggaggaaatatgagagctaggagggaatttaggagtctctgaattgaaagtattcgtttcagtgaggaggaaactgaagtttagagacgtagagtaaacttattgtragaggaacctatgtaatatgtcttagaaagctctctttcaaaatcattatccaaaaaggaaagaatgggccacttaaaggagtattgatttattaatcgggaaatttgcttatggaaaataggcaaaacttgcttcgaaatgcttatcacaatccacctaaaatttctgttggcagcatcattatctgtagctgcttcagtggtgacactaataaattcacattacagaatagtagtaaaggatttatttttcttttacattttatattatggtcaccaattgtgagctctgaagtaaaa

SEQ ID NO: 19 - DNA, 651 bp, Homo sapiens
agactgcataatgtgcctttccagggggtgtttctctctattgattatttgaactgttaaacttgattacattttactttaattgtaccatttgaaattagattcaggtaagatttcaaacttattaaataaatggcccataggacatttgggggaatgtctcaaaaaagaaaaatgtaaatagaatctacatataaaagtgatcaatttaacaagctttaaggggaaggcaaagtaaaacaatatgatgtaatttgcagccaccagaccggcaaaaatattcaaatattgataatatccagttttagcaagaaagaatgtggggaaaaattagcaatgaaatagttatgaaagtactttggcaatakttggcatatgttctgatcccacaatcctgcttctagaaagcatttctgtaaaaataagagcacagattaggacacacatttacagcctgtgctatatgaacaaagctgaaattaactgggactaccgaataaataaaatacattatatttgcaaaatatataattcatagctaatatgacattttaatttttatataaaaatatatttttatatctgcccatatgcatatgcatgcatgcatacccagacatgtgtatacacacatttacatacctggaagga

SEQ ID NO: 20 - DNA, 728 bp, Homo sapiens
taaaatacattatatttgcaaaatatataattcatagctaatatgacattttaatttttatataaaaatatatttttatatctgcccatatgcatatgcatgcatgcatacccagacatgtgtatacacacatttacatacctggaaggatgttcccgatgtgttaaatggaaagagctagttgaagggtagaataaatgatatgataacgtttttgtttctagagaagggaaagatactctatatgaacatatatttatattgttgttggaaaaatttaaaarttgtgggaaaatccccacaaactgccatcattggctcacttgggaaagtagaggtggaaaggcagtgagctatgattagtttatataccttggtgttatttcagttttacaacaaacatatattactttttgtaatataggaaactataggtttgtaactaggaaaatatatataaatttcaagaggacagatttcagattaatatgaataattttctaataggcaggattatttggatttagcgagggctcttcaaggggtcactagtctttcttaattgtgagcggtcaagcataagttagatgaggacagtgttaggaaggagattctggtataagatgcaaagttggacaatgtagcctcattggtctttttaaattatgacatgccaggcttctacaaagtccacatttcaaggcgtttctgcgtttggccaaatgaga

SEQ ID NO: 21 - DNA, 601 bp, Homo sapiens
agtccaagttatcctcatgtgcttttcttcctcactaggttttaaggtcctagagagtatacactgcctcttagtcttcttcatctatctcaaagtgcctggctgagtgctttacatgaagtatccaataattcttgaccatcagacctggggggtggaaccagcagggccatttagccagggctgcaagcccaaacagatctctattcttcagctgcaagttagtgcccaagccacatagggaataggatgatacctcattacacatgctgatgttagctttaaactatgcctgccctckgttttcctaaaagctgtgttactgccaatctcaaaagctgctgttcatagtcattcactgttggacattaatgacaaatcactttcatgaactgacatccatttaaagggattttttaaaaatgtgtttatctcataactgctcctgtttatatgaggatcctgtcttctttgagattataatgacaacaaatgttattcgttttctgcactattcatataaacaacgtaactgggcataatactttcatgatatcatgtcattactaataaatcacctttttaaaacatctctatgata

SEQ ID NO: 22 - DNA, 772 bp, Homo sapiens
gaaagaagcccttaggccaagaatagtgaactgttacagtcacaggcagagggtaagaagagagagggaggctgctgagaggatgttggcaaggcaggtagtatctgctatgagaagttattaattattccctcatattttttttcagtttttattacatcctttatttttcggcattagtgtcagtataccaacaagttgcatttgccaggacttttgtggtgacaagtgacgaaaattccagtcacactattttgatcaaagaaaggatytcagagacaggtactcaagtgttgacaggatttgtctctctagctgtcacttctgcttctctttgtgagacaatgtcaatcctgcctcccacagagcagcattcaccagctggaaggtaagttagccattaaggcatttaattgaaacactgcactaattcatcaaatacttgctgagctacatatttatatcatcagggaaatgcaaattaaaacaacaagatacccacacacccattatgaaatggcaaaaatctggaacactgacaacaccaaatgctggctgagacgtggagcatcaggaactctgactgaaggtacagccactttggaagacagttttgcagtttcttataaaactaaccttactctcactataccagccaccaatcacaacattcctttgtatttacccaaaggagttgaagtcttatgtccacacaaaaatctgcacacagatgtttatagtagttttattcatagttacaaaaacttggaag

SEQ ID NO: 23 - DNA, 511 bp, Homo sapiens
agtctttaatccattttgatttgatttttgcatacagtgacaactaggagtctagttttattcttgtgcatatggttatccagttttcccagcaccacttattgaagacactgtcttttctccagtgtatgttcctggcaccattatcaaaaattagtttatggtaggtggtggatttctttctgggttatctattctgttccattggcctatgtgtctgcttttatgccagtactgctgttctgatcactaaacytctatagtataatttgaaatcaggcaacatgattcctctagtttgttcttttggattaagaaagttttggctcttgtggttccatatcaattttaggatttttttttctatttttgtgaagaatgtctttggtattttgatagggattgcactgagtctgtggattgctttggatagtatggacattttaataatatcaattcttccaatccatgaacatggaatatctttccattttttggtggcctcttccat

SEQ ID NO: 24 - DNA, 601 bp, Homo sapiens
tttatttatgaagcattttttcttaagaagttaaaaacataaaaccagtgatacaccaaggtatttaatggagggggaagagtgggctcccgaagacaccagggcaacatctctcatccttaaaggctgctgggagttaatggatggaagttaattaatgggaaagtagcgcaagtatttctcatcccaaatcagtaggatgatctgccctcttattttgcaggagtgggaagaagagggagcttggagaagctttgagcaggtcctgaataggcaagtgaggggcttgccttaaccctayaggattctcagtctccacgtctacctcccacaacatgtgcaaatgcttacattcatggtgggtttctccctctcccttggatccccaaagcagcaagagctggtgtggagcactccccagtctaggctgggggacgcaaggagaagccatcctcacagcagtctcttcctgagagatgctaaggcggtggagagactgcataatgtgcctttccagggggtgtttctctctattgattatttgaactgttaaacttgattacattttactttaattgtaccatttgaaattagattcagg

SEQ ID NO: 25 - DNA, 791 bp, Homo sapiens
gtgtaaactgtgtgaatctaggcaagctccttaaagtctctggactctacttcacaggttttttgtgggattcaaatgagttatatgtgcagctcttggaataatacttggcatatagcaagcacaatgtgtgctcatcatttttatttccattttatgggtttttttcccttgtaacctgatttagaagttgtatttgtacatttcttcatgtttaacgtatttgttcaggttaaattgaaatattttacatatagaaactgaggttgggttacctcagaaacagagcttgagacaaggattttttttttttttttttttttttggtggtgattctaggaagcaccagtagaaaagaggcaaagagattcagggaagggaaggaagtcagttcagggtggttcccaaagggagctactgtagtcaactgagactcagcccactatagacctctgggtgatggtgtagcccataccccaaagttatcctgcccaagggacraagaagttggggtatctatcctgcgactatctttagcactgtctgagcactgctcccagggcattaaacccctagctcttccagtcttcctcatgtgaaaatagaaagaagcccttaggccaagaatagtgaactgttacagtcacaggcagagggtaagaagagagagggaggctgctgagaggatgttggcaaggcaggtagtatctgctatgagaagttattaattattccctcatattttttttcagtttttattacatcctttatttttcggcattagtgtcagt

SEQ ID NO: 26 - DNA, 928 bp, Homo sapiens
gtgtaggggacagagggcatacggaaaatctctgtaccctccttttaattttgctgtgaacctaaaagtgctcaaaaaaaataaagcctattaaaaaatacttgttgatgtgcaagacattcttctaggcactgaagaaacagcaagaactaacaaaaaagggacaaaactcctgtccccatgggccttacattgtagtggagaagattaacataaacaaacatgtaattgtgtaatacaatgtcaggttgtgattatgatttgaaaaaggaaagcaggagaatggaatagtgctattttagataggggggttggggaagacttttctgaggaaagaacatttgagcagagacctgactgaaggtggtgagggagtcatggacacgactgggaaccatgtcccaggcagagaagagccaaatggaaaaagtcaagacagacgccccttcagcgagggctgagtcatagcaggggtcatgtgtctggacctgaggagcaggmagtggggttggaaagataaccaggggccagatcatgcccccagaaagcattttgggttttattctagaggaaatggggtactctctactgggttttgaacaagagagtgacatgatctgagatatattttaatgggatcactgtggtcagcaaatggaaatttggctctaatgggacaagggcagaaactgagaggccaatttaggaggcttctgtactcatccaggaaaatccaactgtggggctccaacagttcaaatgaattcccacccaaagagtcagaaaaaatatggcaacacgccccctcacaaatcatgtgtaccatataagccagcttctatagaggaaggaaaggtactggatggacaaataacagggcccatcacatagttgtaatttacaaattacctcacaaaaagtggttatt

SEQ ID NO: 27 - DNA, 831 bp, Homo sapiens
tctagggaagaatgcttccttaccagttctggcttctgcctattcttggcactccttggcttgtggcagcacaactccactctctgcttccatcttcacatgcccaacttccttccatttatgtgtatctgtgccaaatttccctcttcttataaggacatctgtcattggattagggtttaccctaatgaatttggggaggaccctattcaatccactacaaccaccctttatgtacacgtagctggtttctctgtcaattatattttagagtgaggacgttgcttctcctctaacaagatattataataacaattattgtcaaattatttaatgaatgcttactatatgacagttacatgcattaactcatttaaccctctgacaattctatgaaataggtgctatttttatttctattttgcagatgagcagccagagagagtttacatagggcaaatatcaccattacctagcaagaacaaaataagaggaataagmagtccccttgtattttggttacttaaaagggatggatctcaagacaaaggaaaatggttgggtgcacgaggggccagatgctggaaccagttctgaagaagtgttcctggggccaagaggatctgagaggtggccaggtgtgaagactgaacaagctgagcgttaagaacagcaaagttggccaggcatggtggtgcatacctgtagtctcagctccctgggaggctgaggtgggaggaatatgaaggcccaggagttcaaatccagcctgggcaacacaatgagaccctgtcttaaaaaaaaaaaaaaatcagcaagctgggaaataaa

SEQ ID NO: 28 - DNA, 1202 bp, Homo sapiens
taattcattgtgacccctcagaccatcctccggataaacagcattgagattgctctgtgtttgttgtagtcaccgagttagtatttgcagaaatataaaaataaactcttgctttccaaggaaaaaaagaatcttgggtatggccacccccaataatgtgtaatgggctagtgtaaaattatactaatgagcaactagtgagcacatgctgtacttaacagctcttgttcaggattcagttagacttagatctctttagctgcaaaactttgggaatgttatttatagtttccaagcctcattgataagattgttgtgaagattaaatagaatgcatataaaatgcagctcagttggtgaaggcactttcacctttgatccttcatcaccatctgcccaaaagaagccctgtcatggagcagccagattctcattttaggtaaacagaaaaggataaggcacttctggccttgtattttctcccagagcactcagatgctgattatattacagacaaatcaagatttctcaaccctcttcaattctttcaatcaattatccatttagtgtaactatgtgataatgtctaacacataaattatcatgaaaaatgtgaaagctactaaactaaaaaaaaaaaaattctttttagtagcaaggattttgtatggggaagcctggctttgtgggaaatgatttgataaacttacactggaaactgaaccttagggaatggattccattccagtcaaatcttcaaaggaaaagaggaagctactctggataataagagtgaagaattggaagttcctgggaggaaatcctggaaaggaaaagaaattggtactgtgtagaggaaagagaaaactctcccctctccatgatggtgcagctgaggcagaactttggaaaaagaaaatctctggaatgctgacaatcgtgtttccctaaaaaaccctccgacaccttcagaaactattctgaattgctgagtattaatgcttttgtgtgagtatgttattttgaggagttaagctctatgtcttgataagaatgtatcaaaaatagacctcgcacatcaayccaggagtcagaggtcacaaaggagactgacaaatgggtcatggtgagaactatgaccacccgtgtccatatagcttaactagcagaactgaagctgaatgccaccttggtcaagatga

SEQ ID NO: 29 - DNA, 924 bp, Homo sapiens
aaaatgtcctgttacatgacaaatttaaaacaatacattttagaatttaccttgacaacactctcagagaagattattttagaaactattgataaattaaaaatctaagtgaattatatgcctaaaagctttcttttaagtgatacttgaggggaaaaaaacgtcatcccaacatttttagacattgaactttacaagtgtagaaatggtcacagaaagcctatgttattctgaaatatattttgtttcagctatgtttgtgaaaattgaccagctacttgacaaatctagattttcttaaaggcactcaactaaatgctattgtctcctaggacttgtcttggccatyttgattatcataactctccaatataggctttaggatttccaaattcatactctgaagcccaaaattattcccactataatttagagttagcctttgaaataacttatagaaagcattaattgattccatatctaggggccttctgagttgtttataacttatatatatctacatatatatttattgataaaattttatttttaatataatttacaaccagatttctcttacaaaaaagattcaatctattttaaaagtatgataatcaattatattatataatttgtgccacaattcatacttatctattgatttagaaaccacattcaagataatcctctctaccaagaattggcccccagcgtagcagcaaagcaccattaactatcatttccaccgacagctgaagttgtggttttgcattcagcacttttttccttgtgtggagtatagaacaaaagatgttactaatgtaataatgtgagtcatcatccaaatctgtggttactactaccatgaaaagttttctttctcagtaggaaagtgtcattggtcattcccaagatgttacagat

SEQ ID NO: 30 - DNA, 1308 bp, Homo sapiens; identical to SEQ ID NO: 3

SEQ ID NO: 31 - DNA, 550 bp, Homo sapiens
ctattttgatcaaagaaaggatctcagagacaggtactcaagtgttgacaggatttgtctctctagctgtcacttctgcttctctttgtgagacaatgtcaatcctgcctcccacagagcagcattcaccagctggaaggtaagttagccattaaggcatttaattgaaacactgcactaattcatcaaatacttgctgagctacatatttatatcatcagggaaatgcaaattaaaacaacaagatacccacacacccattatgaaatggcaaaaatctggaacactgacaacwccaaatgctggctgagacgtggagcatcaggaactctgactgaaggtacagccactttggaagacagttttgcagtttcttataaaactaaccttactctcactataccagccaccaatcacaacattcctttgtatttacccaaaggagttgaagtcttatgtccacacaaaaatctgcacacagatgtttatagtagttttattcatagttacaaaaacttggaagtaaccatgatatccttcagcagatgaa

SEQ ID NO: 32 - DNA, 514 bp, Homo sapiens
ctattttgatcaaagaaaggatctcagagacaggtactcaagtgttgacaggatttgtctctctagctgtcacttctgcttctctttgtgagacaatgtcaatcctgcctcccacagagcagcattcaccagctggaaggtaagttagccattaaggcatttaattgaaacactgcactaattcatcaaatacttgctgagctacatatttatatcatcagggaaatgcaaattaaaacaacaagatacccacacaccmattatgaaatggcaaaaatctggaacactgacaacaccaaatgctggctgagacgtggagcatcaggaactctgactgaaggtacagccactttggaagacagttttgcagtttcttataaaactaaccttactctcactataccagccaccaatcacaacattcctttgtatttacccaaaggagttgaagtcttatgtccacacaaaaatctgcacacagatgtttatagtagttttattcatagttacaaaa

SEQ ID NO: 33 - DNA, 3738 bp, Homo sapiens
cttcctcagaggaacatgaaagaatgcacaagtgtaagtctcctagcgttctagcatcccaaaaagagtcccatacaattagtaaacaacagcaatgcaaggactcaaaaataataagtctttggtatttgatctaaattttttcactggtttttcatttttatagctttaatgccatgagttttgtctaggattttttttttttttgcatatgtgcatccaattgttccagcaatatttgttgaacaatctatgctctctccattgaattacctttactctgtcaaaactcagtggactatatttgtatgagtctatttctgggctctctgttcagttctattgatttatatggctattctttcaccagtaccattttgtactaattactgtgccttatagtaggttttcaagttaaatagtatgagtcctccaaatttgttcttcttcagtatagggttagctattctatgttttttccctttccacataaatttcaaaatttgttggtatctacaaaatacttgctgggattttgttgaatctatagatgaagctaataagaaataacatcttaatgatatggagtcttccaatccatgaacatggaatgtttctccatttacctagatcttctttgatgtttttcatcagtgcattgtaatttactacatagaggtcatgtacatattttgttagatttatacctattccatgttttgggtgctattgtaaatgatgtttttaacttcaaattttaattgttcagtgctggtatattggaaagcaattaacttttgtgtattcgccttgtatcctgtcaccttgcaacactcatttattagttccaagaactttttgtcagttccttgagatttcctgcacagacaattatgtcactatgaacagtttaatttcttcttttccaatctgtataccttctgttttcttctacaaatatgttaggttaaatggaaaagaattaaggttgaagatgaaattaaggttggtaatcacctggcctccagatgaggagattatcctggattatctgggtaarccgatatgaaagcaaaggttcttataaatgggtaatataggcagagagagagaaccagagagatggcagcatgaaaaggactcagctgacaaggaggaagcagactgcgagccaagtagtgcaggcagcctctagaaattaaaaaagataaggaaacagattctcttctcagagcctccagaaggaacacagagcttccctacaccttaattttagtcactgagactgattttggacttatgacatccggaactggaaaataacagatttgtgttgtttcaagccaccaagtttgtggtaatttgttacaacagcaatgggaaactaacatacatatcttctgaaaataagcctgttgtaattttttgttcttccacaggtaaagtggtgttttttccctttggctctttcaagtttttctctttgtttttctgccatttgaatatgatattctgtcttagaccattttgtgctgctattacagaacacctgagactgagtaatctataatgagcaggcattaatttgtctcacagttctggaggctgggaaacctaaggccaaggggctgcacctggtgaagaccttcttgctgcatcacaacctggcaaaaggcatcacatagatgagagagagcaatagagcttgagagagaaaggttcagaggaggaggagaaggaggctgaatttattctaaaagtaaacccactcttaggataactaacccattctcaataatgacattaatccattcatgagggcacagccgtcatgacctaatcacctcttaaaggtccctgtctcaacactattgtgttggagattaagtttacaatacctgaacttcttacaaaccacagcacattcttaggggtagttttatggcaatttgttctgcccagtattctatgaggatctattgtttcgctacgtattttgaaattgccaaaaaaaaaaaaaaaggaaaaaagaaaaaagatattgtcccctccccagttcttggaagttcttttttgtgtgtttttttctattctgtttgtttgcttgttcttcattttctctctctgcatttcagtttgggaagtttctatttacctatcttcaagctcactgattctcttcaagctcactgatacgttgtgtttactggagcctatcgaagacaatcttcatttctgtcacagtattttttatttatagtatttctatttgattcttttcttagaatttccatctctctactgacattacccatctgttcttgcatgttgtctactttctcccttaacatattaattttagttattttaaatttcttacctggtaatcccaaactctatgtcatatccgagtctggttttgatgtttgctgtatcgcttcaggctgtgttttctctcacctttccgtgtgccagaggcttcaagttctctggcattcttgcctttgtctcccatctttaccttgtgcttccgtaactactcctacttagacagagtctgtgccttgcagctctttcacctgtgatccactgttattactggagccctgtggtatgtagtaaagtatggggaaagggaagtgttttataatctttaaatctcagcattttagtgggcctgtgtctcaggactgtgatcttcacaagtgtttcttcttgtatagctttaggtgtaacaggacaactagaagggactcaagttagagaaacatccttcccccacagccctctcacaggagtctggtaaagcctttcccctggagagcagacctttgtttctggacatacttcagaaggttactcgtcccctccccctgccagagccacaggggtatctttgtcagaacttcaccaggagaacttggtgggattcctgtaggtatgctcacgaaaacaaggaggacccatcacagttcggcccccaggtgtttctcactcccatgctagtccacactcagcctccagcaagtcatcaaaattaccatttaagtgttttaacaagttaattactccagtggattcaggtccaagtaagcagatcttggctgtgaatttctggatttgcctactctccagattttattgtggcagtttgtcctgcaaattccgttctatgatggaactaaaaaactcgctggttttatttgtccagcttttccttgttttaaaggctggagtaacaacttccatgctctgtatatgttggagctaaaattggaagtctgtcacgatggttttttttcttttttttctttttttttttttttcctgagatagagtctcactctgtcatccaggctagcgtgcagtggcatgatctcagctcaccacaacctccacctcccgggttcaagcgattctcctgcctcagcctcctgagtagctggaactacaggcatgtgccaccatgtccagctaatttttgtatttttagtagagatgaggttttaccatgttggtcagaatggtctcaatctcttcacctcaggtgatccgcccgcctcggcctcccacagtgctgggattacaggtgtaagccaccacacccagcccatgatggtttttttcattgaggcctcagttggaaaattcaaatgcttggagctacaatcatctaagagcttgctcacacacatctgatgatttgtgctgatgctgagtggaagccttactggaact

SEQ ID NO: 34 - DNA, 511 bp, Homo sapiens
aggagaaggaggctgaatttattctaaaagtaaacccactcttaggataactaacccattctcaataatgacattaatccattcatgagggcacagccgtcatgacctaatcacctcttaaaggtccctgtctcaacactattgtgttggagattaagtttacaatacctgaacttcttacaaaccacagcacattcttaggggtagttttatggcaatttgttctgcccagtattctatgaggatctattgtttygctacgtattttgaaattgccaaaaaaaaaaaaaaaggaaaaaagaaaaaagatattgtcccctccccagttcttggaagttcttttttgtgtgtttttttctattctgtttgtttgcttgttcttcattttctctctctgcatttcagtttgggaagtttctatttacctatcttcaagctcactgattctcttcaagctcactgatacgttgtgtttactggagcctatcgaagacaatcttcatttctgtca

SEQ ID NO: 35 - DNA, 617 bp, Homo sapiens
agtatatgtgtttagcattttttcaaaaagtgttttgtgacacacaatattggctctttttccctgcacctgaaggcctaaattatagaacattagtttgctgggtctctattagttcaccaatggatgctgatgtctcaatttttcaaaagctttccagtgacttatgcaaagccctcaggaaaactgagtagcaaataggattagcatatttgtaaagacccagaagtaatgcattaacatgctgaggtgtcataagccccartgaatatgttgataattagtgcttcttagagagcagctagatcaccttcctccatgctaatgatgtgcaaataatccttggtgaatctgaacatctgctagtgggtgtccccaagcaggatgcaatgacaggagacagatttatcaacattgctgttggattccaccaaaaacatactccagcccataaaaccttctatcaggcataatcatattcctagccataattttgctattgtttgcaatcctattttttttctatctatactaattaaagtcttggtgcacccaaagtagtttgtataaattacatgaactcataaaaatttcagtgttcatttgacatgaatcgt

SEQ ID NO: 36 - DNA, 617 bp, Homo sapiens
agtatatgtgtttagcattttttcaaaaagtgttttgtgacacacaatattggctctttttccctgcacctgaaggcctaaattatagaacattagtttgctgggtctctattagttcaccaatggatgctgatgtctcaatttttcaaaagctttccagtgacttatgcaaagccctcaggaaaactgagtagcaaataggattagcatatttgtaaagacccagaagtaatgcattaacatgctgaggtgtcataagccccaatgaatatgttgataattagtgcttcttagagagcagctagatcaccttcctccatgctaatgatgtgcaaataatccttggtgaatctgaacatctgctagtgggtgtccccaagcaggatgcaatgacaggagacagatttatcaacattgctgttggattccaccaaaaacatactccagcccataaaaccttctatcaggcataatcatattcctagccataattttgctattgtttgcaatcctattttttttctatctayactaattaaagtcttggtgcacccaaagtagtttgtataaattacatgaactcataaaaatttcagtgttcatttgacatgaatcgt

SEQ ID NO: 37 - DNA, 692 bp, Homo sapiens
gcttgctcaaaaggactagatgccaccatggggaccccgctcaccagtggtggcctcgtcttttatagatggattcctcaaaatcacacttgccgcccttgtctccaagatttggatcacggtctagatgctcccaccctttctcctcaaaactgccacaccacagtctcaccaaccgccagggtgctctgccctttcgtgaccaaatccctgaagctaggtgagttttgcaagccctatgcacggcagtcccactccctgatgatcctaagatgtaagacctcacagaatttacaytggagcctaagtaacagtgcaccatgccaaagaagacagagttaaactaaacacaatccagggattctgacttactgacttcttttgcaacttgcttcctctctcagttcttctagaaatggaaaatttttcagatcaagaaaaactgaacagaacttgccagaatgacgaagctaatgacagtgattcattgatttattcaacaaaaatcacaataataataataggcatttacagagcattgtttctcttccaagcattttgtatgcattttatttaatcttcacaacaatcttatcaatgaggcttggagactataagtaacttccctaaagtttcacagctaaagagatctaagtctaactgaatcccaaacaagtacagcacgtgcttgc

SEQ ID NO: 38 - DNA, 692 bp, Homo sapiens
gcttgctcaaaaggactagatgccaccatggggaccccgctcaccagtggtggcctcgtcttttatagatggattcctcaaaatcacacttgccgcccttgtctccaagatttggatcacggtctagatgctcccaccctttctcctcaaaactgccacaccacagtctcaccaaccgccagggtgctctgccctttcgtgaccaaatccctgaagctaggtgagttttgcaagccctatgcacggcagtcccactccctgatgatcctaagatgtaagacctcacagaatttacattggagcctaagtaacagtgcaccatgccaaagaagacagagttaaactaaacacaatccagggattctgacttactgacttcttttgcaacttgcttcctctctcagttcttctagaaatggaaaatttttcagatcaagaaaaactgaacagaacttgccagaatgacgaagctaatgacagtgattcattgatttattcaacaaaaatcacaataataataataggcatttacagagcattgtytctcttccaagcattttgtatgcattttatttaatcttcacaacaatcttatcaatgaggcttggagactataagtaacttccctaaagtttcacagctaaagagatctaagtctaactgaatcccaaacaagtacagcacgtgcttgc

SEQ ID NO: 39 - DNA, 637 bp, Homo sapiens
atatctcttcatgtctcacagtctggccaaactgagatcaacctcagagagagggaatgttttatccagccctagattaaaatttatctcctggggcttcattacacatggtatgattaatcactgctcaagatgatcgatggtgggaatttccaatcccttccccagaagtatggtctagaactgtggtccaggcaagacagcttcacagtagcccttcatstgtttatacatcaaagtctgcatcaatgaatcttaattcaaacaagagtggagacaccagtagcagatcatattagctgtagttgtgccaaaattaaccaaatttacttccaatcttgcttcatcaaagattcaaaagttttagcatcagatctcacccactgtcacttagttacccaataatcaaaataatgacccctgattattgtcaatgactcaaatgatcctttgtgtgatctatttcacataatcctactgttgctgttggaggtttatgtgaaattgttgagaaggaagccccagatgtctaaccctctctatccttgacattcatgatcttgccaccactacacgttccacagctggtagaactaatctcactatatccattatcttgtttatgtcggtaatcata

SEQ ID NO: 40 - DNA, 1269 bp, Homo sapiens
tattagctgtagttgtgccaaaattaaccaaatttacttccaatcttgcttcatcaaagattcaaaagttttagcatcagatctcacccactgtcacttagttacccaataatcaaaataatgacccctgattattgtcaatgactcaaatgatcctttgtgtgatctatttcacataatcctactgttgctgttggaggkttatgtgaaattgttgagaaggaagccccagatgtctaaccctctctatccttgacattcatgatcttgccaccactacacgttccacagctggtagaactaatctcactatatccattatcttgtttatgtcggtaatcataagaagtctcagactcacaacacatccctatgcacgttcccttttatgacaacctctcttactttgtctcactgggtcttctggaatttacagcccagaactaaccaaatccatcatatcctcaacttcctccttctcaaaagataaactggccctcccctaaggacattgcttcccctgaattcctctcaagtgtggctgattttctcttccgtcttctgggcccagcacactggtctgctggttcttctaccactccaggaaaatctcttcagagcctttgcaaggatataccttttgcctggaattcaaatagctacctctcttacttcttaaagcgcttcctcaaatggcatctcctcaataaggccacatataattacccttttaaacctctagccctcccactccaccatacataggactcctaatttccctgtcctgctctcctttaactatttttccatgacaccaatcaccttagagaatgccatgcaatagacttacatattatgtattttgttcattgtttatctgtcccttgcttaatttcaaattccccaggggcagggatccttggctgttttgcttgagtaaggtatctcaaggtctacaacagcaccaggcatgtagtaggtactccagtaaatatttcttgagtgaaagaatcaattcgtaccacaatattatgaggaacacacaattatttttctttttcagacaataaagctgaaacttagacaatatacataatgtattaaaggtcacactacttgtaatggaataacttggattccaatccaagccttctccctagagcttacacccactactccacccccactggtgtttgccagcattggatgagggagaggaagattctgaaatgaaggaggaaataggaaggtggagaaggggctagaaat

SEQ ID NO: 41 - DNA, 628 bp, Homo sapiens
ttactcttccaaaccaaaactctgggagtgacaggtagggagagaggagggagtgggatataaacttagaatctccctttcacagacagcctttgcagaaagtccaacttaytcccaggaatggccaagtctttctcagagctgggatgcaatctccctcacccagtcgcacacccctgggccctgcctacaacagtccagggaagcacctttagccctcctttacttctttttgaatcttctaccagcctgctttcctgtctccccttccactcccatctaatcaatgtagaaatggcctctcatttcacttctgagaagccatttcctgtcatctctttaaagtctaccgctttcccactgactgtctctaataagcagaaagcaaatgtctagccctccttgtcagcataattaggaaactgcttcctctggacgtgcctgaagtccctatgttgctaagagcaagactcttcatgttttgccatttgggacgtaactgttttggtgagcagtgtgcaaatcagtttttaacaccaacattctggtctagtctttgagacaggaaaaagatgaaaatacatatgtttccacattttagggtagaaaacccagtctgtggtttccc

SEQ ID NO: 42 - DNA, 617 bp, Homo sapiens
agtatatgtgtttagcattttttcaaaaagtgttttgtgacacacaatattggctctttttccctgcacctgaaggcctaaattrtagaacattagtttgctgggtctctattagttcaccaatggatgctgatgtctcaatttttcaaaagctttccagtgacttatgcaaagccctcaggaaaactgagtagcaaataggattagcatatttgtaaagacccagaagtaatgcattaacatgctgaggtgtcataagccccaatgaatatgttgataattagtgcttcttagagagcagctagatcaccttcctccatgctaatgatgtgcaaataatccttggtgaatctgaacatctgctagtgggtgtccccaagcaggatgcaatgacaggagacagatttatcaacattgctgttggattccaccaaaaacatactccagcccataaaaccttctatcaggcataatcatattcctagccataattttgctattgtttgcaatcctattttttttctatctatactaattaaagtcttggtgcacccaaagtagtttgtataaattacatgaactcataaaaatttcagtgttcatttgacatgaatcgt

SEQ ID NO: 43 - DNA, 919 bp, Homo sapiens
aagctcaccaatgaggtgacatttttgcacagacctgaaggatccttacaatgactaaggagtagagagtaaaaagattattgattttggttttgtaatttatgtggatgtagaaacaggcttggggatgttaaatatttttagtagcatcacataattatcatgaaagaagttaaagccatgatctagaagattttacaattctctgattcacctgttgtgccttattttctctcaggtaagcttcttagttatctggttacttttaacaaatggcagaaacaacttcttaactatggaagattatgttcttttgatttaccaaattatttatccatatatgcagagaatatattttctgaatgaaaaattgggcagcaaactctgaaaagttctaacatgctcagagggacaatgggacyacataattgaagttggcaccaaaccatgaatatctggtcatcataataatatagatgccttggacataacagcaagcactaaccacaaagtaatggtgtactttgcccataagaaagaaacaaatgtgtgactgaaatcagcttttctcactctattgcatggaatatatagtatttcctcaacatattagttttcctgttttaaacttacaaaagtgttttcttatttaacaagtttaagaaaatggtgcaaactatattttctgtatggggaattataaagcccatcagaatgttacaggttggagaagttccacattaaaacaactctttaactttgtttaatatgagtttctagaagattgtttcttccaagaatacattggccttgtaggcacttagtcagatcaaatgccttgttacctagaaaacagtttggaaaacaccagttcacacaaatggttatcttgagatgaagcagagctagaaaagtgtat

SEQ ID NO: 44 - DNA, 944 bp, Homo sapiens
ttggttttgtaatttatgtggatgtagaaacaggcttggggatgttaaatatttttagtagcatcacataattatcatgaaagaagttaaagccatgatctagaagattttacaattctctgattcacctgttgtgccttattttctctcaggtaagcttcttagttatctggttacttttaacaaatggcagaaacaacttcttaactatggaagattatgttcttttgatttaccaaattatttatccatatatgcagagaatatattttctgaatgaaaaattgggcagcaaactctgaaaagttctaacatgctcagagggacaatgggaccacataattgaagttggcaccaaaccatgaatatctggtcatcataataatatagatgccttggacataacagcaagcactaaccacaaagtaatggtgtactttgcccataagaaagaaacaaatgtgtgactgaaatcagcttttctcactctattgcayggaatatatagtatttcctcaacatattagttttcctgttttaaacttacaaaagtgttttcttatttaacaagtttaagaaaatggtgcaaactatattttctgtatggggaattataaagcccatcagaatgttacaggttggagaagttccacattaaaacaactctttaactttgtttaatatgagtttctagaagattgtttcttccaagaatacattggccttgtaggcacttagtcagatcaaatgccttgttacctagaaaacagtttggaaaacaccagttcacacaaatggttatcttgagatgaagcagagctagaaaagtgtattattaatgaagaagaagaaaaacaacaactactggatttctcttcaaagaataagaaaaacatttaaggaagcaaaatgctgatatgataaatatgtttggaggagattag

SEQ ID NO: 45 - DNA, 999 bp, Homo sapiens
aaaatgtaaaatggcttaaacctaatagaagtttactttttgctcatgtaaagtcaaaaatagatgtaacagaacaggagctatctcttctctaagcaggaccaggatcctttcatcctgtggctccaccatcttcaccatcttcaatacttggactgtgaggtcactgtgcatgttcgtatcaagttggtcaacagagaagaaacatggaagatggcccatggaggatggcacacatcacttccactcacattccatgggctagaactcacaaataaatttgatgaacagcaagccagcctctgttccaaaagtcttcctagacagaatgtacataagctgatttagtatctgcacagtctctgcagtgatgcctctctttgttgctgcttattaaagtgttaacaggatcaaggattgacccagaaatggaatattaaaaagaaagttatgctataaattccactgagggttttgtcatttcaagagtgcttctgaaygtccctgttgaggtcatttttttctctgttttgccaaaaaaaatctgccctcattttaatgacaatctagtttttttgttttgttttgttcttttttttttcttttttgaatctcattaccttcaatatgtttggtcaggttggattggtaaatctggcacatggggttgcctgtacctcatcatgaaatccaaaggatacctagagggtccttctaccagtttttttttactcagcactgtaggattaatgccagcaggcagtcaactcatccgtgttcattagactcactttctagggtttgattctggagcagagtggtacaaagataagaacaaaagcattggaatttaccaatttgttcctgcatggtgctctgcagaagggctgagtagtttctgcggcagagaccttctgggattgcctggtagattgtctgtattgaacatggttcctcagctatgtcttccatccatgagctcctccatatgccttcatt

SEQ ID NO: 46 - DNA, 1194 bp, Homo sapiens
gtgcggcagcctgacatgggtcctctgaaccttagcctagcaaggaggtgcccatgtggaagaatggcctggagtagggtgtcagagtccaggcaaggtgaggaggacatctgtgtcctgggatggcccagcatgagtgttagagtgaagtaagaatggcatctgcataggggaagacatattagtgcagatgggaaattaattaagtaattatattaaagataatgggagccagttttcctcactattgaaggaaggtacagtacagaaaaggagaaaattagaataaaccctatgatattgaaatagaattagatgtatcagtattaacttatgctcttcaatatatagaggtagatatagaaataaataatagatagaaatattagttcaccctaactctgtccattgagggggcctgggagtagtaacatctcgataacaatgagaacactgatcacccatatcttgacttctaaataccattctttgctagaatgaaccagaactccttggagaattggctgatcccagaacaggggtagtgaaagtacatgaagtgctagaaaaaaaagaagtattcagaggatgatggaaacatgttaaaaggaacagaaaccagcttgaagggactcccactagtgaaatatgagaaaatttgagcatcaaaataaatagtgatagtaatgtattatgacctattgaatataataggaaaccatgagtatatattgatataaatgaatacaccaaaagtttgatgaggaatggtatagccacatcattgcaaaatatctccctacaaaatatttattaattacaaattggaaaggagtaattttatggtagagaagcttagcagataccatcttaatcaaggaataaaagtgaacatccttagtaatgagataaatgaaaagggtatcctacctgataggwtgcaagaaaacgaacatagcacaccttttgtgatatctctgtgaaagatgcataacctattctagtcatgagaaaacatacaaatgcaaactaagaagcattctacaaaatatcttgtctgtagtcttcaaagtatcaaagttgtataagttaaggaaagactaaggactgaagaacagttttgttctgaaatgaattatagagacatgatggctaaatgcaatgcaagtttctaaactgaatccttgtgcagta

SEQ ID NO: 47 - DNA, 601 bp, Homo sapiens
gattggaagtaaatttggttaattttggcacaactacagctaatatgatctgctactggtgtctccactcttgtttgaattaagattcattgatgcagactttgatgtataaacacatgaagggctactgtgaagctgtcttgcctggaccacagttctagaccatacttctggggaagggattggaaattcccaccatcgatcatcttgagcagtgattaatcataccatgtgtaatgaagccccaggagataaattttaatctagggctggataaaacattccctctctctgaggttgrtctcagtttggccagactgtgagacatgaagagatataaactgtattaggtgctgtgattatagcagggaatgagacagggagaagatcctttaagagaacttgagttgagactggcctatgcagtggttgtcaattattctctatgttgtatgtttcttctcttatgaacacacctagtttcagaagtgtgatggagcttgtaggagggatggaccatgctttagactaagacaccttgggggctgattcctctcccaatgccagcaggggcaggtatctcccaaatcttataagcagc

SEQ ID NO: 48 - DNA, 832 bp, Homo sapiens
ataggtaaaatctttctagaatgaggaggagcacctgagggatcagtacatgatgaccatggggattagtgcataatgtagtctgatgataggatatttaaagcaggaagacactaaagagtttcaagaagaagagagggagaatggggtgtgccttgatgaaacacaagaatggtacttaaacgacctccacctacatgcccagggtgcaaaagaaaagggaaagaaaacagatgcatctagagaaatctgcaaaggaaccaggtctccaagggacagtctggtcagttacagtaagaaagcaaagttcagagaaaatgttaaagatataagggatcttgctggtgactgacagtgagttcaggggacacactgaaagggtttcagaagctggagataggtggaagatgaagtgagggaaaaggaagtgcagtgccatcacggaaatgaaagccttgggacggaggggtcacctggatgtcctgggcttcttgggccctycgtcctaaacaagcataaagagcatcacgggattatccttggtagtctcaaagctgagagtcatggggaggctgtgaacattgaagatcctaccagggacacaaaattacgggtcccttcttcaatcctgcctgtggttagcaggaggttgagggagcgatggtcctatttcccagaggaataagagctctgggctccttcaggaaacctggggaagaggatgcccaagtctgcatgaataccaacagatgaggccatcggaagaagggctcctaagaaagagaaaccacacacagaaaggaagaagtgaatatgacccatgctcacacac

SEQ ID NO: 49 - DNA, 1602 bp, Homo sapiens
tcatctttattggcacatgccaggacttgataaccttagtttgtaatgtgaatcctatttaaaagtatttaaaagtatttccactacaacttaagaaactgtcatccagtgcaaagctcagggtagacagcagagagttggatttagccatgattgattggagtttttccaggaaaatacgatgaaggaagacaagaacaaatgacagaccatggaattgaggctcgataatgagagaagtaaagacataaagtggagaggaaccgtgaaaagatgctaggaataatgttttttttcaattccattggaatttaatgacagctagagtgggttatagaaagggcaagctgaaaagtcatagagtaggagtcatgtcattgagataatgtggggaatgggggctgttgctattacaatgcaacttctaggatcctcccaatgggaagaattggctaaagtaagataaagggcaagatctgagtggaagggagatcaggaatggagagaccagcgtgtttgaagtaccacatgtacacatattgaagtgtatgatatgaggtggtgttggagagtgtgacagtgagcaataggtaaaatctttctagaatgaggaggagcacctgagggatcagtacatgatgaccatggggattagtgcataatgtagtctgatgataggatatttaaagcaggaagacactaaagagtttcaagaagaagagagggagaatggggtgtgccttgatgaaacacaagaatggtacttaaacgacctccacctacatgcccagggtgcaaaagaaaagggaaagaaaacagatgcatctagagaaatctgcaaaggaaccaggtctccaagggacagtctggtcagttacagtaagaaagcaaagttcagagaaaatgttaaagatataagggatcttgctggtgactgacagtgagttcaggggacacactgaaagggtttcagaagctggagataggtggaagatgaagtgagggaaaaggaagtgcagtgccatcacggaaatgaaagccttgggacggaggggtcacctggatgtcctgggcttcttgggccctccgtcctaaacaagcataaagagcatcacgggattatccttggtagtctcaaagctgagagtcatggggaggctgtgaacattgaagatcctaccagggacacaaaattacgggtcccttcttcaatcctgcctgtggttagcaggaggttgagggagcgatggtcctatttcccagaggaataagagctctgggctccttcaggaaacctggggaagaggatgyccaagtctgcatgaataccaacagatgaggccatcggaagaagggctcctaagaaagagaaaccacacacagaaaggaagaagtgaatatgacccatgctcacacaccaacatgcctatagccaggaggaaatatgagagctaggagggaatttaggagtctctgaattgaaagtattcgtttcagtgaggaggaaactgaagtttagagacgtagaataaacttattgtaagaggaacctatgtaatatgtcttagaaagctctctttcaaaatcattatccaaaaagga

SEQ ID NO: 50 - DNA, 3307 bp, Homo sapiens
aatcctacaagaaacatttcattattcccacttagaagctaagaaaatgaaagttaagagagattagcttcatatgacgaggaataaaaaccacatttttctttaggtttagtttattcatctatttctagttccttgcagtgtaacattaggctgtttatttgggatctttcttcttttttaatgtagatgtttattgctttaaacttccctcttggaactgtttttgctgcatcccataagttttggtatgttgtgcttccatttttatttgtctccagatttttaaaaaatgtctcttttaatttatttgttgatccattggttatttagaaacatgttgtttaatttccacatatttgtaaattttccaaaattcctcctattattgatttttagtttcataccattgttgttggaaaagatacttgataagatttcaatcttcttaaattcgttaagacttgttctgtggtctaacatatgatctatcctggggaatgttgaagcaaatgtgtattctgctgctgttggataaaatgtcatgtatatgtctgttagttccatttggtatatccaatgtttccttatagatattctgtcaagataatctgttcattgttgaaatcccctactattattgtcttgcagtcaatctctttcttcaggtctattaatattggctttatatatctaggagctctgacattaggcacaaatatatttacaattattatatcttcttgatgaattaatccctttatcattagataatgaagttctttgtcacatttcacagtttttgacttaaagtctattttttttttgacataaccatagctctccctgctcttttttggtttccatttgcctggaatatttttgttcatcctttcattttcaacatatgtttgtcctttaaggtgaagtgagtctcttgaaggcagcatattattattttttcacccattcagccattctgtgtgtgtctttggttagagaatttaatccatttatattcaaggtaattattgataggtaaggacttactcctgtcattttgttaattgttttctgattgctttgtagattctttgtttctttctttctcactggctgtcttcctttctgattagataattctttctagtatgctttcattccttaaagttttatcttttgtttatctactatacatttttgctttgtggttaccctgaggctaacataaaatatcttatagttataaaaggttattttaagctaacaacttaactttgaccacattaaaaaacttaacactattrctccaccatgccccacatgttttgttttttatgtcacaatttacatctttttttattgcgtatcccttaacaaagtattgtagctattattatttttagtagtttcatctcatcttcatagtatgaatataagtgatctatcacttatattcatagtatgaatataagtgatctaatctcaaccaccattagattattgagtattctgaatttcactgcatctttattttaccagtgagttttatactttgataaattttcatgttaataattaatattcttctatttcagcttgaagaactccctgtagcatttcttataagacaggcctggtggtgatcaaattcctcagctgacgttaagtctgggaaagtctttctttctccttcatttctaaaggacagctttaccaggcaatatattcttaattgacaggtttttttttcccccctgcagcacattgaatacatcatccaactttctcctggcctgtaaggttctgctgagaaatctgcttctagccttattgaaacttccttatatgttattttcttcatttctctagctgctttcaggatcctctctttgtctttgattttttgtgggtttttttttttttttgcgggggagggggttgtttgttagtttctcgggttttgtgtttatttttccttttgtttcttttttgtttatttgttttgttttttgagacagggtttagctctgtcatccaggctggagtgcaggggcacgatcttggctcaccacagcctcaacctcccaggctcaagtgaccctgccatctcaaccccctgagtagctgggactacaggtgcatatcaccacacctggctaattttcgtatttttatattttcattttttgtagagacagggtcttgccatgttgcccaggttggtctcaaactcctaggctcaagtgatttgcctgccttggcatcccaaagtgctgggattacaggcatgagccactgcacctggccttctttgtcttttgatttttgacagtttgattacctgtcttggggtagtctagtttagattgaatctgatcagaaaactttgactttcctgtagttggatatttatctctttcccttgatttggacattttctgctagtattctttaaataagttttctgcttttttgtctttctattctccttcttgaacttctgcaacttgaatatttgccattttgatgctttcccataaatctcatatgctttcttctttccttgttattctgtattctttttctcctctgatggtatattttcaaataacctgtcttcaacttcacaatttttcttctgcttaagactttttttaaattttttcatcttaatttgtgagggtatatagtaggtgtatatatttatgtggtacatgagatgttttggtataggcatgcaatgcacaataatcatttcatggaaaatgaggcgtccatcctttcaggcatttatcctcgtattacaatctaattatactttttagttacttttaaacgtacaattacattatttctcactatagtcacgctgctgtgctatcacatattctttctattttttgtacccattaaccatccccactcccctatcccaaatcccctactacccttcccagcctctggcaactatcctcctactttctatctccatgggttcaattgttttgatttttagattccacaaataagtgagcacatccaatgtttatctttctgtgcctgacttatttcacttagcataatgacctccatttccacccatgtcatttgcaaatgacaggatctcataccttttatggctgaatagtactccattgtgtataagtaccacattttttttatccattcatctgttgatggacacttaggttgcttccaagtcttagattctgaacagtgctgcaacaaacatagaagtgcagatatgtctttgatatactgatttcctttatttggggtatat

SEQ ID NO: 51 - DNA, 491 bp, Homo sapiens
gagcgatggtcctatttcccagaggaataagagctctgggctccttcaggaaacctggggaagaggatgcccaagtctgcatgaataccaacagatgaggccatcggaagaagggctcctaagaaagagaaaccacacacagaaaggaagaagtgaatatgacccatgctcacacaccaacatgcctatagccaggaggaaatatgagagctaggagggaatttaggagtctctgaattgaaagtattcgtttcagtgaggaggaaactgaagtttagagacgtagartaaacttattgtaagaggaacctatgtaatatgtcttagaaagctctctttcaaaatcattatccaaaaaggaaagaatgggccacttaaaggagtattgatttattaatcgggaaatttgcttatggaaaataggcaaaacttgcttcgaaatgcttatcacaatccacctaaaatttctgttggcagcatcattatctgta

SEQ ID NO: 52 - DNA, 601 bp, Homo sapiens
agcacagattaggacacacatttacagcctgtgctatatgaacaaagctgaaattaactgggactaccgaataaataaaatacattatatttgcaaaatatataattcatagctaatatgacattttaatttttatataaaaatatatttttatatctgcccatatgcatatacatgcatgcatacccagacatgtgtatacacacatttacatacctggaaggatgttcccgatgtgttaaatggaaagagctagttgaagggtagaataaatgatatgataacgtttttgtttctagaraagggaaagatactctatatgaacatatatttatattgttgttggaaaaatttaaaaattgtgggaaaatccccacaaactgccatcattggctcacttgggaaagtagaggtggaaaggcagtgagctatgattagtttatataccttggtgttatttcagttttacaacaaacatatattactttttgtaatataggaaactataggtttgtaactaggaaaatatatataaatttcaagaggacagatttcagattaatatgaataattttctaataggcaggattatttggattta

SEQ ID NO: 53 - DNA, 906 bp, Homo sapiens
aatttcccatctgcactaatatgtcttcccctatgcagatgccattcttacttcactctaacactcatgctgggccatcccaggacacagatgtcctcctcaccttgcctggactctgacaccctactccaggccattcttccacatgggcacctccttgctaggctaaggttcagaggacccatgtcaggctgccgcactgtgatagactgcataatggacccccaaagatgtccacatcctagtcccctgaatctgggactgttgctttatatggcaaaaaaaaacaaacaaacaaaaaaaaaaaaaaaaacttctcagttaaaaatcttaagaaggaggagacagtatccygaattattcaagaggtcttaatgtaatcacgagggtccttataagaggaaatcgggagtatcagagtcagaaagaatgagagagcctggaagatactcttctgctggcttttaaaaaggaagaggccactagctgaggaatgagagtggcctctagcagttggaaaagtcaaggaaacagattgtctcctaaagtctccagaaggaacaagccctgccgatgccttgattttagcccactgagcctagttttggatttctgccctccatgatgataagataataaacgtgggttttttttttcagcaactaagtttgttgtcacttattaccaaagcaataggaaactaatatgctcacccacctcttcaaggaccttctcctcattctgctcaggttctgacaccttccacaccaggtttcctccctacacactgtgcctggaatttggctgcccatagtgggcaattacagatgtctaccttactctgatgcacttaattgatttagaataatattgttcaaatgggaaggagagaagaatgaagagaac

SEQ ID NO: 54 - DNA, 812 bp, Homo sapiens
cagtgatctcaagaaatgagttgtcctcagggtagcccctgaaatggcaatggcatgaggctttgaaaacttgtatatttttccaatggaaacttactcctgtatctctcatgataaaagttctatacagcagactggcaggttcacgttctctcctatgctacctggcagaggaattctgagtccatgatgagccaatarataagtttcttttctcaccagtgtttaacctgtcattattaccatgtcaccaatccctgaaccaatttaagaagtatcagaattaaattcccatccattgatttttcaaatggaaatattttttaatggttgtaaaattatgtgggatcttttaaataaaaaaaaaaacagaaaatacagaacagcataagaaaagaaaaaaatcacctatgatttcactttccacaggtaaatatagttagcttttggagacaaagtatttcattatttttttctacctacataacacagtgcctggcacaaaattttttcccaataaacttctgtggattaacgaaatatgagccaaagtgatttaatgattaagttcaaaggctccggagtcaggctgtgtgtgttcaatcatgactttgctccttactgtcttgtcgattattagtgcattacttaacttctctgggtatcactttcctcgtctgtaaaaaggtgatataaataataactgtctcaaaagatttcatgagtataaattatgtcaacatgtgtaatagtgcaatgccttgcatgtggtaagagctcattaaatgcacagtcattattactagtggctt

SEQ ID NO: 55 - DNA, 601 bp, Homo sapiens
tccctaaacttactgattaaaaacaaaaatagctaagccccaattacccagaattcctggtgcccctaacccacccaagatcagttactcatattgatgattcttgttaccccagagttttcagtgccttctacatagtaccctcaatgagagaaaaatattaatttgaaaatatttgaaatgatcttgtcaaactccttggaagattaatcatatgccattgattagaaaccagagaaaagcaaggctgcaaaattatggcttcatgcatgcacaggtgtggaagtttccataaaattcwattagtccaatggtcatagggctgagtgggtgatagccatctccccaccctccaagtaattttggaaatgtactgtgagcaactctgattgtcacaataatttcgtaggttctactggagtattgtgggtggggaagtcaatgaggttagaactcctgcaatgcatgagacagttttgtgcaatgaagaattgccccatgtctcatgcaatttacacacataatttatcttaatttatacagtggccctgtaggtagtgtatttatctccatctggcagattataatggaggttaatggg

SEQ ID NO: 56 - DNA, 601 bp, Homo sapiens
atgcaggggcctctagagaccccactacaacatctaagataattctccacctaaagtagtgaaaaatcatgttggacaccagaaagctcttagcaaggctcaataattaattactgatgttattttcacatggaaagaaatattcttggtaaatcagaataaatttcttgaaacttcatgtaaaattcataattgtgttaaggtaattttgagccactgtctgtgtatgccgttctgtgggatacacagagtatacctttgtgaggctccagggacattctttccacttcgtacttctttytaaatcacaaggtaagatcttatgagatgcaaagattaatttgttttcctccaccaacttaaatttttctccctttctttactacctgtaggattttagcactgaataaataataggcttgaaggtgaactattttcatgagcccatatgcattaggacaaaaactgaattctatggtttaaccaggacataatatacatcaatatggtctttgaatggcttacaaaggaaaaaaaacatttcctgggttattggaagcagcatggtgtcaaagtagttaaacagattctatctctgtgg

SEQ ID NO: 57 - DNA, 772 bp, Homo sapiens
tcatgtttaacgtatttgttcaggttaaattgaaatattttacatatagaaactgaggttgggttacctcagaaacagagcttgagacaaggattttttttttttttttttttttttggtggtgattctaggaagcaccagtagaaaagaggcaaagagattcagggaagggaaggaagtcagttcagggtggttcccaaagggagctactgtagtcaactgagactcagcccactatagacctctgggtgatggtgtagcccataccccaaagttatcctgcccaagggacgaagaagttggggtatctatcctgcgactatctttagcactgtctgagcactgctcccagggcattaaacccctagctcttccagtcttcctcatgtgaaaatagaaagaagcccttaggccaagaatagtgaactgttacagtcacaggcagagggtaagaagagagagggaggctgctgagaggatgttggcaaggcaggtagtatytgctatgagaagttattaattattccctcatattttttttcagtttttattacatcctttatttttcggcattagtgtcagtataccaacaagttgcatttgccaggacttttgtggtgacaagtgacgaaaattccagtcacactattttgatcaaagaaaggatctcagagacaggtactcaagtgttgacaggatttgtctctctagctgtcacttctgcttctctttgtgagacaatgtcaatcctgcctcccacagagcagcattc

SEQ ID NO: 58 - DNA, 772 bp, Homo sapiens
tagaagttgtatttgtacatttcttcatgtttaacgtatttgttcaggttaaattgaaatattttacatatagaaactgaggttgggttacctcagaaacagagcttgagacaaggattttttttttttttttttttttttggtggtgattctaggaagcaccagtagaaaagaggcaaagagattcagggaagggaaggaagtcagttcagggtggttcccaaagggagctactgtagtcaactgagactcagcccactatagacctctgggtgatggtgtagcccataccccaaagttatcctgcccaagggacgaagaagttggggtatctatcctgcgactatctttagcactgtctgagcactgctcccagggcattaaacccctagctcttccagtcttcctcatgtgaaaatagaaagaagcccttaggccaagaatagtgaactgttacagtcacaggcagagggtaagaagagagagggaggctgctgagargatgttggcaaggcaggtagtatctgctatgagaagttattaattattccctcatattttttttcagtttttattacatcctttatttttcggcattagtgtcagtataccaacaagttgcatttgccaggacttttgtggtgacaagtgacgaaaattccagtcacactattttgatcaaagaaaggatctcagagacaggtactcaagtgttgacaggatttgtctctctagctgtcacttctgcttctctttgtgagacaatgtcaat

SEQ ID NO: 59 - DNA, 2666 bp, Homo sapiens
ctatcaggcttatattcctagtgtctaggaaattgccaagcctaaaagaaaagatgtactaatgtggggttcctgccagtgaaactcaccagtttcaagtatcaccctactaagaggcttgtaagtcaggaagtccaatcaatatatttaatgtacccaatccaacaatttggactttgttataaaacataaacaacattaatgaacagaaaaatacttgaaaaaatactttaggataaaatacaaagtctaaaaaacaaacagaaaaaataaataaaagaaatgaagtttaatgccgagaaaaataacaaagagaaaaaaatttaaaaagtaatagaagattcatggaacaataacatttagagaagaagaagctcttagaacttaaaagctggtggtagagccaggtgcagtggctcatgcctgtaatcccagcactttgggaggccaaggtgggcagatcactcgaggtcaggagttcgagacaagcctggccaacatagtgaaaccccgtccctactaaaaatataaaaattagccaagcatggtggcacacatctgtagtctcagctatttaggaggctgaggctggaggattactggagcccgagaggcagaggttgcagtgacccaagatcgcaccactacactccagcctgggtgacagagtgagactcagtctcaaaaaaaatggtggcagaagtttaaaagcaatagaagggttgaaatataaagttgaagaaatctctaagaaagaacaaaatgaccaagaactggaaaaatataaagaaattcacgaaaactaaagaatctacttagaaatccaacacttaagtaacaggtgctccagaaagagaaaatatgtaattgaaggaagaaaattttcagaagattatttgtataatttttccatagctgaagaatgtgagtttccaaaatgaaaaacccaacgaatgcccagcccaatgagtttaaaaaataaaaataaaaagacaggccttggagtgcatttttaaatttcggagaatcttgtatgtgagaagatcctcaaagttcaagagagaaaacataggttgtaaacattataaatacaaaggatgcagaaacagaatgtcaccggacttctcaatagctattctggaagctagaggttgatggagcaatgtttttaaatattggaataaaatagtgtccaaactagaatttcacgctatgycaaacaattaatagttaggatgagacaattttttttattcatgggagatttcatgactttatgtcccatgtgccctttctcatgaagcatcttgagaaagtcaagaaagtgtttaacctaaataaagaaattaaattaaggaagaagacctgggatccaggaaacaaaggatttaacacaggaaaaagctagactattttctagcaggtggtgagggaagtcccaagaggatcattgtgcagcaggcctacagagcaaccagcactggttggaactaaaggactgggaagcccaggagaaatgtctccaagaaaagaaatggaattaatatgaacattacgaagaaatttcacccctgacagagactggggtaggggaaggtaaattaatgatgagtatgtggaaaactaagaaaaccaaccaaacaaagccaattattaacttcaggaaaagcaaatattgtgcacgaaaaaaatgtaatattgtaccacaaatgtcatgaacaagaattacctaatcatagtcatgtccattttaccacctaaagtgtaatatagctataatgggaagacagaggacaaaggggctaagtgtatatgtgtatagggtagagtaagtcatagtcatattacctgaaatgggaaaaattcaatgtaagaaataggtagttttactgggtaagtagaagttgagctaagaaatgaagctaaaggaattgaaagtgatagcctcagagaagtatgttttagagatggaactgcatgaatcagagttactggctttttgttataagccttgtggtatttggaacatctgggagtccccaaagccaccttcatttctgacaccagctgaaagtttggaaccagccccaggttcaataattcactagaaggactcatagaactaagaaaaaccattatactcatgattatggtttattacagcaaaagaatacagattaaaatcagcagaggaaagaggtccatagggcagggctcaggagcactccatgcttagagcttccagtcattctctaccagtagagaagtggacagtgctaacttttcccagccatgatgtgtgacaatatacacagagtactgcagactaggggagcttacttgagtcttgctgtccggagactttattgagcttggtcacatagacaagattgacacctgtatgattgactttggtctctagccctttcagaggtcaatttgatactttgtggcccaaggctcccaccatagatcacattgttagcatagattatgtcgcagggcttaaggcctctaggaaaccaaagacactcttatcaggcaggacattccaagggcatagaggttacatccccagtgttggagacaaagaccaaacctctcttcggatgaagttaatcctgtactgcataatattcctttattttttcccttttaaactgttt

SEQ ID NO: 60 - DNA, 743 bp, Homo sapiens
gggtggctcatgcctgtaatcctagcactttgggaggccaaggcaggtggatcacttgagctcaggagtttgagaccagcctaagcaacaaggcaaaactctgtctcaacaaaaaatgcaaaaattagcctggtgtggtgccttgcacctgtagtcccagctacttggggggctgaggtgggaagatcacttgagcccaggaagtcggggctgcagtgagctgagatggtgaggctgcactccagcctgggtaacagagtgagaccctgtctcaaaaaatcaattaatcaataaagtgttgttgatgtttatgaaacccttagagctctaccaggcatacagtgaactacgatgttgttgatgatgataatcatctttattggcacatgccaggacttgataaccttagtttgtaatgtgaatcctatttaaaagtatttaaaagtatttccactacaacttaagaaactgtcatccagtgcaaagcycagggtagacagcagagagttggatttagccatgattgattggagtttttccaggaaaatacgatgaaggaagacaagaacaaatgacagaccatggaattgaggctcgataatgagagaagtaaagacataaagtggagaggaaccgtgaaaagatgctaggaataatgttttttttcaattccattggaatttaatgacagctagagtgggttatagaaagggcaagctgaaaagtcatagagtaggagtcat

SEQ ID NO: 61 - DNA, 601 bp, Homo sapiens
gggaagtcaatgaggttagaactcctgcaatgcatgagacagttttgtgcaatgaagaattgccccatgtctcatgcaatttacacacataatttatcttaatttatacagtggccctgtaggtagtgtatttatctccatctggcagattataatggaggttaatggggagccttcatcttccctacctgcttgaaaatctctatccctagaactaatcattttggttcaacgtatgcagacaatattcctccctcaatttttctagattgttcacatctccatggggcatatgcagggrcctctagagaccccactacaacatctaagataattctccacctaaagtagtgaaaaatcatgttggacaccagaaagctcttagcaaggctcaataattaattactgatgttattttcacatggaaagaaatattcttggtaaatcagaataaatttcttgaaacttcatgtaaaattcataattgtgttaaggtaattttgagccactgtctgtgtatgccgttctgtgggatacacagagtatacctttgtgaggctccagggacattctttccacttcgtacttctttctaaatcac

SEQ ID NO: 62 - DNA, 974 bp, Homo sapiens
ataccattctgggccaaggccaaagaaagcccctgagaatccttccagctctctcttcccttgctgcagtaatgataagggtcacatgttttgaggacacgaaacatggcagatagaatacatgctacctctacattctttcagaatccgtaagacaaaaataacaacataaaaggctataaagcctcaacaacaaaaaaagccaaaagcaaatgagaaatgtcaatgaagttatggaagatggaaagaagatgagcaagtggtgagtaacttcaactttagatttctccactkcggcaagtaccaagtagaggaaatttagttcacactgcagattagtagaaaactcaggaattgtgttattaatcacttctgaagaaggaagttcagggtgggattgaaaataagacaattggttgaaaaatgtatataagatgtagttagatccctcgtatcccacttagccacaccactgcccccgtatacctgtttgaagactggaagtttaccttccagcaaggttctggatatcttctggatatttagcatagctgagaaggaagtaagtaccttcataaggtttggatttatttgaaagtcatcatactgagcagtgagaacacgaggcttccagaatgcttactatcaggcttatattcctagtgtctaggaaattgccaagcctaaaagaaaagatgtactaatgtggggttcctgccagtgaaactcaccagtttcaagtatcaccctactaagaggcttgtaagtcaggaagtccaatcaatatatttaatgtacccaatccaacaatttggactttgttataaaacataaacaacattaatgaacagaaaaatacttgaaaaaatactttaggataaaatacaaagtctaaaaaacaaacagaaaaaataaataaaagaaatgaagtttaatgccgagaaaaataacaaagagaaaaaaatttaaaaagt

SEQ ID NO: 63 - DNA, 690 bp, Homo sapiens
gcttgtaggctggctggccaggggaaactaccagtccgctttgtgcaagtgaattctcaaaccctatctgagcacaggaatcacctgggcgtcaaacaggagaaagttaatatcctactctattctcccacaaatttctataggactaataaargaaaagaaaggaaagaaaatgtgaaaatgcctaatttatctacttagtttttactcataaaacttttagcactggaatagaccaaggagattgaataagccgattgtttgcactttgcagaaagggagaccaaggcccaggtagttaagtcactcacctaacatcccacagggagtcctatgctcatgacaaaatagtgtcactatctaacagttaaagataagagttaaaactcgtgaaacggaagtgggtaaatgataacatttagtctctaaatgtcctctcgacaaaagaatgtcatatcaataaagataacacttagttcaaacacttgaaatgaaagtggctaaatgataacatctatcaaaatgctgaggtcaaccaacaggtctcttcaggggtgttcatggtggtgacggttttctggctctgcccaattgggatgctaccttcagatcagaccctgcatagaaggaagagactcttcctgagaaaggggcttcatgattaggcacagcagactgctgtgatcaagg

SEQ ID NO: 64 - DNA, 1032 bp, Homo sapiens
caacagggacattcagaagcactcttgaaatgacaaaaccctcagtggaatttatagcataactttctttttaatattccatttctgggtcaatccttgatcctgttaacactttaataagcagcaacaaagagaggcatcactgcagagactgtgcagatactaaatcagcttatgtacattctgtctaggaagactttyggaacagaggctggcttgctgttcatcaaatttatttgtgagttctagcccatggaatgtgagtggaagtgatgtgtgccatcctccatgggccatcttccatgtttcttctctgttgaccaacttgatacgaacatgcacagtgacctcacagtccaagtattgaagatggtgaagatggtggagccacaggatgaaaggatcctggtcctgcttagagaagagatagctcctgttctgttacatctatttttgactttacatgagcaaaaagtaaacttctattaggtttaagccattttacattttaatatagctactgaaacctcgcatcttgactacagcttttatgtaaataagaaatatggcctgtaatcccagctgtttgggaggctgaggcaggaggatcacttgaggccaggagttaaaggctgcagtgtactatggtcagaccactgcactccagcttggatgacagagaccttgtctttaaaagaaaaagaaaaatgtatatttcatattttaaaataaatttttggctgggcacagtggctcatgcctgtaatcccagtgcctcagaaggccgaggcaggaagatcttttgaagcctggaattcaaaaccaacctaggcaacatattgagaccttgtatcaaaaaaatattttttttaattagctggtcatggtgtgttgtgcctgtagtcccaactactcaagagaccaaggtgggaggatcgcttgagcccaaaaattcaaggctgcactgagctgtgatcacgtcattgtgctccagcctgggcaacagcctaagcaactctgtctctaaaatat

SEQ ID NO: 65 - DNA, 1843 bp, Homo sapiens
gctctcgaggagcctttgatttggtgggagcatcagacaagggagtcaaaggtttcaatacagtgtgacaagtggcattctacaagtattaacaggtatcatgacagcaagaagaattcagagaaggaatctcatttgactagggatgggagtgagaatatgagaggtggcaaaaatgaacagatgggtagggtcacaggyaatatgcacaagacctctcttctcatgaagcttacattttagtagagtcaaagaaaggaagataataaacaaggcaatcaacaaagaaacaagataatttcaaagcatgaggataatatgaaggaaataacaaaggtgatttggaattactaggagtggatggagatccttcctcagctgggttgggaacgtcatgtcaaaggaagagacccttgagctgacacgtaaatgaaaggaacggactgtgggaaggcctggggaagggtactccagggagaggagctagcatctacaaatgcccaagacagagctgaacttgcacttttcagaagcagaaaggtcagctaagagacaacacaggccaggagacaaggtcagagagaaaggctaggcaattaatgtaggtctttcttggccagataataaggtttattctcagtgcaagggaagccattgaaaggcatcaaacaggaagggatatgctttgatttacacttcttaagttctctctagaagctcaatgaagctggattcaggggcaaggtatgagtggaaacaatgagaccagttagaaggaggactcttccagtgtccaggtgagacatggcagtgacctgggccagggtatactaatggggataggagaagcggaaggatttgagatatattggggcggtagaactgcaagaatgtgctgatgaatttggtttgggatatgagggaaaagaagaaataaaaaatccctgtaattgcaaaaatggccctagcaattgagtaggtgacaatttatcatataataataacaacttatgcgtataaagtttttattatatagcagtcatggctctaacctctttacatatattacctcacatgaaccccacaacaaccctacaagataggtactattctcatccctattgtacagacaagggaagagagggacggacagattaacctcactttgttgttaaattacagcctctatgtgaagctttatcggcttcagagtctgtgtgcttaaccatgatatctttacgttttgtattaccaggttgtggaatactagagaatgaactgattttagaaggagaaacaaattttccggttttgacatattgtttttgagatgtcttacatggaaatatcgagtacataattgaatgtgtgagcatggaattcagggactaggtcaaccctggagacattagcacactgatagtatttaaagccatggggttgaattagctgtatagagagcaatagagtacatggagattacaagaagccacaactagccctgagtcctccaatctgtagtgttctgatagagaagaaactcacttgcaagatcaagaagcagcatctaagtgaggcagaaagaatcccagaggagagtgtggattttcagaactgagtgattaacatgttggcttgattctcagccagtctctgtcctcatggtggcaagatggctgcagcaattccaaccaatactcttccaagcttatagttcatagaaaagagaaagactcattttccagaactcatttataaatcctggaatccactctgattgggccttgttgggtcataggcccattcctgaatcttcaccaatcattgtgactagaggacccta

SEQ ID NO: 66 - DNA, 724 bp, Homo sapiens
caactaacatgccaaaactcaaagagttgaaaagcactcctgaaggtaaatatacccttctataaccgttatcaaataagacataattgtctatatatttgtccatcttatccttccaacttcatttcacactccagttttatttgtttgtcgaacactaattgtcttttttttctcatcagccctaacatattgtaaagwtccatttgtaactactttaatatccacattatcatgcatctttcagtaaagtaaaaaattgtccaagtttctccattctcagagttttgttttttggttttttttttttttgtttgtttgtttttgagacggagtctcactctgtcgcccaggctggagtgcagtggcgcgatctcggctcactgcaagctccgcctcccgggttcatgccattttcctgcctcagcctcccgagtagctgggactacaggcgcccgccaccgcgcccggctaattttttgtatttttagtggagacggggtttcaccgtgttagccgggatggtctcgatctcctgacctcgtgatccgcccaccttggcctcccaaagtgctgggattacaggcgtgagccaccgcacccggcccattctcacagttttactacttctgtatgctgacagcctgtccatctctacctctaggacagacctctctccagaacctctgatccacccagcccactgcggtgtagacggcctaga

SEQ ID NO: 67 - DNA, 401 bp, Homo sapiens
gctggacaggatggacaccctctccaagaccctgggggagcaggacaaagccagtgctccccagaggtggtcactcccaggaggaaaagcagagagatgtggaaggggctgggtacatgtgccctgtttgtcctcccaaacacagcaggcagaagagtcactccacccagggcaaagtgaaggagagggtggagggagatygggaatgctgtgctcatagatctctcttgacaagaatggggagaaaagttccacaccaaaggagggcaaagccagagaaatagggaagaggtctcgggatctgcacagtgagtttgtggagcgtaaactccacgtcagtttatgtggctacacataaagataactccaataaaccaccttcagggagcctgctcgaagta

SEQ ID NO: 68 - DNA, 852 bp, Homo sapiens
tatttttccaaactaataatggaagtggtattagggtaatatatttataggtgagattccagggctgatttagtaaatattaatttctaatactttgtcattcccactgcattattctcctatagctgtcacaacaaatcaccataaaccgggcagcttaaaacaacagaaatttgttctctctcagttctggaggctagaagcctgaaaccaaggtgtcggtagcaccatgctcccatgcttccttctagggaagaatgcttccttaccagttctggcttctgcctattcttggcactccttggcttgtggcagcacaactccactctctgcttccatcttcacatgcccaacttccttccatttatgtgtatctgtgccaaatttccctcttcttataaggacatctgtcattggattagggtttaccctaatgaatttggggaggaccctattcaatccactacaaccaccctttatgtacacgtagctggtttctctgtcaattatattttagagtgaggackttgcttctcctctaacaagatattataataacaattattgtcaaattatttaatgaatgcttactatatgacagttacatgcattaactcatttaaccctctgacaattctatgaaataggtgctatttttatttctattttgcagatgagcagccagagagagtttacatagggcaaatatcaccattacctagcaagaacaaaataagaggaataagcagtccccttgtattttggttacttaaaagggatggatctcaagacaaaggaaaatggttgggtgcacgaggggccagatgctggaaccagttctgaagaagtgtt

SEQ ID NO: 69 - DNA, 601 bp, Homo sapiens
gctgaggtgggaggaatatgaaggcccaggagttcaaatccagcctgggcaacacaatgagaccctgtcttaaaaaaaaaaaaaatcagcaagctgggaaataaacttggggcacactgggcacttcgtcatgaggaaaccaaaatctcctgccttggcaagcttcaggagccatataaggactgagccagcctcacccattacactgtgtagggacactcttcagcaacgacatcatgtggcagaagaaaacatggccataggggattccttcattgtgcaattacctataagaagaagraaaggaagaaaagaggaagaagaacgaggaggaggaggaggtctaaaaaggaaatgcttaaattcttgctgaaaggtgagtgaattttggagttcaatgtaacaaccaataaataacatctctcttctcttcttggttctgtgcccattgaaaaatacgacaaagagtgaaacaaatggaaaagcaaagtattatcctctttctgataaagcaaataacagagaatgtagctctaatttgtgggcaaatgggggtcttaaaactgaacctcagaatttaatatttaaccgacttctggtg

SEQ ID NO: 70 - DNA, 613 bp, Homo sapiens
cacattttcagattaaatggacaaacgcttgacttctatttcatatatatccatatacaaaaaaaatcagaaagtggtatagaaattgtatttactgaacattaagcacaacccatttatttctatttaaatagcaccaaaacctcagtaacatttaacaggttaacaatatagacttgagtcatattgagtctgacattgagtcagacctagatttatatcatgctctgccacagatactctggtatctttaagctaattacatatccccaagcctcagctgtccccaactgcaagatggccatgatgacagatgagaacagataactcagagtgtggctatgagaactaaatgawttaacgcctgtaaaacatttagaaaaatgcctagcatgtggtaagtgctcattaaacatagctatatttaaatatttctaaaatattgccaaatccagatgctaatgactagggcatcctaaaagacagatttagaaaggaaattgctgtctatattctgaacagtacagtaactgtgttttgactttgtcatttgccacttccatccagtgcttttctggtagcatgctggaaaatgaaccacagcacactaaca

(19) United States
(12) Patent Application Publication
(10) Pub. No.: US 2010/0041600 A1
(43) Pub. Date: Feb. 18, 2010

(54) INTERFERON GAMMA POLYMORPHISMS AS INDICATORS OF SUBJECT OUTCOME IN CRITICALLY ILL SUBJECTS

(76) Inventors: James A. Russel, Vancouver (CA); Keith R. Walley, North Vancouver (CA); Anan Wattanathum, Bumaby (CA)

Correspondence Address: BROWDY AND NEIMARK, P.L.L.C.;624 NINTH STREET, NW, SUITE 300, WASHINGTON, DC, 20001-5303, US

(21) Appl. No.: 12/304,104

(22) PCT Filed: Jun. 11, 2007
(86) PCT No.: PCT/CA07/01042

§ 371 Date: Oct. 9, 2009

Related U.S. Application Data
(60) Provisional application No. 60/812,071, filed on Jun. 9, 2006.

(51) Int. Cl. C12Q 1/68; A61K 38/16; C07H 21/00; C40B 40/06; A61P 29/00

(52) U.S. Cl. 514/12; 435/6; 536/23.1; 506/16

(57) ABSTRACT

The invention provides methods, nucleic acids, compositions and kits for predicting a subject's outcome with an inflammatory condition and a subject's response to treatment with activated protein C or protein C like compound to identify subjects having a greater benefit from treatment with activated protein C. The method generally comprises determining a interferon gamma (IFNG) gene polymorphism genotype(s) of a subject for one or more polymorphisms in the IFNG gene or associated sequence, comparing the determined genotype with known genotypes for the polymorphism that correspond with an improved response polymorphism to identify potential subjects having an inflammatory condition who are more likely to benefit from treatment with activated protein C or protein C like compound and subsequent to treatment recover from the inflammatory condition. The invention also provides for methods of treating such subjects with an anti-inflammatory agent or anti-coagulant agent based on the subject's genotype.

### FIELD OF THE INVENTION

The field of the invention relates to the assessment and/or treatment of subjects with an inflammatory condition.

### BACKGROUND OF THE INVENTION

Interferon-gamma (IFNG) is a pleiotropic T helper-1 (Th1) cytokine that plays a pivotal role in defense against infectious pathogens and in the induction of immune-mediated inflammatory responses (BILLIAU A. et al. Ann N Y Acad. Sci. (1998) 856:22-32). The IFNG sequence maps to chromosome 12q14. A representative Homo sapiens IFNG sequence is listed in GenBank under accession number AF375790 (7665 bp-AF375790.2 GI:14278712). The human IFNG gene has 4 exons.

IFNG is considered a pro-inflammatory cytokine, since it has been shown to augment tumor necrosis factor activity (DINARELLO CA. Chest. (2000) 118(2):503-8). An increase in IFNG occurs within the first 24 hours of the development of sepsis (LAINEE P. et al. Crit Care Med. (2005) 33(4):797-805) but, subsequently, monocytes from patients having sepsis demonstrate decreased IFNG production (RIGATO O. and SALOMAO R. Shock. (2003) 19(2): 113-6). Administration of IFNG is beneficial in restoring immunoregulation in humans and improving survival in some models of sepsis (KOX W J. et al. Arch Intern Med. (1997) 157(4):389-93; DOCKE W D. et al. Nat Med. (1997) 3(6):678-81; HOTCHKISS R S. et al. Proc Natl Acad Sci USA. (2003) 100(11):6724-9) but administration of an IFNG antibody is beneficial in other relevant models of sepsis (LAINEE P. et al. Crit Care Med. (2005) 33(4):797-805; YIN K. et al. Shock. (1999) 12(3):215-21; ZISMAN D A. et al. Shock. (1997) 8(5):349-56; REDMOND H P. et al. Ann Surg. (1991) 214(4):502-8, discussion 508-9).

Associations between interferon gamma polymorphisms (single nucleotide polymorphisms (SNP) and microsatellites) and complex disease susceptibility and outcome have been reported in numerous Caucasian, Asian and African populations across a wide variety of indications (e.g. cancer, transplant, tuberculosis, sepsis following traumatic injury). Table 1A outlines some studies. For example in a critically ill cohort (n=61), Stassen et al. (Surgery. (2002) 132(2):289-92) reported that homozygotes for the (CA)12 allele of the interferon gamma intron 1 (CA)n microsatellite (starting at position 66838790) are more at risk for developing sepsis after traumatic injury (p=0.06).

TABLE 1A
Associations between IFNG polymorphisms and disease susceptibility (or survival where
specifically noted). Build 35 chromosomal position, the associated allele or genotype and rs#
are given for each polymorphism.

| | SNP/microsatellite | | | | |
|---|---|---|---|---|---|
| Disease | genotype | Population | n | p | Reference |
| Acute graft-versus- | IFNG.66838790.(CA)13 | Unspecified | 80 sibling | 0.02 | CAVET J. |
| host disease in | (CA)13 | | donor- | | et al. Blood. |
| bone-marrow- | (donor genotype) | | recipient | | (2001) |
| transplant | | | pairs | | 98(5): 1594-600 |
| recipients | | | | | |
| Allograft fibrosis | IFNG.66838790.(CA)12 | Unspecified | 82 patients | 0.005 | AWAD M. |
| in lung-transplant | | | | | et al. Hum |
| recipients | | | | | Immunol. |
| | | | | | (1999) |
| | | | | | 60(4): 343-6 |
| Autologous bone | IFNG.66838790.(CA)12 | Mixed | 87 patients | 0.011 | WU JM. et |
| marrow transplant | associated with | | | | al. Biol |
| in breast cancer | decreased survival | | | | Blood |
| patients (survival | | | | | Marrow |
| not susceptibility) | | | | | Transplant. |
| | | | | | (2005) |
| | | | | | 11(6): 455-64 |
| Breast cancer | IFNG.66838789.TT | Iranian | 223 patients | <0.002 | KAMALI- |
| | (i.e. rs2430561) | | 267 controls | | SARVEST |
| | | | | | ANI E. et al. |
| | | | | | Cancer Lett. |
| | | | | | (2005) |
| | | | | | 223(1): 113-9 |
| Bronchiolitis | IFNG.66838789.TT | Unspecified | 93 patients | 0.039 | LU KC. et |
| obliterans | (i.e. rs2430561) | | | | al. |
| syndrome | | | | | Transplantation |
| following lung | | | | | (2002) |
| transplantation | | | | | 74(9): 1297-302 |
| Brucellosis | IFNG.66838789.AA | Spanish | 83 patients | 0.023 | BRAVO MJ. |
| | (i.e. rs2430561) | | 101 controls | | et al. |
| | | | | | Eur J |
| | | | | | Immunogen |
| | | | | | et. (2003) |
| | | | | | (6): 433-5 |
| Cerebral malaria | −183T* | Malian | 240 families | 0.009 | CABANTOUS S. |
| | −183GT* | | | 0.013 | et al. |
| | INFG.66838790.(CA)14 | | | 0.073 | J Infect Dis. |
| | (CA)14 | | | | (2005) |
| | | | | | 192(5): 854-60 |
| Coeliac disease | IFNG.66838789.T | Sicilian | 110 patients | 0.0045 | LIO D. et al. |
| | (i.e. rs2430561) | | 220 controls | | Dig Liver |
| | | | | | Dis. (2005) |
| | | | | | 37(10): 756-60 |
| Early rejection in | IFNG.66838789.T | Unspecified | 118 patients | odds | TINCKAM K. |
| renal transplant | (i.e. rs2430561) | | | ratio CI | et al. |
| recipients | | | | 1.1-3.2 | Transplantion. |
| | | | | | (2005) |
| | | | | | 79(7): 836-41 |
| Endometriosis | IFNG.66838790.(CA)n | Japanese | 185 patients | 0.0436 | KITAWAKI J. |
| | | | 176 controls | | et al. |
| | | | | | Hum |
| | | | | | Reprod. |
| | | | | | (2004) |
| | | | | | 19(8): 1765-9 |
| Idiopathic | IIFNG.66838789.T | Mixed | 125 patients | 0.004 | STANFORD MR. |
| intermediate uveitis | (i.e. rs2430561) | (United | 100 controls | | et al. |
| | | Kingdom) | | | Br J |
| | | | | | Ophthalmol. |
| | | | | | (2005) |
| | | | | | 89(8): 1013-6 |
| Immunoglobulin A | IFNG.66838790.(CA)13 | Japanese | 96 patients | 0.01 | MASUTANI K. |
| nephropathy | | | 61 controls | | et al. |
| | | | | | Am J |
| | | | | | Kidney Dis. |
| | | | | | (2003) |
| | | | | | 41(2): 371-9 |
| Intrauterine | IFNG.66838789.AA | Chinese | 46 patients | 0.023 | YU H et al. |
| Hepatitis B | (i.e. rs2430561) | | 73 controls | | Zhonghua |
| Infection | | | | | Er Ke Za |
| | | | | | Zhi. (2004) |
| | | | | | 42(6): 421-3 |
| Melanoma (stage | IFNG.A66838789.TT | Unspecified | 90 patients | 0.003 | LIU D. et al. |
| IV) treated with | (i.e. rs2430561) | | | | Clin Cancer |
| biochemotherapy | | | | | Res. (2005) |
| (survival not | | | | | 11(3): 1237-46 |
| susceptibility) | | | | | |
| Multiple sclerosis | IFNG.66834490.A | Ireland, | 64 male | 0.019 | KANTARCI OH. |
| (in men not | (i.e. rs2069727) | USA | patients | (Ireland) | et al. |
| women) | IFNG.66838790.(CA)13 | | (USA) | −0.044 | Genes |
| | | | 147 male | (USA) | Immun. |
| | | | patients | 0.05 | (2005) |
| | | | (Ireland) | | 6(2): 153-61 |
| Oral lichen planus | UTR 5644 A/T | Caucasian | 44 patients | 0.0022 | CARROZZO M. |
| | | | 140 controls | | et al. J |
| | | | | | Invest |
| | | | | | Dermatol. |
| | | | | | (2004) |
| | | | | | 122(1): 87-94. |
| | | | | | Erratumin: |
| | | | | | J Invest |
| | | | | | Dermatol. |
| | | | | | (2004) |
| | | | | | 123(4): 805 |
| Pancreatic cancer | IFNG.66838790.(CA)12 | Unspecified | 57 patients | 0.0198 | HALMA MA. |
| (survival not | (increased survival) | | | | et al. |
| susceptibility) | | | | | Hum |
| | | | | | Immunol. |
| | | | | | (2004) |
| | | | | | 65(11): 1405-8 |
| Pulmonary | IFNG.66838789.A | Spanish | 113 patients | 0.0017 | LOPEZ- |
| tuberculosis | (i.e. rs2430561) | | 100 controls | | MADERUELO D. |
| | | | | | et al. |
| | | | | | Am J Respir |
| | | | | | Crit Care |
| | | | | | Med. (2003) |
| | | | | | 167(7): 970-5 |
| Sepsis in trauma | IFNG.66838790.(CA)12 | mixed | 61, of whom | 0.06 | STASSEN NA. |
| patients | | | 30 became | | et al. |
| | | | septic | | Surgery. |
| | | | | | (2002) |
| | | | | | 132(2): 289-92 |
| Severe hepatic | +2109A | Two | 105 patients | 0.035 | CHEVILLARD C. |
| fibrosis in human | +3810G | villages | | 0.035 | et al. |
| hepatic | | (Taweela | | | J Immunol. |
| schistosomiasis | | and | | | (2003) |
| | | Umzukra) | | | 171(10): 5596-601 |
| | | or the | | | |
| | | Gezira area | | | |
| Rheumatoid | IFNG.66838790.(CA)13 | Caucasian | 60 severe | | KHANI- |
| arthritis | | | patients | | HANJANI A. |
| | | | 39 mild | | et al. |
| | | | patients | | Lancet. |
| | | | 65 controls | | (2000) |
| | | | | | 356(9232): 820-5 |
| Trichiasis | IFNG.66841278.T | Gambian | 651 patients | 0.08 | NATIVIDAD A. |
| | (i.e. rs2069705) | | 664 controls | 0.001 | et al. |
| | IFNG.66836429.C | | | | Genes |
| | (i.e. rs2069718) | | | | Immun. |
| | | | | | (2005) |
| | | | | | 6(4): 332-40 |
| Tuberculosis | IFNG.66838789.AA | Chinese | 385 patients | <0.001 | TSO HW. et |
| | (i.e. rs2430561) | | 451 controls | | al. Genes |
| | IFNG.66838790.(CA)n | | | | Immun. |
| | on-12 | | | | (2005) |
| | | | | | 6(4): 358-63 |
| Type I Diabetes | IFNG.66838790.(CA)13 | Caucasian | 236 patients | <0.0001 | JAHROMI M. |
| | | | ? controls | | et al. J |
| | | | | | Interferon |
| | | | | | Cytokine |
| | | | | | Res. (2000) |
| | | | | | 20(2): 187-90 |
| Wegener's | IFNG.66838789.TT | Caucasian | 32 patients | 0.027 | SPRIEWALD BM. |
| granulomatosis | (i.e. rs2430561) | | 91 controls | | et al. |
| | | | | | Ann Rheum |
| | | | | | Dis. (2005) |
| | | | | | 64(3): 457-61 |
| IgA nephropathy | IFNG.66838790.(CA)13 | | 53 patients | 0.006 | SCHENA FP. |
| | IFNG.66838789.A | | 45 trios 4 | 0.04 | et al. |
| | (i.e. rs2430561) | | incomplete | | Eur J Hum |
| | | | trios | | Genet. |
| | | | 36 | | (2006) |
| | | | discordant | | 14(4): 488-96 |
| | | | siblings | | |
| Tuberculosis | IFNG.66838789.T | Sicilian | n = 253 | 0.012 | ETOKEBE GE. |
| culture-positivity | (i.e. rs2430561) | | patients | | et al. |
| | | | | | Scand J |
| | | | | | Immunol. |
| | | | | | (2006) |
| | | | | | 63(2): 142-150 |
| Lung function in | IFNG.66837463.TT | non- | n = 530 with | 0.008 | He JQ. et al. |
| smokers | (i.e. rs1861493) | Hispanic | highest | 0.002 | Hum Genet. |
| | IFNG.66834490.GG | whites | baseline | | (2006) |
| | (i.e. rs2069727) | | lung | | 119(4): 365-375 |
| | | | function | | |
| | | | n = 531 with | | |
| | | | lowest | | |
| | | | baseline | | |
| | | | lung | | |
| | | | function | | |
| Hepatitis B | −183(GG and GT) | Chinese | | 0.01 | Qi S. et al. J |
| infection | | | | | Clin Lab |
| | | | | | Anal. |
| | | | | | (2005) |
| | | | | | 19(6): 276-81 |
| Immologic | IFNG.66838789.A | Chinese? | | <0.05 | Zhu QR. et |
| tolerance after | (i.e. rs2430561) | | | | al. Chin |
| intrauterine | | | | | Med J |
| infection of | | | | | (Engl.) |
| hepatitis B virus | | | | | (2005) |
| | | | | | 118(19): 1604-9 |

*Denotes polymorphisms where chromosomal position could not be determined.

The risk of developing sepsis and the risk of dying once sepsis has already developed are two very separate clinical endpoints. Many studies have demonstrated an association between genotype and developing sepsis but not outcome from sepsis [Gordon A C et al, Mannose-binding lectin polymorphisms in severe sepsis; relationship to levels, incidence and outcome Shock 2006; 25 (1) 88-93.] and similarly vice versa [Westendorp R G et al, Variation in plasminogen-activator-inhibitor-1 gene and risk of meningococcal septic shock. Lancet 1999; 354: 561-63]. It has also been shown that the same SNP may have different effects at different stages of the inflammatory response [Mancoha S et al. TNF□ +252 A: TNF□ −308 G haplotype has a different effect on outcome in patients with SIRS, sepsis and septic shock. Critical Care Medicine 2003; 31(12 Supplement):A3.]. This may be due to the dynamic nature of the inflammatory and anti-inflammatory responses in sepsis. In fact, an excessive inflammatory or an excessive anti-inflammatory response may be harmful or beneficial at different timepoints [Bone R C. Sir Isaac Newton, sepsis, SIRS, and CARS. Critical Care Medicine 1996; 24:1125-1128].

Linkage disequilibrium (LD) has been reported between several polymorphisms in the interferon gamma gene. The IFNG.66838790.(CA)n intron 1 microsatellite was first identified in 1982 by GRAY and GOULD (Nature. (1982) 298:859-863). PRAVICA et al. (Eur J Immunogenet. (1999) 26:1-3) report polymorphisms at the IFNG.66838790.(CA)n microsatellite which correlate with in vitro production of interferon gamma and later (PRAVICA V. et al. Hum Immunol. (2000) 61:863-866) reported an association between the IFNG.66838790.(CA)12 allele and the T allele of IFNG.66838789.T/A in a UK population (n=50 PCR products). Recently, T S O et al. (Genes Immun. (2005) 6(4):358-63) reported an association between IFNG.66838790.(CA)12 allele and IFNG.66838789.T allele in a Chinese population (n=796 individuals). Further IFNG linkage analysis has been reported (KOCH O. et al. Genes Immun. (2005) 6, 312-318; KANTARCI et al. Genes Immun. (2005) 6(2):153-61; and NATIVIDAD et al. Genes Immun. (2005) 6(4):332-40).

### SUMMARY OF THE INVENTION

This invention is based in part on the surprising discovery that interferon gamma (IFNG) SNPs are predictive or indicative of subject outcome, wherein subject outcome is the ability of the subject to recover from an inflammatory condition based on having a particular IFNG genotype as compared to a subject not having that genotype.

This invention is also based in part on the surprising discovery of IFNG SNPs having an association with improved prognosis or subject outcome, in subjects with an inflammatory condition. Furthermore, various IFNG SNPs are provided which may be useful for subject screening, as an indication of subject outcome, or for prognosis for recovery from an inflammatory condition.

This invention is also based in part on the identification the particular nucleotide (allele) at the site of a given SNP may be associated with a decreased likelihood of recovery from an inflammatory condition (‘risk genotype’ or “adverse response genotype” (ARG)) or an increased likelihood of recovery from an inflammatory condition (‘decreased risk genotype’ or “improved response genotype” (IRG)). Furthermore, this invention is in part based on the discovery that the risk genotype or allele may be predictive of increased responsiveness to the treatment of the inflammatory condition with activated protein C or protein C like compound.

This invention is also based in part on the surprising discovery that IFNG SNPs alone or in combination are useful in predicting the response a subject with an inflammatory condition will have to activated protein C or protein C like compound treatment. Whereby the subjects having an improved response genotype are more likely to benefit from and have an improved response to activated protein C or protein C like compound treatment and subjects having a non-improved response genotype are less likely to benefit from the same treatment. Furthermore, there are provided herein IFNG SNPs and SNPs in linkage disequilibrium thereto, which are also useful in predicting the response a subject with an inflammatory condition will have to activated protein C or protein C like compound treatment.

In accordance with one aspect of the invention, methods are provided for obtaining a prognosis for a subject having, or at risk of developing, an inflammatory condition, the method including determining a genotype of said subject which includes one or more polymorphic sites in the subject's IFNG sequence, wherein said genotype is indicative of an ability of the subject to recover from the inflammatory condition. The method may further involve determination of the genotype for one or more polymorphic sites in the IFNG gene sequences for the subject. The genotypes at particular SNPs of the IFNG sequence may be taken alone or in combination.

In accordance with a further aspect of the invention, a method is provided for obtaining a prognosis for a subject having, or at risk of developing, an inflammatory condition, the method comprising determining a genotype of said subject which includes one or more polymorphic sites in the subject's interferon gamma (IFNG) gene sequence selected from one or more of the following: rs1861493; rs2069718; and rs2069727 or one or more polymorphic sites in linkage disequilibrium thereto, wherein said genotype is indicative of an ability of the subject to recover from the inflammatory condition.

Oligonucleotides or peptide nucleic acids, arrays, addressable collections of oligonucleotides or peptide nucleic acids and a computer readable medium including a plurality of digitally encoded genotype correlations are provided as described herein. There may be may be two or more oligonucleotides or peptide nucleic acids. Alternatively, there may be three or more oligonucleotides or peptide nucleic acids, four or more oligonucleotides or peptide nucleic acids or five or more oligonucleotides or peptide nucleic acids, or six or more oligonucleotides or peptide nucleic acids, or seven or more oligonucleotides or peptide nucleic acids, or eight or more oligonucleotides or peptide nucleic acids, or nine or more oligonucleotides or peptide nucleic acids or ten or more oligonucleotides or peptide nucleic acids.

Sequence variations may be assigned to a gene if mapped within 2 kb or more of an mRNA sequence feature.

In accordance with a further aspect of the invention, a method is provided for obtaining a prognosis for a subject having, or at risk of developing, an inflammatory condition, the method including determining a genotype of said subject which includes one or more polymorphic sites in the subject's interferon gamma (IFNG) gene sequence, wherein said genotype is indicative of an ability of the subject to recover from the inflammatory condition.

The one or more polymorphic sites in linkage disequilibrium thereto may be selected from one or more of the following: rs2069705; rs2069733; rs10467155; rs7973244; rs7137993; rs12315837; rs4913277; rs2080414; rs7956817; rs2069718; rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763; rs2193046; rs1861493; rs10878774; rs10878786; rs10878784; rs971545; rs12301088; rs7969024; rs11177081; rs12317232; rs11177083; rs10878766; rs7969592; rs10878781; rs2870950; and rs10492197. The method may further include comparing the genotype so determined with known genotypes which are known to be indicative of a prognosis for recovery from: the subject's type of inflammatory condition; or another inflammatory condition. The method may further include obtaining IFNG gene sequence information for the subject.

Genotype may be determined using a nucleic acid sample from the subject. The method may further include obtaining the nucleic acid sample from the subject. The genotype may be determined using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data.

The genotype of the subject may be indicative of increased risk of death or organ dysfunction from the inflammatory condition. The genotype may be indicative of a prognosis of severe cardiovascular or respiratory dysfunction. The genotype may be selected from the following risk genotypes: rs2069705C; rs2069727A; rs2069733-; rs2069718T; rs1861494C; and rs1861493G or one or more polymorphic sites in linkage disequilibrium thereto.

The genotype of the subject may be indicative of decreased risk of death or organ dysfunction from the inflammatory condition. The genotype may be indicative of a prognosis of mild cardiovascular or respiratory dysfunction. The genotype may be selected from the following reduced risk genotypes: rs2069705T; rs2069727G; rs2069733G; rs2069718C; rs1861494T; and rs1861493A or one or more polymorphic sites in linkage disequilibrium thereto.

The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis. The inflammatory condition may be SIRS. The inflammatory condition may be sepsis. The inflammatory condition may be septic shock.

In accordance with a further aspect of the invention, a method is provided for identifying a polymorphism in a IFNG gene sequence that correlates with prognosis of recovery from an inflammatory condition, the method including: (a) obtaining IFNG gene sequence information from a group of subjects having an inflammatory condition; (b) identifying at least one polymorphic nucleotide position in the IFNG gene sequence in the subjects; (c) determining a genotypes at the polymorphic site for individual subjects in the group; (d) determining recovery capabilities of individual subjects in the group from the inflammatory condition; and (e) correlating the genotypes determined in step (c) with the recovery capabilities determined in step (d) thereby identifying said IFNG gene sequence polymorphisms that correlate with recovery.

In accordance with a further aspect of the invention, a method is provided for identifying a subject having an improved response genotype (IRG) in a interferon gamma (IFNG) gene sequence, the method including determining a genotype of said subject at one or more polymorphic sites in the subject's IFNG gene sequence, wherein said genotype is indicative of the subject's response to activated protein C or protein C like compound administration.

The polymorphic site may be rs2069718 or one or more polymorphic sites in linkage disequilibrium thereto. The improved response genotype may be rs2069718C or one or more polymorphic sites in linkage disequilibrium thereto. The one or more polymorphic sites in linkage disequilibrium thereto may be selected from one or more of the following polymorphic sites: rs2069705; rs2069733; rs2069727; rs1861493; rs10467155; rs7973244; rs7137993; rs12315837; rs4913277; rs2080414; rs7956817; rs2069718; rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763; rs2193046; rs1861493; rs10878774; rs10878786; rs10878784; rs971545; rs12301088; rs7969024; rs11177081; rs12317232; rs11177083; rs10878766; rs7969592; rs10878781; rs2870950; and rs10492197.

The method may further include comparing the genotype so determined with known genotypes which are known to be indicative of the subject's response to activated protein C or protein C like compound administration.

The method may further include obtaining IFNG gene sequence information for the subject. The genotype may be determined using a nucleic acid sample from the subject. The method may further include obtaining the nucleic acid sample from the subject.

Genotype of the subject may indicative of the subject's response to activated protein C or protein C like compound administration. The subject may be critically ill with an inflammatory condition.

The method may further include selectively administering activated protein C or protein C like compound to a subject having one or more improved response genotype(s) in their IFNG gene sequences.

The method may further include selectively not administering activated protein C or protein C like compound to a subject not having one or more improved response genotype(s) in their IFNG gene.

In accordance with a further aspect of the invention, a method is provided for identifying a polymorphism in a IFNG gene sequence that correlates with an improved response to activated protein C or protein C like compound administration, the method including: (a) obtaining IFNG gene sequence information from a group of subjects having an inflammatory condition; (b) identifying at least one polymorphic nucleotide position in the IFNG gene sequence in the subjects; (c) determining a genotype at the polymorphic site for individual subjects in the group; (d) determining response to activated protein C or protein C like compound administration; and (e) correlating the genotypes determined in step (c) with the response to activated protein C or protein C like compound administration in step (d) thereby identifying said IFNG gene sequence polymorphisms that correlate with response to activated protein C or protein C like compound administration.

In accordance with a further aspect of the invention, a kit for determining a genotype at a defined nucleotide position within a polymorphic site in a IFNG gene sequence in a subject to predict a subject's response to activated protein C or protein C like compound administration, the kit including: (a) a restriction enzyme capable of distinguishing alternate nucleotides at the polymorphic site; or (b) a labeled oligonucleotide having sufficient complementary to the polymorphic site so as to be capable of hybridizing distinctively to said alternate.

The polymorphic site may be selected from one or more of the following: rs2069705; rs2069727; rs2069733; rs2069718; rs1861494; rs1861493; rs10467155; rs7973244; rs7137993; rs12315837; rs4913277; rs2080414; rs7956817; rs2069718; rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763; rs2193046; rs1861493; rs10878774; rs10878786; rs10878784; rs971545; rs12301088; rs7969024; rs11177081; rs12317232; rs11177083; rs10878766; rs7969592; rs10878781; rs2870950; and rs10492197.

The kit may further include an oligonucleotide or a set of oligonucleotides operable to amplify a region including the polymorphic site. The kit may further include a polymerization agent. The kit may further include instructions for using the kit to determine genotype.

In accordance with a further aspect of the invention, a method is provided for selecting a group of subjects for determining the efficacy of a candidate drug known or suspected of being useful for the treatment of an inflammatory condition, the method including determining a genotype at one or more polymorphic sites in a IFNG gene sequence for each subject, wherein said genotype is indicative of the subject's response to the candidate drug and sorting subjects based on their genotype. The method may further include, administering the candidate drug to the subjects or a subset of subjects and determining each subject's ability to recover from the inflammatory condition. The method may further include comparing subject response to the candidate drug based on genotype of the subject.

In accordance with a further aspect of the invention, a method is provided for treating an inflammatory condition in a subject in need thereof, the method including administering to the subject activated protein C or protein C like compound, wherein said subject has an improved response genotype in their IFNG gene sequence.

In accordance with a further aspect of the invention, a method is provided for treating an inflammatory condition in a subject in need thereof, the method including: selecting a subject having an improved response genotype in their IFNG gene sequence; and administering to said subject activated protein C or protein C like compound.

In accordance with a further aspect of the invention, a method is provided for treating a subject with an inflammatory condition by administering activated protein C, the method including administering the activated protein C or protein C like compound to subjects that have an improved response genotype in their IFNG gene sequence, wherein the improved response genotype is predictive of increased responsiveness to the treatment of the inflammatory condition with activated protein C or protein C like compound.

In accordance with a further aspect of the invention, a method is provided for identifying a subject with increased responsiveness to treatment of an inflammatory condition with activated protein C or protein C like compound, including the step of screening a population of subjects to identify those subjects that have an improved response genotype in their IFNG gene sequence, wherein the identification of a subject with an improved response genotype in their IFNG gene sequence is predictive of increased responsiveness to the treatment of the inflammatory condition with the activated protein C or protein C like compound.

In accordance with a further aspect of the invention, a method is provided for selecting a subject for the treatment of an inflammatory condition with an activated protein C or protein C like compound, including the step of identifying a subject having an improved response genotype in their IFNG gene sequence, wherein the identification of a subject with the improved response genotype is predictive of increased responsiveness to the treatment of the inflammatory condition with the activated protein C or protein C like compound.

In accordance with a further aspect of the invention, a method is provided for treating an inflammatory condition in a subject, the method including administering an activated protein C or protein C like compound to the subject, wherein said subject has an improved response genotype in their IFNG gene sequence.

In accordance with a further aspect of the invention, a method is provided for treating an inflammatory condition in a subject, the method including: identifying a subject having an improved response genotype in their IFNG gene sequence; and administering activated protein C or protein C like compound to the subject.

In accordance with a further aspect of the invention, a use of an activated protein C or protein C like compound in the manufacture of a medicament for the treatment of an inflammatory condition is provided, wherein the subjects treated have an improved response genotype in their IFNG gene sequence.

In accordance with a further aspect of the invention, a use of an activated protein C or protein C like compound in the manufacture of a medicament for the treatment of an inflammatory condition in a subset of subjects is provided, wherein the subset of subjects have an improved response genotype in their IFNG gene sequence.

The method or use may further include determining the subject's APACHE II score as an assessment of subject risk. The method or use may further include determining the number of organ system failures for the subject as an assessment of subject risk.

The subject's APACHE II score may be indicative of an increased risk when ≧25. 2 or more organ system failures may be indicative of increased subject risk.

The inflammatory condition may be systemic inflammatory response syndrome. The inflammatory condition may be sepsis. The inflammatory condition may be septic shock.

The polymorphic site may be selected from one or more of the following: rs2069705; rs2069727; rs2069733; rs2069718; rs1861494; rs1861493; rs10467155; rs7973244; rs7137993; rs12315837; rs4913277; rs2080414; rs7956817; rs2069718; rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763; rs2193046; rs1861493; rs10878774; rs10878786; rs10878784; rs971545; rs12301088; rs7969024; rs11177081; rs12317232; rs11177083; rs10878766; rs7969592; rs10878781; rs2870950; and rs10492197.

The improved response genotype may be selected from one or more of the following: rs2069705T; rs2069727G; rs2069733G; rs2069718C; rs1861494T; and rs1861493A or a genotype in linkage disequilibrium thereto. The activated protein C or protein C like compound may be drotecogin alfa activated.

In accordance with a further aspect of the invention, there are provided two or more oligonucleotides or peptide nucleic acids of about 10 to about 400 nucleotides that hybridize specifically to a sequence contained in a human target sequence consisting of a subject's IFNG gene sequence, a complementary sequence of the target sequence or RNA equivalent of the target sequence and wherein the oligonucleotides or peptide nucleic acids are operable in determining the presence or absence of two or more improved response genotype(s) in their IFNG gene sequence selected from of the following polymorphic sites: rs2069705; rs2069727; rs2069733; rs2069718; rs1861494; rs1861493; rs10467155; rs7973244; rs7137993; rs12315837; rs4913277; rs2080414; rs7956817; rs2069718; rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763; rs2193046; rs1861493; rs10878774; rs10878786; rs10878784; rs971545; rs12301088; rs7969024; rs11177081; rs12317232; rs11177083; rs10878766; rs7969592; rs10878781; rs2870950; and rs10492197.

The improved response genotype may be selected from one or more of the following: rs2069705T; rs2069727G; rs2069733G; rs2069718C; rs1861494T; and rs1861493A or a genotype in linkage disequilibrium thereto.

In accordance with a further aspect of the invention, there are provided two or more oligonucleotides or peptide nucleic acids selected from the group consisting of:

(a) an oligonucleotide or peptide nucleic acid that hybridizes under high stringency conditions to a nucleic acid molecule including SEQ ID NO:1 having a G at position 260 but not to a nucleic acid molecule including SEQ ID NO:1 having an A at position 260;

(b) an oligonucleotide or peptide nucleic acid that hybridizes under high stringency conditions to a nucleic acid molecule including SEQ ID NO:1 having an A at position 260 but not to a nucleic acid molecule including SEQ ID NO:1 having a G at position 260;

(c) an oligonucleotide or peptide nucleic acid that hybridizes under high stringency conditions to a nucleic acid molecule including SEQ ID NO:2 having a T at position 201 but not to a nucleic acid molecule including SEQ ID NO:2 having a C at position 201;

(d) an oligonucleotide or peptide nucleic acid that hybridizes under high stringency conditions to a nucleic acid molecule including SEQ ID NO:2 having an C at position 201 but not to a nucleic acid molecule including SEQ ID NO:2 having a T at position 201;

(e) an oligonucleotide or peptide nucleic acid that hybridizes under high stringency conditions to a nucleic acid molecule including SEQ ID NO:3 having an A at position 201 but not to a nucleic acid molecule including SEQ ID NO:3 having a G at position 201;

(f) an oligonucleotide or peptide nucleic acid that hybridizes under high stringency conditions to a nucleic acid molecule including SEQ ID NO:3 having a G at position 201 but not to a nucleic acid molecule including SEQ ID NO:3 having an A at position 201;

(g) an oligonucleotide or peptide nucleic acid that hybridizes under high stringency conditions to a nucleic acid molecule including SEQ ID NO:4 having a T at position 473 but not to a nucleic acid molecule including SEQ ID NO:4 having a C at position 473;

(h) an oligonucleotide or peptide nucleic acid that hybridizes under high stringency conditions to a nucleic acid molecule including SEQ ID NO:4 having a C at position 473 but not to a nucleic acid molecule including SEQ ID NO:4 having a T at position 473;

(i) an oligonucleotide or peptide nucleic acid that hybridizes under high stringency conditions to a nucleic acid molecule including SEQ ID NO:5 having a T at position 709 but not to a nucleic acid molecule including SEQ ID NO:5 having a C at position 709;

(j) an oligonucleotide or peptide nucleic acid that hybridizes under high stringency conditions to a nucleic acid molecule including SEQ ID NO:5 having a C at position 709 but not to a nucleic acid molecule including SEQ ID NO:5 having a T at position 709;

(k) an oligonucleotide or peptide nucleic acid that hybridizes under high stringency conditions to a nucleic acid molecule including SEQ ID NO:6 having a G at position 402 but not to a nucleic acid molecule including SEQ ID NO:6 having a T at position 402;

(l) an oligonucleotide or peptide nucleic acid that hybridizes under high stringency conditions to a nucleic acid molecule including SEQ ID NO:6 having a T at position 402 but not to a nucleic acid molecule including SEQ ID NO:6 having a G at position 402;

(m) an oligonucleotide or peptide nucleic acid that hybridizes under high stringency conditions to a nucleic acid molecule comprising SEQ ID NO:7 having a C at position 734 but not to a nucleic acid molecule comprising SEQ ID NO:7 having a T at position 734;

(n) an oligonucleotide or peptide nucleic acid that hybridizes under high stringency conditions to a nucleic acid molecule comprising SEQ ID NO:7 having a T at position 734 but not to a nucleic acid molecule comprising SEQ ID NO:7 having a C at position 734;

(o) an oligonucleotide or peptide nucleic acid that hybridizes under high stringency conditions to a nucleic acid molecule comprising SEQ ID NO:8 having a C at position 201 but not to a nucleic acid molecule comprising SEQ ID NO:8 having a T at position 201;

(p) an oligonucleotide or peptide nucleic acid that hybridizes under high stringency conditions to a nucleic acid molecule comprising SEQ ID NO:8 having a T at position 201 but not to a nucleic acid molecule comprising SEQ ID NO:8 having a C at position 201;

(q) an oligonucleotide or peptide nucleic acid that hybridizes under high stringency conditions to a nucleic acid molecule comprising SEQ ID NO:9 having a C at position 278 but not to a nucleic acid molecule comprising SEQ ID NO:9 having a T at position 278;

(r) an oligonucleotide or peptide nucleic acid that hybridizes under high stringency conditions to a nucleic acid molecule comprising SEQ ID NO:9 having a T at position 278 but not to a nucleic acid molecule comprising SEQ ID NO:9 having a C at position 278;

(s) an oligonucleotide or peptide nucleic acid that hybridizes under high stringency conditions to a nucleic acid molecule comprising SEQ ID NO:10 having a G at position 501 but not to a nucleic acid molecule comprising SEQ ID NO:10 having an A at position 501;

(t) an oligonucleotide or peptide nucleic acid that hybridizes under high stringency conditions to a nucleic acid molecule comprising SEQ ID NO:10 having an A at position 501 but not to a nucleic acid molecule comprising SEQ ID NO:10 having a G at position 501;

(u) an oligonucleotide or peptide nucleic acid that hybridizes under high stringency conditions to a nucleic acid molecule comprising SEQ ID NO:11 having a G at position 201 but not to a nucleic acid molecule comprising SEQ ID NO:11 having an A at position 201;

(v) an oligonucleotide or peptide nucleic acid that hybridizes under high stringency conditions to a nucleic acid molecule comprising SEQ ID NO:11 having an A at position 201 but not to a nucleic acid molecule comprising SEQ ID NO:11 having a G at position 201;

(w) an oligonucleotide or peptide nucleic acid that hybridizes under high stringency conditions to a nucleic acid molecule comprising SEQ ID NO:12 having a C at position 1303 but not to a nucleic acid molecule comprising SEQ ID NO:12 having a T at position 1303;

(x) an oligonucleotide or peptide nucleic acid that hybridizes under high stringency conditions to a nucleic acid molecule comprising SEQ ID NO:12 having a T at position 1303 but not to a nucleic acid molecule comprising SEQ ID NO:12 having a C at position 1303;

(y) an oligonucleotide or peptide nucleic acid that hybridizes under high stringency conditions to a nucleic acid molecule comprising SEQ ID NO:13 having a C at position 304 but not to a nucleic acid molecule comprising SEQ ID NO:13 having a T at position 304;

(z) an oligonucleotide or peptide nucleic acid that hybridizes under high stringency conditions to a nucleic acid molecule comprising SEQ ID NO:13 having a T at position 304 but not to a nucleic acid molecule comprising SEQ ID NO:13 having a C at position 304;

(aa) an oligonucleotide or peptide nucleic acid that hybridizes under high stringency conditions to a nucleic acid molecule comprising SEQ ID NO:14 having a G at position 1958 but not to a nucleic acid molecule comprising SEQ ID NO:14 having a T at position 1958;

(bb) an oligonucleotide or peptide nucleic acid that hybridizes under high stringency conditions to a nucleic acid molecule comprising SEQ ID NO:14 having a T at position 1958 but not to a nucleic acid molecule comprising SEQ ID NO:14 having a G at position 1958;

(cc) an oligonucleotide or peptide nucleic acid that hybridizes under high stringency conditions to a nucleic acid molecule comprising SEQ ID NO:15 having a G at position 272 but not to a nucleic acid molecule comprising SEQ ID NO:15 having a T at position 272;

(dd) an oligonucleotide or peptide nucleic acid that hybridizes under high stringency conditions to a nucleic acid molecule comprising SEQ ID NO:15 having a T at position 272 but not to a nucleic acid molecule comprising SEQ ID NO:15 having a G at position 272;

(ee) an oligonucleotide or peptide nucleic acid that hybridizes under high stringency conditions to a nucleic acid molecule comprising SEQ ID NO:16 having a G at position 201 but not to a nucleic acid molecule comprising SEQ ID NO:16 having an A at position 201;

(ff) an oligonucleotide or peptide nucleic acid that hybridizes under high stringency conditions to a nucleic acid molecule comprising SEQ ID NO:16 having an A at position 201 but not to a nucleic acid molecule comprising SEQ ID NO:16 having a G at position 201;

(gg) an oligonucleotide or peptide nucleic acid that hybridizes under high stringency conditions to a nucleic acid molecule comprising SEQ ID NO:17 having a C at position 501 but not to a nucleic acid molecule comprising SEQ ID NO:17 having a T at position 501;

(hh) an oligonucleotide or peptide nucleic acid that hybridizes under high stringency conditions to a nucleic acid molecule comprising SEQ ID NO:17 having a T at position 501 but not to a nucleic acid molecule comprising SEQ ID NO:17 having a C at position 501;

(ii) an oligonucleotide or peptide nucleic acid that hybridizes under high stringency conditions to a nucleic acid molecule comprising SEQ ID NO:18 having a G at position 301 but not to a nucleic acid molecule comprising SEQ ID NO:18 having an A at position 301;

(jj) an oligonucleotide or peptide nucleic acid that hybridizes under high stringency conditions to a nucleic acid molecule comprising SEQ ID NO:18 having an A at position 301 but not to a nucleic acid molecule comprising SEQ ID NO:18 having a G at position 301;

(kk) an oligonucleotide or peptide nucleic acid that hybridizes under high stringency conditions to a nucleic acid molecule comprising SEQ ID NO:19 having a G at position 368 but not to a nucleic acid molecule comprising SEQ ID NO:19 having a T at position 368;

(ll) an oligonucleotide or peptide nucleic acid that hybridizes under high stringency conditions to a nucleic acid molecule comprising SEQ ID NO:19 having a T at position 368 but not to a nucleic acid molecule comprising SEQ ID NO:19 having a G at position 368;

(mm) an oligonucleotide or peptide nucleic acid that hybridizes under high stringency conditions to a nucleic acid molecule comprising SEQ ID NO:20 having a G at position 284 but not to a nucleic acid molecule comprising SEQ ID NO:20 having an A at position 284;

(nn) an oligonucleotide or peptide nucleic acid that hybridizes under high stringency conditions to a nucleic acid molecule comprising SEQ ID NO:20 having an A at position 284 but not to a nucleic acid molecule comprising SEQ ID NO:20 having a G at position 284;

(oo) an oligonucleotide or peptide nucleic acid that hybridizes under high stringency conditions to a nucleic acid molecule comprising SEQ ID NO:21 having a G at position 301 but not to a nucleic acid molecule comprising SEQ ID NO:21 having a T at position 301;

(pp) an oligonucleotide or peptide nucleic acid that hybridizes under high stringency conditions to a nucleic acid molecule comprising SEQ ID NO:21 having a T at position 301 but not to a nucleic acid molecule comprising SEQ ID NO:21 having a G at position 301;

(qq) an oligonucleotide or peptide nucleic acid that hybridizes under high stringency conditions to a nucleic acid molecule comprising SEQ ID NO:22 having a C at position 272 but not to a nucleic acid molecule comprising SEQ ID NO:22 having a T at position 272;

(rr) an oligonucleotide or peptide nucleic acid that hybridizes under high stringency conditions to a nucleic acid molecule comprising SEQ ID NO:22 having a T at position 272 but not to a nucleic acid molecule comprising SEQ ID NO:22 having a C at position 272;

(ss) an oligonucleotide or peptide nucleic acid that hybridizes under high stringency conditions to a nucleic acid molecule comprising SEQ ID NO:23 having a C at position 256 but not to a nucleic acid molecule comprising SEQ ID NO:23 having a T at position 256;

(tt) an oligonucleotide or peptide nucleic acid that hybridizes under high stringency conditions to a nucleic acid molecule comprising SEQ ID NO:23 having a T at position 256 but not to a nucleic acid molecule comprising SEQ ID NO:23 having a C at position 256;

(uu) an oligonucleotide or peptide nucleic acid that hybridizes under high stringency conditions to a nucleic acid molecule comprising SEQ ID NO:24 having a C at position 301 but not to a nucleic acid molecule comprising SEQ ID NO:24 having a T at position 301;

(vv) an oligonucleotide or peptide nucleic acid that hybridizes under high stringency conditions to a nucleic acid molecule comprising SEQ ID NO:24 having a T at position 301 but not to a nucleic acid molecule comprising SEQ ID NO:24 having a C at position 301;

(ww) an oligonucleotide or peptide nucleic acid that hybridizes under high stringency conditions to a nucleic acid molecule comprising SEQ ID NO:25 having a G at position 501 but not to a nucleic acid molecule comprising SEQ ID NO:25 having an A at position 501;

(xx) an oligonucleotide or peptide nucleic acid that hybridizes under high stringency conditions to a nucleic acid molecule comprising SEQ ID NO:25 having an A at position 501 but not to a nucleic acid molecule comprising SEQ ID NO:25 having a G at position 501;

(yy) an oligonucleotide or peptide nucleic acid that hybridizes under high stringency conditions to a nucleic acid molecule comprising SEQ ID NO:26 having an A at position 501 but not to a nucleic acid molecule comprising SEQ ID NO:26 having a C at position 501;

(zz) an oligonucleotide or peptide nucleic acid that hybridizes under high stringency conditions to a nucleic acid molecule comprising SEQ ID NO:26 having a C at position 501 but not to a nucleic acid molecule comprising SEQ ID NO:26 having an A at position 501;

(aaa) an oligonucleotide or peptide nucleic acid that hybridizes under high stringency conditions to a nucleic acid molecule comprising SEQ ID NO:27 having an A at position 501 but not to a nucleic acid molecule comprising SEQ ID NO:27 having a C at position 501;

(bbb) an oligonucleotide or peptide nucleic acid that hybridizes under high stringency conditions to a nucleic acid molecule comprising SEQ ID NO:27 having a C at position 501 but not to a nucleic acid molecule comprising SEQ ID NO:27 having an A at position 501;

(ccc) an oligonucleotide or peptide nucleic acid that hybridizes under high stringency conditions to a nucleic acid molecule comprising SEQ ID NO:28 having a C at position 1083 but not to a nucleic acid molecule comprising SEQ ID NO:28 having a T at position 1083;

(ddd) an oligonucleotide or peptide nucleic acid that hybridizes under high stringency conditions to a nucleic acid molecule comprising SEQ ID NO:28 having a T at position 1083 but not to a nucleic acid molecule comprising SEQ ID NO:28 having a C at position 1083;

(eee) an oligonucleotide or peptide nucleic acid that hybridizes under high stringency conditions to a nucleic acid molecule comprising SEQ ID NO:29 having a C at position 349 but not to a nucleic acid molecule comprising SEQ ID NO:29 having a T at position 349;

(fff) an oligonucleotide or peptide nucleic acid that hybridizes under high stringency conditions to a nucleic acid molecule comprising SEQ ID NO:29 having a T at position 349 but not to a nucleic acid molecule comprising SEQ ID NO:29 having a C at position 349;

(ggg) an oligonucleotide or peptide nucleic acid that hybridizes under high stringency conditions to a nucleic acid molecule comprising SEQ ID NO:30 having a G at position 201 but not to a nucleic acid molecule comprising SEQ ID NO:30 having an A at position 201;

(hhh) an oligonucleotide or peptide nucleic acid that hybridizes under high stringency conditions to a nucleic acid molecule comprising SEQ ID NO:30 having an A at position 201 but not to a nucleic acid molecule comprising SEQ ID NO:30 having a G at position 201;

(iii) an oligonucleotide or peptide nucleic acid that hybridizes under high stringency conditions to a nucleic acid molecule comprising SEQ ID NO:31 having an A at position 295 but not to a nucleic acid molecule comprising SEQ ID NO:31 having a T at position 295;

(jjj) an oligonucleotide or peptide nucleic acid that hybridizes under high stringency conditions to a nucleic acid molecule comprising SEQ ID NO:31 having a T at position 295 but not to a nucleic acid molecule comprising SEQ ID NO:31 having an A at position 295;

(kkk) an oligonucleotide or peptide nucleic acid that hybridizes under high stringency conditions to a nucleic acid molecule comprising SEQ ID NO:32 having an A at position 259 but not to a nucleic acid molecule comprising SEQ ID NO:32 having a C at position 259;

(lll) an oligonucleotide or peptide nucleic acid that hybridizes under high stringency conditions to a nucleic acid molecule comprising SEQ ID NO:32 having a C at position 259 but not to a nucleic acid molecule comprising SEQ ID NO:32 having an A at position 259;

(mmm) an oligonucleotide or peptide nucleic acid that hybridizes under high stringency conditions to a nucleic acid molecule comprising SEQ ID NO:33 having a G at position 1060 but not to a nucleic acid molecule comprising SEQ ID NO:33 having an A at position 1060;

(nnn) an oligonucleotide or peptide nucleic acid that hybridizes under high stringency conditions to a nucleic acid molecule comprising SEQ ID NO:33 having an A at position 1060 but not to a nucleic acid molecule comprising SEQ ID NO:33 having a G at position 1060;

(ooo) an oligonucleotide or peptide nucleic acid that hybridizes under high stringency conditions to a nucleic acid molecule comprising SEQ ID NO:34 having a C at position 256 but not to a nucleic acid molecule comprising SEQ ID NO:34 having a T at position 256;

(ppp) an oligonucleotide or peptide nucleic acid that hybridizes under high stringency conditions to a nucleic acid molecule comprising SEQ ID NO:34 having a T at position 256 but not to a nucleic acid molecule comprising SEQ ID NO:34 having a C at position 256;

(qqq) an oligonucleotide or peptide nucleic acid that hybridizes under high stringency conditions to a nucleic acid molecule comprising SEQ ID NO:35 having a G at position 265 but not to a nucleic acid molecule comprising SEQ ID NO:35 having an A at position 265;

(rrr) an oligonucleotide or peptide nucleic acid that hybridizes under high stringency conditions to a nucleic acid molecule comprising SEQ ID NO:35 having an A at position 265 but not to a nucleic acid molecule comprising SEQ ID NO:35 having a G at position 265;

(sss) an oligonucleotide or peptide nucleic acid that hybridizes under high stringency conditions to a nucleic acid molecule comprising SEQ ID NO:36 having a C at position 530 but not to a nucleic acid molecule comprising SEQ ID NO:36 having a T at position 530;

(ttt) an oligonucleotide or peptide nucleic acid that hybridizes under high stringency conditions to a nucleic acid molecule comprising SEQ ID NO:36 having a T at position 530 but not to a nucleic acid molecule comprising SEQ ID NO:36 having a C at position 530;

(uuu) an oligonucleotide or peptide nucleic acid that hybridizes under high stringency conditions to a nucleic acid molecule comprising SEQ ID NO:37 having a C at position 297 but not to a nucleic acid molecule comprising SEQ ID NO:37 having a T at position 297;

(vvv) an oligonucleotide or peptide nucleic acid that hybridizes under high stringency conditions to a nucleic acid molecule comprising SEQ ID NO:37 having a T at position 297 but not to a nucleic acid molecule comprising SEQ ID NO:37 having a C at position 297;

(www) an oligonucleotide or peptide nucleic acid that hybridizes under high stringency conditions to a nucleic acid molecule comprising SEQ ID NO:38 having a C at position 543 but not to a nucleic acid molecule comprising SEQ ID NO:38 having a T at position 543;

(xxx) an oligonucleotide or peptide nucleic acid that hybridizes under high stringency conditions to a nucleic acid molecule comprising SEQ ID NO:38 having a T at position 543 but not to a nucleic acid molecule comprising SEQ ID NO:38 having a C at position 543;

(yyy) an oligonucleotide or peptide nucleic acid that hybridizes under high stringency conditions to a nucleic acid molecule comprising SEQ ID NO:39 having a G at position 223 but not to a nucleic acid molecule comprising SEQ ID NO:39 having a C at position 223;

(zzz) an oligonucleotide or peptide nucleic acid that hybridizes under high stringency conditions to a nucleic acid molecule comprising SEQ ID NO:39 having a C at position 223 but not to a nucleic acid molecule comprising SEQ ID NO:39 having a G at position 223;

(aaaa) an oligonucleotide or peptide nucleic acid that hybridizes under high stringency conditions to a nucleic acid molecule comprising SEQ ID NO:40 having a G at position 201 but not to a nucleic acid molecule comprising SEQ ID NO:40 having a T at position 201;

(bbbb) an oligonucleotide or peptide nucleic acid that hybridizes under high stringency conditions to a nucleic acid molecule comprising SEQ ID NO:40 having a T at position 201 but not to a nucleic acid molecule comprising SEQ ID NO:40 having a G at position 201;

(cccc) an oligonucleotide or peptide nucleic acid that hybridizes under high stringency conditions to a nucleic acid molecule comprising SEQ ID NO:41 having a C at position 112 but not to a nucleic acid molecule comprising SEQ ID NO:41 having a T at position 112;

(dddd) an oligonucleotide or peptide nucleic acid that hybridizes under high stringency conditions to a nucleic acid molecule comprising SEQ ID NO:41 having a T at position 112 but not to a nucleic acid molecule comprising SEQ ID NO:41 having a C at position 112;

(eeee) an oligonucleotide or peptide nucleic acid that hybridizes under high stringency conditions to a nucleic acid molecule comprising SEQ ID NO:42 having a G at position 85 but not to a nucleic acid molecule comprising SEQ ID NO:42 having an A at position 85;

(ffff) an oligonucleotide or peptide nucleic acid that hybridizes under high stringency conditions to a nucleic acid molecule comprising SEQ ID NO:42 having an A at position 85 but not to a nucleic acid molecule comprising SEQ ID NO:42 having a G at position 85;

(gggg) an oligonucleotide or peptide nucleic acid that hybridizes under high stringency conditions to a nucleic acid molecule comprising SEQ ID NO:43 having a C at position 422 but not to a nucleic acid molecule comprising SEQ ID NO:43 having a T at position 422;

(hhhh) an oligonucleotide or peptide nucleic acid that hybridizes under high stringency conditions to a nucleic acid molecule comprising SEQ ID NO:43 having a T at position 422 but not to a nucleic acid molecule comprising SEQ ID NO:43 having a C at position 422;

(iiii) an oligonucleotide or peptide nucleic acid that hybridizes under high stringency conditions to a nucleic acid molecule comprising SEQ ID NO:44 having a C at position 497 but not to a nucleic acid molecule comprising SEQ ID NO:44 having a T at position 497;

(jjjj) an oligonucleotide or peptide nucleic acid that hybridizes under high stringency conditions to a nucleic acid molecule comprising SEQ ID NO:44 having a T at position 497 but not to a nucleic acid molecule comprising SEQ ID NO:44 having a C at position 497;

(kkkk) an oligonucleotide or peptide nucleic acid that hybridizes under high stringency conditions to a nucleic acid molecule comprising SEQ ID NO:45 having a C at position 500 but not to a nucleic acid molecule comprising SEQ ID NO:45 having a T at position 500;

(llll) an oligonucleotide or peptide nucleic acid that hybridizes under high stringency conditions to a nucleic acid molecule comprising SEQ ID NO:45 having a T at position 500 but not to a nucleic acid molecule comprising SEQ ID NO:45 having a C at position 500;

(mmmm) an oligonucleotide or peptide nucleic acid that hybridizes under high stringency conditions to a nucleic acid molecule comprising SEQ ID NO:46 having an A at position 939 but not to a nucleic acid molecule comprising SEQ ID NO:46 having a T at position 939;

(nnnn) an oligonucleotide or peptide nucleic acid that hybridizes under high stringency conditions to a nucleic acid molecule comprising SEQ ID NO:46 having a T at position 939 but not to a nucleic acid molecule comprising SEQ ID NO:46 having an A at position 939;

(oooo) an oligonucleotide or peptide nucleic acid that hybridizes under high stringency conditions to a nucleic acid molecule comprising SEQ ID NO:47 having a G at position 301 but not to a nucleic acid molecule comprising SEQ ID NO:47 having an A at position 301;

(pppp) an oligonucleotide or peptide nucleic acid that hybridizes under high stringency conditions to a nucleic acid molecule comprising SEQ ID NO:47 having an A at position 301 but not to a nucleic acid molecule comprising SEQ ID NO:47 having a G at position 301;

(qqqq) an oligonucleotide or peptide nucleic acid that hybridizes under high stringency conditions to a nucleic acid molecule comprising SEQ ID NO:48 having a C at position 501 but not to a nucleic acid molecule comprising SEQ ID NO:48 having a T at position 501;

(rrrr) an oligonucleotide or peptide nucleic acid that hybridizes under high stringency conditions to a nucleic acid molecule comprising SEQ ID NO:48 having a T at position 501 but not to a nucleic acid molecule comprising SEQ ID NO:48 having a C at position 501;

(ssss) an oligonucleotide or peptide nucleic acid that hybridizes under high stringency conditions to a nucleic acid molecule comprising SEQ ID NO:49 having a C at position 1311 but not to a nucleic acid molecule comprising SEQ ID NO:49 having a T at position 1311;

(tttt) an oligonucleotide or peptide nucleic acid that hybridizes under high stringency conditions to a nucleic acid molecule comprising SEQ ID NO:49 having a T at position 1311 but not to a nucleic acid molecule comprising SEQ ID NO:49 having a C at position 1311;

(uuuu) an oligonucleotide or peptide nucleic acid that hybridizes under high stringency conditions to a nucleic acid molecule comprising SEQ ID NO:50 having a G at position 1307 but not to a nucleic acid molecule comprising SEQ ID NO:50 having an A at position 1307;

(vvvv) an oligonucleotide or peptide nucleic acid that hybridizes under high stringency conditions to a nucleic acid molecule comprising SEQ ID NO:50 having an A at position 1307 but not to a nucleic acid molecule comprising SEQ ID NO:50 having a G at position 1307;

(wwww) an oligonucleotide or peptide nucleic acid that hybridizes under high stringency conditions to a nucleic acid molecule comprising SEQ ID NO:51 having a G at position 288 but not to a nucleic acid molecule comprising SEQ ID NO:51 having an A at position 288;

(xxxx) an oligonucleotide or peptide nucleic acid that hybridizes under high stringency conditions to a nucleic acid molecule comprising SEQ ID NO:51 having an A at position 288 but not to a nucleic acid molecule comprising SEQ ID NO:51 having a G at position 288;

(yyyy) an oligonucleotide or peptide nucleic acid that hybridizes under high stringency conditions to a nucleic acid molecule comprising SEQ ID NO:52 having a G at position 301 but not to a nucleic acid molecule comprising SEQ ID NO:52 having an A at position 301;

(zzzz) an oligonucleotide or peptide nucleic acid that hybridizes under high stringency conditions to a nucleic acid molecule comprising SEQ ID NO:52 having an A at position 301 but not to a nucleic acid molecule comprising SEQ ID NO:52 having a G at position 301;

(aaaaa) an oligonucleotide or peptide nucleic acid that hybridizes under high stringency conditions to a nucleic acid molecule comprising SEQ ID NO:53 having a C at position 354 but not to a nucleic acid molecule comprising SEQ ID NO:53 having a T at position 354;

(bbbbb) an oligonucleotide or peptide nucleic acid that hybridizes under high stringency conditions to a nucleic acid molecule comprising SEQ ID NO:53 having a T at position 354 but not to a nucleic acid molecule comprising SEQ ID NO:53 having a C at position 354;

(ccccc) an oligonucleotide or peptide nucleic acid that hybridizes under high stringency conditions to a nucleic acid molecule comprising SEQ ID NO:54 having a G at position 201 but not to a nucleic acid molecule comprising SEQ ID NO:54 having an A at position 201;

(ddddd) an oligonucleotide or peptide nucleic acid that hybridizes under high stringency conditions to a nucleic acid molecule comprising SEQ ID NO:54 having an A at position 201 but not to a nucleic acid molecule comprising SEQ ID NO:54 having a G at position 201;

(eeeee) an oligonucleotide or peptide nucleic acid that hybridizes under high stringency conditions to a nucleic acid molecule comprising SEQ ID NO:55 having an A at position 301 but not to a nucleic acid molecule comprising SEQ ID NO:55 having a T at position 301;

(fffff) an oligonucleotide or peptide nucleic acid that hybridizes under high stringency conditions to a nucleic acid molecule comprising SEQ ID NO:55 having a T at position 301 but not to a nucleic acid molecule comprising SEQ ID NO:55 having an A at position 301;

(ggggg) an oligonucleotide or peptide nucleic acid that hybridizes under high stringency conditions to a nucleic acid molecule comprising SEQ ID NO:56 having a C at position 301 but not to a nucleic acid molecule comprising SEQ ID NO:56 having a T at position 301;

(hhhhh) an oligonucleotide or peptide nucleic acid that hybridizes under high stringency conditions to a nucleic acid molecule comprising SEQ ID NO:56 having a T at position 301 but not to a nucleic acid molecule comprising SEQ ID NO:56 having a C at position 301;

(iiiii) an oligonucleotide or peptide nucleic acid that hybridizes under high stringency conditions to a nucleic acid molecule comprising SEQ ID NO:57 having a C at position 501 but not to a nucleic acid molecule comprising SEQ ID NO:57 having a T at position 501;

(jjjjj) an oligonucleotide or peptide nucleic acid that hybridizes under high stringency conditions to a nucleic acid molecule comprising SEQ ID NO:57 having a T at position 501 but not to a nucleic acid molecule comprising SEQ ID NO:57 having a C at position 501;

(kkkkk) an oligonucleotide or peptide nucleic acid that hybridizes under high stringency conditions to a nucleic acid molecule comprising SEQ ID NO:58 having a G at position 501 but not to a nucleic acid molecule comprising SEQ ID NO:58 having an A at position 501;

(lllll) an oligonucleotide or peptide nucleic acid that hybridizes under high stringency conditions to a nucleic acid molecule comprising SEQ ID NO:58 having an A at position 501 but not to a nucleic acid molecule comprising SEQ ID NO:58 having a G at position 501;

(mmmmm) an oligonucleotide or peptide nucleic acid that hybridizes under high stringency conditions to a nucleic acid molecule comprising SEQ ID NO:59 having a C at position 1216 but not to a nucleic acid molecule comprising SEQ ID NO:59 having a T at position 1216;

(nnnnn) an oligonucleotide or peptide nucleic acid that hybridizes under high stringency conditions to a nucleic acid molecule comprising SEQ ID NO:59 having a T at position 1216 but not to a nucleic acid molecule comprising SEQ ID NO:59 having a C at position 1216;

(ooooo) an oligonucleotide or peptide nucleic acid that hybridizes under high stringency conditions to a nucleic acid molecule comprising SEQ ID NO:60 having a C at position 488 but not to a nucleic acid molecule comprising SEQ ID NO:60 having a T at position 488;

(ppppp) an oligonucleotide or peptide nucleic acid that hybridizes under high stringency conditions to a nucleic acid molecule comprising SEQ ID NO:60 having a T at position 488 but not to a nucleic acid molecule comprising SEQ ID NO:60 having a C at position 488;

(qqqqq) an oligonucleotide or peptide nucleic acid that hybridizes under high stringency conditions to a nucleic acid molecule comprising SEQ ID NO:61 having a G at position 301 but not to a nucleic acid molecule comprising SEQ ID NO:61 having an A at position 301;

(rrrrr) an oligonucleotide or peptide nucleic acid that hybridizes under high stringency conditions to a nucleic acid molecule comprising SEQ ID NO:61 having an A at position 301 but not to a nucleic acid molecule comprising SEQ ID NO:61 having a G at position 301;

(sssss) an oligonucleotide or peptide nucleic acid that hybridizes under high stringency conditions to a nucleic acid molecule comprising SEQ ID NO:62 having a G at position 294 but not to a nucleic acid molecule comprising SEQ ID NO:62 having a T at position 294;

(ttttt) an oligonucleotide or peptide nucleic acid that hybridizes under high stringency conditions to a nucleic acid molecule comprising SEQ ID NO:62 having a T at position 294 but not to a nucleic acid molecule comprising SEQ ID NO:62 having a G at position 294;

(uuuuu) an oligonucleotide or peptide nucleic acid that hybridizes under high stringency conditions to a nucleic acid molecule comprising SEQ ID NO:63 having a G at position 154 but not to a nucleic acid molecule comprising SEQ ID NO:63 having an A at position 154;

(vvvvv) an oligonucleotide or peptide nucleic acid that hybridizes under high stringency conditions to a nucleic acid molecule comprising SEQ ID NO:63 having an A at position 154 but not to a nucleic acid molecule comprising SEQ ID NO:63 having a G at position 154;

(wwwww) an oligonucleotide or peptide nucleic acid that hybridizes under high stringency conditions to a nucleic acid molecule comprising SEQ ID NO:64 having a C at position 201 but not to a nucleic acid molecule comprising SEQ ID NO:64 having a T at position 201;

(xxxxx) an oligonucleotide or peptide nucleic acid that hybridizes under high stringency conditions to a nucleic acid molecule comprising SEQ ID NO:64 having a T at position 201 but not to a nucleic acid molecule comprising SEQ ID NO:64 having a C at position 201;

(yyyyy) an oligonucleotide or peptide nucleic acid that hybridizes under high stringency conditions to a nucleic acid molecule comprising SEQ ID NO:65 having a C at position 201 but not to a nucleic acid molecule comprising SEQ ID NO:65 having a T at position 201;

(zzzzz) an oligonucleotide or peptide nucleic acid that hybridizes under high stringency conditions to a nucleic acid molecule comprising SEQ ID NO:65 having a T at position 201 but not to a nucleic acid molecule comprising SEQ ID NO:65 having a C at position 201;

(aaaaaa) an oligonucleotide or peptide nucleic acid that hybridizes under high stringency conditions to a nucleic acid molecule comprising SEQ ID NO:66 having an A at position 201 but not to a nucleic acid molecule comprising SEQ ID NO:66 having a T at position 201;

(bbbbbb) an oligonucleotide or peptide nucleic acid that hybridizes under high stringency conditions to a nucleic acid molecule comprising SEQ ID NO:66 having a T at position 201 but not to a nucleic acid molecule comprising SEQ ID NO:66 having an A at position 201;

(cccccc) an oligonucleotide or peptide nucleic acid that hybridizes under high stringency conditions to a nucleic acid molecule comprising SEQ ID NO:67 having a C at position 201 but not to a nucleic acid molecule comprising SEQ ID NO:67 having a T at position 201;

(dddddd) an oligonucleotide or peptide nucleic acid that hybridizes under high stringency conditions to a nucleic acid molecule comprising SEQ ID NO:67 having a T at position 201 but not to a nucleic acid molecule comprising SEQ ID NO:67 having a C at position 201;

(eeeeee) an oligonucleotide or peptide nucleic acid that hybridizes under high stringency conditions to a nucleic acid molecule comprising SEQ ID NO:68 having a G at position 527 but not to a nucleic acid molecule comprising SEQ ID NO:68 having a T at position 527;

(ffffff) an oligonucleotide or peptide nucleic acid that hybridizes under high stringency conditions to a nucleic acid molecule comprising SEQ ID NO:68 having a T at position 527 but not to a nucleic acid molecule comprising SEQ ID NO:68 having a G at position 527;

(gggggg) an oligonucleotide or peptide nucleic acid that hybridizes under high stringency conditions to a nucleic acid molecule comprising SEQ ID NO:69 having a G at position 301 but not to a nucleic acid molecule comprising SEQ ID NO:69 having an A at position 301;

(hhhhhh) an oligonucleotide or peptide nucleic acid that hybridizes under high stringency conditions to a nucleic acid molecule comprising SEQ ID NO:69 having an A at position 301 but not to a nucleic acid molecule comprising SEQ ID NO:69 having a G at position 301; and

(iiiiii) an oligonucleotide or peptide nucleic acid that hybridizes under high stringency conditions to a nucleic acid molecule comprising SEQ ID NO:70 having an A at position 357 but not to a nucleic acid molecule comprising SEQ ID NO:70 having a T at position 357;

(jjjjj) an oligonucleotide or peptide nucleic acid that hybridizes under high stringency conditions to a nucleic acid molecule comprising SEQ ID NO:70 having a T at position 357 but not to a nucleic acid molecule comprising SEQ ID NO:70 having an A at position 357.

65. An array of oligonucleotides or peptide nucleic acids attached to a solid support, the array comprising two or more of the oligonucleotides or peptide nucleic acids set out in claim 64.

In accordance with a further aspect of the invention, an array of oligonucleotides or peptide nucleic acids attached to a solid support are provided, the array including two or more of the oligonucleotides or peptide nucleic acids set out herein.

In accordance with a further aspect of the invention, a composition including an addressable collection of two or more oligonucleotides or peptide nucleic acids, the two or more oligonucleotides or peptide nucleic acids selected from the oligonucleotides or peptide nucleic acids set out herein.

In accordance with a further aspect of the invention, an array of oligonucleotides or peptide nucleic acids attached to a solid support are provided, the array including three or more of the oligonucleotides or peptide nucleic acids set out herein.

In accordance with a further aspect of the invention, a composition including an addressable collection of two or more oligonucleotides or peptide nucleic acids, the three or more oligonucleotides or peptide nucleic acids selected from the oligonucleotides or peptide nucleic acids set out herein.

In accordance with a further aspect of the invention, an array of oligonucleotides or peptide nucleic acids attached to a solid support are provided, the array including four or more of the oligonucleotides or peptide nucleic acids set out herein.

In accordance with a further aspect of the invention, a composition including an addressable collection of two or more oligonucleotides or peptide nucleic acids, the four or more oligonucleotides or peptide nucleic acids selected from the oligonucleotides or peptide nucleic acids set out herein.

In accordance with a further aspect of the invention, an array of oligonucleotides or peptide nucleic acids attached to a solid support are provided, the array including five or more of the oligonucleotides or peptide nucleic acids set out herein.

In accordance with a further aspect of the invention, a composition including an addressable collection of two or more oligonucleotides or peptide nucleic acids, the five or more oligonucleotides or peptide nucleic acids selected from the oligonucleotides or peptide nucleic acids set out herein.

In accordance with a further aspect of the invention, a composition including an addressable collection of two or more oligonucleotides or peptide nucleic acids, the two or more oligonucleotides or peptide nucleic acids consisting essentially of two or more nucleic acid molecules set out in SEQ ID NO:1-70 or compliments, fragments, variants, or analogs thereof.

In accordance with a further aspect of the invention, a composition including an addressable collection of two or more oligonucleotides or peptide nucleic acids, the two or more oligonucleotides or peptide nucleic acids consisting essentially of two or more nucleic acid molecules set out in TABLES 1D and 1E or compliments, fragments, variants, or analogs thereof.

The oligonucleotides or peptide nucleic acids as set out herein may further include one or more of the following: a detectable label; a quencher; a mobility modifier; a contiguous non-target sequence situated 5′ or 3′ to the target sequence or 5′ and 3′ to the target sequence.

### BRIEF DESCRIPTION OF THE DRAWINGS

FIG. 1 shows Kaplan-Meier survival curves for a cohort of patients who had systematic inflammatory response syndrome (SIRS) by genotype of interferon gamma rs2069718 (CC/CT=dashed, TT=solid).

FIG. 2 shows Kaplan-Meier survival curves for a cohort of patients who had severe sepsis by genotype of interferon gamma rs2069718 ((CC/CT=dashed, TT=solid).

FIG. 3 shows Kaplan-Meier survival curves for a cohort of patients who had septic shock by genotype of interferon gamma rs2069718C/T (CC/CT=dashed, TT=solid).

FIG. 4 shows Kaplan-Meier survival curves of a cohort of patients who had systematic inflammatory response syndrome (SIRS) by genotype of interferon gamma rs1861493 A/G (GG=dashed vs. AA/AG=solid).

FIG. 5 shows Kaplan-Meier survival curves of a cohort of patients who had severe sepsis by genotype of interferon gamma rs1861493 A/G (GG=dashed vs. AA/AG=solid).

FIG. 6 shows Kaplan-Meier survival curves of a cohort of patients who had septic shock by genotype of interferon gamma rs1861493 A/G (GG=dashed vs. AA/AG=solid).

FIG. 7 shows Kaplan-Meier survival curves of a cohort of patients who had systematic inflammatory syndrome (SIRS) by genotype of interferon gamma rs2069727 A/G (AA=dashed, AG/GG=solid).

FIG. 8 shows Kaplan-Meier survival curves of a cohort of patients who had severe sepsis by genotype of interferon gamma rs2069727 A/G (AA=dashed, AG/GG=solid).

FIG. 9 shows Kaplan-Meier survival curves of a cohort of patients who had septic shock by genotype of interferon gamma rs2069727 A/G (AA=dashed, AG/GG=solid).

### DETAILED DESCRIPTION OF THE INVENTION

### 1. Definitions

In the description that follows, a number of terms are used extensively, the following definitions are provided to facilitate understanding of the invention.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). “Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, while a nucleotide represented by the symbol D may be either A or G or T/U, while a nucleotide represented by the symbol B may be either G or C or T/U, and a nucleotide represented by the symbol H may be either A or C or T/U.

A “polymorphic site” or “polymorphism site” or “polymorphism” or “single nucleotide polymorphism site” (SNP site) or single nucleotide polymorphism” (SNP) as used herein is the locus or position with in a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, enhancers and introns) of genes. Polymorphisms may occur at a single nucleotide site (SNPs) or may involve an insertion or deletion as described herein.

A “risk genotype” as used herein refers to an allelic variant (genotype) at one or more polymorphic sites within the interferon gamma gene (i.e. IFNG) sequences described herein as being indicative of a decreased likelihood of recovery from an inflammatory condition or an increased risk of having a poor outcome. The risk genotype may be determined for either the haploid genotype or diploid genotype, provided that at least one copy of a risk allele is present. Risk genotype may be an indication of an increased risk of not recovering from an inflammatory condition. Subjects having one copy (heterozygotes—for example rs1861493 GA) or two copies (homozygotes—for example rs1861493 GG) of the risk allele may be considered to have the “risk genotype” even though the degree to which the subjects risk of not recovering from an inflammatory condition may increase, depending on whether the subject is a homozygote rather than a heterozygote. Such “risk alleles” or “risk genotypes” may be selected from the following: rs1861493GA; rs1861493GG; rs2069718TC; rs2069718TT; rs2069727AG; rs2069727AA; or a polymorphic site in linkage disequilibrium thereto.

A “decreased risk genotype” as used herein refers to an allelic variant (genotype) at one or more polymorphic sites within the interferon gamma gene (i.e. IFNG) sequences described herein as being indicative of an increased likelihood of recovery from an inflammatory condition or a decreased risk of having a poor outcome. The decreased risk genotype may be determined for either the haploid genotype or diploid genotype, provided that at least one copy of a risk allele is present. Decreased risk genotype may be an indication of an increased likelihood of recovering from an inflammatory condition. Subjects having one copy (heterozygotes) or two copies (homozygotes) of the decreased risk allele (for example rs2069718CT, rs2069718CC) are considered to have the “decreased risk genotype” even though the degree to which the subject's risk of not recovering from an inflammatory condition may increase, depending on whether the subject is a homozygote rather than a heterozygote. Such “decreased risk alleles” or “decreased risk genotypes” or “reduced risk genotypes” or “survival genotypes” may be selected from the following: rs1861493AA; rst861493AG; rs2069718CT; rs2069718CC; rs2069727GG; rs2069727GA; or a polymorphic site in linkage disequilibrium thereto.

An “improved response genotype” (IRG) or improved response polymorphic variant as used herein refers to an allelic variant or genotype at one or more polymorphic sites within the interferon gamma associated polymorphisms selected from interferon gamma (IFNG) as described herein as being predictive of a subject's improved survival in response to activated protein C(XIGRIS™) treatment (for example rs2069718C), or a polymorphic site in linkage disequilibrium thereto.

An “adverse response genotype” (ARG) or adverse response polymorphic variant as used herein refers to an allelic variant or genotype at one or more polymorphic sites within the Inteferon Gamma associated polymorphisms selected from Interferon Gamma (IFNG) as described herein as being predictive of a subject's decreased survival in response to activated protein C(XIGRIS™) treatment (for example rs2069718T), or a polymorphic site in linkage disequilibrium thereto.

A “clade” is a group of haplotypes that are closely related phylogenetically. For example, if haplotypes are displayed on a phylogenetic (evolutionary) tree a clade includes all haplotypes contained within the same branch.

As used herein “haplotype” is a set of alleles of closely linked loci on a chromosome that tend to be inherited together. Such allele sets occur in patterns, which are called haplotypes. Accordingly, a specific SNP or other polymorphism allele at one SNP site is often associated with a specific SNP or other polymorphism allele at a nearby second SNP site or other polymorphism site. When this occurs, the two SNPs or other polymorphisms are said to be in linkage disequilibrium because the two SNPs or other polymorphisms are not just randomly associated (i.e. in linkage equilibrium).

In general, the detection of nucleic acids in a sample depends on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High stringency hybridization is relied upon for the success of numerous techniques routinely performed by molecular biologists, such as high stringency PCR, DNA sequencing, single strand conformational polymorphism analysis, and in situ hybridization. In contrast to Northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

“Oligonucleotides” as used herein are variable length nucleic acids, which may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GLLAM S. et al. Nucleic Acid Res. (1975) 2(5):613-624; BONORA G M. et al. Nucleic Acid Res. (1990) 18(11):3155-9; LASHKARI D A. et al. PNAS (1995) 92(17):7912-5; MCGALL G. et al. PNAS (1996) 93(24):13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

An “addressable collection” as used herein is a combination of nucleic acid molecules or peptide nucleic acids capable of being detected by, for example, the use of hybridization techniques or by any other means of detection known to those of ordinary skill in the art. A DNA microarray would be considered an example of an “addressable collection”.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like single nucleotide polymorphisms (SNPs), are often useful in tracking meiotic recombination events as positional markers on chromosomes.

The pattern of a set of markers along a chromosome is referred to as a “Haplotype”. Accordingly, groups of alleles on the same small chromosomal segment tend to be transmitted together. Haplotypes along a given segment of a chromosome are generally transmitted to progeny together unless there has been a recombination event. Absent a recombination event, haplotypes can be treated as alleles at a single highly polymorphic locus for mapping.

Furthermore, the preferential occurrence of a disease gene in association with specific alleles of linked markers, such as SNPs or other polymorphisms, is called “Linkage Disequilibrium” (LD). This sort of disequilibrium generally implies that most of the disease chromosomes carry the same mutation and the markers being tested are relatively close to the disease gene(s).

For example, in SNP-based association analysis and linkage disequilibrium mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs may be tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For an LD based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs).

In population genetics linkage disequilibrium refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as position 260 of SEQ ID NO:1 (see Akey, J. et al. (2001). Haplotypes vs. single marker linkage disequilibrium tests: what do we gain? European Journal of Human Genetics. 9:291-300; and Zhang, K. et al. (2002). Haplotype block structure and its applications to association studies: power and study designs. American Journal of Human Genetics. 71:1386-1394). This viewpoint is further substantiated by Khoury et al. ((1993). Fundamentals of Genetic Epidemiology. New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for D′ of ≧0.5 or r2≧0.5) are potentially useful in predicting the identity of an allele of interest (i.e. associated with the condition of interest). A high degree of linkage disequilibrium may be represented by an absolute value for D′ of ≧0.6 or r2≧0.6. Alternatively, a high degree of linkage disequilibrium may be represented by an absolute value for D′ of ≧0.7 or r2≧0.7 or by an absolute value for D′ of ≧0.8 or r2≧0.8. Additionally, a high degree of linkage disequilibrium may be represented by an absolute value for D′ of ≧0.85 or r2≧0.85 or by an absolute value for D′ of ≧0.9 or r2≧0.9. Accordingly, two SNPs that have a high degree of LD may be equally useful in determining the identity of the allele of interest or disease allele. Therefore, we may assume that knowing the identity of the allele at one SNIP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably. For example, in the population from which the tagged SNPs were identified from the SNP identified by rs1861493 is in “linkage disequilibrium” with the SNP identified by rs2069718, whereby when the genotype of rs1861493 is A the genotype of rs2069718 is C. Similarly, when the genotype of rs1861493 is G the genotype of rs2069718 is T. Accordingly, the determination of the genotype at rs1861493 will provide the identity of the genotype at rs2069718 or any other locus in “linkage disequilibrium” therewith. Particularly, where such a locus is has a high degree of linkage disequilibrium thereto.

Linkage disequilibrium is useful for genotype-phenotype association studies. For example, if a specific allele at one SNP site (e.g. “A”) is the cause of a specific clinical outcome (e.g. call this clinical outcome “B”) in a genetic association study then, by mathematical inference, any SNP (e.g. “C”) which is in significant linkage disequilibrium with the first SNP, will show some degree of association with the clinical outcome. That is, if A is associated (˜) with B, i.e. A˜B and C˜A then it follows that C˜B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on linkage disequilibrium between A and C.

Until the mechanism underlying the genetic contribution to a specific clinical outcome is fully understood, linkage disequilibrium helps identify potential candidate causal SNPs and also helps identify a range of SNPs that may be clinically useful for prognosis of clinical outcome or of treatment effect. If one SNP within a gene is found to be associated with a specific clinical outcome, then other SNPs in linkage disequilibrium will also have some degree of association and therefore some degree of prognostic usefulness. By way of prophetic example, if multiple polymorphisms were tested for individual association with an improved response to vasopressin receptor agonist administration in our SIRS/severe sepsis/septic shock cohort of ICU subjects, wherein the multiple polymorphisms had a range of linkage disequilibrium with IFNG polymorphism rs1861493 and it was assumed that rs1861493 was the causal polymorphism, and we were to order the polymorphisms by the degree of linkage disequilibrium with rs1861493, we would expect to find that polymorphisms with high degrees of linkage disequilibrium with rs1861493 would also have a high degree of association with this specific clinical outcome. As linkage disequilibrium decreased, we would expect the degree of association of the polymorphism with this specific clinical outcome to also decrease. Accordingly, logic dictates that if A˜B and C˜A, then C˜B. That is, any polymorphism, whether already discovered or as yet undiscovered, that is in linkage disequilibrium with one of the improved response genotypes described herein will likely be a predictor of the same clinical outcomes that rs1861493 is a predictor of. The similarity in prediction between this known or unknown polymorphism and rs1861493 would depend on the degree of linkage disequilibrium between such a polymorphism and rs1861493.

Numerous sites have been identified as polymorphic sites in the Interferon Gamma associated gene (see TABLE 1B). Furthermore, the polymorphisms in TABLE 1B are linked to (in linkage disequilibrium with) numerous polymorphisms as set out in TABLE 1C below and may also therefore be indicative of subject prognosis.

TABLE 1B
Polymorphisms in the interferon gamma gene (IFNG) genotyped in a cohort of
critically ill subjects. Minor Allele Frequencies (MAFs) for Caucasians were taken from
Seattle SNPs (http://www.pga.gs.washington).

| | | May 2004 | | | |
|---|---|---|---|---|---|
| | | Chromosomal | Seattle | | Minor |
| | | position | SNPs | Minor | Allele |
| Official Gene Name | rs# | (Build 35) | Identifier | allele | Frequency |
| interferon gamma (IFNG) | rs1861493 | 66837463 | 3890 | G | 0.39 |
| interferon gamma (IFNG) | rs2069718 | 66836429 | 4925 | T | 0.34 |
| interferon gamma (IFNG) | rs2069727 | 66834490 | 6864 | G | 0.40 |

TABLE 1C
Polymorphisms in linkage disequilibrium with those listed in TABLE 1B above,
as identified using the Haploview program (BARRETT JC. et al. Bioinformatics (2005)
21(2): 263-5 (http://www.broad.mit.edu/mpg/haploview/)). Linkage Disequilibrium between
markers was defined using r² and D′ whereby all SNPs available on Hapmap.org (phase II)
were included. A minimum r² of 0.5 was used as the cutoff to identify LD SNPs. The rs
designation (NCBI) and chromosomal position (March 2006 Build 36) are reported.

| | | | Polymorphisms | | | | | Distance |
|---|---|---|---|---|---|---|---|---|
| Tag | Chromosome | Survival | in | Chromosome | LD | | | from Tag |
| Polymorphisms | Position | Allele | LD | Position | Allele | D′ | r\hat{ }2 | SNP |
| rs1861493 | 66837463 | A | rs10467155 | 66796339 | | 0.781 | 0.562 | 41124 |
| | | | rs7973244 | 66799614 | A | 0.86 | 0.626 | 37849 |
| | | | rs7137993 | 66857621 | A | 1 | 0.677 | 20158 |
| | | | rs12315837 | 66859270 | A | 1 | 0.688 | 21807 |
| | | | rs4913277 | 66868439 | T | 1 | 0.708 | 30976 |
| | | | rs2080414 | 66858084 | T | 1 | 0.71 | 20621 |
| | | | rs7956817 | 66860201 | A | 1 | 0.71 | 22738 |
| | | | rs2069718 | 66836429 | C | 1 | 0.712 | 1034 |
| | | | rs1076025 | 66857393 | A | 1 | 0.715 | 19930 |
| | | | rs12312186 | 66857437 | A | 1 | 0.715 | 19974 |
| | | | rs7137814 | 66857645 | T | 1 | 0.715 | 20182 |
| | | | rs2098395 | 66827012 | A | 0.891 | 0.718 | 10451 |
| | | | rs9888319 | 66860800 | A | 1 | 0.72 | 23337 |
| | | | rs7298410 | 66867470 | C | 1 | 0.72 | 30007 |
| | | | rs2058739 | 66869539 | C | 1 | 0.72 | 32076 |
| | | | rs2216164 | 66820607 | G | 1 | 0.72 | 16856 |
| | | | rs2041864 | 66824756 | T | 1 | 0.72 | 12707 |
| | | | rs2870951 | 66870812 | C | 1 | 0.745 | 33349 |
| | | | rs2193047 | 66822895 | C | 1 | 0.772 | 14568 |
| | | | rs741344 | 66883353 | | 0.917 | 0.809 | 45890 |
| | | | rs4913405 | 66804144 | A | 1 | 0.819 | 33319 |
| | | | rs6581794 | 66831989 | C | 0.959 | 0.883 | 5474 |
| | | | rs10784683 | 66856790 | G | 1 | 0.89 | 19327 |
| | | | rs1118866 | 66807018 | T | 1 | 0.911 | 30445 |
| | | | rs10784684 | 66859200 | C | 0.956 | 0.914 | 21737 |
| | | | rs9888400 | 66863314 | A | 1 | 0.915 | 25851 |
| | | | rs7138107 | 66848710 | C | 1 | 0.921 | 11247 |
| | | | rs1861494 | 66837676 | T | 1 | 0.925 | 213 |
| | | | rs2098394 | 66858048 | A | 1 | 0.925 | 20585 |
| | | | rs10878779 | 66867288 | C | 1 | 0.925 | 29825 |
| | | | rs2193045 | 66820787 | G | 1 | 0.926 | 16676 |
| | | | rs2193049 | 66833189 | G | 1 | 0.926 | 4274 |
| | | | rs2870952 | 66852156 | C | 1 | 0.927 | 14693 |
| | | | rs2193048 | 66823141 | C | 1 | 0.927 | 14322 |
| | | | rs2870953 | 66830897 | A | 1 | 0.927 | 6566 |
| | | | rs3181034 | 66833004 | G | 1 | 0.927 | 4459 |
| | | | rs759488 | 66873422 | C | 1 | 0.957 | 35959 |
| | | | rs2193050 | 66833460 | G | 1 | 0.957 | 4003 |
| | | | rs4913418 | 66877134 | A | 1 | 0.961 | 39671 |
| | | | rs10784688 | 66866836 | C | 1 | 0.962 | 29373 |
| | | | rs10748099 | 66873606 | C | 1 | 0.962 | 36143 |
| | | | rs6581795 | 66846082 | A | 1 | 1 | 8619 |
| | | | rs7302488 | 66847146 | T | 1 | 1 | 9683 |
| | | | rs759487 | 66852346 | C | 1 | 1 | 14883 |
| | | | rs7959933 | 66866416 | C | 1 | 1 | 28953 |
| | | | rs4913278 | 66868663 | T | 1 | 1 | 31200 |
| | | | rs4913415 | 66868881 | G | 1 | 1 | 31418 |
| | | | rs2216163 | 66817223 | C | 1 | 1 | 20240 |
| | | | rs7132697 | 66819108 | A | 1 | 1 | 18355 |
| | | | rs7302226 | 66819540 | G | 1 | 1 | 17923 |
| | | | rs7133554 | 66819832 | C | 1 | 1 | 17631 |
| | | | rs2111059 | 66827938 | T | 1 | 1 | 9525 |
| | | | rs10878763 | 66829965 | G | 1 | 1 | 7498 |
| | | | rs2069705* | 66841278 | T | | | |
| | | | rs2069733* | 66836499 | G | | | |
| rs2069718 | 66836429 | C | rs2193046 | 66821052 | C | 1 | 0.501 | 15377 |
| | | | rs741344 | 66883353 | | 0.9 | 0.568 | 46924 |
| | | | rs4913405 | 66804144 | A | 1 | 0.578 | 32285 |
| | | | rs759488 | 66873422 | C | 0.948 | 0.664 | 36993 |
| | | | rs4913418 | 66877134 | A | 0.953 | 0.667 | 40705 |
| | | | rs10748099 | 66873606 | C | 0.955 | 0.675 | 37177 |
| | | | rs10784688 | 66866836 | C | 0.955 | 0.676 | 30407 |
| | | | rs2193050 | 66833460 | G | 1 | 0.679 | 2969 |
| | | | rs7959933 | 66866416 | C | 1 | 0.698 | 29987 |
| | | | rs7302226 | 66819540 | G | 1 | 0.699 | 16889 |
| | | | rs4913415 | 66868881 | G | 1 | 0.702 | 32452 |
| | | | rs10784684 | 66859200 | C | 1 | 0.71 | 22771 |
| | | | rs1861493 | 66837463 | A | 1 | 0.712 | 1034 |
| | | | rs7302488 | 66847146 | T | 1 | 0.712 | 10717 |
| | | | rs759487 | 66852346 | C | 1 | 0.712 | 15917 |
| | | | rs4913278 | 66868663 | T | 1 | 0.712 | 32234 |
| | | | rs2216163 | 66817223 | C | 1 | 0.712 | 19206 |
| | | | rs7132697 | 66819108 | A | 1 | 0.712 | 17321 |
| | | | rs7133554 | 66819832 | C | 1 | 0.712 | 16597 |
| | | | rs2111059 | 66827938 | T | 1 | 0.712 | 8491 |
| | | | rs10878763 | 66829965 | G | 1 | 0.712 | 6464 |
| | | | rs10784683 | 66856790 | G | 0.956 | 0.727 | 20361 |
| | | | rs6581795 | 66846082 | A | 1 | 0.728 | 9653 |
| | | | rs6581794 | 66831989 | C | 1 | 0.732 | 4440 |
| | | | rs7138107 | 66848710 | C | 1 | 0.755 | 12281 |
| | | | rs1118866 | 66807018 | T | 1 | 0.762 | 29411 |
| | | | rs2098394 | 66858048 | A | 1 | 0.766 | 21619 |
| | | | rs10878779 | 66867288 | C | 1 | 0.766 | 30859 |
| | | | rs2193049 | 66833189 | G | 1 | 0.769 | 3240 |
| | | | rs9888400 | 66863314 | A | 1 | 0.77 | 26885 |
| | | | rs2870952 | 66852156 | C | 1 | 0.771 | 15727 |
| | | | rs2193048 | 66823141 | C | 1 | 0.771 | 13288 |
| | | | rs2870953 | 66830897 | A | 1 | 0.771 | 5532 |
| | | | rs3181034 | 66833004 | G | 1 | 0.771 | 3425 |
| | | | rs10467155 | 66796339 | | 1 | 0.786 | 40090 |
| | | | rs1861494 | 66837676 | T | 1 | 0.793 | 1247 |
| | | | rs2193045 | 66820787 | G | 1 | 0.797 | 15642 |
| | | | rs7973244 | 66799614 | A | 1 | 0.849 | 36815 |
| | | | rs2870951 | 66870812 | C | 0.963 | 0.895 | 34383 |
| | | | rs2193047 | 66822895 | C | 1 | 0.93 | 13534 |
| | | | rs7137993 | 66857621 | A | 1 | 0.962 | 21192 |
| | | | rs12315837 | 66859270 | A | 1 | 0.964 | 22841 |
| | | | rs1076025 | 66857393 | A | 1 | 1 | 20964 |
| | | | rs12312186 | 66857437 | A | 1 | 1 | 21008 |
| | | | rs7137814 | 66857645 | T | 1 | 1 | 21216 |
| | | | rs2080414 | 66858084 | T | 1 | 1 | 21655 |
| | | | rs7956817 | 66860201 | A | 1 | 1 | 23772 |
| | | | rs9888319 | 66860800 | A | 1 | 1 | 24371 |
| | | | rs7298410 | 66867470 | C | 1 | 1 | 31041 |
| | | | rs4913277 | 66868439 | T | 1 | 1 | 32010 |
| | | | rs2058739 | 66869539 | C | 1 | 1 | 33110 |
| | | | rs2216164 | 66820607 | G | 1 | 1 | 15822 |
| | | | rs2041864 | 66824756 | T | 1 | 1 | 11673 |
| | | | rs2069705* | 66841278 | T | | | |
| | | | rs2069733* | 66836499 | G | | | |
| rs2069727 | 66834490 | G | rs10878774 | 66866539 | A | 1 | 0.759 | 32049 |
| | | | rs10878786 | 66877192 | A | 0.955 | 0.874 | 42702 |
| | | | rs10878784 | 66876775 | G | 0.961 | 0.889 | 42285 |
| | | | rs971545 | 66877952 | G | 0.965 | 0.931 | 43462 |
| | | | rs12301088 | 66876215 | T | 1 | 0.962 | 41725 |
| | | | rs7969024 | 66865170 | T | 1 | 0.965 | 30680 |
| | | | rs11177081 | 66856562 | G | 1 | 0.966 | 22072 |
| | | | rs12317232 | 66865390 | A | 1 | 0.966 | 30900 |
| | | | rs11177083 | 66857812 | T | 1 | 1 | 23322 |
| | | | rs10878766 | 66857864 | G | 1 | 1 | 23374 |
| | | | rs7969592 | 66865916 | G | 1 | 1 | 31426 |
| | | | rs10878781 | 66868894 | G | 1 | 1 | 34404 |
| | | | rs2870950 | 66870973 | T | 1 | 1 | 36483 |
| | | | rs10492197 | 66871874 | T | 1 | 1 | 37384 |
| | | | rs2193046 | 66821052 | C | 1 | 1 | 13438 |
| | | | rs2069705* | 66841278 | T | | | |
| | | | rs2069733* | 66836499 | G | | | |

Polymorphisms in linkage disequilibrium with those listed in TABLE 1B above, as identified using the Haploview program (BARRETT JC. et al. Bioinformatics (2005) 21(2): 263-5 (http://www.broad.mit.edu/mpg/haploview/)) and the LD function in the Genetics Package in R (R Core Development Group, 2005 - R Development Core Team (www.R-project.org) are listed in TABLE 1C. Linkage Disequilibrium was determined using all SNPs available on Hapmap.org except rs2069705* and rs2069733*, which were genotyped by the Seattle SNPs PGA on http://pga.gs.washington.edu. A minimum r² of 0.5 was used as the cutoff to identify LD SNPs.

It will be appreciated by a person of skill in the art that further linked polymorphic sites and combined polymorphic sites may be determined. The haplotype of interferon gamma associated genes can be created by assessing polymorphisms in protein interferon gamma genes in normal subjects using a program that has an expectation maximization algorithm (i.e. PHASE). A constructed haplotype of interferon gamma genes may be used to find combinations of SNP's that are in linkage disequilibrium (LD) with the haplotype tagged SNPs (htSNPs) identified herein. Accordingly, the haplotype of an individual could be determined by genotyping other SNPs or other polymorphisms that are in LD with the htSNPs identified herein. Single polymorphic sites or combined polymorphic sites in LD may also be genotyped for assessing subject response to activated protein C or protein C like compound or protein C like compound treatment.

It will be appreciated by a person of skill in the art, that determination of the survival allele or risk allele in linked polymorphic sites may be determined using haplotype structure. This prediction is based on an expectation maximization algorithm that is heavily dependent on sample size. Given the high r-squared observed in the linked polymorphic sites it would be appreciated by a person of skill in the art that the survival allele or risk allele may be routinely determined given a sufficiently large cohort. Accordingly, the allele designations provided herein for polymorphic sites in linkage disequilibrium may be adjusted.

An “rs” prefix designates a SNP in the database is found at the NCBI SNP database (http://www.ncbi.nlm.nih.gov/entrez/query.fcgi?db=Snp). The “rs” numbers are the NCBI|rsSNP ID form.

TABLE 1D below shows the flanking sequences for a selection of interferon gamma SNPs providing their rs designations, position within the sequence and corresponding SEQ ID NO designations. Each polymorphism is bold and underlined within the flanking sequence.

TABLE 1D
Flanking sequence for the IFNG SNPs genotyped.

| | | SEQ | |
|---|---|---|---|
| | | ID | |
| GENE | SNP | NO: | FLANKING SEQUENCE |
| IFNG | rs1861493 | 1 | TCTGAAAGTTGATAGAGAGTTGATAGAACAATCTTTTCATAAGGTATAAATT |
| | (position 260) | | CTATCTATTTTTCCCTAAAAACAAACAAACAGCAACCATTCTTGCTTCTAAT |
| | | | TGGGCAGTACAATCTGATAGGTTGGCTAGAGACTTGCAGTGGGGTGTCCCTG |
| | | | GTACCTATTCAAAGACTGTAGCTTTCTTCTATCTCATTCTCATTTTCTATTC |
| | | | TTTGCATTGTAGAGTTTTGGAGCAAAGAAGGTCATCAAACTTATACAGTGAR |
| | | | CCTAACAGTTTCCTTTTAAGATGAGGAAACTGAGCCCCAGCCAGCCATGTGA |
| | | | TTCATCACAGTTCCTTGGTGGCTGAGTTGGGAGGAGAACACACATCTTCTCA |
| | | | GCTCCTCCCACTGCTCTTTCCATTAAGACAGACACCCTCTCATTCAAAGTAA |
| | | | GAGAATTTCCATCATATGAGCAAGGGACAATGAGAGAACTGCTTCTCAGTAC |
| | | | TCCCCGCTTCTTCCTCACCTACTTCCTCTTCACTGGATTTGTCAACTCACCT |
| | | | GTCTTTACGCAATAGTTACAATGCCAGCATTTCTCTACATTACATACTTCAG |
| | | | CGATTCTCTTACTGGCTTTGCAGTCACCCAAACACGAATGGAAATA |
| IFNG | rs2069718 | 2 | GGCAATCTTGAGTGAGCTCTATTAATTATTATTCTCTTTGGCTCAGTTGCTA |
| | | | AGCTATTTTATGCATGTTATGCCCTTTGACAATTAGTCTTTAGCTGTAATCC |
| | | | CCCAGCCATCCTCAGAAATGTGGTGAGTAGCCATAGTGTTCCCAAGATTAGA |
| | | | AAAAATGTAATGGCAGAGCCAAGAGGAAGGTAAATGGTCCACATYTTATGAA |
| | | | GCATCATCTAAATGGCCCTATTGGTTAGAGTGAGGAGATGCAAGTAGTTCAA |
| | | | TTTGCTTGCCTAGAAGGCAGGGTACTGGAAAAGTTGTTGCAATTCTTAATTT |
| | | | TAAACTTTATATATCAGTAAGCCATATATAAATATGATTGGGGGTGTTTATT |
| | | | TTAAAATCTATTATGGAAATTGAGAGACTGACCTAA |
| IFNG | rs2069727 | 3 | TGTGGTATTTCTTTCCACTAGCATTTTGTTGGCTTTCGCTTTTCCAGTTAGC |
| | | | AGCTCTTTGAATTATCTTTCTAAGATACAGATTTAATTATGTCACTATTCAA |
| | | | TTCAGAGGTTCTGCTATGGAATGTAGTTTAAACTGCTTAGCTTGGCACACAG |
| | | | AGATTTATTTCTAGCCCCTTCTCCACCTTCCTATTTCCTCCTTCRTTTCAGA |
| | | | ATCTTCCTCTCCCTCATCCAATGCTGGCAAACACCAGTGGGGGTGGAGTAGT |
| | | | GGGTGTAAGCTCTAGGGAGAAGGCTTGGATTGGAATCCAAGTTATTCCATTA |
| | | | CAAGTAGTGTGACCTTTAATACATTATGTATATTGTCTAAGTTTCAGCTTTA |
| | | | TTGTCTGAAAAAGAAAAATAATTGTGTGTTCCTCATAATATTGTGGTACGAA |
| | | | TTGATTCTTTCACTCAAGAAATATTTACTGGAGTACCTACTACATGCCTGGT |
| | | | GCTGTTGTAGACCTTGAGATACCTTACTCAAGCAAAACAGCCAAGGATCCCT |
| | | | GCCCCTGGGGAATTTGAAATTAAGCAAGGGACAGATAAACAATGAACAAAAT |
| | | | ACATAATATGTAAGTCTATTCCATGGCATTCTCTAAGGTGATTGGTGTCATG |
| | | | GAAAAATAGTTAAAGGAGAGCAGGACAGGGAAATTAGGAGTCCTATGTATGG |
| | | | TGGAGTGGGAGGGCTAGAGGTTTAAAAGGGTAATTATATCTGGCCTTATTGA |
| | | | GGAGATGCCATTTGAGGAAGCGCTTTAAGAAGTAAGAGAGGTAGCTATTTGA |
| | | | ATTCCAGGCAAAAGGTATATCCTTGCAAAGGCTCTGAAGAGATTTTCCTGGA |
| | | | GTGGTAGAAGAACCAGCAGACCAGTGTGCTGGGCCCAGAAGACGGAAGAGAA |
| | | | AATCAGCCACACTTGAGAGGAATTCAGGGGAAGCAATGTCCTTAGGGGAGGG |
| | | | CCAGTTTATCTTTTGAGAAGGAGGAAGTTGAGGATATGATGGATTTGGTTAG |
| | | | TTCTGGGCTGTAAATTCCAGAAGACCCAGTGAGACAAAGTAAGAGAGGTTGT |
| | | | CATAAAAGGGAACGTGCATAGGGATGTGTTGTGAGTCTGAGACTTCTTATGA |
| | | | TTACCGACATAAACAAGATAATGGATATAGTGAGATTAGTTCTACCAGCTGT |
| | | | GGAACGTGTAGTGGTGGCAAGATCATGAATGTCAAGGATAGAGAGGGTTAGA |
| | | | CATCTGGGGCTTCCTTCTCAACAATTTCACATAAACCTCCAACAGCAACAGT |
| | | | AGGATTATGTGAAATAGATCACACAAAGGATCATTTGAGTCATTGACAATAA |
| | | | TCAGGGGT |

The Sequences given in TABLE 1D (SEQ ID NO:1-3) above and in TABLE 1E (SEQ ID NO:4-70) would be useful to a person of skill in the art in the design of primers and probes or other oligonucleotides for the identification of interferon gamma gene SNP alleles and or genotypes as described herein.

TABLE 1E below shows the flanking sequences for a selection of interferon gamma gene SNPs in LD with the tagged SNPs in TABLE 1D, providing their rs designations, alleles and corresponding SEQ ID NO designations. Each SNP position in the flanking sequence is given and identified in bold and underlined. Tagged SNPs that are also in LD are not repeated in TABLE 1E.

TABLE 1E

Flanking sequence for a selection of SNPs in linkage
disequilibrium with the SNPs identified in Table 1D.

| | | | |
|---|---|---|---|
| | | SEQ | |
| | | ID | |
| GENE | SNP | NO: | FLANKING SEQUENCE |

| | | | |
|---|---|---|---|
| IFNG | rs1861494 | 4 | TCTGAAAGTTGATAGAGAGTTGATAGAACAATCTTTTCATAAGGTAT |
| | (position | | AAATTCTATCTATTTTTCCCTAAAAACAAACAAACAGCAACCATTCT |
| | 473) | | TGCTTCTAATTGGGCAGTACAATCTGATAGGTTGGCTAGAGACTTGC |
| | | | AGTGGGGTGTCCCTGGTACCTATTCAAAGACTGTAGCTTTCTTCTAT |
| | | | CTCATTCTCATTTTCTATTCTTTGCATTGTAGAGTTTTGGAGCAAAG |
| | | | AAGGTCATCAAACTTATACAGTGAGCCTAACAGTTTCCTTTTAAGAT |
| | | | GAGGAAACTGAGCCCCAGCCAGCCATGTGATTCATCACAGTTCCTTG |
| | | | GTGGCTGAGTTGGGAGGAGAACACACATCTTCTCAGCTCCTCCCACT |
| | | | GCTCTTTCCATTAAGACAGACAGCCTCTCATTCAAAGTAAGAGAATT |
| | | | TCCATCATATGAGCAAGGGACAATGAGAGAACTGCTTCTCAGTACTC |
| | | | CCYGCTTCTTCCTCACCTACTTCCTCTTCACTGGATTTGTCAACTCA |
| | | | CCTGTCTTTACGCAATAGTTACAATGCCAGCATTTCTCTACATTACA |
| | | | TACTTCAGCGATTCTCTTACTGGCTTTGCAAAGTCACCCAAACACGA |
| | | | ATGGAAATA |
| IFNG | rs2069705 | 5 | ACTTGTATAGAGAATCTAAGATTAATTTTAAGGAGGATAATTTTGGA |
| | (position | | AAAACTCAGGGAGATGGTAATTTTTAAGCCGGGCTTGGATGGATGGC |
| | 709) | | TACTACTCTCAGGGGCACAAATGAGGGGAAAAAGAACTCAAGACCAA |
| | | | AGAAACAGCATGAGCAAAGGTCCAGGGTACTTTTTTTTTTTTTTTTT |
| | | | AAAGAAATGACTAGGCCGGGTGCGGTGGCTCACGCCTGTAATCCCAG |
| | | | CACTTTGGGAGGCCAAGGCGGGCGGATCACGAGGTCAGGAGATCGAG |
| | | | ACCATCCTGATTAACACAGTGAAACCCCGTCTGTACTAAAAATAGCA |
| | | | CAAAAAAAAAAAAAAAAAAAAAAATTAGCCGGGCGTGGCGAGTGCCT |
| | | | GTAGTCCCAGCTACTCGGGAGGCTGAGGCGGGAGAATGGCGTGAATC |
| | | | CGGGAGGCAGAGCTTGCAGTGAGCCGAGATTGCGCCACTGCACTCCA |
| | | | GCCCTGGGTGACAGAGCAAGACTCCGTCTCAAAAAAAAAAAAAAAAA |
| | | | AAAGAAATGACTAGTCATCCAATGTGCCAAAATAATAATAAACTTTT |
| | | | ATTAGTGATTACTATATGCCAGGAAAAATTCCTAGCACTTTATGAGG |
| | | | ATTACCTGATTTAATTTTCAACTGAAGCATGGAAGAAGATACTATTA |
| | | | TCAAGCCAGTTTTACAGGTAAGGAGACTGAGTCATAGAAGATTTAAG |
| | | | AAGYTAACTCACAATCATATAGCTAGATAGTAGAGGAGTCAGGAATC |
| | | | AAGTTTGCCCCATAACTGCAATACTGTTATGTACACAGTACAGGTAG |
| | | | AAATGCAAAGTGGGTTTGAACCAAAGAGTGGAGGGCTTTTTGTGCCA |
| | | | TCCCAAAGTGTTGTACTTCATAAATAAATTACAAAGGAGGAGAAAGA |
| | | | ATCCTATTTTTTTTTG |
| IFNG | rs2069733 | 6 | GAGAGACATGGCAACAGGTCTCCTTTGGTTATAAACTAGACACTCAG |
| | (position | | CACTTGTTTCTAATCCAGTGGTGCCCCTGGCTTACTGTTCAGTCCTG |
| | 401) | | GATAAGTCTCTTAGTTTCTTGGTGATGATTTGAACATTGGAAAGTAA |
| | | | AATCTGTCACTTGCAAACACACAGCTTGTCGAAAATTTTTTCTACTC |
| | | | TGCAGGAACTGGGCCTTAAAAAATGAAAAAAAAATCTGTGGTTTCTT |
| | | | CCTTCTGGAAGCTACAAACCTCCTGTTTCTTGATGGGCAATCTTGAG |
| | | | TGAGCTCTATTAATTATTATTCTCTTTGGCTCAGTTGCTAAGCTATT |
| | | | TTATGCATGTTATGCCCTTTGACAATTAGTCTTTAGCTGTAATCCCC |
| | | | CAGCCATCCTCAGAAATGTGGTGA(-/G) |
| | | | GTAGCCATAGTGTTCCCAAGATTAGAAAAAATGTAATGGCAGAGCCA |
| | | | AGAGGAAGGTAAATGGTCCACATTTTATGAAGCATCATCTAAATGGC |
| | | | CCTATTGGTTAGAGTGAGGAGATGCAAGTAGTTCAATTTGCTTGCCT |
| | | | AGAAGGCAGGGTACTGGAAAAGTTGTTGCAATTCTTAATTTTAAACT |
| | | | TTATATATCAGTAAGCCATATATAAATATGATTGGGGGTGTTTATTT |
| | | | TAAAATCTATTATGGAAATTGAGAGACTGACCTAATCTGGGAGAAAT |
| | | | TAAAAATTACAGTTTTCACTCGTTTTGGATTTGGTGTTTTCTAGGGT |
| | | | ACCTAACCTAGATCAGTGGTTCTCAAACTTAGGTGGATGTCAGAATC |
| | | | ACCTGGGGAGCTTAGTGAATGCAC |
| IFNG | rs10467155 | 7 | GACCAGACTTTGCCTAGGTTGAGGACCACTGGGAGCCAATTGATTTT |
| | (position | | CACAGCTCTAAGAAAAGCCACAGTTAGAACAGGGTTGATTTCAATTC |
| | 734) | | TACAGTGGGCATACCTCAGAGATACTGTGGGTTCAGTTCCAAATCAC |
| | | | CACAATAAAGCAAATATCACAATAAAGTGAGTCACACAAATTTTTTG |
| | | | GTTTCCCAGTGCATATAGAAGTTATGTTTACACTATACTATAGTCTA |
| | | | TTAAGTATGCAATAATATTATGTCTAAAAAACAATGTACATATCTTA |
| | | | ATTTAAAAATACTTTACAGGCTAGCGTTGGTGGCTCATGCCTATAAT |
| | | | CCTAGCACTTTGAGAGGCAGTCGTGGGAGAATCACTTGAAGCCAGGA |
| | | | GTTCAAGACCAGACTGGGCAACATAGCAAGACCCAGTCTCTACCAAA |
| | | | AAAATTTAAACATTAGCTCGGCATGATGGCATGCGCCTCTAGTCCTA |
| | | | GATAGTCAGGAGAATGAGGCAAGGGGATCTCTTGAGCCCAGGAGTTC |
| | | | GAAATTACAGTGAACTCTGATCATTCCACTGTACTCTAGTCCAGGTG |
| | | | ACAGAGTGAGACCATGTCTCGAAAACATAAAAGATATTTTATTGCTA |
| | | | AATATCGAAAATGATTATCTGAGCCTTTGGCAAGTTGTAATAGTTTT |
| | | | TGCTGCTGGAGGGTCTTGCCTAGATGTTGATGGCTACTAGCTGATCA |
| | | | GGATGGTGGTTGTGGAAGGTTGGGGTGGYTATGGCAATTTGTTGAAA |
| | | | TAAGACAACAATGTGCTTTGCTGTATTGATTGACTCTTCCTTTCATA |
| | | | AAAGATTTCTCTGTGGCATGCAACACTGCTTGATAGCATATTACCCA |
| | | | CGGTAGAACTTCTTTCAAAATTGGAGCCAATCCTCTCAAATCCTGCC |
| | | | ACTGCTCTATCAACTAAGTTTATGTAATATTCTAAATCCTTTGCCAT |
| | | | CATTTCAACAGTGTTCACAGCATCTTCACCAAGAGTAGATTTCATCT |
| | | | CAAGAAATCACCTTCTCTGTTCATCTCTAAGAAGCAACTCCTCACAT |
| | | | ACTCAAATTTTATCAGGAGGTTGCAGCAATTCACTTGCAGCTTCAGG |
| | | | CTCCACTTCTGCTTCTTTTGCTATTTCCACCACATGTGCAGTTACTT |
| | | | TCTCCACTAAAGTCTTGAATCCCTTAAAGTCATCCACGAGGGTTGGA |
| | | | ATTAACTTCTTCCAAACTCCTATTAATGTTTATATTTTAAACTCCTC |
| | | | TCATGAATCATGAAAGTTCTTAATAGCAGCCAGAATTGTGAATTTTT |
| | | | TCCGGGTGATTCTCAGTTCACTTTTCCCAGATCTATTCATGGAATCA |
| | | | CTATCTATGGCAGCTATAGGCTTTTAAAATTTATTTCTTAAATAATA |
| | | | CAACCTGAAAGTTGAAATTACTCCTTGATCCATGGGCTGCAGAATAA |
| | | | AGCCTAACACAGAAGGCATGAGCTCTTGGGTGACTAGGTGCATTGTC |
| | | | AATGAGAAGTGACATTTTGAAAGAAATATTTTTTTCTGAGCTGTAGG |
| | | | TCTCCACAGTTGGCTTAAAATATTCAGTAAACCATGCTGTAAACAGA |
| | | | TGTGCTGTTATCCACGCTTTGTTGTTCCATTAACAGAGCACAGGCAG |
| | | | AGTAGATTTAACTGATGTTAAATTCTTAAGGACTTTAAGATTTTGGG |
| | | | AAGGATATATAAGCATGGGTTTCCACTTAAAGTCACAGCCACATTAG |
| | | | CCCCCCTAACAAGAGAGTCAATCTGTCCTTTAAAGCTTTGAAACCAG |
| | | | GACTTGACTTCTCCTCTCTGGCTATGAAACTCCTAGATGGCATATTC |
| | | | TTCCAATATAAGGCTATTTCATCTGCATTTAAAATCCATTGTTTAGT |
| | | | GTAGCCACCTTCAACATTGAACTTAGCTACATCTTTTGCATAACTTG |
| | | | CTGCAACCTCTCCATCAGTACATGCAGCTTCACCTTGCACATTTGTG |
| | | | TTATAGAGACAGCTTCTTTCCTTAAATTTCATGAACCGACTTCTGCT |
| | | | TCCTTCAAACATTTCTTCTGTAGCTTCTTCACCTCTCTTAGCCTTCA |
| | | | CAGAATTGAACAGATTTAGGATTTTGCTCTGGTTTAGGCTTTAGCTT |
| | | | AAGAGAATGTTGTGGCTGGTTTGGTCTTCTATCCAGGCTACTGAAAC |
| | | | TTTCTTCATAGCACCAATAAGATAGTTTTACTTTCTTGTCACTAATG |
| | | | TGTTCATTGATGTCACACTTTTAATTTCCTTCAAGAACTTTTCCTTT |
| | | | GCATTCACCACTTGGCTAACTGTTTGGTGCAAGAGGACTGGCTTTCA |
| | | | GACCATCTCGGCTTTGGACATGCCTTTCTCACTAAGCTTAATCATTT |
| | | | CTAGCTTTTGATTTAAAGTGAGAAAACATGTGACTCTTCCTTTCACT |
| | | | TGAACACTTACGGGACATTGTAGGGTGATTAATTGTCCTGCTTTCAA |
| | | | TATTGTTGTGTCCCAGAGAATAGGGAGGCTCAAGAAGAGGGAGAAAA |
| | | | ACAGGGAACACCTGGTTGGTGGAGCAGTTAGAACACACACAACATTT |
| | | | ATCGATTAAGATCTCTGTCTTACAGGGGCACAGATCTCGGCGCCCCA |
| | | | AAACAATTACAATAGTGACATCAAAGATCACTGATCACAGATCACCA |
| | | | CAACACATATAATAATAATGAAAAGGTTTGAAACATTATGAGAATTA |
| | | | TCAAAATGTGGCACAGAGATACAAAGTGAGCATATGCTGTTGGAAAA |
| | | | ACAGAGCCAATAGACCAGGTGGATATAAGGGGTTACCACAAACCTTC |
| | | | AATTAGTA |
| IFNG | rs10492197 | 8 | TGTGATTGAAGATTACCTATAAATACATGCTGAGCTTTCTCTATGTA |
| | (POSITION | | CCTGATTTTGTGGAAACTATTTACGGTTCTGCTGTTTTATTCTGATA |
| | 201) | | TAGCTTTCCAAGTGTTTCCTCAAATTTTACTACATTGTGTATTTTAC |
| | | | TCATTTAGCCAACAAAGATTTATTTGTTTTACTTATTAAGTGTCAGG |
| | | | CTCTGTCCTAAAYGTTAAACAGGTGAACATACCATTCTTGATAGGGG |
| | | | GACACAGAAATAAACAAAGGAGTAAACATAAAGGATGTCAGAATAAC |
| | | | AAGAACAAACAAGCAGGAGTGGGGGGGTTTCAGGGACTGGGGAAGGG |
| | | | CGGGGACTGGTTTGCTCTTAAAAAAAAGGCTGATCAGAGCTGGGCAC |
| | | | AGTGGCTCATTCCTGTAATCCCAGCACTTTGGGAGGCTGAGGTGGGT |
| | | | GGATCACGAGGTCAGGAGATTGAGACCATCCTGGCTAACACTGTGAA |
| | | | ACCCTGTCTCTACTAAAAATACAAAAAAATTAGCCGGGTGTGGTGGC |
| | | | AGGCACCTGTAGTCCCAGCTACTTGGGAGGCTGAGGCAGGAGAATGG |
| | | | CGTGAATCTGGGAGGCGGAGCTTGCAGTGAGCTGAGATCACACCACT |
| | | | GCACTACAGCCTGGGTGATAGAGGAGACTCTGTCTCCGAAAAAAAAA |
| | | | AAAAAAAGGGCTGGTCAGGAAAAAGCTCACCAATGAGGTGACATTTT |
| | | | TGCACAGACCTGAAGGATCCTTACAATGACTAAGGAGTAGAGAGTAA |
| | | | AAAGATTATTGATTTTGGTTTTGTAATTTATGTGGATGTAGAAACAG |
| | | | GCTTGGGGATGTTAAATATTTTTA |
| IFNG | rs10748099 | 9 | CAAGAAGAATTCAGAGAAGGAATCTCATTTGACTAGGGATGGGAGTG |
| | (POSITION | | AGAATATGAGAGGTGGCAAAAATGAACAGATGGGTAGGGTCACAGGT |
| | 278) | | AATATGCACAAGACCTCTCTTCTCATGAAGCTTACATTTTAGTAGAG |
| | | | TCAAAGAAAGGAAGATAATAAACAAGGCAATCAACAAAGAAACAAGA |
| | | | TAATTTCAAAGCATGAGGATAATATGAAGGAAATAACAAAGGTGATT |
| | | | TGGAATTACTAGGAGTGGATGGAGATCCTTCCTCAGCTGGGTYGGGA |
| | | | ACGTCATGTCAAAGGAAGAGACCCTTGAGCTGACACGTAAATGAAAG |
| | | | GAACGGACTGTGGGAAGGCCTGGGGAAGGGTACTCCAGGGAGAGGAG |
| | | | CTAGCATCTACAAATGCCCAAGACAGAGCTGAACTTGCACTTTTCAG |
| | | | AAGCAGAAAGGTCAGCTAAGAGACAACACAGGCCAGGAGACAAGGTC |
| | | | AGAGAGAAAGGCTAGGCAATTAATGTAGGTCTTTCTTGGCCAGATAA |
| | | | TAAGGTTTATTCTCAGTGCAAGGGAAGCCATTGAAAGGCATCAAACA |
| | | | GGAAGGGATATGCTTTGATTTACACTTCTTAAGTTCTCTCTAGAAGC |
| | | | TCAATGAAGCTGGATTCAGGGGCAAGGTATGAGTGGAAACAATGAGA |
| | | | CCAGTTAGAAGGAGGACTCTTCCAGTGTCCAGGTGAGACATGGCAGT |
| | | | GACCTGGGCCAGGGTATACTAATGGGGATAGGAGAAGCGGAAGGATT |
| | | | TGAGATATATTGGGGCGGTAGAACTGCAAGAATGTGCTGATGAATTT |
| | | | GGTTTGGGATATGAGGGAAAAGAAGAAATAAAAAATCCCTGTAATTG |
| | | | CAAAAATGGCCCTAGCAATTGAGTAGGTGACAATTTATCATATAATA |
| | | | ATAACAACTTATGCGTATAAAGTTTTTATTATATAGCAGTCATGGCT |
| | | | CTAACCTCTTTACATATATTACCTCACATGAACCCCACAACAACCCT |
| | | | ACAAGATAGGTACTATTCTCATCCCTATTGTACAGACAAGGGAAGAG |
| | | | AGGGACGGACAGATTAACCTCACTTTGTTGTTAAATTACAGCCTCTA |
| | | | TGTGAAGCTTTATCGGCTTCAGAGTCTGTGTGCTTAACCATGATATC |
| | | | TTTACGTTTTGTATTACCAGGTTGTGGAATACTAGAGAATGAACTGA |
| | | | TTTTAGAAGGAGAAACAAATTTTCCGGTTTTGACATATTGTTTTTGA |
| | | | GATGTCTTACATGGAAATATCGAGTACATAATTGAATGTGTGAGCAT |
| | | | GGAATTCAGGGACTAGGTCAACCCTGGAGACATTAGCACACTGATAG |
| | | | TATTTAAAGCCATGGGGTTGAATTAGCTGTATAGAGAGCAATAGAGT |
| | | | ACATGGAGATTACAAGAAGCCACAACTAGCCCTGAGTCCTCCAATCT |
| | | | GTAGTGTTCTGATAGAGAAGAAACTCACTTGCAAGATCAAGAAGCAG |
| | | | CATCTAAGTGAGGCAGAAAGAATCCCAGAGGAGAGTGTGGATTTTCA |
| | | | GAACTGAGTGATTAACATGTTGGCTTGATTCTCAGCCAGTCTCTGTC |
| | | | CTCATGGTGGCAAGATGGCTGCAGCAATTCCAACCAATACTCTTCCA |
| | | | AGCTTATAGTTCATAGAAAAGAGAAAGACTCATTTTCCAGAACTCAT |
| | | | TTATAAATCCTGGAATCCACTCTGATTGGGCCTTGTTGGGTCATAGG |
| | | | CCCATTCCTGAATCTTCACCAATCATTGTGACTAGAGGACCCTAGAG |
| | | | TAGG |
| IFNG | rs1076025 | 10 | GGAGCAAGACTGAGTTTGAGTCCAGGCTCCATCTTTTACCAGCTGTG |
| | (POSITION | | TAAACTGTGTGAATCTAGGCAAGCTCCTTAAAGTCTCTGGACTCTAC |
| | 501) | | TTCACAGGTTTTTTGTGGGATTCAAATGAGTTATATGTGCAGCTCTT |
| | | | GGAATAATACTTGGCATATAGCAAGCACAATGTGTGCTCATCATTTT |
| | | | TATTTCCATTTTATGGGTTTTTTTCCCTTGTAACCTGATTTAGAAGT |
| | | | TGTATTTGTACATTTCTTCATGTTTAACGTATTTGTTCAGGTTAAAT |
| | | | TGAAATATTTTACATATAGAAACTGAGGTTGGGTTACCTCAGAAACA |
| | | | GAGCTTGAGACAAGGATTTTTTTTTTTTTTTTTTTTTTTTGGTCGTG |
| | | | ATTCTAGGAAGCACCAGTAGAAAAGAGGCAAAGAGATTCAGGGAAGG |
| | | | GAAGGAAGTCAGTTCAGGGTGGTTCCCAAAGGGAGCTACTGTAGTCA |
| | | | ACTGAGACTCAGCCCACTATAGACCTCTGGRTGATGGTGTAGCCCAT |
| | | | ACCCCAAAGTTATCCTGCCCAAGGGACGAAGAAGTTGGGGTATCTAT |
| | | | CCTGCGACTATCTTTAGCACTGTCTGAGCACTGCTCCCAGGGCATTA |
| | | | AACCCCTAGCTCTTCCAGTCTTCCTCATGTGAAAATAGAAAGAAGCC |
| | | | CTTAGGCCAAGAATAGTGAACTGTTACAGTCACAGGCAGAGGGTAAG |
| | | | AAGAGAGAGGGAGGCTGCTGAGAGGATGTTGGCAAGGCAGGTAGTAT |
| | | | CTGCTATGAGAAGTTATTAATTATTCCCTCATATTTTTTTTCAGTTT |
| | | | TTATTACATCCTTTATTTTTCGGCATTAGTGTCAGT |
| IFNG | rs10784683 | 11 | ATCTCAAAAGCTGCTGTTCATAGTCATTCACTGTTGGACATTAATGA |
| | (POSITION | | CAAATCACTTTCATGAACTGACATCCATTTAAAGGGATTTTTTAAAA |
| | 201) | | ATGTGTTTATCTCATAACTGCTCCTGTTTATATGACGATCCTGTCTT |
| | | | CTTTGAGATTATAATGACAACAAATGTTATTCGTTTTCTGCACTATT |
| | | | CATATAAACAACRTAACTGGGCATAATACTTTCATGATATCATGTCA |
| | | | TTACTAATAAATCACCTTTTTAAAACATCTCTATGATAGTATCATGG |
| | | | TTAACAAACAGCACAGACAAAGGAGCAAGACTGAGTTTGAGTCCAGG |
| | | | CTCCATCTTTTACCAGCTGTGTAAACTGTGTGAATCTAGGCAAGCTC |
| | | | CTTAAAGTCTCTGGACTCTACTTCACAGGTTTTTTGTGGGATTCAAA |
| | | | TGAGTTATATGTGCAGCTCTTGGAATAATACTTGGCATATAGCAAGC |
| | | | ACAATGTGTGCTCATCATTTTTATTTCCATTTTATGGGTTTTTTTCC |
| | | | CTTGTAACCTGATTTAGAAGTTGTATTTGTACATTTCTTCATGTTTA |
| | | | ACGTATTTGTTCAGGTTAAATTGAAATATTTTACATATA |
| IFNG | rs10784684 | 12 | CTCCCACAGAGCAGCATTCACCAGCTGGAAGGTAAGTTAGCCATTAA |
| | (POSITION | | GGCATTTAATTGAAACACTGCACTAATTCATCAAATACTTGCTGAGC |
| | 1303) | | TACATATTTATATCATCAGGGAAATGCAAATTAAAACAACAAGATAC |
| | | | CCACACACCCATTATGAAATGGCAAAAATCTGGAACACTGACAACAC |
| | | | CAAATGCTGGCTGAGACGTGGAGCATCAGGAACTCTGACTGAAGGTA |
| | | | CAGCCACTTTGGAAGACAGTTTTGCAGTTTCTTATAAAACTAACCTT |
| | | | ACTCTCACTATACCAGCCACCAATCACAACATTCCTTTGTATTTACC |
| | | | CAAAGGAGTTGAAGTCTTATGTCCACACAAAAATCTGCACACAGATG |
| | | | TTTATAGTAGTTTTATTCATAGTTACAAAAACTTGGAAGTAACCATG |
| | | | ATATCCTTCAGCAGATGAATGGTTTCATAACTGTGGTGTATCCATAC |
| | | | AGTGGAATGTTATTCAGCCTAAAAAGAAGTGAGCTGTCAAGCAATAA |
| | | | AAAGACATGGAGGAACCTTCAATACATATCACTAAGTGAGAGAAACC |
| | | | AGTCTGAAGAGACTACACACTGCATGATTCAACCATATGACAGTCTG |
| | | | AAAAAGAAAGATCAGTGATTGCCAGAGGTTGGCAAGAGGAATGAAAA |
| | | | GGTGAACACAGAGCATTCTTAGGACATGCAAACACTTTGTGTGGGAC |
| | | | TCAGAATGAGAGATACACATTCTGCCTTTGTTCAAACCCATAGAAGT |
| | | | TTCAACACTGAGAGTGCAAACCATGGACTTTGGATGATGATGATGCC |
| | | | ATTGTAGGTTCATCAGTGGTAACGAGCGTACCTCTCTCATGGGAGAT |
| | | | GTTGATTATGGGGAGAGGCTGTATATGTGTAGGGGACAGAGGGCATA |
| | | | CGGAAAATCTCTGTACCCTCCTTTTAATTTTGCTGTGAACCTAAAAG |
| | | | TGCTCAAAAAAAATAAAGCCTATTAAAAAATACTTGTTGATGTGCAA |
| | | | GACATTCTTCTAGGCACTGAAGAAACAGCAAGAACTAACAAAAAAGG |
| | | | GACAAAACTCCTGTCCCCATGGGCCTTACATTGTAGTGGAGAAGATT |
| | | | AACATAAACAAACATGTAATTGTGTAATACAATGTCAGGTTGTGATT |
| | | | ATGATTTGAAAAAGGAAAGCAGGAGAATGGAATAGTGCTATTTTAGA |
| | | | TAGGGGGGTTGGGGAAGACTTTTCTGAGGAAAGAACATTTGAGCAGA |
| | | | GACCTGACTGAAGGTGGTGAGGGAGTCATGGACACGACTGGGAACCA |
| | | | TGTCCCAGGCAGAGAAGAGCCAAATGGAAAAAGYCAAGACAGACGCC |
| | | | CCTTCAGCGAGGGCTGAGTCATAGCAGGGGTCATGTGTCTGGACCTG |
| | | | AGGAGCAGGCAGTGGGGTTGGAAAGATAACCAGGGGCCAGATCATGC |
| | | | CCCCAGAAAGCATTTTGGGTTTTATTCTAGAGGAAATGGGGTACTCT |
| | | | CTACTGGGTTTTGAACAAGAGAGTGACATGATCTGAGATATATTTTA |
| | | | ATGGGATCACTGTGGTCAGCAAATGGAAATTTGGCTCTAATGGGACA |
| | | | AGGGCAGAAACTGAGAGGCCAATTTAGGAGGCTTCTGTACTCATCCA |
| | | | GGAAAATCCAACTGTGGGGCTCCAACAGTTCAAATGAATTAACAAAA |
| | | | AAAGAGTCAGAAAAAATATGGCAACACGCCCCCTCACAAATCATGTG |
| | | | TACCATATA |
| IFNG | rs10784688 | 13 | TGGAGCGTAAACTCCACGTCAGTTTATGTGGCTACACATAAAGATAA |
| | (POSITION | | CTCCAATAAACCACCTTCAGGGAGCCTGCTCGAAGTACTTGCCATGC |
| | 304) | | TGGCTCCTTACACGGTTTCACTTAACATAATGTTGTTACATAAGTAC |
| | | | ATCTACCTATCTTTTTTTTCTTTTCTTTTTGTTTGATTCATGCCCTT |
| | | | TTTTTTCTTTTCTTTTTGTTTGATTCATGCCCTTTTTTTTTTTTTTT |
| | | | TTTTTTTCTTGAGTGAATCCTAAGTCAGGAGGCAGTAGGGGTTAGCA |
| | | | ATTTAAACCCCAGACAAAAAAYTCTGGTTCAAATCCTAGCTCCATCT |
| | | | TCACTAATTGTGTGACAATGGGCAAGTTACTTAGCTTTTTAGGATCT |
| | | | TACTTTCCTCAATTAAAAGTAGGGAAGAAAATAGCACCTATCCCATA |
| | | | GAGTTGCTGTGAAGAATAAGTGTTGTTGGGTGGCTCATGCCTGTAAT |
| | | | CCTAGCACTTTGGGAGGCCAAGGCAGGTGGATCACTTGAGCTCAGGA |
| | | | GTTTGAGACCAGCCTAAGCAACAAGGCAAAACTCTGTCTCAACAAAA |
| | | | AATGCAAAAATTAGCCTGGTGTGGTGCCTTGCACCTGTAGTCCCAGC |
| | | | TACTTGGGGGGCTGAGGTGGGAAGATCACTTGAGCCCAGGAAGTCGG |
| | | | GGCTGCAGTGAGCTGAGATGGTGAGGCTGCACTCCAGCCTGGGTAAC |
| | | | AGAGTGAGACCCTGTCTCAAAAAATCAATTAATCAATAAAGTGTTGT |
| | | | TGATGTTTATGAAACCCTTAGAGCTCTACCAGGCATACAGTGAACTA |
| | | | CGATG |
| IFNG | rs10878763 | 14 | GTTCTTGGAAGTTCTTTTTTGTGTGTTTTTTTCTATTCTGTTTGTTT |
| | (POSITION | | GCTTGTTCTTCATTTTCTCTCTCTGCATTTCAGTTTGGGAAGTTTCT |
| | 1958) | | ATTTACCTATCTTCAAGCTCACTGATTCTCTTCAAGCTCACTGATAC |
| | | | GTTGTGTTTACTGGAGCCTATCGAAGACAATCTTCATTTCTGTCACA |
| | | | GTATTTTTTATTTATAGTATTTCTATTTGATTCTTTTCTTAGAATTT |
| | | | CCATCTCTCTACTGACATTACCCATCTGTTCTTGCATGTTGTCTACT |
| | | | TTCTCCCTTAACATATTAATTTTAGTTATTTTAAATTTCTTACCTGG |
| | | | TAATCCCAAACTCTATGTCATATCCGAGTCTGGTTTTGATGTTTGCT |
| | | | GTATCGCTTCAGGCTGTGTTTTCTCTCACCTTTCCGTGTGCCAGAGG |
| | | | CTTCAAGTTCTCTGGCATTCTTGCCTTTGTCTCCCATCTTTACCTTG |
| | | | TGCTTCCGTAACTACTCCTACTTAGACAGAGTCTGTGCCTTGCAGCT |
| | | | CTTTCACCTGTGATCCACTGTTATTACTGGAGCCCTGTGGTATGTAG |
| | | | TAAAGTATGGGGAAAGGGAAGTGTTTTATAATCTTTAAATCTCAGCA |
| | | | TTTTAGTGGGCCTGTGTCTCAGGACTGTGATCTTCACAAGTGTTTCT |
| | | | TCTTGTATAGCTTTAGGTGTAACAGGACAACTAGAAGGGACTCAAGT |
| | | | TAGAGAAACATCCTTCCCCCACAGCCCTCTCACAGGAGTCTGGTAAA |
| | | | GCCTTTCCCCTGGAGAGCAGACCTTTGTTTCTGGACATACTTCAGAA |
| | | | GGTTACTCGTCCCCTCCCCCTGCCAGAGCCACAGGGGTATCTTTGTC |
| | | | AGAACTTCACCAGGAGAACTTGGTGGGATTCCTGTAGGTATGCTCAC |
| | | | GAAAACAAGGAGGACCCATCACAGTTCGGCCCCCAGGTGTTTCTCAC |
| | | | TCCCATGCTAGTCCACACTCAGCCTCCAGCAAGTCATCAAAATTACC |
| | | | ATTTAAGTGTTTTAACAAGTTAATTACTCCAGTGGATTCAGGTCCAA |
| | | | GTAAGCAGATCTTGGCTGTGAATTTCTGGATTTGCCTAcTCTCCAGA |
| | | | TTTTATTGTGGCAGTTTGTCCTGCAAATTCCGTTCTATGATGGAACT |
| | | | AAAAAACTCGCTGGTTTTATTTGTCCAGCTTTTCCTTGTTTTAAAGG |
| | | | CTGGAGTAACAACTTCCATGCTCTGTATATGTTGGAGCTAAAATTGG |
| | | | AAGTCTGTCACGATGGTTTTTTTTCTTTTTTTTCTTTTTTTTTTTTT |
| | | | TTCCTGAGATAGAGTCTCACTCTGTCATCCAGGCTAGCGTGCAGTGG |
| | | | CATGATCTCAGCTCACCACAACCTCCACCTCCCGGGTTCAAGCGATT |
| | | | CTCCTGCCTCAGCCTCCTGAGTAGCTGGAACTACAGGCATGTGCCAC |
| | | | CATGTCCAGCTAATTTTTGTATTTTTAGTAGAGATGAGGTTTTACCA |
| | | | TGTTGGTCAGAATGGTCTCAATCTCTTCACCTCAGGTGATCCGCCCG |
| | | | CCTCGGCCTCCCACAGTGCTGGGATTACAGGTGTAAGCCACCACACC |
| | | | CAGCCCATGATGGTTTTTTTCATTGAGGCCTCAGTTGGAAAATTCAA |
| | | | ATGCTTGGAGCTACAATCATCTAAGAGCTTGCTCACACACATCTGAT |
| | | | GATTTGTGCTGATGCTGAGTGGAAGCCTTACTGGAACTCTTGGCCAG |
| | | | AATATGCACACATGGTTTCCCCATGCAGCCTGAACATCTCAACATGA |
| | | | TGTTGGGTTCTGAGGGCAAAAGTCTTGAGATGGAGAGAAGCCAGGTA |
| | | | GAGACTGCACCCTAGACTTCAAAGGATGTGACTTCATTTCCATTTCA |
| | | | CTTCACTGGTAAGCAAAGTCACAAGCCCCCGCCCAGTATTTAGGGGA |
| | | | GGACCATACCCTCATCTTTAAGTTGGGGGAGTGTCAGTCACATTACA |
| | | | AGAAGAGCATGGGGATGGGGTGAATATATAKGTGTGATTACTTTTGG |
| | | | AAATTTCACCTGTTGCAAGTTAAATATGGGGAATTCTGAGTCATCAA |
| | | | GAATTTTAGACCTCACCAGTCTGTGACTCTGAAATAATCTCAGAGTG |
| | | | ACTTTTTCGTATTTATATTTTGAAAAAATATTGCAGGCTGGGCGCCT |
| | | | TCAAATCCCAGCACTTTGGGAGGCCAAGGTGGGTGAATCACTG |
| IFNG | rs10878766 | 15 | GTAAGAAGAGAGAGGGAGGCTGCTGAGAGGATGTTGGCAAGGCAGGT |
| | (POSITION | | AGTATCTGCTATGAGAAGTTATTAATTATTCCCTCATATTTTTTTTC |
| | 272) | | AGTTTTTATTACATCCTTTATTTTTCGGCATTAGTGTCAGTATACCA |
| | | | ACAAGTTGCATTTGCCAGGACTTTTGTGGTGACAAGTGACGAAAATT |
| | | | CCAGTCACACTATTTTGATCAAAGAAAGGATCTCAGAGACAGGTACT |
| | | | CAAGTGTTGACAGGATTTGTCTCTCTAGCTGTCACTKCTGCTTCTCT |
| | | | TTGTGAGACAATGTCAATCCTGCCTCCCACAGAGCAGCATTCACCAG |
| | | | CTGGAAGGTAAGTTAGCCATTAAGGCATTTAATTGAAACACTGCACT |
| | | | AATTCATCAAATACTTGCTGAGCTACATATTTATATCATCAGGGAAA |
| | | | TGCAAATTAAAACAACAAGATACCCACACACCCATTATGAAATGGCA |
| | | | AAAATCTGGAACACTGACAACACCAAATGCTGGCTGAGACGTGGAGC |
| | | | ATCAGGAACTCTGACTGAAGGTACAGCCACTTTGGAAGACAGTTTTG |
| | | | CAGTTTCTTATAAAACTAACCTTACTCTCACTATACCAGCCACCAAT |
| | | | CACAACATTCCTTTGTATTTACCCAAAGGAGTTGAAGTCTTATGTCC |
| | | | ACACAAAAATCTGCACACAGATGTTTATAGTAGTTTTATTCATAGTT |
| | | | ACAAAAACTTGGAAGTAACCATGATATCCTTCAGCAGATGAATCGTT |
| | | | TCATAACTGTGGTGTATCCA |
| IFNG | rs10878774 | 16 | CTGTTTGTCCTCCCAAACACAGCAGGCAGAAGAGTCACTCCACCCAG |
| | (POSITION | | GGCAAAGTGAAGGAGAGGGTGGAGGGAGATTGGGAATGCTGTGCTCA |
| | 201) | | TAGATCTCTCTTGACAAGAATGGGGAGAAAAGTTCCACACCAAAGGA |
| | | | GGGCAAAGCCAGAGAAATAGGGAAGAGGTCTCGGGATCTGCACAGTG |
| | | | AGTTTGTGGAGCRTAAACTCCACGTCAGTTTATGTGGCTACACATAA |
| | | | AGATAACTCCAATAAACCACCTTCAGGGAGCCTGCTCGAAGTACTTG |
| | | | GCATGCTGGCTCCTTACACGGTTTCACTTAACATAATGTTGTTACAT |
| | | | AAGTACATCTACCTATCTTTTTTTTCTTTTCTTTTTGTTTGATTCAT |
| | | | GCCCTTTTTTTTCTTTTCTTTTTGTTTGATTCATGCCCTTTTTTTTT |
| | | | TTTTTTTTTTTTTCTTGAGTGAATCCTAAGTCAGGAGGCAGTAGGGG |
| | | | TTAGCAATTTAAAGCCCAGACAAAAAATTCTGGTTCAAATCCTAGCT |
| | | | CCATCTTCACTAATTGTGTGACAATGGGCAAGTTACTTAGCTTTTTA |
| | | | GGATCTTACTTTCCTCAATTAAAAGTAGGGAAGAAAATAGCACCTAT |
| | | | CCCATAGAGTTGCTGTGAAGAATAAGTGTTGTTGGGTGGCTCATGCC |
| | | | TGTAATCCTAGCACTTTGGGAGGCCAAGGCAGGTGGATCACTT |
| IFNG | rs10878779 | 17 | TAAGTCAGGAGGCAGTAGGGGTTAGCAATTTAAAGCCCAGACAAAAA |
| | (POSITION | | ATTCTGGTTCAAATCCTAGCTCCATCTTCACTAATTGTGTGACAATG |
| | 501) | | GGCAAGTTACTTAGCTTTTTAGGATCTTACTTTCCTCAATTAAAAGT |
| | | | AGGGAAGAAAATAGCACCTATCCCATAGAGTTGCTGTGAAGAATAAG |
| | | | TGTTGTTGGGTGGCTCATGCCTGTAATCCTAGCACTTTGGGAGGCCA |
| | | | AGGCAGGTGGATCACTTGAGCTCAGGAGTTTGAGACCAGCCTAAGCA |
| | | | ACAAGGCAAAACTCTGTCTCAACAAAAAATGCAAAAATTAGCCTGGT |
| | | | GTGGTGCCTTGCACCTGTAGTCCCAGCTACTTGGGGGGCTGAGGTGG |
| | | | GAAGATCACTTGAGCCCAGGAAGTCGGGGCTGCAGTGAGCTGAGATG |
| | | | GTGAGGCTGCACTCCAGCCTGGGTAACAGAGTGAGACCCTGTCTCAA |
| | | | AAAATCAATTAATCAATAAAGTGTTGTTGAYGTTTATGAAACCCTTA |
| | | | GAGCTCTACCAGGCATACAGTGAACTACGATGTTGTTGATGATGATA |
| | | | ATCATCTTTATTGGCACATGCCAGGACTTGATAACCTTAGTTTGTAA |
| | | | TGTGAATCCTATTTAAAAGTATTTAAAAGTATTTCCACTACAACTTA |
| | | | AGAAACTGTCATCCAGTGCAAAGCTCAGGGTAGACAGCAGAGAGTTG |
| | | | GATTTAGCCATGATTGATTGGAGTTTTTCCAGGAAAATACGATGAAG |
| | | | GAAGACAAGAACAAATGACAGACCATGGAATTGAGGCTCGATAATGA |
| | | | GAGAAGTAAAGACATAAAGTGGAGAGGAACCGTGAAAAGATGCTAGG |
| | | | AATAATGTTTTTTTTCAATTCCATTGGAATTTAATGACAGCTAGAGT |
| | | | GGGTTATAGAAAGGGCAAGCTGAAAAGTCATAGAGTAGGAGTCATGT |
| | | | CATTGAGATAATGTGGGGAATGGGGGCTGTTGCTATTACAATGCAAA |
| | | | TTCTAGGATCCTCC |
| IFNG | rs10878781 | 18 | GAGCGATGGTCCTATTTCCCAGAGGAATAAGAGCTCTGGGCTCCTTC |
| | (POSITION | | AGGAAACCTGGGGAAGAGGATGTCCAAGTCTGCATGAATACCAACAG |
| | 301) | | ATGAGGCCATCGGAAGAAGGGCTCCTAAGAAAGAGAAACCACACACA |
| | | | GAAAGGAAGAAGTGAATATGACCCATGCTCACACACCAACATGCCTA |
| | | | TAGCCAGGAGGAAATATGAGAGCTAGGAGGGAATTTAGGAGTCTCTG |
| | | | AATTGAAAGTATTCGTTTCAGTGAGCAGGAAACTGAAGTTTAGAGAC |
| | | | GTAGAGTAAACTTATTGTRAGAGGAACCTATGTAATATGTCTTAGAA |
| | | | AGCTCTCTTTCAAAATCATTATCCAAAAAGGAAAGAATGGGCCACTT |
| | | | AAAGGAGTATTGATTTATTAATCGGGAAATTTGCTTATGGAAAATAG |
| | | | GCAAAACTTGCTTCGAAATGCTTATCACAATCCACCTAAAATTTCTG |
| | | | TTGGCAGCATCATTATCTGTAGCTGCTTCAGTGGTGACACTAATAAA |
| | | | TTCACATTACAGAATAGTAGTAAAGGATTTATTTTTCTTTTACATTT |
| | | | TATATTATGGTCACCAATTGTGAGCTCTGAAGTAAAA |
| IFNG | rs10878784 | 19 | AGACTGCATAATGTGCCTTTCCAGGGGGTGTTTCTCTCTATTGATTA |
| | (POSITION | | TTTGAACTGTTAAACTTGATTACATTTTACTTTAATTGTACCATTTG |
| | 368) | | AAATTAGATTCAGGTAAGATTTCAAACTTATTAAATAAATGGCCCAT |
| | | | AGGACATTTGGGGGAATGTCTCAAAAAAGAAAAATGTAAATAGAATC |
| | | | TACATATAAAAGTGATCAATTTAACAAGCTTTAAGGGGAAGGCAAAG |
| | | | TAAAACAATATGATGTAATTTGCAGCCACCAGACCGGCAAAAATATT |
| | | | CAAATATTGATAATATCCAGTTTTAGCAAGAAAGAATGTGGGGAAAA |
| | | | ATTAGCAATGAAATAGTTATGAAAGTACTTTGGCAATAKTTGGCATA |
| | | | TGTTCTGATCCCACAATCCTGCTTCTAGAAAGCATTTCTGTAAAAAT |
| | | | AAGAGCACAGATTAGGACACACATTTACAGCCTGTGCTATATGAACA |
| | | | AAGCTGAAATTAACTGGGACTACCGAATAAATAAAATACATTATATT |
| | | | TGCAAAATATATAATTCATAGCTAATATGACATTTTAATTTTTATAT |
| | | | AAAAATATATTTTTATATCTGCCCATATGCATATGCATGCATGCATA |
| | | | CCCAGACATGTGTATACACACATTTACATACCTGGAAGGA |
| IFNG | rs10878786 | 20 | TAAAATACATTATATTTGCAAAATATATAATTCATAGCTAATATGAC |
| | (POSITION | | ATTTTAATTTTTATATAAAAATATATTTTTATATCTGCCCATATGCA |
| | 284) | | TATGCATGCATGCATACCCAGACATGTGTATACACACATTTACATAC |
| | | | CTGGAAGGATGTTCCCGATGTGTTAAATGGAAAGAGCTAGTTGAAGG |
| | | | GTAGAATAAATGATATGATAACGTTTTTGTTTCTAGAGAAGGGAAAG |
| | | | ATACTCTATATGAACATATATTTATATTGTTGTTGGAAAAATTTAAA |
| | | | ARTTGTGGGAAAATCCCCACAAACTGCCATCATTGGCTCACTTGGGA |
| | | | AAGTAGAGGTGGAAAGGCAGTGAGCTATGATTAGTTTATATACCTTG |
| | | | GTGTTATTTCAGTTTTACAACAAACATATATTACTTTTTGTAATATA |
| | | | GGAAACTATAGGTTTGTAACTAGGAAAATATATATAAATTTCAAGAG |
| | | | GACAGATTTCAGATTAATATGAATAATTTTCTAATAGGCAGGATTAT |
| | | | TTGGATTTAGCGAGGGCTCTTCAAGGGGTCACTAGTCTTTCTTAATT |
| | | | GTGAGCGGTCAAGCATAAGTTAGATGAGGACAGTGTTAGGAAGGAGA |
| | | | TTCTGGTATAAGATGCAAAGTTGGACAATGTAGCCTCATTGGTCTTT |
| | | | TTAAATTATGACATGCCAGGCTTCTACAAAGTCCACATTTCAAGGCG |
| | | | TTTCTGCGTTTGGCCAAATGAGA |
| IFNG | rs11177081 | 21 | AGTCCAAGTTATCCTCATGTGCTTTTCTTCCTCACTAGGTTTTAAGG |
| | (POSITION | | TCCTAGAGAGTATACACTGCCTCTTAGTCTTCTTCATCTATCTCAAA |
| | 301) | | GTGCCTGGCTGAGTGCTTTACATGAAGTATCCAATAATTCTTGACCA |
| | | | TCAGACCTGGGGGGTGGAACCAGCAGGGCCATTTAGCCAGGGCTGCA |
| | | | AGCCCAAACAGATCTCTATTCTTCAGCTGCAAGTTAGTGCCCAAGCC |
| | | | ACATAGGGAATAGGATGATACCTCATTACACATGCTGATGTTAGCTT |
| | | | TAAACTATGCCTGCCCTCKGTTTTCCTAAAAGCTGTGTTACTGCCAA |
| | | | TCTCAAAAGCTGCTGTTCATAGTCATTCACTGTTGGACATTAATGAC |
| | | | AAATCACTTTCATGAACTGACATCCATTTAAAGGGATTTTTTAAAAA |
| | | | TGTGTTTATCTCATAACTGCTCCTGTTTATATGAGGATCCTGTCTTC |
| | | | TTTGAGATTATAATGACAACAAATGTTATTCGTTTTCTGCACTATTC |
| | | | ATATAAACAACGTAACTGGGCATAATACTTTCATGATATCATGTCAT |
| | | | TACTAATAAATCACCTTTTTAAAACATCTCTATGATA |
| IFNG | rs11177083 | 22 | GAAAGAAGCCCTTAGGCCAAGAATAGTGAACTGTTACAGTCACAGGC |
| | (POSITION | | AGAGGGTAAGAAGAGAGAGGGAGGCTGCTGAGAGGATGTTGGCAAGG |
| | 272) | | CAGGTAGTATCTGCTATGAGAAGTTATTAATTATTCCCTCATATTTT |
| | | | TTTTCAGTTTTTATTACATCCTTTATTTTTCGGCATTAGTGTCAGTA |
| | | | TACCAACAAGTTGCATTTGCCAGGACTTTTGTGGTGACAAGTGACGA |
| | | | AAATTCCAGTCACACTATTTTGATCAAAGAAAGGATYTCAGAGACAG |
| | | | GTACTCAAGTGTTGACAGGATTTGTCTCTCTAGCTGTCACTTCTGCT |
| | | | TCTCTTTGTGAGACAATGTCAATCCTGCCTCCCACAGAGCAGCATTC |
| | | | ACCAGCTGGAAGGTAAGTTAGCCATTAAGGCATTTAATTGAAACACT |
| | | | GCACTAATTCATCAAATACTTGCTGAGCTACATATTTATATCATCAG |
| | | | GGAAATGCAAATTAAAACAACAAGATACCCACACACCCATTATGAAA |
| | | | TGGCAAAAATCTGGAACACTGACAACACCAAATGCTGGCTGAGACGT |
| | | | GGAGCATCAGGAACTCTGACTGAAGGTACAGCCACTTTGGAAGACAG |
| | | | TTTTGCAGTTTCTTATAAAACTAACCTTACTCTCACTATACCAGCCA |
| | | | CCAATCACAACATTCCTTTGTATTTACCCAAAGGAGTTGAAGTCTTA |
| | | | TGTCCACACAAAAATCTGCACACAGATGTTTATAGTAGTTTTATTCA |
| | | | TAGTTACAAAAACTTGGAAG |
| IFNG | rs1118866 | 23 | AGTCTTTAATCCATTTTGATTTGATTTTTGCATACAGTGACAACTAG |
| | (POSITION | | GAGTCTAGTTTTATTCTTGTGCATATGGTTATCCAGTTTTCCCAGCA |
| | 256) | | CCACTTATTGAAGACACTGTCTTTTCTCCAGTGTATGTTCCTGGCAC |
| | | | CATTATCAAAAATTAGTTTATGGTAGGTGGTGGATTTCTTTCTGGGT |
| | | | TATCTATTCTGTTCCATTGGCCTATGTGTCTGCTTTTATGCCAGTAC |
| | | | TGCTGTTCTGATCACTAAACYTCTATAGTATAATTTGAAATCAGGCA |
| | | | ACATGATTCCTCTAGTTTGTTCTTTTGGATTAAGAAAGTTTTGGCTC |
| | | | TTGTGGTTCCATATCAATTTTAGGATTTTTTTTTCTATTTTTGTGAA |
| | | | GAATGTCTTTGGTATTTTGATAGGGATTGCACTGAGTCTGTGGATTG |
| | | | CTTTGGATAGTATGGACATTTTAATAATATCAATTCTTCCAATCCAT |
| | | | GAACATGGAATATCTTTCCATTTTTTGGTGGCCTCTTCCAT |
| IFNG | rs12301088 | 24 | TTTATTTATGAAGCATTTTTTCTTAAGAAGTTAAAAACATAAAACCA |
| | (POSITION | | GTGATACACCAAGGTATTTAATGGAGGGGGAAGAGTGGGCTCCCGAA |
| | 301) | | GACACCAGGGCAACATCTCTCATCCTTAAAGGCTGCTGGGAGTTAAT |
| | | | GGATGGAAGTTAATTAATGGGAAAGTAGCGCAAGTATTTCTCATCCC |
| | | | AAATCAGTAGGATGATCTGCCCTCTTATTTTGCAGGAGTGGGAAGAA |
| | | | GAGGGAGCTTGGAGAAGCTTTGAGCAGGTCCTGAATAGGCAAGTGAG |
| | | | GGGCTTGCCTTAACCCTAYAGGATTCTCAGTCTCCACGTCTACCTCC |
| | | | CACAACATGTGCAAATGCTTACATTCATGGTGGGTTTCTCCCTCTCC |
| | | | CTTGGATCCCCAAAGCAGCAAGAGCTGGTGTGGAGCACTCCCCAGTC |
| | | | TAGGCTGGGGGACGCAAGGAGAAGCCATCCTCACAGCAGTCTCTTCC |
| | | | TGAGAGATGCTAAGGCGGTGGAGAGACTGCATAATGTGCCTTTCCAG |
| | | | GGGGTGTTTCTCTCTATTGATTATTTGAACTGTTAAACTTGATTACA |
| | | | TTTTACTTTAATTGTACCATTTGAAATTAGATTCAGG |
| IFNG | rs12312186 | 25 | GTGTAAACTGTGTGAATCTAGGCAAGCTCCTTAAAGTCTCTGGACTC |
| | (POSITION | | TACTTCACAGGTTTTTTGTGGGATTCAAATGAGTTATATGTGCAGCT |
| | 501) | | CTTGGAATAATACTTGGGATATAGCAAGCACAATGTGTGCTCATCAT |
| | | | TTTTATTTCCATTTTATGGGTTTTTTTCCCTTGTAACCTGATTTAGA |
| | | | AGTTGTATTTGTACATTTCTTCATGTTTAACGTATTTGTTCAGGTTA |
| | | | AATTGAAATATTTTACATATAGAAACTGAGGTTGGGTTACCTCAGAA |
| | | | ACAGAGCTTGAGACAAGGATTTTTTTTTTTTTTTTTTTTTTTTGGTG |
| | | | GTGATTCTAGGAAGCACCAGTAGAAAAGAGGCAAAGAGATTCAGGGA |
| | | | AGGGAAGGAAGTCAGTTCAGGGTGGTTCCCAAAGGGAGCTACTGTAG |
| | | | TCAACTGAGACTCAGCCCACTATAGACCTCTGGGTGATGGTGTAGCC |
| | | | CATACCCCAAAGTTATCCTGCCCAAGGGACRAAGAAGTTGGGGTATC |
| | | | TATCCTGCGACTATCTTTAGCACTGTCTGAGCACTGCTCCCAGGGCA |
| | | | TTAAACCCCTAGCTCTTCCAGTCTTCCTCATGTGAAAATAGAAAGAA |
| | | | GCCCTTAGGCCAAGAATAGTGAACTGTTACAGTCACAGGCAGAGGGT |
| | | | AAGAAGAGAGAGGGAGGCTGCTGAGAGGATGTTGGCAAGGCAGGTAG |
| | | | TATCTGCTATGAGAAGTTATTAATTATTCCCTCATATTTTTTTTCAG |
| | | | TTTTTATTACATCCTTTATTTTTCGGCATTAGTGTCAGT |
| IFNG | rs12315837 | 26 | GTGTAGGGGACAGAGGGCATACGGAAAATCTCTGTACCCTCCTTTTA |
| | (POSITION | | ATTTTGCTGTGAACCTAAAAGTGCTCAAAAAAAATAAAGCCTATTAA |
| | 501) | | AAAATACTTGTTGATGTGCAAGACATTCTTCTAGGCACTGAAGAAAC |
| | | | AGCAAGAACTAACAAAAAAGGGACAAAACTCCTGTCCCCATGGGCCT |
| | | | TACATTGTAGTGGAGAAGATTAACATAAACAAACATGTAATTGTGTA |
| | | | ATACAATGTCAGGTTGTGATTATGATTTGAAAAAGGAAAGCAGGAGA |
| | | | ATGGAATAGTGCTATTTTAGATAGGGGCGTTGGGGAAGACTTTTCTG |
| | | | AGGAAAGAACATTTGAGCACAGACCTGACTGAAGGTGGTGAGGGAGT |
| | | | CATGGACACGACTCGGAACCATGTCCCAGGCAGAGAAGAGCCAAATG |
| | | | GAAAAAGTCAAGACAGACGCCCCTTCAGCGAGGGCTGAGTCATAGCA |
| | | | GGGGTCATGTGTCTGGACCTGAGGAGCAGGMAGTGGGGTTGGAAAGA |
| | | | TAACCAGGGGCCAGATCATGCCCCCACAAAGCATTTTGGGTTTTATT |
| | | | CTAGAGGAAATGGGGTACTCTCTACTGGGTTTTGAACAAGAGAGTGA |
| | | | CATGATCTGAGATATATTTTAATGGGATCACTGTGGTCAGCAAATGG |
| | | | AAATTTGGCTCTAATGGGACAAGGGCAGAAACTGAGAGGCCAATTTA |
| | | | GGAGGCTTCTGTACTCATCCAGGAAAATCCAACTGTGGGGCTCCAAC |
| | | | AGTTCAAATGAATTCCCACCCAAAGAGTCAGAAAAAATATGGCAACA |
| | | | CGCCCCCTCACAAATCATCTGTACCATATAAGCCAGCTTCTATAGAG |
| | | | GAAGGAAAGGTACTGGATGGACAAATAACAGGGCCCATCACATAGTT |
| | | | GTAATTTACAAATTACCTCACAAAAAGTGGTTATT |
| IFNG | rs12317232 | 27 | TCTAGGGAAGAATGCTTCCTTACCAGTTCTGGCTTCTGCCTATTCTT |
| | (POSITION | | GGCACTCCTTGGCTTGTGGCAGCACAACTCCACTCTCTGCTTCCATC |
| | 501) | | TTCACATGCCCAACTTCCTTCCATTTATGTGTATCTGTGCCAAATTT |
| | | | CCCTCTTCTTATAAGGACATCTGTCATTGGATTAGGGTTTACCCTAA |
| | | | TGAATTTGGGGAGGACCCTATTCAATCCACTACAACCACCCTTTATG |
| | | | TACACGTAGCTGGTTTCTCTGTCAATTATATTTTAGAGTGAGGACGT |
| | | | TGCTTCTCCTCTAACAAGATATTATAATAACAATTATTGTCAAATTA |
| | | | TTTAATGAATGCTTACTATATGACAGTTACATGCATTAACTCATTTA |
| | | | ACCCTCTGACAATTCTATGAAATAGGTGCTATTTTTATTTCTATTTT |
| | | | GCAGATGAGCAGCCAGAGAGAGTTTACATAGGGCAAATATCACCATT |
| | | | ACCTAGCAAGAACAAAATAAGAGGAATAAGMAGTCCCCTTGTATTTT |
| | | | GGTTACTTAAAAGGGATGGATCTCAAGACAAAGGAAAATGGTTGGGT |
| | | | GCACGAGGGGCCAGATGCTGGAACCAGTTCTGAAGAAGTGTTCCTGG |
| | | | GGCCAAGAGGATCTGAGAGGTGGCCAGGTGTGAAGACTGAACAAGCT |
| | | | GAGCGTTAAGAACAGCAAAGTTGGCCAGGCATGGTGGTGCATACCTG |
| | | | TAGTCTCAGCTCCCTGGGAGGCTGAGGTGGGAGGAATATGAAGGCCC |
| | | | AGGAGTTCAAATCCAGCCTGGGCAACACAATGAGACCCTGTCTTAAA |
| | | | AAAAAAAAAAAATCAGCAAGCTGGGAAATAAA |
| IFNG | rs2041864 | 28 | TAATTCATTGTGACCCCTCAGACCATCCTCCGGATAAACAGCATTGA |
| | (POSITION | | GATTGCTCTGTGTTTGTTGTAGTCACCGAGTTAGTATTTGCAGAAAT |
| | 1083) | | ATAAAAATAAACTCTTGCTTTCCAAGGAAAAAAAGAATCTTGGGTAT |
| | | | GGCCACCCCCAATAATGTGTAATGGGCTAGTGTAAAATTATACTAAT |
| | | | GAGCAACTAGTGAGCACATGCTGTACTTAACAGCTCTTGTTCAGGAT |
| | | | TCAGTTAGACTTAGATCTCTTTAGCTGCAAAACTTTGGGAATGTTAT |
| | | | TTATAGTTTCCAAGCCTCATTGATAAGATTGTTGTGAAGATTAAATA |
| | | | GAATGCATATAAAATGCAGCTCAGTTGGTGAAGGCACTTTCACCTTT |
| | | | GATCCTTCATCACCATCTGCCCAAAAGAAGCCCTGTCATGGAGCAGC |
| | | | CAGATTCTCATTTTAGGTAAACAGAAAAGGATAAGGCACTTCTGGCC |
| | | | TTGTATTTTCTCCCAGAGCACTCAGATGCTGATTATATTACAGACAA |
| | | | ATCAAGATTTCTCAACCCTCTTCAATTCTTTCAATCAATTATCCATT |
| | | | TAGTGTAACTATGTGATAATGTCTAACACATTAATTATCATGAAAAA |
| | | | TGTGAAAGCTACTAAACTAAAAAAAAAAAAATTCTTTTTAGTAGCAA |
| | | | GGATTTTGTATGGGGAAGCCTGGCTTTGTGGGAAATGATTTGATAAA |
| | | | CTTACACTGGAAACTGAACCTTAGGGAATGGATTCCATTCCAGTCAA |
| | | | ATCTTCAAAGGAAAAGAGGAAGCTACTCTGGATAATAAGAGTGAAGA |
| | | | ATTGGAAGTTCCTGGGAGGAAATCCTGGAAAGGAAAAGAAATTGGTA |
| | | | CTGTGTAGAGGAAAGAGAAAACTCTCCCCTCTCCATGATGGTGCAGC |
| | | | TGAGGCAGAACTTTGGAAAAAGAAAATCTCTGGAATGCTGACAATCG |
| | | | TGTTTCCCTAAAAAACCCTCCGACACCTTCAGAAACTATTCTGAATT |
| | | | GCTGAGTATTAATGCTTTTGTGTGAGTATGTTATTTTGAGGAGTTAA |
| | | | GCTCTATGTCTTGATAAGAATGTATCAAAAATAGACCTCGCACATCA |
| | | | AYCCAGGAGTCAGAGGTCACAAAGGAGACTGACAAATGGGTCATGGT |
| | | | GAGAACTATGACCACCCGTGTCCATATAGCTTAACTAGCAGAACTGA |
| | | | AGCTGAATGCCACCTTGGTCAAGATGA |
| IFNG | rs2058739 | 29 | AAAATGTCCTGTTACATGACAAATTTAAAACAATACATTTTAGAATT |
| | (POSITION | | TACCTTGACAACACTCTCAGAGAAGATTATTTTAGAAACTATTGATA |
| | 349) | | AATTAAAAATCTAAGTGAATTATATGCCTAAAAGCTTTCTTTTAAGT |
| | | | GATACTTGAGGGGAAAAAAACGTCATCCCAACATTTTTAGACATTGA |
| | | | ACTTTACAAGTGTAGAAATGGTCACAGAAAGCCTATGTTATTCTGAA |
| | | | ATATATTTTGTTTCAGCTATGTTTGTGAAAATTGACCAGCTACTTGA |
| | | | CAAATCTAGATTTTCTTAAAGGCACTCAACTAAATGCTATTGTCTCC |
| | | | TAGGACTTGTCTTGGCCATYTTGATTATCATAACTCTCCAATATAGG |
| | | | CTTTAGGATTTCCAAATTCATACTCTGAAGCCCAAAATTATTCCCAC |
| | | | TATAATTTAGAGTTAGCCTTTGAAATAACTTATAGAAAGCATTAATT |
| | | | GATTCCATATCTAGGGGCCTTCTGAGTTGTTTATAACTTATATATAT |
| | | | CTACATATATATTTATTGATAAAATTTTATTTTTAATATAATTTACA |
| | | | ACCAGATTTCTCTTACAAAAAAGATTCAATCTATTTTAAAAGTATGA |
| | | | TAATCAATTATATTATATAATTTGTGCCACAATTCATACTTATCTAT |
| | | | TGATTTAGAAACCACATTCAAGATAATCCTCTCTACCAAGAATTGGC |
| | | | CCCCAGCGTAGCAGCAAAGCACCATTAACTATCATTTCCACCGACAG |
| | | | CTGAAGTTGTGGTTTTGCATTCAGCACTTTTTTCCTTGTGTGGAGTA |
| | | | TAGAACAAAAGATGTTACTAATGTAATAATGTGAGTCATCATCCAAA |
| | | | TCTGTGGTTACTACTACCATGAAAAGTTTTCTTTCTCAGTAGGAAAG |
| | | | TGTCATTGGTCATTCCCAAGATGTTACAGAT |
| IFNG | rs2069727 | 30 | TGTGGTATTTCTTTCCACTAGCATTTTGTTCGCTTTCGCTTTTCCAG |
| | (POSITION | | TTAGCAGCTCTTTGAATTATCTTTCTAAGATACAGATTTAATTATGT |
| | 201) | | CACTATTCAATTCAGAGGTTCTGCTATGGAATGTAGTTTAAACTGCT |
| | | | TAGCTTGGCACACAGAGATTTATTTCTAGCCCCTTCTCCACCTTCCT |
| | | | ATTTCCTCCTTCRTTTGAGAATCTTCCTCTCCCTCATCCAATGCTGG |
| | | | CAAACACCAGTGGGGGTGGAGTAGTGGGTGTAAGCTCTAGGGAGAAG |
| | | | GCTTGGATTGGAATCCAAGTTATTCCATTACAAGTAGTGTGACCTTT |
| | | | AATACATTATGTATATTGTCTAAGTTTCAGCTTTATTGTCTGAAAAA |
| | | | GAAAAATAATTGTGTGTTCCTCATAATATTGTGGTACGAATTGATTC |
| | | | TTTCACTCAAGAAATATTTACTGGAGTACCTACTACATGCCTGGTGC |
| | | | TGTTGTAGACCTTGAGATACCTTACTCAAGCAAAACAGCCAAGGATC |
| | | | CCTGCCCCTGGGGAATTTGAAATTAAGCAAGGGACAGATAAACAATG |
| | | | AACAAAATACATAATATGTAAGTCTATTGCATGGCATTCTCTAAGGT |
| | | | GATTGGTGTCATGGAAAAATAGTTAAAGGAGAGCAGGACAGGGAAAT |
| | | | TAGGAGTCCTATGTATGGTGGAGTGGGAGGGCTAGAGGTTTAAAAGG |
| | | | GTAATTATATGTGGCCTTATTGAGGAGATGCCATTTGAGGAAGCGCT |
| | | | TTAAGAAGTAAGAGAGGTAGCTATTTGAATTCCAGGCAAAAGGTATA |
| | | | TCCTTGCAAAGGCTCTGAAGAGATTTTCCTGGAGTGGTAGAAGAACC |
| | | | AGCAGACCAGTGTGCTGGGCCCAGAAGACGGAAGAGAAAATCAGCCA |
| | | | CACTTGAGAGGAATTCAGGGGAAGCAATGTCCTTAGGGGAGGGCCAG |
| | | | TTTATCTTTTGAGAAGGAGGAAGTTGAGGATATGATGGATTTGGTTA |
| | | | GTTCTGGGCTGTAAATTCCAGAAGACCCAGTGAGACAAAGTAAGAGA |
| | | | GGTTGTCATAAAAGGGAACGTGCATAGGGATGTGTTGTGAGTCTGAG |
| | | | ACTTCTTATGATTACCGACATAAACAAGATAATGGATATAGTGAGAT |
| | | | TAGTTCTACCAGCTGTGGAACGTGTAGTGGTGGCAAGATCATGAATG |
| | | | TCAAGGATAGAGAGGGTTAGACATCTGGGGCTTCCTTCTCAACAATT |
| | | | TCACATAAACCTCCAACAGCAACAGTAGGATTATGTGAAATAGATCA |
| | | | CACAAAGGATCATTTGAGTCATTGACAATAATCAGGGGT |
| IFNG | rs2080414 | 31 | CTATTTTGATCAAAGAAAGGATCTCAGAGACAGGTACTCAAGTGTTG |
| | (POSITION | | ACAGGATTTGTCTCTCTAGCTGTCACTTCTGCTTCTCTTTGTGAGAC |
| | 295) | | AATGTCAATCCTGCCTCCCACAGAGCAGCATTCACCAGCTGGAAGGT |
| | | | AAGTTAGCCATTAAGGCATTTAATTGAAACACTGCACTAATTCATCA |
| | | | AATACTTGCTGAGCTACATATTTATATCATCAGGGAAATGCAAATTA |
| | | | AAACAACAAGATACCCACACACCCATTATGAAATGGCAAAAATCTGG |
| | | | AACACTGACAACWCCAAATGCTGGCTGAGACGTGGAGCATCAGGAAC |
| | | | TCTGACTGAAGGTACAGCCACTTTGGAAGACAGTTTTGCAGTTTCTT |
| | | | ATAAAACTAACCTTACTCTCACTATACCAGCCACCAATCACAACATT |
| | | | CCTTTGTATTTACCCAAAGGAGTTGAAGTCTTATGTCCACACAAAAA |
| | | | TCTGCACACACATGTTTATAGTAGTTTTATTCATAGTTACAAAAACT |
| | | | TGGAAGTAACCATGATATCCTTCAGCAGATGAA |
| IFNG | rs2098394 | 32 | CTATTTTGATCAAAGAAAGGATCTCAGAGACAGGTACTCAAGTGTTG |
| | (POSITION | | ACAGGATTTGTCTCTCTAGCTGTCACTTCTGCTTCTCTTTGTGAGAC |
| | 259) | | AATGTCAATCCTGCCTCCCACAGAGCAGCATTCACCAGCTGGAAGGT |
| | | | AAGTTAGCCATTAAGGCATTTAATTGAAACACTGCACTAATTCATCA |
| | | | AATACTTGCTGAGCTACATATTTATATCATCAGGGAAATGCAAATTA |
| | | | AAACAACAAGATACCCACACACCMATTATGAAATGGCAAAAATCTGG |
| | | | AACACTGACAACACCAAATGCTGGCTGAGACGTGGAGCATCAGGAAC |
| | | | TCTGACTGAAGGTACAGCCACTTTGGAAGACAGTTTTGCAGTTTCTT |
| | | | ATAAAACTAACCTTACTCTCACTATACCAGCCACCAATCACAACATT |
| | | | CCTTTGTATTTACCCAAAGGAGTTGAAGTCTTATGTCCACACAAAAA |
| | | | TCTGCACACAGATGTTTATAGTAGTTTTATTCATAGTTACAAAA |
| IFNG | rs2098395 | 33 | CTTCCTCAGAGGAACATGAAAGAATGCACAAGTGTAAGTCTCCTAGC |
| | (POSITION | | GTTCTAGCATCCCAAAAAGAGTCCCATACAATTAGTAAACAACAGCA |
| | 1060) | | ATGCAAGGACTCAAAAATAATAAGTCTTTGGTATTTGATCTAAATTT |
| | | | TTTCACTGGTTTTTCATTTTTATAGCTTTAATGCCATGAGTTTTGTC |
| | | | TAGGATTTTTTTTTTTTTTGCATATGTGCATCCAATTGTTCCAGCAA |
| | | | TATTTGTTGAACAATCTATGCTCTCTCCATTGAATTACCTTTACTCT |
| | | | GTCAAAACTCAGTGGACTATATTTGTATGAGTCTATTTCTGGGCTCT |
| | | | CTGTTCAGTTCTATTGATTTATATGGCTATTCTTTCACCAGTACCAT |
| | | | TTTGTACTAATTACTGTGCCTTATAGTAGGTTTTCAAGTTAAATAGT |
| | | | ATGAGTCCTCCAAATTTGTTCTTCTTCAGTATAGGGTTAGCTATTCT |
| | | | ATGTTTTTTCCCTTTCCACATAAATTTCAAAATTTGTTGGTATCTAC |
| | | | AAAATACTTGCTGGGATTTTGTTGAATCTATAGATGAAGCTAATAAG |
| | | | AAATAACATCTTAATGATATGGAGTCTTCCAATCCATGAACATGGAA |
| | | | TGTTTCTCCATTTACCTAGATCTTCTTTGATGTTTTTCATCAGTGCA |
| | | | TTGTAATTTACTACATAGAGGTCATGTACATATTTTGTTAGATTTAT |
| | | | ACCTATTCCATGTTTTGGGTGCTATTGTAAATGATGTTTTTAACTTC |
| | | | AAATTTTAATTGTTCAGTGCTGGTATATTGGAAAGCAATTAACTTTT |
| | | | GTGTATTCGCCTTGTATCCTGTCACCTTGCAACACTCATTTATTAGT |
| | | | TCCAAGAACTTTTTGTCAGTTCCTTGAGATTTCCTGCACAGACAATT |
| | | | ATGTCACTATGAACAGTTTAATTTCTTCTTTTCCAATCTGTATACCT |
| | | | TCTGTTTTCTTCTACAAATATGTTAGGTTAAATGGAAAAGAATTAAG |
| | | | GTTGAAGATGAAATTAAGGTTGGTAATCACCTGGCCTCCAGATGAGG |
| | | | AGATTATCCTGGATTATCTGGGTAARCCGATATGAAAGCAAAGGTTC |
| | | | TTATAAATGGGTAATATAGGCAGAGAGAGAGAACCAGAGAGATGGCA |
| | | | GCATGAAAAGGACTCAGCTGACAAGGAGGAAGCAGACTGCGAGCCAA |
| | | | GTAGTGCAGGCAGCCTCTAGAAATTAAAAAAGATAAGGAAACAGATT |
| | | | CTCTTCTCAGAGCCTCCAGAAGGAACACAGAGCTTCCCTACACCTTA |
| | | | ATTTTAGTCACTGAGACTGATTTTGGACTTATGACATCCGGAACTGG |
| | | | AAAATAACAGATTTGTGTTGTTTCAAGCCACCAAGTTTGTGGTAATT |
| | | | TGTTACAACAGCAATGGGAAACTAACATACATATCTTCTGAAAATAA |
| | | | GCCTGTTGTAATTTTTTGTTCTTCCACAGGTAAAGTGGTGTTTTTTC |
| | | | CCTTTGGCTCTTTCAAGTTTTTCTCTTTGTTTTTCTGCCATTTGAAT |
| | | | ATGATATTCTGTCTTAGACCATTTTGTGCTGCTATTACAGAACACCT |
| | | | GAGACTGAGTAATCTATAATGAGCAGGCATTAATTTGTCTCACAGTT |
| | | | CTGGAGGCTGGGAAACCTAAGGCCAAGGGGCTGCACCTGGTGAAGAC |
| | | | CTTCTTGCTGCATCACAACCTGGCAAAAGGCATCACATAGATGAGAG |
| | | | AGAGCAATAGAGCTTGAGAGAGAAAGGTTCAGAGGAGGAGGAGAAGG |
| | | | AGGCTGAATTTATTCTAAAAGTAAACCCACTCTTAGGATAACTAACC |
| | | | CATTCTCAATAATGACATTAATCCATTCATGAGGGCACAGCCGTCAT |
| | | | GACCTAATCACCTCTTAAAGGTCCCTGTCTCAACACTATTGTGTTGG |
| | | | AGATTAAGTTTACAATACCTGAACTTCTTACAAACCACAGCACATTC |
| | | | TTAGGGGTAGTTTTATGGCAATTTGTTCTGCCCAGTATTCTATGAGG |
| | | | ATCTATTGTTTCGCTACGTATTTTGAAATTGCCAAAAAAAAAAAAAA |
| | | | AGGAAAAAAGAAAAAAGATATTGTCCCCTCCCCAGTTCTTGGAAGTT |
| | | | CTTTTTTGTGTGTTTTTTTCTATTCTGTTTGTTTGCTTGTTCTTCAT |
| | | | TTTCTCTCTCTGCATTTCAGTTTGGGAAGTTTCTATTTACCTATCTT |
| | | | CAAGCTCACTGATTCTCTTCAAGCTCACTGATACGTTGTGTTTACTG |
| | | | GAGCCTATCGAAGACAATCTTCATTTCTGTCACAGTATTTTTTATTT |
| | | | ATAGTATTTCTATTTGATTCTTTTCTTAGAATTTCCATCTCTCTACT |
| | | | GACATTACCCATCTGTTCTTGCATGTTGTCTACTTTCTCCCTTAACA |
| | | | TATTAATTTTAGTTATTTTAAATTTCTTACCTGGTAATCCCAAACTC |
| | | | TATGTCATATCCGAGTCTGGTTTTGATGTTTGCTGTATCGCTTCAGG |
| | | | CTGTGTTTTCTCTCACCTTTCCGTGTGCCAGAGGCTTCAAGTTCTCT |
| | | | GGCATTCTTGCCTTTGTCTCCCATCTTTACCTTGTGCTTCCGTAACT |
| | | | ACTCCTACTTAGACAGAGTCTGTGCCTTGCAGCTCTTTCACCTGTGA |
| | | | TCCACTGTTATTACTCGAGCCCTGTGGTATGTAGTAAAGTATGGGGA |
| | | | AAGGGAAGTGTTTTATAATCTTTAAATCTCAGCATTTTAGTGGGCCT |
| | | | GTGTCTCAGGACTGTGATCTTCACAAGTGTTTCTTCTTGTATAGCTT |
| | | | TAGGTGTAACAGGACAACTAGAAGGGACTCAAGTTAGAGAAACATCC |
| | | | TTCCCCCACAGCCCTCTCACAGGAGTCTGGTAAAGCCTTTCCCCTGG |
| | | | AGAGCAGACCTTTGTTTCTGGACATACTTCAGAAGGTTACTCGTCCC |
| | | | CTCCCCCTGCCAGAGCCACAGGGGTATCTTTGTCAGAACTTCACCAG |
| | | | GAGAACTTGGTGGGATTCCTGTAGGTATGCTCACGAAAACAAGGAGG |
| | | | ACCCATCACAGTTCGGCCCCCAGGTGTTTCTCACTCCCATGCTAGTC |
| | | | CACACTCAGCCTCCAGCAAGTCATCAAAATTACCATTTAAGTGTTTT |
| | | | AACAAGTTAATTACTCCAGTGGATTCAGGTCCAAGTAAGCAGATCTT |
| | | | GGCTGTGAATTTCTGGATTTGCCTACTCTCCAGATTTTATTGTGGCA |
| | | | GTTTGTCCTGCAAATTCCGTTCTATGATGGAACTAAAAAACTCGCTG |
| | | | GTTTTATTTGTCCAGCTTTTCCTTGTTTTAAAGGCTGGAGTAACAAC |
| | | | TTCCATGCTCTGTATATGTTGGAGCTAAAATTGGAAGTCTGTCACGA |
| | | | TGGTTTTTTTTCTTTTTTTTCTTTTTTTTTTTTTTTCCTGAGATAGA |
| | | | GTCTCACTCTGTCATCCAGGCTAGCGTGCAGTGGCATGATCTCAGCT |
| | | | CACCACAACCTCCACCTCCCGGGTTCAAGCGATTCTCCTGCCTCAGC |
| | | | CTCCTGAGTAGCTGGAACTACAGGCATGTGCCACCATGTCCAGCTAA |
| | | | TTTTTGTATTTTTAGTAGAGATGAGGTTTTACCATGTTGGTCAGAAT |
| | | | GGTCTCAATCTCTTCACCTCAGGTGATCCGCCCGCCTCGGCCTCCCA |
| | | | CAGTGCTGGGATTACAGGTGTAAGCCACCACACCCAGCCCATGATGG |
| | | | TTTTTTTCATTGAGGCCTCAGTTGGAAAATTCAAATGCTTGGAGCTA |
| | | | CAATCATCTAAGAGCTTGCTCACACACATCTGATGATTTGTGCTGAT |
| | | | GCTGAGTGGAAGCCTTACTGGAACT |
| IFNG | rs2111059 | 34 | AGGAGAAGGAGGCTGAATTTATTCTAAAAGTAAACCCACTCTTAGGA |
| | (POSITION | | TAACTAACCCATTCTCAATAATGACATTAATCCATTCATGAGGGCAC |
| | 256) | | AGCCGTCATGACCTAATCACCTCTTAAAGGTCCCTGTCTCAACACTA |
| | | | TTGTGTTGGAGATTAAGTTTACAATACCTGAACTTCTTACAAACCAC |
| | | | AGCACATTCTTAGGGGTAGTTTTATGGCAATTTGTTCTGCCCAGTAT |
| | | | TCTATGAGGATCTATTGTTTYGCTACGTATTTTGAAATTGCCAAAAA |
| | | | AAAAAAAAAAGGAAAAAAGAAAAAAGATATTGTCCCCTCCCCAGTTC |
| | | | TTGGAAGTTCTTTTTTGTGTGTTTTTTTCTATTCTGTTTGTTTGCTT |
| | | | GTTCTTCATTTTCTCTCTCTGCATTTCAGTTTGGGAAGTTTCTATTT |
| | | | ACCTATCTTCAAGCTCACTGATTCTCTTCAAGCTCACTGATACGTTG |
| | | | TGTTTACTGGAGCCTATCGAAGACAATCTTCATTTCTGTCA |
| IFNG | rs2193045 | 35 | AGTATATGTGTTTAGCATTTTTTCAAAAAGTGTTTTGTGACACACAA |
| | (POSITION | | TATTGGCTCTTTTTCCCTGCACCTGAAGGCCTAAATTATAGAACATT |
| | 265) | | AGTTTGCTGGGTCTCTATTAGTTCACCAATGGATGCTGATGTCTCAA |
| | | | TTTTTCAAAAGCTTTCCAGTGAGTTATGCAAAGCCCTCAGGAAAACT |
| | | | GAGTAGCAAATAGGATTAGCATATTTGTAAAGACCCAGAAGTAATGC |
| | | | ATTAACATGCTGAGGTGTCATAAGCCCCARTGAATATGTTGATAATT |
| | | | AGTGCTTCTTAGAGAGCAGCTAGATCACCTTCCTCCATGCTAATGAT |
| | | | GTGCAAATAATCCTTGGTGAATCTGAACATCTGCTAGTGGGTGTCCC |
| | | | CAAGCAGGATGCAATGACAGGAGACAGATTTATCAACATTGCTGTTG |
| | | | GATTCCACCAAAAACATACTCCAGCCCATAAAACCTTCTATCAGGCA |
| | | | TAATCATATTCCTAGCCATAATTTTGCTATTGTTTGCAATCCTATTT |
| | | | TTTTTCTATCTATACTAATTAAAGTCTTGGTGCACCCAAAGTAGTTT |
| | | | GTATAAATTACATGAACTCATAAAAATTTCAGTGTTCATTTGACATG |
| | | | AATCGT |
| IFNG | rs2193046 | 36 | AGTATATGTGTTTAGCATTTTTTCAAAAAGTGTTTTGTGACACACAA |
| | (POSITION | | TATTGGCTCTTTTTCCCTGCACCTGAAGGCCTAAATTATAGAACATT |
| | 530) | | AGTTTGCTGGGTCTCTATTAGTTCACCAATGGATGCTGATGTCTCAA |
| | | | TTTTTCAAAAGCTTTCCAGTGACTTATGCAAAGCCCTCAGGAAAACT |
| | | | GAGTAGCAAATAGGATTAGCATATTTGTAAAGACCCAGAAGTAATGC |
| | | | ATTAACATGCTGAGGTGTCATAAGCCCCAATCAATATGTTGATAATT |
| | | | AGTGCTTCTTAGAGAGCACCTACATCACCTTCCTCCATGCTAATGAT |
| | | | GTGCAAATAATCCTTGGTGAATCTGAACATCTGCTAGTGGGTGTCCC |
| | | | CAAGCAGGATGCAATGACAGGAGACAGATTTATCAACATTGCTGTTG |
| | | | GATTCCACCAAAAACATACTCCAGCCCATAAAACCTTCTATCAGGCA |
| | | | TAATCATATTCCTAGCCATAATTTTGCTATTGTTTGCAATCCTATTT |
| | | | TTTTTCTATCTAYACTAATTAAAGTCTTGGTGCACCCAAAGTAGTTT |
| | | | GTATAAATTACATGAACTCATAAAAATTTCAGTGTTCATTTGACATG |
| | | | AATCGT |
| IFNG | rs2193047 | 37 | GCTTGCTCAAAAGGACTAGATGCCACCATGGGGACCCCGCTCACCAG |
| | (POSITION | | TGGTGGCCTCGTCTTTTATAGATGGATTCCTCAAAATCACACTTGCC |
| | 297) | | GCCCTTGTCTCCAAGATTTGGATCACGGTCTAGATGCTCCCACCCTT |
| | | | TCTCCTCAAAACTGCCACACCACAGTCTCACCAACCGCCAGGGTGCT |
| | | | CTGCCCTTTCGTGACCAAATCCCTGAAGCTAGGTGAGTTTTGCAAGC |
| | | | CCTATGCACGGCAGTCCCACTCCCTGATGATCCTAAGATGTAAGACC |
| | | | TCACAGAATTTACAYTGGAGCCTAAGTAACAGTGCACCATGCCAAAG |
| | | | AAGACAGAGTTAAACTAAACACAATCCAGGGATTCTGACTTACTGAC |
| | | | TTCTTTTGCAACTTGCTTCCTCTCTCAGTTCTTCTAGAAATGGAAAA |
| | | | TTTTTCAGATCAAGAAAAACTGAACAGAACTTGCCAGAATGACGAAG |
| | | | CTAATGACAGTGATTCATTGATTTATTCAACAAAAATCACAATAATA |
| | | | ATAATAGGCATTTACAGAGCATTGTTTCTCTTCCAAGCATTTTGTAT |
| | | | GCATTTTATTTAATCTTCACAACAATCTTATCAATGAGGCTTGGAGA |
| | | | CTATAAGTAACTTCCCTAAAGTTTCACAGCTAAAGAGATCTAAGTCT |
| | | | AACTGAATCCCAAACAAGTACAGCACGTGCTTGC |
| IFNG | rs2193048 | 38 | GCTTGCTCAAAAGGACTAGATGCCACCATGGGGACCCCGCTCACCAG |
| | (POSITION | | TGGTGGCCTCGTCTTTTATAGATGGATTCCTCAAAATCACACTTGCC |
| | 543) | | GCCCTTGTCTCCAAGATTTGGATCACGGTCTAGATGCTCCCACCCTT |
| | | | TCTCCTCAAAACTGCCACACCACAGTCTCACCAACCGCCAGGGTGCT |
| | | | CTGCCCTTTCGTGACCAAATCCCTGAAGCTAGGTGAGTTTTGCAAGC |
| | | | CCTATGCACGGCAGTCCCACTCCCTGATGATCCTAAGATGTAAGACC |
| | | | TCACAGAATTTACATTGGAGCCTAAGTAACAGTGCACCATGCCAAAG |
| | | | AAGACAGAGTTAAACTAAACACAATCCAGGGATTCTGACTTACTGAC |
| | | | TTCTTTTGCAACTTGCTTCCTCTCTCAGTTCTTCTAGAAATGGAAAA |
| | | | TTTTTCAGATCAAGAAAAACTGAACAGAACTTGCCAGAATGAAGAAG |
| | | | CTAATGACAGTGATTCATTGATTTATTCAACAAAAATCACAATAATA |
| | | | ATAATAGGCATTTACAGAGCATTGTYTCTCTTCCAAGCATTTTGTAT |
| | | | GCATTTTATTTAATCTTCACAACAATCTTATCAATGAGGCTTGGAGA |
| | | | CTATAAGTAACTTCCCTAAAGTTTCACAGCTAAAGAGATCTAAGTCT |
| | | | AACTGAATCCCAAACAAGTACAGCACCTGCTTGC |
| IFNG | rs2193049 | 39 | ATATCTCTTCATGTCTCACAGTCTGGCCAAACTGAGATCAACCTCAG |
| | (POSITION | | AGAGAGGGAATGTTTTATCCAGCCCTAGATTAAAATTTATCTCCTGG |
| | 223) | | GGCTTCATTACACATGGTATGATTAATCACTGCTCAAGATGATCGAT |
| | | | CGTGGGAATTTCCAATCCCTTCCCGAGAAGTATGGTCTAGAACTGTG |
| | | | GTCCAGGCAAGACAGCTTCACAGTAGCCCTTCATSTGTTTATACATC |
| | | | AAAGTCTGCATCAATGAATCTTAATTCAAACAAGAGTGGAGACACCA |
| | | | GTAGCAGATCATATTAGCTGTAGTTGTGCCAAAATTAACCAAATTTA |
| | | | CTTCCAATCTTGCTTCATCAAAGATTCAAAAGTTTTAGCATCAGATC |
| | | | TCACCCACTGTCACTTAGTTACCCAATAATCAAAATAATGACCCCTG |
| | | | ATTATTGTCAATGACTCAAATGATCCTTTGTGTGATCTATTTCACAT |
| | | | AATCCTACTGTTGCTGTTGGAGGTTTATGTGAAATTGTTGAGAAGGA |
| | | | AGCCCCAGATGTCTAACCCTCTCTATCCTTGACATTCATGATCTTGC |
| | | | CACCACTACACGTTCCACAGCTGGTAGAACTAATCTCACTATATCCA |
| | | | TTATCTTGTTTATGTCGGTAATCATA |
| IFNG | rs2193050 | 40 | TATTAGCTGTAGTTGTGCCAAAATTAACCAAATTTACTTCCAATCTT |
| | (POSITION | | GCTTCATCAAAGATTCAAAAGTTTTAGCATCAGATCTCACCCACTGT |
| | 201) | | CACTTAGTTACCCAATAATCAAAATAATGACCCCTGATTATTGTCAA |
| | | | TGACTCAAATGATCCTTTGTGTGATCTATTTCACATAATCCTACTGT |
| | | | TGCTGTTGGAGGKTTATGTGAAATTGTTGAGAAGGAAGCCCCAGATG |
| | | | TCTAACCCTCTCTATCCTTGACATTCATGATCTTGCCACCACTACAC |
| | | | GTTCCACAGCTGGTAGAACTAATCTCACTATATCCATTATCTTGTTT |
| | | | ATGTCGGTAATCATAAGAAGTCTCAGACTCACAACACATCCCTATGC |
| | | | ACGTTCCCTTTTATGACAACCTCTCTTACTTTGTCTCACTGGGTCTT |
| | | | CTGGAATTTACAGCCCAGAACTAACCAAATCCATCATATCCTCAACT |
| | | | TCCTCCTTCTCAAAAGATAAACTGGCCCTCCCCTAAGGACATTGCTT |
| | | | CCCCTGAATTCCTCTCAAGTGTGGCTGATTTTCTCTTCCGTCTTCTG |
| | | | GGCCCAGCACACTGGTCTGCTGGTTCTTCTACCACTCCAGGAAAATC |
| | | | TCTTCAGAGCCTTTGCAAGGATATACCTTTTGCCTGGAATTCAAATA |
| | | | GCTACCTCTCTTACTTCTTAAAGCGCTTCCTCAAATGGCATCTCCTC |
| | | | AATAAGGCCACATATAATTACCCTTTTAAACCTCTAGCCCTCCCACT |
| | | | CCACCATACATAGGACTCCTAATTTCCCTGTCCTGCTCTCCTTTAAC |
| | | | TATTTTTCCATGACACCAATCACCTTAGAGAATGCCATGCAATAGAC |
| | | | TTACATATTATGTATTTTGTTCATTGTTTATCTGTCCCTTGCTTAAT |
| | | | TTCAAATTCCCCAGGGGCAGGGATCCTTGGCTGTTTTGCTTGAGTAA |
| | | | GGTATCTCAAGGTCTACAACAGCACCAGGCATGTAGTAGGTACTCCA |
| | | | GTAAATATTTCTTGAGTGAAAGAATCAATTCGTACCACAATATTATG |
| | | | AGGAACACACAATTATTTTTCTTTTTCAGACAATAAAGCTGAAACTT |
| | | | AGACAATATACATAATGTATTAAAGGTCACACTACTTGTAATGGAAT |
| | | | AACTTGGATTCCAATCCAAGCCTTCTCCCTAGAGCTTACACCCACTA |
| | | | CTCCACCCCCACTGGTGTTTGCCAGCATTGGATGAGGGAGAGGAAGA |
| | | | TTCTGAAATGAAGGAGGAAATAGGAAGGTGGAGAAGGGGCTAGAAAT |
| IFNG | rs2216163 | 41 | TTACTCTTCCAAACCAAAACTCTGGGAGTGACAGGTAGGGAGAGAGG |
| | (POSITION | | AGGGAGTGGGATATAAACTTAGAATCTCCCTTTCACAGACAGCCTTT |
| | 112) | | GCAGAAAGTCCAACTTAYTCCCAGGAATGGCCAAGTCTTTCTCAGAG |
| | | | CTGGGATCCAATCTCCCTCACCCAGTCGCACACCCCTGGGCCCTGCC |
| | | | TACAACAGTCCAGGGAAGCACCTTTAGCCCTCCTTTACTTCTTTTTG |
| | | | AATCTTCTACCAGCCTGCTTTCCTGTCTCCCCTTCCACTCCCATCTA |
| | | | ATCAATGTAGAAATGGCCTCTCATTTCACTTCTGAGAAGCCATTTCC |
| | | | TGTCATCTCTTTAAAGTCTACCGCTTTCCCACTGACTGTCTCTAATA |
| | | | AGCAGAAAGCAAATGTCTAGCCCTCCTTGTCAGCATAATTAGGAAAC |
| | | | TGCTTCCTCTGGACGTGCCTGAAGTCCCTATGTTGCTAAGAGCAAGA |
| | | | CTCTTCATGTTTTGCCATTTGGGACGTAACTGTTTTGGTGAGCAGTG |
| | | | TGCAAATCAGTTTTTAACACCAACATTCTGGTCTAGTCTTTGAGACA |
| | | | GGAAAAAGATGAAAATACATATGTTTCCACATTTTAGGGTAGAAAAC |
| | | | CCAGTCTGTGGTTTCCC |
| IFNG | rs2216164 | 42 | AGTATATGTGTTTAGCATTTTTTCAAAAAGTGTTTTGTGACACACAA |
| | (POSITION | | TATTGGCTCTTTTTCCCTGCACCTGAAGGCCTAAATTRTAGAACATT |
| | 85) | | AGTTTGCTGGGTCTCTATTAGTTCACCAATGGATGCTGATGTCTCAA |
| | | | TTTTTCAAAAGCTTTCCAGTGACTTATGCAAAGCCCTCAGGAAAACT |
| | | | GAGTAGCAAATAGGATTAGCATATTTGTAAAGACCCAGAAGTAATGC |
| | | | ATTAACATGCTGAGGTGTCATAAGCCCCAATGAATATGTTGATAATT |
| | | | AGTGCTTCTTAGAGAGCAGCTAGATCACCTTCCTCCATGCTAATGAT |
| | | | GTGCAAATAATCCTTGGTGAATCTGAACATCTGCTAGTGGGTGTCCC |
| | | | CAAGCAGGATGCAATGACAGGAGACAGATTTATCAACATTGCTGTTG |
| | | | GATTCCACCAAAAACATACTCCAGCCCATAAAACCTTCTATCAGGCA |
| | | | TAATCATATTCCTAGCCATAATTTTGCTATTGTTTGCAATCCTATTT |
| | | | TTTTTCTATCTATACTAATTAAAGTCTTGGTGCACCCAAAGTAGTTT |
| | | | GTATAAATTACATGAACTCATAAAAATTTCAGTGTTCATTTGACATG |
| | | | AATCGT |
| IFNG | rs2870950 | 43 | AAGCTCACCAATGAGGTGACATTTTTGCACAGACCTGAAGGATCCTT |
| | (POSITION | | ACAATGACTAAGGAGTAGAGAGTAAAAAGATTATTGATTTTGGTTTT |
| | 422) | | GTAATTTATGTGGATGTAGAAACAGGCTTGGGGATGTTAAATATTTT |
| | | | TAGTAGCATCACATAATTATCATGAAAGAAGTTAAAGCCATGATCTA |
| | | | GAAGATTTTACAATTCTCTGATTCACCTGTTGTGCCTTATTTTCTCT |
| | | | CAGGTAAGCTTCTTAGTTATCTGGTTACTTTTAACAAATGGCAGAAA |
| | | | CAACTTCTTAACTATGGAAGATTATGTTCTTTTGATTTACCAAATTA |
| | | | TTTATCCATATATGCAGAGAATATATTTTCTGAATGAAAAATTGGGC |
| | | | AGCAAACTCTGAAAAGTTCTAACATGCTCAGAGGGACAATGGGACYA |
| | | | CATAATTGAAGTTGGCACCAAACCATGAATATCTGGTCATCATAATA |
| | | | ATATAGATGCCTTGGACATAACAGCAAGCACTAACCACAAAGTAATG |
| | | | GTGTACTTTGCCCATAAGAAAGAAACAAATGTGTGACTGAAATCAGC |
| | | | TTTTCTCACTCTATTGCATGGAATATATAGTATTTCCTCAACATATT |
| | | | AGTTTTCCTGTTTTAAACTTACAAAAGTGTTTTCTTATTTAACAAGT |
| | | | TTAAGAAAATGGTGCAAACTATATTTTCTGTATGGGGAATTATAAAG |
| | | | CCCATCAGAATGTTACAGGTTGGAGAAGTTCCACATTAAAACAACTC |
| | | | TTTAACTTTGTTTAATATGAGTTTCTAGAAGATTGTTTCTTCCAAGA |
| | | | ATACATTGGCCTTGTAGGCACTTAGTCAGATCAAATGCCTTGTTACC |
| | | | TAGAAAACAGTTTGGAAAACACCAGTTCACACAAATGGTTATCTTGA |
| | | | GATGAAGCAGAGCTAGAAAAGTGTAT |
| IFNG | rs2870951 | 44 | TTGGTTTTGTAATTTATGTGGATGTAGAAACAGGCTTGGGGATGTTA |
| | (POSITION | | AATATTTTTAGTAGCATCACATAATTATCATGAAAGAAGTTAAAGCC |
| | 497) | | ATGATCTAGAAGATTTTACAATTCTCTGATTCACCTGTTGTGCCTTA |
| | | | TTTTCTCTCAGGTAAGCTTCTTAGTTATCTGGTTACTTTTAACAAAT |
| | | | GGCAGAAACAACTTCTTAACTATGGAAGATTATGTTCTTTTGATTTA |
| | | | CCAAATTATTTATCCATATATGCAGAGAATATATTTTCTGAATGAAA |
| | | | AATTGGGCAGCAAACTCTGAAAAGTTCTAACATGCTCAGAGGGACAA |
| | | | TGGGACCACATAATTGAAGTTGGCACCAAACCATGAATATCTGGTCA |
| | | | TCATAATAATATAGATGCCTTGGACATAACAGCAAGCACTAACCACA |
| | | | AAGTAATGGTGTACTTTGCCCATAAGAAAGAAACAAATGTGTGACTG |
| | | | AAATCAGCTTTTCTCACTCTATTGCAYGGAATATATAGTATTTCCTC |
| | | | AACATATTAGTTTTCCTGTTTTAAACTTACAAAAGTGTTTTCTTATT |
| | | | TAACAAGTTTAAGAAAATGGTGCAAACTATATTTTCTGTATGGGGAA |
| | | | TTATAAAGCCCATCAGAATGTTACAGGTTGGAGAAGTTCCACATTAA |
| | | | AACAACTCTTTAACTTTGTTTAATATGAGTTTCTAGAAGATTGTTTC |
| | | | TTCCAAGAATACATTGGCCTTGTAGGCACTTAGTCAGATCAAATGCC |
| | | | TTGTTACCTAGAAAACAGTTTGGAAAACACCAGTTCACACAAATGGT |
| | | | TATCTTGAGATGAAGCAGAGCTAGAAAAGTGTATTATTAATGAAGAA |
| | | | GAAGAAAAACAACAACTACTGGATTTCTCTTCAAAGAATAAGAAAAA |
| | | | CATTTAAGGAAGCAAAATGCTGATATGATAAATATGTTTGGAGGAGA |
| | | | TTAG |
| IFNG | rs2870952 | 45 | AAAATGTAAAATGGCTTAAACCTAATAGAAGTTTACTTTTTGCTCAT |
| | (POSITION | | GTAAAGTCAAAAATAGATGTAACAGAACAGGAGCTATCTCTTCTCTA |
| | 500) | | AGCAGGACCAGGATCCTTTCATCCTGTGGCTCCACCATCTTCACCAT |
| | | | CTTCAATACTTGGACTGTGAGGTCACTGTGCATGTTCGTATCAAGTT |
| | | | GGTCAACAGAGAAGAAACATGGAAGATGGCCCATGGAGGATGGCACA |
| | | | CATCACTTCCACTCACATTCCATGGGCTAGAACTCACAAATAAATTT |
| | | | GATGAACAGCAAGCCAGCCTCTGTTCCAAAAGTCTTCCTAGACAGAA |
| | | | TGTACATAAGCTGATTTAGTATCTGCACAGTCTCTGCAGTGATGCCT |
| | | | CTCTTTGTTGCTGCTTATTAAAGTGTTAACAGGATCAAGGATTGACC |
| | | | CAGAAATGGAATATTAAAAAGAAAGTTATGCTATAAATTCCACTGAG |
| | | | GGTTTTGTCATTTCAAGAGTGCTTCTGAAYGTCCCTGTTGAGGTCAT |
| | | | TTTTTTCTCTGTTTTGCCAAAAAAAATCTGCCCTCATTTTAATGACA |
| | | | ATCTAGTTTTTTTGTTTTGTTTTGTTCTTTTTTTTTTCTTTTTTGAA |
| | | | TCTCATTACCTTCAATATGTTTGGTCAGGTTGGATTGGTAAATCTGG |
| | | | CACATGGGGTTGCCTGTACCTCATCATGAAATCCAAAGGATACCTAG |
| | | | AGGGTCCTTCTACCAGTTTTTTTTTACTCAGCACTGTAGGATTAATG |
| | | | CCAGCAGGCAGTCAACTCATCCGTGTTCATTAGACTCACTTTCTAGG |
| | | | GTTTGATTCTGGAGCAGAGTGGTACAAAGATAAGAACAAAAGCATTG |
| | | | GAATTTACCAATTTGTTCCTGCATGGTGCTCTGCAGAAGGGCTGAGT |
| | | | AGTTTCTGCGGCAGACACCTTCTGGGATTGCCTGGTAGATTGTCTGT |
| | | | ATTGAACATGGTTCCTCAGCTATGTCTTCCATCCATGAGCTCCTCCA |
| | | | TATGCCTTCATT |
| IFNG | rs2870953 | 46 | GTGCGGCAGCCTGACATGGGTCCTCTGAACCTTAGCCTAGCAAGGAG |
| | (POSITION | | GTGCCCATGTGGAAGAATGGCCTGGAGTAGGGTGTCAGAGTCCAGGC |
| | 939) | | AAGGTGAGGAGGACATCTGTGTCCTGGGATGGCCCAGCATGAGTGTT |
| | | | AGAGTGAAGTAAGAATGGCATCTGCATAGGGGAAGACATATTAGTGC |
| | | | AGATGGGAAATTAATTAAGTAATTATATTAAAGATAATGGGAGCCAG |
| | | | TTTTCCTCACTATTGAAGGAAGGTACAGTACAGAAAAGGAGAAAATT |
| | | | AGAATAAACCCTATGATATTGAAATAGAATTAGATGTATCAGTATTA |
| | | | ACTTATGCTCTTCAATATATAGAGGTAGATATAGAAATAAATAATAG |
| | | | ATAGAAATATTAGTTCACCCTAACTCTGTCCATTGAGGGGGCCTGGG |
| | | | AGTAGTAACATCTCGATAACAATGAGAACACTGATCACCCATATCTT |
| | | | GACTTCTAAATACCATTCTTTGCTAGAATGAACCAGAACTCCTTGGA |
| | | | GAATTGGCTGATCCCAGAACAGGGGTAGTGAAAGTACATGAAGTGCT |
| | | | AGAAAAAAAAGAAGTATTCAGAGGATGATGGAAACATGTTAAAAGGA |
| | | | ACAGAAACCAGCTTGAAGGGACTCCCACTAGTGAAATATGAGAAAAT |
| | | | TTGAGCATCAAAATAAATAGTGATAGTAATGTATTATGACCTATTGA |
| | | | ATATAATAGGAAACCATGAGTATATATTGATATAAATGAATACACCA |
| | | | AAAGTTTGATGAGGAATGGTATAGCCACATCATTGCAAAATATCTCC |
| | | | CTACAAAATATTTATTAATTACAAATTGGAAAGGAGTAATTTTATGG |
| | | | TAGAGAAGCTTAGCAGATACCATCTTAATCAAGGAATAAAAGTGAAC |
| | | | ATCCTTAGTAATGAGATAAATGAAAAGGGTATCCTACCTGATAGGWT |
| | | | GCAAGAAAACGAACATAGCACACCTTTTGTGATATCTCTGTGAAAGA |
| | | | TGCATAACCTATTCTAGTCATGAGAAAACATACAAATGCAAACTAAG |
| | | | AAGCATTCTACAAAATATCTTGTCTGTAGTCTTCAAAGTATCAAAGT |
| | | | TGTATAAGTTAAGGAAAGACTAAGGACTGAAGAACAGTTTTGTTCTG |
| | | | AAATGAATTATAGAGACATGATGGCTAAATGCAATGCAAGTTTCTAA |
| | | | ACTGAATCCTTGTGCAGTA |
| IFNG | rs3181034 | 47 | GATTGGAAGTAAATTTGGTTAATTTTGGCACAACTACAGCTAATATG |
| | (POSITION | | ATCTGCTACTGGTGTCTCCACTCTTGTTTGAATTAAGATTCATTGAT |
| | 301) | | GCAGACTTTGATGTATAAACACATGAAGGGCTACTGTGAAGCTGTCT |
| | | | TGCCTGGACCACAGTTCTAGACCATACTTCTGGGGAAGGGATTGGAA |
| | | | ATTCCCACCATCGATCATCTTGAGCAGTGATTAATCATACCATGTGT |
| | | | AATGAAGCCCCAGGAGATAAATTTTAATCTAGGGCTGGATAAAACAT |
| | | | TCCCTCTCTCTGAGGTTGRTCTCAGTTTGGCCAGACTGTGAGACATG |
| | | | AAGAGATATAAACTGTATTAGGTGCTGTGATTATAGCAGGGAATGAG |
| | | | ACAGGGAGAAGATCCTTTAAGACAACTTGAGTTGAGACTGGCCTATG |
| | | | CAGTGGTTGTCAATTATTCTCTATGTTGTATGTTTCTTCTCTTATGA |
| | | | ACACACCTAGTTTCAGAAGTGTGATGGAGCTTGTAGGAGGGATGGAC |
| | | | CATGCTTTAGACTAAGACACCTTGGGGGCTGATTCCTCTCCCAATGC |
| | | | CAGCAGGGGCAGGTATCTCCCAAATCTTATAAGCAGC |
| IFNG | rs4913277 | 48 | ATAGGTAAAATCTTTCTAGAATGAGGAGGAGCACCTGAGGGATCAGT |
| | (POSITION | | ACATGATGACCATGGGGATTAGTGCATAATGTAGTCTGATGATAGGA |
| | 501) | | TATTTAAAGCAGGAAGACACTAAAGAGTTTCAAGAAGAAGAGAGGGA |
| | | | GAATGGGGTGTGCCTTGATGAAACACAAGAATGGTACTTAAACGACC |
| | | | TCCACCTACATGCCCACGGGTGCAAAACAAAGGGAAAGAAAACAGAT |
| | | | GCATCTAGAGAAATCTGCAAAGGAACCAGGTCTCCAAGGGACAGTCT |
| | | | GGTCAGTTACAGTAAGAAAGCAAAGTTCAGAGAAAATGTTAAAGATA |
| | | | TAAGGGATCTTGCTGGTGACTGACAGTGAGTTCAGGGGACACACTGA |
| | | | AAGGGTTTCAGAAGCTGGAGATAGGTGGAAGATGAAGTGAGGGAAAA |
| | | | GGAAGTGCAGTGCCATCACGGAAATGAAAGCCTTGGGACGGAGGGGT |
| | | | CACCTGGATGTCCTGGGCTTCTTGGGCCCTYCGTCCTAAACAAGCAT |
| | | | AAAGAGCATCACGGGATTATCCTTGGTAGTCTCAAAGCTGAGAGTCA |
| | | | TGGGGAGGCTGTGAACATTGAAGATCCTACCAGGGACACAAAATTAC |
| | | | GGGTCCCTTCTTCAATCCTGCCTGTGGTTAGCAGGAGGTTGAGGGAG |
| | | | CGATGGTCCTATTTCCCAGAGGAATAAGAGCTCTGGGCTCCTTCAGG |
| | | | AAACCTGGGGAAGAGGATGCCCAAGTCTGCATGAATACCAACAGATG |
| | | | AGGCCATCGGAAGAAGGGCTCCTAAGAAAGAGAAACCACACACAGAA |
| | | | AGGAAGAAGTGAATATGACCCATGCTCACACAC |
| IFNG | rs4913278 | 49 | TCATCTTTATTGGCACATGCCAGGACTTGATAACCTTAGTTTGTAAT |
| | (POSITION | | GTGAATCCTATTTAAAAGTATTTAAAAGTATTTCCACTACAACTTAA |
| | 1311) | | GAAACTCTCATCCAGTGCAAAGCTCAGGGTAGACAGCAGAGAGTTGG |
| | | | ATTTAGCCATGATTGATTGGAGTTTTTCCAGGAAAATACGATGAAGG |
| | | | AAGACAAGAACAAATGACAGACCATGGAATTGAGGCTCGATAATGAG |
| | | | AGAAGTAAAGACATAAAGTGGAGAGGAACCGTGAAAAGATGCTAGGA |
| | | | ATAATGTTTTTTTTCAATTCCATTGGAATTTAATGACAGCTAGAGTG |
| | | | GGTTATAGAAAGGGCAAGCTGAAAAGTCATAGAGTAGGAGTCATGTC |
| | | | ATTGAGATAATGTGGGGAATGGGGGCTGTTGCTATTACAATGCAACT |
| | | | TCTAGGATCCTCCCAATGGGAAGAATTGGCTAAAGTAAGATAAAGGG |
| | | | CAAGATCTGAGTGGAAGGGAGATCAGGAATGGAGAGACCAGCGTGTT |
| | | | TGAAGTACCACATGTACACATATTGAAGTGTATGATATGAGGTGGTG |
| | | | TTGGAGAGTGTGACAGTGAGCAATAGGTAAAATCTTTCTAGAATGAG |
| | | | GAGGAGCACCTGAGGGATCAGTACATGATGACCATGGGGATTAGTGC |
| | | | ATAATGTAGTCTGATGATAGGATATTTAAAGCAGGAAGACACTAAAG |
| | | | AGTTTCAAGAAGAAGAGAGGGAGAATGGGGTGTGCCTTGATGAAACA |
| | | | CAAGAATGGTACTTAAACGACCTCCACCTACATGCCCAGGGTGCAAA |
| | | | AGAAAAGGGAAAGAAAACAGATGCATCTAGAGAAATCTGCAAAGGAA |
| | | | CCAGGTCTCCAAGGGACAGTCTGGTCAGTTACAGTAAGAAAGCAAAG |
| | | | TTCAGAGAAAATGTTAAAGATATAAGGGATCTTGCTGGTGACTGACA |
| | | | GTGAGTTCAGGGGACACACTGAAAGGGTTTCAGAAGCTGGAGATAGG |
| | | | TGGAAGATGAAGTGAGGGAAAAGGAAGTGCAGTGCCATCACGGAAAT |
| | | | GAAAGCCTTGGGACGGAGGGGTCACCTGGATGTCCTGGGCTTCTTGG |
| | | | GCCCTCCGTCCTAAACAAGCATAAAGAGCATCACGGGATTATCCTTG |
| | | | GTAGTCTCAAAGCTGAGAGTCATGGGGAGGCTGTGAACATTGAAGAT |
| | | | CCTACCAGGGACACAAAATTACGGGTCCCTTCTTCAATCCTGCCTGT |
| | | | GGTTAGCAGGAGGTTGAGGGAGCGATGGTCCTATTTCCCAGAGGAAT |
| | | | AAGAGCTCTGGGCTCCTTCAGGAAACCTGGGGAAGAGGATGYCCAAG |
| | | | TCTGCATGAATACCAACAGATGAGGCCATCGGAAGAAGGGCTCCTAA |
| | | | GAAAGAGAAACCACACACAGAAAGGAAGAAGTGAATATGACCCATGC |
| | | | TCACACACCAACATGCCTATAGCCAGGAGGAAATATGAGAGCTAGGA |
| | | | GGGAATTTAGGAGTCTCTGAATTGAAAGTATTCGTTTCAGTGAGGAG |
| | | | GAAACTGAAGTTTAGACACGTAGAATAAACTTATTGTAAGAGGAACC |
| | | | TATGTAATATGTCTTAGAAAGCTCTCTTTCAAAATCATTATCCAAAA |
| | | | AGGA |
| IFNG | rs4913405 | 50 | AATCCTACAAGAAACATTTCATTATTCCCACTTAGAAGCTAAGAAAA |
| | (POSITION | | TGAAAGTTAAGAGAGATTAGCTTCATATGACGAGGAATAAAAACCAC |
| | 1307) | | ATTTTTCTTTAGGTTTAGTTTATTCATCTATTTCTAGTTCCTTGCAG |
| | | | TGTAACATTAGGCTGTTTATTTGGGATCTTTCTTCTTTTTTAATGTA |
| | | | GATGTTTATTGCTTTAAACTTCCCTCTTGGAACTGTTTTTGCTGCAT |
| | | | CCCATAAGTTTTGGTATGTTGTGCTTCCATTTTTATTTGTCTCCAGA |
| | | | TTTTTAAAAAATGTCTCTTTTAATTTATTTGTTGATCCATTGGTTAT |
| | | | TTAGAAACATGTTGTTTAATTTCCACATATTTGTAAATTTTCCAAAA |
| | | | TTCCTCCTATTATTGATTTTTAGTTTCATACCATTGTTGTTGGAAAA |
| | | | GATACTTGATAAGATTTCAATCTTCTTAAATTCGTTAAGACTTGTTC |
| | | | TGTGGTCTAACATATGATCTATCCTGGGGAATGTTGAAGCAAATGTG |
| | | | TATTCTGCTGCTGTTGGATAAAATGTCATGTATATGTCTGTTAGTTC |
| | | | CATTTGGTATATCCAATGTTTCCTTATAGATATTCTGTCAAGATAAT |
| | | | CTGTTCATTGTTGAAATCCCCTACTATTATTGTCTTGCAGTCAATCT |
| | | | CTTTCTTCAGGTCTATTAATATTGGCTTTATATATCTACGAGCTCTG |
| | | | ACATTAGGCACAAATATATTTACAATTATTATATCTTCTTGATGAAT |
| | | | TAATCCCTTTATCATTAGATAATGAAGTTCTTTGTCACATTTCACAG |
| | | | TTTTTGACTTAAAGTCTATTTTTTTTTTGACATAACCATAGCTCTCC |
| | | | CTGCTCTTTTTTGGTTTCCATTTGCCTGGAATATTTTTGTTCATCCT |
| | | | TTCATTTTCAACATATGTTTGTCCTTTAAGGTGAAGTGAGTCTCTTG |
| | | | AAGGCAGCATATTATTATTTTTTCACCCATTCAGCCATTCTGTGTGT |
| | | | GTCTTTGGTTAGAGAATTTAATCCATTTATATTCAAGGTAATTATTG |
| | | | ATAGGTAAGGACTTACTCCTGTCATTTTGTTAATTGTTTTCTGATTG |
| | | | CTTTGTAGATTCTTTGTTTCTTTCTTTCTCACTGGCTGTCTTCCTTT |
| | | | CTGATTAGATAATTCTTTCTAGTATGCTTTCATTCCTTAAAGTTTTA |
| | | | TCTTTTGTTTATCTACTATACATTTTTGCTTTGTGGTTACCCTGAGG |
| | | | CTAACATAAAATATCTTATAGTTATAAAAGGTTATTTTAAGCTAACA |
| | | | ACTTAACTTTGACCACATTAAAAAACTTAACACTATTRCTCCACCAT |
| | | | GCCCCACATGTTTTGTTTTTTATGTCACAATTTACATCTTTTTTTAT |
| | | | TGCGTATCCCTTAACAAAGTATTGTAGCTATTATTATTTTTAGTAGT |
| | | | TTCATCTCATCTTCATAGTATGAATATAAGTGATCTATCACTTATAT |
| | | | TCATAGTATGAATATAAGTGATCTAATCTCAACCACCATTAGATTAT |
| | | | TGAGTATTCTGAATTTCACTGCATCTTTATTTTACCAGTGAGTTTTA |
| | | | TACTTTGATAAATTTTCATGTTAATAATTAATATTCTTCTATTTCAG |
| | | | CTTGAAGAACTCCCTGTAGCATTTCTTATAAGACAGGCCTGGTGGTG |
| | | | ATCAAATTCCTCAGCTGACGTTAAGTCTGGGAAAGTCTTTCTTTCTC |
| | | | CTTCATTTCTAAAGGACAGCTTTACCAGGCAATATATTCTTAATTGA |
| | | | CAGGTTTTTTTTTCCCCCCTGCAGCACATTGAATACATCATCCAACT |
| | | | TTCTCCTGGCCTGTAAGGTTCTGCTGAGAAATCTGCTTCTAGCCTTA |
| | | | TTGAAACTTCCTTATATGTTATTTTCTTCATTTCTCTAGCTGCTTTC |
| | | | AGGATCCTCTCTTTGTCTTTGATTTTTTGTGGGTTTTTTTTTTTTTT |
| | | | TGCGGGGGAGGGGGTTGTTTGTTAGTTTCTCGGGTTTTGTGTTTATT |
| | | | TTTCCTTTTGTTTCTTTTTTGTTTATTTGTTTTGTTTTTTGAGACAG |
| | | | GGTTTAGCTCTGTCATCCAGGCTGGAGTGCAGGGGCACGATCTTGGC |
| | | | TCACCACAGCCTCAACCTCCCAGGCTCAAGTGACCCTGCCATCTCAA |
| | | | CCCCCTGAGTAGCTGGGACTACAGGTGCATATCACCACACCTGGCTA |
| | | | ATTTTCCTATTTTTATATTTTCATTTTTTGTAGAGACAGGGTCTTGC |
| | | | CATGTTGCCCAGGTTGGTCTCAAACTCCTAGGCTCAAGTGATTTGCC |
| | | | TGCCTTGGCATCCCAAAGTGCTGGGATTACAGGCATGAGCCACTGCA |
| | | | CCTGGCCTTCTTTGTCTTTTGATTTTTGACAGTTTGATTACCTGTCT |
| | | | TGGGGTAGTCTAGTTTAGATTGAATCTGATCAGAAAACTTTGACTTT |
| | | | CCTGTAGTTGGATATTTATCTCTTTCCCTTGATTTGGACATTTTCTG |
| | | | CTAGTATTCTTTAAATAAGTTTTCTGCTTTTTTGTCTTTCTATTCTC |
| | | | CTTCTTGAACTTCTGCAACTTGAATATTTGCCATTTTGATGCTTTCC |
| | | | CATAAATCTCATATGCTTTCTTCTTTCCTTGTTATTCTGTATTCTTT |
| | | | TTCTCCTCTGATGGTATATTTTCAAATAACCTGTCTTCAACTTCACA |
| | | | ATTTTTCTTCTGCTTAAGACTTTTTTTAAATTTTTTCATCTTAATTT |
| | | | GTGAGGGTATATAGTAGGTGTATATATTTATGTGGTACATGAGATGT |
| | | | TTTGGTATAGGCATGCAATGCACAATAATCATTTCATGGAAAATGAG |
| | | | GCGTCCATCCTTTCAGGCATTTATCCTCGTATTACAATCTAATTATA |
| | | | CTTTTTAGTTACTTTTAAACGTACAATTACATTATTTCTCACTATAG |
| | | | TCACGCTGCTGTGCTATCACATATTCTTTCTATTTTTTGTACCCATT |
| | | | AACCATCCCCACTCCCCTATCCCAAATCCCCTACTACCCTTCCCAGC |
| | | | CTCTGGCAACTATCCTCCTACTTTCTATCTCCATGGGTTCAATTGTT |
| | | | TTGATTTTTAGATTCCACAAATAAGTGAGCACATCCAATGTTTATCT |
| | | | TTCTGTGCCTGACTTATTTCACTTAGCATAATGACCTCCATTTCCAC |
| | | | CCATGTCATTTGCAAATGACAGGATCTCATACCTTTTATGGCTGAAT |
| | | | AGTACTCCATTGTGTATAAGTACCACATTTTTTTTATCCATTCATCT |
| | | | GTTGATGGACACTTAGGTTGCTTCCAAGTCTTAGATTCTGAACAGTG |
| | | | CTGCAACAAACATAGAAGTGCAGATATGTCTTTGATATACTGATTTC |
| | | | CTTTATTTGGGGTATAT |
| IFNG | rs4913415 | 51 | GAGCGATGGTCCTATTTCCCAGAGGAATAAGAGCTCTGGGCTCCTTC |
| | (POSITION | | AGGAAACCTGGGGAAGAGGATGCCCAAGTCTGCATGAATACCAACAG |
| | 288) | | ATGAGGCCATCGGAAGAAGGGCTCCTAAGAAAGAGAAACCACACACA |
| | | | GAAAGGAAGAAGTGAATATGACCCATGCTCACACACCAACATGCCTA |
| | | | TAGCCAGGAGGAAATATGAGAGCTAGGAGGGAATTTAGGAGTCTCTG |
| | | | AATTGAAAGTATTCGTTTCAGTGAGGAGGAAACTGAAGTTTAGAGAC |
| | | | GTAGARTAAACTTATTGTAAGAGCAACCTATGTAATATGTCTTAGAA |
| | | | AGCTCTCTTTCAAAATCATTATCCAAAAAGGAAAGAATGGGCCACTT |
| | | | AAAGGAGTATTGATTTATTAATCGGGAAATTTGCTTATGGAAAATAG |
| | | | GCAAAACTTGCTTCGAAATGCTTATCACAATCCACCTAAAATTTCTG |
| | | | TTGGCAGCATCATTATCTGTA |
| IFNG | rs4913418 | 52 | AGCACAGATTAGGACACACATTTACAGCCTGTGCTATATGAACAAAG |
| | (POSITION | | CTGAAATTAACTGGGACTACCGAATAAATAAAATACATTATATTTGC |
| | 301) | | AAAATATATAATTCATAGCTAATATGACATTTTAATTTTTATATAAA |
| | | | AATATATTTTTATATCTGCCCATATGCATATACATGCATGCATACCC |
| | | | AGACATGTGTATACACACATTTACATACCTGGAAGGATGTTCCCGAT |
| | | | GTGTTAAATGGAAAGAGCTAGTTGAAGGGTAGAATAAATGATATGAT |
| | | | AACGTTTTTGTTTCTAGARAAGGGAAAGATACTCTATATGAACATAT |
| | | | ATTTATATTGTTGTTGGAAAAATTTAAAAATTGTGGGAAAATCCCCA |
| | | | CAAACTGCCATCATTGGCTCACTTGGGAAAGTAGAGGTGGAAAGGCA |
| | | | GTGAGCTATGATTAGTTTATATACCTTGGTGTTATTTCAGTTTTACA |
| | | | ACAAACATATATTACTTTTTGTAATATAGGAAACTATAGGTTTGTAA |
| | | | CTAGGAAAATATATATAAATTTCAAGAGGACAGATTTCAGATTAATA |
| | | | TGAATAATTTTCTAATAGGCAGGATTATTTGGATTTA |
| IFNG | rs6581794 | 53 | AATTTCCCATCTGCACTAATATGTCTTCCCCTATGCAGATGCCATTC |
| | (POSITION | | TTACTTCACTCTAACACTCATGCTGGGCCATCCCAGGACACACATGT |
| | 354) | | CCTCCTCACCTTGCCTGGACTCTGACACCCTACTCCAGGCCATTCTT |
| | | | CCACATGGGCACCTCCTTGCTAGGCTAAGGTTCAGAGGACCCATGTC |
| | | | AGGCTGCCGCACTGTGATAGACTGCATAATGGACCCCCAAAGATGTC |
| | | | CACATCCTAGTCCCCTGAATCTGGGACTGTTGCTTTATATGGCAAAA |
| | | | AAAAACAAACAAACAAAAAAAAAAAAAAAAACTTCTCAGTTAAAAAT |
| | | | CTTAAGAAGGAGGAGACACTATCCYGAATTATTCAAGAGGTCTTAAT |
| | | | GTAATCACGAGGGTCCTTATAAGAGGAAATCGGGAGTATCAGAGTCA |
| | | | GAAAGAATGAGAGAGCCTGGAAGATACTCTTCTGCTGGCTTTTAAAA |
| | | | AGGAAGAGGCCACTAGCTGAGGAATGAGAGTGGCCTCTAGCAGTTGG |
| | | | AAAAGTCAAGGAAACAGATTGTCTCCTAAAGTCTCCAGAAGGAACAA |
| | | | GCCCTGCCGATGCCTTGATTTTAGCCCACTGAGCCTAGTTTTGGATT |
| | | | TCTGCCCTCCATGATGATAAGATAATAAACGTGGGTTTTTTTTTTCA |
| | | | GCAACTAAGTTTGTTGTCACTTATTACCAAAGCAATAGGAAACTAAT |
| | | | ATGCTCACCCACCTCTTCAAGGACCTTCTCCTCATTCTGCTCAGGTT |
| | | | CTGACACCTTCCACACCAGGTTTCCTCCCTACACACTGTGCCTGGAA |
| | | | TTTGGCTGCCCATAGTGGCCAATTACACATGTCTACCTTACTCTGAT |
| | | | GCACTTAATTGATTTAGAATAATATTGTTCAAATGGGAAGGAGAGAA |
| | | | GAATCAAGAGAAC |
| IFNG | rs6581795 | 54 | CAGTGATCTCAAGAAATGAGTTGTCCTCAGGGTAGCCCCTGAAATGG |
| | (POSITION | | CAATGGCATGAGGCTTTGAAAACTTGTATATTTTTCCAATGGAAACT |
| | 201) | | TACTCCTGTATCTCTCATGATAAAAGTTCTATACAGCAGACTGGCAG |
| | | | GTTCACGTTCTCTCCTATGCTACCTGGCAGAGGAATTCTGAGTCCAT |
| | | | GATGAGCCAATARATAAGTTTCTTTTCTCACCAGTGTTTAACCTGTC |
| | | | ATTATTACCATGTCACCAATCCCTGAACCAATTTAAGAAGTATCAGA |
| | | | ATTAAATTCCCATCCATTGATTTTTCAAATGGAAATATTTTTTAATG |
| | | | GTTGTAAAATTATGTGGGATCTTTTAAATAAAAAAAAAAACAGAAAA |
| | | | TACAGAACAGCATAAGAAAAGAAAAAAATCACCTATGATTTCACTTT |
| | | | CCACAGGTAAATATAGTTAGCTTTTGGAGACAAAGTATTTCATTATT |
| | | | TTTTTCTACCTACATAACACAGTGCCTGGCACAAAATTTTTTCCCAA |
| | | | TAAACTTCTGTGGATTAACGAAATATGAGCCAAAGTGATTTAATGAT |
| | | | TAAGTTCAAAGGCTCCGGAGTCAGGCTGTGTGTCTTCAATCATGACT |
| | | | TTGCTCCTTACTGTCTTGTCGATTATTAGTGCATTACTTAACTTCTC |
| | | | TGGGTATCACTTTCCTCGTCTGTAAAAAGGTGATATAAATAATAACT |
| | | | GTCTCAAAAGATTTCATGAGTATAAATTATGTCAACATGTGTAATAG |
| | | | TGCAATGCCTTGCATGTGGTAAGAGCTCATTAAATGCACAGTCATTA |
| | | | TTACTAGTGGCTT |
| IFNG | rs7132697 | 55 | TCCCTAAACTTACTGATTAAAAACAAAAATAGCTAAGCCCCAATTAC |
| | (POSITION | | CCAGAATTCCTGGTGCCCCTAACCCACCCAAGATCAGTTACTCATAT |
| | 301) | | TGATGATTCTTGTTACCCCAGAGTTTTCAGTGCCTTCTACATAGTAC |
| | | | CCTCAATGAGAGAAAAATATTAATTTGAAAATATTTGAAATGATCTT |
| | | | GTCAAACTCCTTGGAAGATTAATCATATGCCATTGATTAGAAACCAG |
| | | | AGAAAAGCAAGGCTGCAAAATTATGGCTTCATGCATGCACAGGTGTG |
| | | | GAAGTTTCCATAAAATTCWATTAGTCCAATGGTCATAGGGCTGAGTG |
| | | | GGTGATAGCCATCTCCCCACCCTCCAAGTAATTTTGGAAATGTACTG |
| | | | TGAGCAACTCTGATTGTCACAATAATTTCGTAGGTTCTACTGGAGTA |
| | | | TTGTGGGTGGGGAAGTCAATGAGGTTAGAACTCCTGCAATGCATGAG |
| | | | ACAGTTTTGTGCAATGAAGAATTGCCCCATGTCTCATGCAATTTACA |
| | | | CACATAATTTATCTTAATTTATACAGTGGCCCTGTAGGTAGTGTATT |
| | | | TATCTCCATCTGGCAGATTATAATGGAGGTTAATGGG |
| IFNG | rs7133554 | 56 | ATGCAGGGGCCTCTAGAGACCCCACTACAACATCTAAGATAATTCTC |
| | (POSITION | | CACCTAAAGTAGTGAAAAATCATGTTGGACACCAGAAAGCTCTTAGC |
| | 301) | | AAGGCTCAATAATTAATTACTGATGTTATTTTCACATGGAAAGAAAT |
| | | | ATTCTTGGTAAATCAGAATAAATTTCTTGAAACTTCATGTAAAATTC |
| | | | ATAATTGTGTTAAGGTAATTTTGAGCCACTGTCTGTGTATGCCGTTC |
| | | | TGTGGGATACACAGAGTATACCTTTGTGAGGCTCCAGGGACATTCTT |
| | | | TCCACTTCGTACTTCTTTYTAAATCACAAGGTAAGATCTTATGAGAT |
| | | | GCAAAGATTAATTTGTTTTCCTCCACCAACTTAAATTTTTCTCCCTT |
| | | | TCTTTACTACCTGTAGGATTTTAGCACTGAATAAATAATAGGCTTGA |
| | | | AGGTGAACTATTTTCATGAGCCCATATGCATTAGGACAAAAACTGAA |
| | | | TTCTATGGTTTAACCAGGACATAATATACATCAATATGGTCTTTGAA |
| | | | TGGCTTACAAAGGAAAAAAAACATTTCCTGGGTTATTGGAAGCAGCA |
| | | | TGGTGTCAAAGTAGTTAAACAGATTCTATCTCTGTGG |
| IFNG | rs7137814 | 57 | TCATGTTTAACGTATTTGTTCAGGTTAAATTGAAATATTTTACATAT |
| | (POSITION | | AGAAACTGAGGTTGGGTTACCTCAGAAACAGAGCTTGAGACAAGGAT |
| | 501) | | TTTTTTTTTTTTTTTTTTTTTTTGGTGGTGATTCTAGGAAGCACCAG |
| | | | TAGAAAAGAGGCAAAGAGATTCAGGGAAGGGAAGGAAGTCAGTTCAG |
| | | | GGTGGTTCCCAAAGGGAGCTACTGTAGTCAACTGAGACTCAGCCCAC |
| | | | TATAGACCTCTGGGTGATGGTGTAGCCCATACCCCAAAGTTATCCTG |
| | | | CCCAAGGGACGAAGAAGTTGGGGTATCTATCCTGCGACTATCTTTAG |
| | | | CACTGTCTGAGCACTGCTCCCAGGGCATTAAACCCCTAGCTCTTCCA |
| | | | GTCTTCCTCATGTGAAAATAGAAAGAAGCCCTTAGGCCAAGAATAGT |
| | | | GAACTGTTACAGTCACAGGCAGAGGGTAAGAAGAGAGAGGGAGGCTG |
| | | | CTGAGAGGATGTTGGCAAGGCAGGTAGTATYTGCTATGAGAAGTTAT |
| | | | TAATTATTCCCTCATATTTTTTTTCAGTTTTTATTACATCCTTTATT |
| | | | TTTCGGCATTAGTGTCAGTATACCAACAAGTTGCATTTGCCAGGACT |
| | | | TTTGTGGTGACAAGTGACGAAAATTCCAGTCACACTATTTTGATCAA |
| | | | AGAAAGGATCTCAGAGACAGGTACTCAAGTGTTGACAGGATTTGTCT |
| | | | CTCTAGCTGTCACTTCTGCTTCTCTTTGTGAGACAATGTCAATCCTG |
| | | | CCTCCCACAGAGCAGCATTC |
| IFNG | rs7137993 | 58 | TAGAAGTTGTATTTGTACATTTCTTCATGTTTAACGTATTTGTTCAG |
| | (POSITION | | GTTAAATTGAAATATTTTACATATAGAAACTGAGGTTGGGTTACCTC |
| | 501) | | AGAAACAGAGCTTGAGACAAGGATTTTTTTTTTTTTTTTTTTTTTTT |
| | | | GGTGGTGATTCTAGGAAGCACCAGTAGAAAAGAGGCAAAGAGATTCA |
| | | | GGGAAGGGAAGGAAGTCAGTTCAGGGTGGTTCCCAAAGGGAGCTACT |
| | | | GTAGTCAACTGAGACTCAGCCCACTATAGACCTCTGGGTGATGGTGT |
| | | | AGCCCATACCCCAAAGTTATCCTGCCCAAGGGACGAAGAAGTTGGGG |
| | | | TATCTATCCTGCGACTATCTTTAGCACTGTCTGAGCACTGCTCCCAG |
| | | | GGCATTAAACCCCTAGCTCTTCCAGTCTTCCTCATGTGAAAATAGAA |
| | | | AGAAGCCCTTAGGCCAAGAATAGTGAACTGTTACAGTCACAGGCAGA |
| | | | GGGTAAGAAGAGAGAGGGAGGCTGCTGAGARGATGTTGGCAAGGCAG |
| | | | GTAGTATCTGCTATGAGAAGTTATTAATTATTCCCTCATATTTTTTT |
| | | | TCAGTTTTTATTACATCCTTTATTTTTCGGCATTAGTGTCAGTATAC |
| | | | CAACAAGTTGCATTTGCCAGGACTTTTGTGGTGACAAGTGACGAAAA |
| | | | TTCCAGTCACACTATTTTGATCAAAGAAAGGATCTCAGAGACAGGTA |
| | | | CTCAAGTGTTGACAGGATTTGTCTCTCTAGCTGTCACTTCTGCTTCT |
| | | | CTTTGTGAGACAATGTCAAT |
| IFNG | rs7138107 | 59 | CTATCAGGCTTATATTCCTAGTGTCTAGGAAATTGCCAAGCCTAAAA |
| | (POSITION | | GAAAAGATGTACTAATGTGGGGTTCCTGCCAGTGAAACTCACCAGTT |
| | 1216) | | TCAAGTATCACCCTACTAAGAGGCTTGTAAGTCAGGAAGTCCAATCA |
| | | | ATATATTTAATGTACCCAATCCAACAATTTGGACTTTGTTATAAAAC |
| | | | ATAAACAACATTAATGAACAGAAAAATACTTGAAAAAATACTTTAGG |
| | | | ATAAAATACAAAGTCTAAAAAACAAACAGAAAAAATAAATAAAAGAA |
| | | | ATGAAGTTTAATGCCGAGAAAAATAACAAAGAGAAAAAAATTTAAAA |
| | | | AGTAATAGAAGATTCATGGAACAATAACATTTAGAGAAGAAGAAGCT |
| | | | CTTAGAACTTAAAAGCTGGTGGTAGAGCCAGGTGCAGTGGCTCATGC |
| | | | CTGTAATCCCAGCACTTTGGGAGGCCAAGGTGGGCAGATCACTCGAG |
| | | | GTCAGGAGTTCGAGACAAGCCTGGCCAACATAGTGAAACCCCGTCCC |
| | | | TACTAAAAATATAAAAATTAGCCAAGCATGGTGGCACACATCTGTAG |
| | | | TCTCAGCTATTTAGGAGGCTGAGGCTGGAGGATTACTCGAGCCCGAG |
| | | | AGGCAGAGGTTGCAGTGACCCAAGATCGCACCACTACACTCCAGCCT |
| | | | GGGTGACAGAGTGAGACTCAGTCTCAAAAAAAATGGTGGCAGAAGTT |
| | | | TAAAAGCAATAGAAGGGTTGAAATATAAAGTTGAAGAAATCTCTAAG |
| | | | AAAGAACAAAATGACCAAGAACTGGAAAAATATAAAGAAATTCACGA |
| | | | AAACTAAAGAATCTACTTAGAAATCCAACACTTAAGTAACAGGTGCT |
| | | | CCAGAAAGAGAAAATATGTAATTGAAGGAAGAAAATTTTCAGAAGAT |
| | | | TATTTGTATAATTTTTCCATAGCTGAAGAATGTGAGTTTCCAAAATG |
| | | | AAAAACCCAACGAATGCCCAGCCCAATGAGTTTAAAAAATAAAAATA |
| | | | AAAAGACAGGCCTTGGAGTGCATTTTTAAATTTCGGAGAATCTTGTA |
| | | | TGTGAGAAGATCCTCAAAGTTCAAGAGAGAAAACATAGGTTGTAAAC |
| | | | ATTATAAATACAAAGGATGCAGAAACACAATGTCACCGGACTTCTCA |
| | | | ATAGCTATTCTGGAAGCTAGAGGTTGATGGAGCAATGTTTTTAAATA |
| | | | TTGGAATAAAATAGTGTCCAAACTAGAATTTCACGCTATGYCAAACA |
| | | | ATTAATAGTTAGGATGAGACAATTTTTTTTATTCATGGGAGATTTCA |
| | | | TGACTTTATGTCCCATCTGCCCTTTCTCATGAAGCATCTTGAGAAAG |
| | | | TCAAGAAAGTGTTTAACCTAAATAAAGAAATTAAATTAAGGAAGAAG |
| | | | ACCTGGGATCCACGAAACAAAGGATTTAACACAGGAAAAAGCTAGAC |
| | | | TATTTTCTAGCACGTGGTGAGGGAAGTCCCAAGAGGATCATTGTGCA |
| | | | GCAGGCCTACACAGCAACCAGCACTGGTTGGAACTAAAGGACTGGGA |
| | | | AGCCCAGGAGAAATGTCTCCAAGAAAAGAAATGGAATTAATATGAAC |
| | | | ATTACGAAGAAATTTCACCCCTGACAGAGACTGGGGTAGGGGAAGGT |
| | | | AAATTAATGATGAGTATGTGGAAAACTAAGAAAACCAACCAAACAAA |
| | | | GCCAATTATTAACTTCAGGAAAAGCAAATATTGTCCACGAAAAAAAT |
| | | | GTAATATTGTACCACAAATGTCATGAACAAGAATTACCTAATCATAG |
| | | | TCATGTCCATTTTACCACCTAAAGTGTAATATAGCTATAATGGGAAG |
| | | | ACAGAGGACAAAGGGGCTAAGTGTATATGTGTATAGGGTAGAGTAAG |
| | | | TCATAGTCATATTACCTGAAATGGGAAAAATTCAATGTAAGAAATAG |
| | | | GTAGTTTTACTGGGTAAGTAGAAGTTGAGCTAAGAAATGAAGCTAAA |
| | | | GGAATTGAAAGTGATAGCCTCAGAGAAGTATGTTTTAGAGATGGAAC |
| | | | TGCATGAATCAGAGTTACTGGCTTTTTGTTATAAGCCTTGTGGTATT |
| | | | TGGAACATCTGGGAGTCCCCAAAGCCACCTTCATTTCTGACACCAGC |
| | | | TGAAAGTTTGGAACCAGCCCCAGGTTCAATAATTCACTAGAAGGACT |
| | | | CATAGAACTAAGAAAAACCATTATACTCATGATTATGGTTTATTACA |
| | | | GCAAAAGAATACAGATTAAAATCAGCAGAGGAAAGAGGTCCATAGGG |
| | | | CAGGGCTCAGGAGCACTCCATGCTTAGAGCTTCCAGTCATTCTCTAC |
| | | | CAGTAGAGAAGTGGACAGTGCTAACTTTTCCCAGCCATGATGTGTGA |
| | | | CAATATACACAGAGTACTGCAGACTAGGGGAGCTTACTTGAGTCTTG |
| | | | CTGTCCGGAGACTTTATTGAGCTTGGTCACATAGACAAGATTGACAC |
| | | | CTGTATGATTGACTTTGGTCTCTAGCCCTTTCAGAGGTCAATTTGAT |
| | | | ACTTTGTGGCCCAAGGCTCCCACCATAGATCACATTGTTAGCATAGA |
| | | | TTATGTCGCAGGGCTTAAGGCCTCTAGGAAACCAAAGACACTCTTAT |
| | | | CAGGCAGGACATTCCAAGGGCATAGAGGTTACATCCCCAGTGTTGGA |
| | | | GACAAAGACCAAACCTCTCTTCGGATGAAGTTAATCCTGTACTGCAT |
| | | | AATATTCCTTTATTTTTTCCCTTTTAAACTGTTT |
| IFNG | rs7298410 | 60 | GGGTGGCTCATGCCTGTAATCCTAGCACTTTGGGAGGCCAAGGCAGG |
| | (POSITION | | TGGATCACTTGAGCTCAGGAGTTTGAGACCAGCCTAAGCAACAAGGC |
| | 488) | | AAAACTCTGTCTCAACAAAAAATGCAAAAATTAGCCTGGTGTGGTGC |
| | | | CTTGCACCTGTAGTCCCAGCTACTTGGGGGGCTGAGGTGGGAAGATC |
| | | | ACTTGAGCCCAGGAAGTCGGGGCTGCAGTGAGCTGAGATGGTGAGGC |
| | | | TGCACTCCAGCCTGGGTAACAGAGTGAGACCCTGTCTCAAAAAATCA |
| | | | ATTAATCAATAAAGTGTTGTTGATGTTTATGAAACCCTTAGAGCTCT |
| | | | ACCAGGCATACAGTGAACTACGATGTTGTTGATGATGATAATCATCT |
| | | | TTATTGGCACATGCCAGGACTTGATAACCTTAGTTTGTAATGTGAAT |
| | | | CCTATTTAAAAGTATTTAAAAGTATTTCCACTACAACTTAAGAAACT |
| | | | GTCATCCAGTGCAAAGCYCAGGGTAGACAGCAGAGAGTTGGATTTAG |
| | | | CCATGATTGATTGGAGTTTTTCCAGGAAAATACGATGAAGGAAGACA |
| | | | AGAACAAATGACAGACCATGGAATTGAGGCTCGATAATGAGAGAAGT |
| | | | AAAGACATAAAGTGGAGAGGAACCGTGAAAAGATGCTAGGAATAATG |
| | | | AGAAAGGGCAAGCTGAAAAGTCATAGAGTAGGAGTCAT |
| IFNG | rs7302226 | 61 | GGGAAGTCAATGAGGTTAGAACTCCTGCAATGCATGAGACAGTTTTG |
| | (POSITION | | TGCAATGAAGAATTGCCCCATGTCTCATGCAATTTACACACATAATT |
| | 301) | | TATCTTAATTTATACAGTGGCCCTGTAGGTAGTGTATTTATCTCCAT |
| | | | CTGGCAGATTATAATGGAGGTTAATGGGGAGCCTTCATCTTCCCTAC |
| | | | CTGCTTGAAAATCTCTATCCCTAGAACTAATCATTTTGGTTCAACGT |
| | | | ATGCAGACAATATTCCTCCCTCAATTTTTCTAGATTGTTCACATCTC |
| | | | CATGGGGCATATGCAGGGRCCTCTAGAGACCCCACTACAACATCTAA |
| | | | GATAATTCTCCACCTAAAGTAGTGAAAAATCATGTTGGACACCAGAA |
| | | | AGCTCTTAGCAAGGCTCAATAATTAATTACTGATGTTATTTTCACAT |
| | | | GGAAAGAAATATTCTTGGTAAATCAGAATAAATTTCTTGAAACTTCA |
| | | | TGTAAAATTCATAATTGTGTTAAGGTAATTTTGAGCCACTGTCTGTG |
| | | | TATGCCGTTCTGTGGGATACACAGAGTATACCTTTGTGAGGCTCCAG |
| | | | GGACATTCTTTCCACTTCGTACTTCTTTCTAAATCAC |
| IFNG | rs7302488 | 62 | ATACCATTCTGGGCCAAGGCCAAAGAAAGCCCCTGAGAATCCTTCCA |
| | (POSITION | | GCTCTCTCTTCCCTTGCTGCAGTAATGATAAGGGTCACATGTTTTGA |
| | 294) | | GGACACGAAACATGGCAGATAGAATACATGCTACCTCTACATTCTTT |
| | | | CAGAATCCGTAAGACAAAAATAACAACATAAAAGGCTATAAAGCCTC |
| | | | AACAACAAAAAAAGCCAAAAGCAAATGAGAAATGTCAATGAAGTTAT |
| | | | GGAAGATGGAAAGAAGATGAGCAAGTGGTGAGTAACTTCAACTTTAG |
| | | | ATTTCTCCACTKCGGCAAGTACCAAGTAGAGGAAATTTAGTTCACAC |
| | | | TGCAGATTAGTAGAAAACTCAGGAATTGTGTTATTAATCACTTCTGA |
| | | | AGAAGGAAGTTCAGGGTGGGATTGAAAATAAGACAATTGGTTGAAAA |
| | | | ATGTATATAAGATGTAGTTAGATCCCTCGTATCCCACTTAGCCACAC |
| | | | CACTGCCCCCGTATACCTGTTTGAAGACTGGAAGTTTACCTTCCAGC |
| | | | AAGGTTCTGGATATCTTCTGGATATTTAGCATAGCTGAGAAGGAAGT |
| | | | AAGTACCTTCATAAGGTTTGGATTTATTTGAAAGTCATCATACTGAG |
| | | | CAGTGAGAACACGAGGCTTCCAGAATGCTTACTATCAGGCTTATATT |
| | | | CCTAGTGTCTAGGAAATTGCCAAGCCTAAAAGAAAAGATGTACTAAT |
| | | | GTGGGGTTCCTGCCAGTGAAACTCACCAGTTTCAAGTATCACCCTAC |
| | | | TAAGAGGCTTGTAAGTCAGGAAGTCCAATCAATATATTTAATGTACC |
| | | | CAATCCAACAATTTGGACTTTGTTATAAAACATAAACAACATTAATG |
| | | | AACAGAAAAATACTTGAAAAAATACTTTAGGATAAAATACAAAGTCT |
| | | | AAAAAACAAACAGAAAAAATAAATAAAAGAAATGAAGTTTAATGCCG |
| | | | AGAAAAATAACAAAGAGAAAAAAATTTAAAAAGT |
| IFNG | rs741344 | 63 | GCTTGTAGGCTGGCTGGCCAGGGGAAACTACCAGTCCGCTTTGTGCA |
| | (POSITION | | AGTGAATTCTCAAACCCTATCTGAGCACAGGAATCACCTGGGCGTCA |
| | 154) | | AACAGGAGAAAGTTAATATCCTACTCTATTCTCCCACAAATTTCTAT |
| | | | AGGACTAATAAARGAAAAGAAAGGAAAGAAAATGTCAAAATGCCTAA |
| | | | TTTATCTACTTAGTTTTTACTCATAAAACTTTTAGCACTGGAATAGA |
| | | | CCAAGGAGATTGAATAAGCCGATTGTTTGCACTTTGCAGAAAGGGAG |
| | | | ACCAAGGCCCAGGTAGTTAAGTCACTCACCTAACATCCCACAGGGAG |
| | | | TCCTATGCTCATGACAAAATAGTGTCACTATCTAACAGTTAAAGATA |
| | | | AGAGTTAAAACTCGTGAAACGGAAGTGGGTAAATGATAACATTTAGT |
| | | | CTCTAAATGTCCTCTCGACAAAAGAATGTCATATCAATAAAGATAAC |
| | | | ACTTAGTTCAAACACTTGAAATGAAAGTGGCTAAATGATAACATCTA |
| | | | TCAAAATGCTGAGGTCAACCAACAGGTCTCTTCAGGGGTGTTCATGG |
| | | | TGGTGACGGTTTTCTGGCTCTGCCCAATTGGGATGCTACCTTCAGAT |
| | | | CAGACCCTGCATAGAAGGAAGAGACTCTTCCTGAGAAAGGGGCTTCA |
| | | | TGATTAGGCACAGCAGACTGCTGTGATCAAGG |
| IFNG | rs759487 | 64 | CAACAGGGACATTCAGAAGCACTCTTGAAATGACAAAACCCTCAGTG |
| | (POSITTON | | GAATTTATAGCATAACTTTCTTTTTAATATTCCATTTCTGGGTCAAT |
| | 201) | | CCTTGATCCTGTTAACACTTTAATAAGCAGCAACAAAGAGAGGCATC |
| | | | ACTGCAGAGACTGTGCAGATACTAAATCAGCTTATGTACATTCTGTC |
| | | | TAGGAAGACTTTYGGAACAGAGGCTGGCTTGCTGTTCATCAAATTTA |
| | | | TTTGTGAGTTCTAGCCCATGGAATGTGAGTGGAAGTGATGTGTGCCA |
| | | | TCCTCCATGGGCCATCTTCCATGTTTCTTCTCTGTTGACCAACTTGA |
| | | | TACGAACATGCACAGTGACCTCACAGTCCAAGTATTGAAGATGGTGA |
| | | | AGATGGTGGAGCCACAGGATGAAAGGATCCTGGTCCTGCTTAGAGAA |
| | | | GAGATAGCTCCTGTTCTGTTACATCTATTTTTGACTTTACATGAGCA |
| | | | AAAAGTAAACTTCTATTAGGTTTAAGCCATTTTACATTTTAATATAG |
| | | | CTACTGAAACCTCGCATCTTGACTACAGCTTTTATGTAAATAAGAAA |
| | | | TATGGCCTGTAATCCCAGCTGTTTGGGAGGCTGAGGCAGGAGGATCA |
| | | | CTTGAGGCCAGGAGTTAAAGGCTGCAGTGTACTATGGTCAGACCACT |
| | | | GCACTCCAGCTTGGATGACAGAGACCTTGTCTTTAAAAGAAAAAGAA |
| | | | AAATGTATATTTCATATTTTAAAATAAATTTTTGGCTGGGCACAGTG |
| | | | GCTCATGCCTGTAATCCCAGTGCCTCAGAAGGCCGAGGCAGGAAGAT |
| | | | CTTTTGAAGCCTGGAATTCAAAACCAACCTAGGCAACATATTGAGAC |
| | | | CTTGTATCAAAAAAATATTTTTTTTAATTAGCTGGTCATGGTGTGTT |
| | | | GTGCCTGTAGTCCCAACTACTCAAGAGACCAAGGTGGGAGGATCGCT |
| | | | TGAGCCCAAAAATTCAAGGCTGCACTGAGCTGTGATCACGTCATTGT |
| | | | GCTCCAGCCTGGGCAACAGCCTAAGCAACTCTGTCTCTAAAATAT |
| IFNG | rs759488 | 65 | GCTCTCGAGGAGCCTTTGATTTGGTGGGAGCATCAGACAAGGGAGTC |
| | (POSITION | | AAAGGTTTCAATACAGTGTGACAAGTGGCATTCTACAAGTATTAACA |
| | 201) | | GGTATCATGACAGCAAGAAGAATTCAGAGAAGGAATCTCATTTGACT |
| | | | AGGGATGGGAGTGAGAATATGAGAGGTGGCAAAAATGAACAGATGGG |
| | | | TAGGGTCACAGGYAATATGCACAAGACCTCTCTTCTCATGAAGCTTA |
| | | | CATTTTAGTAGAGTCAAAGAAAGGAAGATAATAAACAAGGCAATCAA |
| | | | CAAAGAAACAAGATAATTTCAAAGCATGAGGATAATATGAAGGAAAT |
| | | | AACAAAGGTGATTTGGAATTACTAGGAGTGGATGGAGATCCTTCCTC |
| | | | AGCTGGGTTGGGAACGTCATGTCAAAGGAAGAGACCCTTGAGCTGAC |
| | | | ACGTAAATGAAAGGAACGGACTGTGGGAAGGCCTGGGGAACGGTACT |
| | | | CCAGGGAGAGGAGCTAGCATCTACAAATGCCCAAGACAGAGCTGAAC |
| | | | TTGCACTTTTCAGAAGCAGAAAGGTCAGCTAAGAGACAACACAGGCC |
| | | | AGGAGACAAGGTCAGAGAGAAAGGCTAGGCAATTAATGTAGGTCTTT |
| | | | CTTGGCCAGATAATAAGGTTTATTCTCAGTCCAAGGGAAGCCATTGA |
| | | | AAGGCATCAAACAGGAAGGGATATGCTTTGATTTACACTTCTTAAGT |
| | | | TCTCTCTAGAAGCTCAATGAAGCTGGATTCAGGGGCAAGGTATGAGT |
| | | | GGAAACAATGAGACCAGTTAGAAGGAGGACTCTTCCAGTGTCCAGGT |
| | | | GAGACATGGCAGTGACCTGGGCCAGGGTATACTAATGGGGATAGGAG |
| | | | AAGCGGAAGGATTTGAGATATATTGGGGCGGTAGAACTGCAAGAATG |
| | | | TGCTGATGAATTTGGTTTGGGATATGAGGGAAAAGAAGAAATAAAAA |
| | | | ATCCCTGTAATTGCAAAAATGGCCCTAGCAATTGAGTAGGTGACAAT |
| | | | TTATCATATAATAATAACAACTTATGCGTATAAAGTTTTTATTATAT |
| | | | AGCAGTCATGGCTCTAACCTCTTTACATATATTACCTCACATGAACC |
| | | | CCACAACAACCCTACAAGATAGGTACTATTCTCATCCCTATTGTACA |
| | | | GACAAGGGAAGAGAGGGACGGACAGATTAACCTCACTTTGTTGTTAA |
| | | | ATTACAGCCTCTATGTGAAGCTTTATCGGCTTCAGAGTCTGTGTGCT |
| | | | TAACCATGATATCTTTACGTTTTGTATTACCAGGTTGTGGAATACTA |
| | | | GAGAATGAACTGATTTTAGAAGGAGAAACAAATTTTCCGGTTTTGAC |
| | | | ATATTGTTTTTGAGATGTCTTACATGGAAATATCGAGTACATAATTG |
| | | | AATGTGTGAGCATGGAATTCAGGGACTAGGTCAACCCTGGAGACATT |
| | | | AGCACACTGATAGTATTTAAAGCCATGGGGTTGAATTAGCTGTATAG |
| | | | AGAGCAATAGAGTACATGGAGATTACAAGAAGCCACAACTAGCCCTG |
| | | | AGTCCTCCAATCTGTAGTGTTCTGATAGAGAAGAAACTCACTTGCAA |
| | | | GATCAAGAAGCAGCATCTAAGTGAGGCAGAAAGAATCCCAGAGGAGA |
| | | | GTGTGGATTTTCAGAACTGAGTGATTAACATGTTGGCTTGATTCTCA |
| | | | GCCAGTCTCTGTCCTCATGGTGGCAAGATGGCTGCAGCAATTCCAAC |
| | | | CAATACTCTTCCAAGCTTATAGTTCATAGAAAAGAGAAAGACTCATT |
| | | | TTCCAGAACTCATTTATAAATCCTGGAATCCACTCTGATTGGGCCTT |
| | | | GTTGGGTCATAGGCCCATTCCTGAATCTTCACCAATCATTGTGACTA |
| | | | GAGGACCCTA |
| IFNG | rs7956817 | 66 | CAACTAACATGCCAAAACTCAAAGAGTTGAAAAGCACTCCTGAAGGT |
| | (POSITION | | AAATATACCCTTCTATAACCGTTATCAAATAAGACATAATTGTCTAT |
| | 201) | | ATATTTGTCCATCTTATCCTTCCAACTTCATTTCACACTCCAGTTTT |
| | | | ATTTGTTTGTCGAACACTAATTGTCTTTTTTTTCTCATCAGCCCTAA |
| | | | CATATTGTAAAGWTCCATTTGTAACTACTTTAATATCCACATTATCA |
| | | | TGCATCTTTCAGTAAAGTAAAAAATTGTCCAAGTTTCTCCATTCTCA |
| | | | GAGTTTTGTTTTTTGGTTTTTTTTTTTTTTGTTTGTTTGTTTTTGAG |
| | | | ACGGAGTCTCACTCTGTCGCCCAGGCTGGAGTGCAGTGCCGCGATCT |
| | | | CGGCTCACTGCAAGCTCCGCCTCCCGGGTTCATGCCATTTTCCTGCC |
| | | | TCAGCCTCCCGAGTAGCTGGGACTACAGGCGCCCGCCACCGCGCCCG |
| | | | GCTAATTTTTTGTATTTTTAGTGGAGACGGGGTTTCACCGTGTTAGC |
| | | | CGGGATGGTCTCGATCTCCTGACCTCGTGATCCGCCCACCTTGGCCT |
| | | | CCCAAAGTGCTGGGATTACAGGCGTGAGCCACCGCACCCGGCCCATT |
| | | | CTCACAGTTTTACTACTTCTGTATGCTGACAGCCTGTCCATCTCTAC |
| | | | CTCTAGGACAGACCTCTCTCCAGAACCTCTGATCCACCCAGCCCACT |
| | | | GCGGTGTAGACGGCCTAGA |
| IFNG | rs7959933 | 67 | GCTGGACAGGATGGACACCCTCTCCAAGACCCTGGGGGAGCAGGACA |
| | (POSITION | | AAGCCAGTGCTCCCCAGAGGTGGTCACTCCCAGGAGGAAAAGCAGAG |
| | 201) | | AGATGTGGAAGGGGCTGGGTACATGTGCCCTGTTTGTCCTCCCAAAC |
| | | | ACAGCAGGCAGAAGAGTCACTCCACCCAGGGCAAAGTGAAGGAGAGG |
| | | | GTGGAGGGAGATYGGGAATGCTGTGCTCATAGATCTCTCTTGACAAG |
| | | | AATGGGGAGAAAAGTTCCACACCAAAGGAGGGCAAAGCCAGAGAAAT |
| | | | AGGGAAGAGGTCTCGGGATCTGCACAGTGAGTTTGTGGAGCGTAAAC |
| | | | TCCACGTCAGTTTATGTGGCTACACATAAAGATAACTCCAATAAACC |
| | | | ACCTTCAGGGAGCCTGCTCGAAGTA |
| IFNG | rs7969024 | 68 | TATTTTTCCAAACTAATAATGGAAGTGGTATTAGGGTAATATATTTA |
| | (POSITION | | TAGGTGAGATTCCAGGGCTGATTTAGTAAATATTAATTTCTAATACT |
| | 527) | | TTGTCATTCCCACTGCATTATTCTCCTATAGCTGTCACAACAAATCA |
| | | | CCATAAACCGGGCAGCTTAAAACAACAGAAATTTGTTCTCTCTCAGT |
| | | | TCTGGAGGCTAGAAGCCTGAAACCAAGGTGTCGGTAGCACCATGCTC |
| | | | CCATGCTTCCTTCTAGGGAAGAATGCTTCCTTACCAGTTCTGGCTTC |
| | | | TGCCTATTCTTGGCACTCCTTGGCTTGTGGCAGCACAACTCCACTCT |
| | | | CTGCTTCCATCTTCACATGCCCAACTTCCTTCCATTTATGTGTATCT |
| | | | GTGCCAAATTTCCCTCTTCTTATAAGGACATCTGTCATTGGATTAGG |
| | | | GTTTACCCTAATGAATTTGGGGAGGACCCTATTCAATCCACTACAAC |
| | | | CACCCTTTATGTACACGTAGCTGGTTTCTCTGTCAATTATATTTTAG |
| | | | AGTGAGGACKTTGCTTCTCCTCTAACAAGATATTATAATAACAATTA |
| | | | TTGTCAAATTATTTAATGAATGCTTACTATATGACAGTTACATGCAT |
| | | | TAACTCATTTAACCCTCTGACAATTCTATGAAATAGGTGCTATTTTT |
| | | | ATTTCTATTTTGCAGATGAGCAGCCAGAGAGAGTTTACATAGGGCAA |
| | | | ATATCACCATTACCTAGCAAGAACAAAATAAGAGGAATAAGCAGTCC |
| | | | CCTTGTATTTTGGTTACTTAAAAGGGATGGATCTCAAGACAAAGGAA |
| | | | AATGGTTGGGTGCACGAGGGGCCAGATGCTGGAACCAGTTCTGAAGA |
| | | | AGTGTT |
| IFNG | rs7969592 | 69 | GCTGAGGTGGGAGGAATATGAAGGCCCAGGAGTTCAAATCCAGCCTG |
| | (POSITION | | GGCAACACAATGAGACCCTGTCTTAAAAAAAAAAAAAATCAGCAAGC |
| | 301) | | TGGGAAATAAACTTGGGGCACACTGGGCACTTCGTCATGAGGAAACC |
| | | | AAAATCTCCTGCCTTGGCAAGCTTCAGGAGCCATATAAGGACTGAGC |
| | | | CAGCCTCACCCATTACACTGTGTAGGGACACTCTTCAGCAACGACAT |
| | | | CATGTGGCAGAAGAAAACATGGCCATAGGGGATTCCTTCATTGTGCA |
| | | | ATTACCTATAAGAAGAAGRAAAGGAAGAAAAGAGGAAGAAGAACGAG |
| | | | GAGGAGGAGGAGGTCTAAAAAGGAAATGCTTAAATTCTTGCTGAAAG |
| | | | GTGAGTGAATTTTGGAGTTCAATGTAACAACCAATAAATAACATCTC |
| | | | TCTTCTCTTCTTGGTTCTGTGCCCATTGAAAAATACGACAAAGAGTG |
| | | | AAACAAATGGAAAAGCAAAGTATTATCCTCTTTCTGATAAAGCAAAT |
| | | | AACAGAGAATGTAGCTCTAATTTGTGGGCAAATGGGGGTCTTAAAAC |
| | | | TGAACCTCAGAATTTAATATTTAACCGACTTCTGGTG |
| IFNG | rs7973244 | 70 | CACATTTTCAGATTAAATGGACAAACGCTTGACTTCTATTTCATATA |
| | (POSITION | | TATCCATATACAAAAAAAATCAGAAAGTGGTATAGAAATTGTATTTA |
| | 357) | | CTGAACATTAAGCACAACCCATTTATTTCTATTTAAATAGCACCAAA |
| | | | ACCTCAGTAACATTTAACAGGTTAACAATATAGACTTGAGTCATATT |
| | | | GAGTCTGACATTGAGTCAGACCTAGATTTATATCATGCTCTGCCACA |
| | | | GATACTCTGGTATCTTTAAGCTAATTACATATCCCCAAGCCTCAGCT |
| | | | GTCCCCAACTGCAAGATGGCCATGATGACAGATGAGAACAGATAACT |
| | | | CAGAGTGTGGCTATGAGAACTAAATGAWTTAACGCCTGTAAAACATT |
| | | | TAGAAAAATGCCTAGCATGTGGTAAGTGCTCATTAAACATAGCTATA |
| | | | TTTAAATATTTCTAAAATATTGCCAAATCCAGATGCTAATGACTAGG |
| | | | GCATCCTAAAAGACAGATTTAGAAAGGAAATTGCTGTCTATATTCTG |
| | | | AACAGTACAGTAACTGTGTTTTGACTTTGTCATTTGCCACTTCCATC |
| | | | CAGTGCTTTTCTGGTAGCATGCTGGAAAATGAACCACAGCACACTAA |
| | | | CA |

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the SNP of interest. For Example, the sequences identified in TABLES 1D and 1E.

A “genotype” is defined as the genetic constitution of an organism, usually in respect to one gene or a few genes or a region of a gene relevant to a particular context (i.e. the genetic loci responsible for a particular phenotype).

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins” or “I”) of a nucleotide relative to a reference allele. Furthermore, a person of skill in the art would appreciate that an insertion or deletion within a given sequence could alter the relative position and therefore the position number of another polymorphism within the sequence. Furthermore, although an insertion or deletion may by some definitions not qualify as a SNP as it may involve the deletion of or insertion of more than a single nucleotide at a given position, as used herein such polymorphisms are also called SNPs as they generally result from an insertion or deletion at a single site within a given sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature>38° C. or <36° C., B) heart rate>90 beats per minute, C) respiratory rate>20 breaths per minute, and D) white blood cell count>12,000 per mm3 or <4,000 mm3. In the following description, the presence of two, three, or four of the “SIRS” criteria were scored each day over the 28 day observation period.

“Sepsis” is defined as the presence of at least two “SIRS” criteria and known or suspected source of infection. Severe sepsis is defined as the presence of at least two “SIRS” criteria, a known or suspected source of infection and at least one new organ dysfunction. Septic shock was defined as sepsis plus one new organ failure by Brussels criteria plus need for vasopressor medication.

Subject outcome or prognosis as used herein refers the ability of a subject to recover from an inflammatory condition and may be used to determine the efficacy of a treatment regimen, for example the administration of activated protein C or protein C like compound. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome, prognosis, or response of a subject to activated protein C or protein C like compound or protein C like compound administration may be accomplished by various methods. For Example, an “APACHE II” score is defined as Acute Physiology and Chronic Health Evaluation and herein was calculated on a daily basis from raw clinical and laboratory variables. Vincent et al. (Vincent J L. Ferreira F. Moreno R. Scoring systems for assessing organ dysfunction and survival. Critical Care Clinics. 16:353-366, 2000) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.” Furthermore, the criteria or indication for administering activated vasopressin (XIGRIS™—drotrecogin alfa (activated)) in the United States is an APACHE II score of ≧25. In Europe, the criteria or indication for administering activated protein C or protein C like compound is an APACHE II score of ≧25 or 2 new organ system failures.

“Activated protein C” as used herein includes Drotrecogin alfa (activated) which is sold as XIGRIS™ by Eli Lilly and Company. Drotrecogin alfa (activated) is a serine protease glycoprotein of approximately 55 kilodalton molecular weight and having the same amino acid sequence as human plasma-derived Activated Protein C. The protein consists of a heavy chain and a light chain linked by a disulfide bond. XIGRIS™, Drotecogin alfa (activated) is currently indicated for the reduction of mortality in adult subjects with severe sepsis (sepsis associated with acute organ dysfunction) who have a high risk of death (e.g., as determined by an APACHE II score of greater>25 or having 2 or more organ system failures).

XIGRIS™ is available in 5 mg and 20 mg single-use vials containing sterile, preservative-free, lyophilized drug. The vials contain 5.3 mg and 20.8 mg of drotrecogin alfa (activated), respectively. The 5 and 20 mg vials of XIGRIS™ also contain 40.3 and 158.1 mg of sodium chloride, 10.9 and 42.9 mg of sodium citrate, and 31.8 and 124.9 mg of sucrose, respectively. XIGRIS™ is recommended for intravenous administration at an infusion rate of 24 mcg/kg/hr for a total duration of infusion of 96 hours. Dose adjustment based on clinical or laboratory parameters is not recommended. If the infusion is interrupted, it is recommended that when restarted the infusion rate should be 24 mcg/kg/hr. Dose escalation or bolus doses of drotrecogin alfa are not recommended. XIGRIS™ may be reconstituted with Sterile Water for Injection and further diluted with sterile normal saline injection. These solutions must be handled so as to minimize agitation of the solution (Product information. XIGRIS™, Drotecogin alfa (activated), Eli Lilly and Company, November 2001).

Drotrecogin alfa (activated) is a recombinant form of human Activated Protein C, which may be produced using a human cell line expressing the complementary DNA for the inactive human Protein C zymogen, whereby the cells secrete protein into the fermentation medium. The protein may be enzymatically activated by cleavage with thrombin and subsequently purified. Methods, DNA compounds and vectors for producing recombinant activated human protein C are described in U.S. Pat. Nos. 4,775,624; 4,992,373; 5,196,322; 5,270,040; 5,270,178; 5,550,036; 5,618,714.

Treatment of sepsis using activated protein C or protein C like compound in combination with a bactericidal and endotoxin neutralizing agent is described in U.S. Pat. No. 6,436,397; methods for processing protein C is described in U.S. Pat. No. 6,162,629; protein C derivatives are described in U.S. Pat. Nos. 5,453,373 and 6,630,138; glycosylation mutants are described in U.S. Pat. No. 5,460,953; and Protein C formulations are described in U.S. Pat. Nos. 6,630,137, 6,436,397, 6,395,270 and 6,159,468.

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO2/FiO2 ratio is less than 300, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28 day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine, phenylephrine). Days alive and free of an International Normalized Ratio (INR)>1.5 are days that a person is alive and does not have an INR>1.5.

TABLE 2A
Brussels Organ Dysfunction Scoring System

| | Free of | | Clinically Significant | | |
|---|---|---|---|---|---|
| | Organ Dysfunction | | Organ Dysfunction | | |
| ORGANS | Normal | Mild | Moderate | Severe | Extreme |
| DAF ORGAN | 1 | | 0 | | |
| DYSFUNCTION | | | | | |
| SCORE | | | | | |
| Cardiovascular | >90 | ≦90 | ≦90 | ≦90 plus | ≦90 plus |
| Systolic BP (mmHg) | | Responsive to | Unresponsive | pH ≦ 7.3 | pH ≦ 7.2 |
| | | fluid | to fluid | | |
| Pulmonary | >400 | 400-301 | 300-201 | 200-101 | ≦100 |
| PₐO₂/F_{I}O₂ (mmHg) | | | Acute lung | ARDS | Severe ARDS |
| | | | injury | | |
| Renal | <1.5 | 1.5-1.9 | 2.0-3.4 | 3.5-4.9 | ≧5.0 |
| Creatinine (mg/Dl) | | | | | |
| Hepatic | <1.2 | 1.2-1.9 | 2.0-5.9 | 6.0-11.9 | ≧12 |
| Bilirubin (mg/dL) | | | | | |
| Hematologic | >120 | 120-81 | 80-51 | 50-21 | ≦20 |
| Platelets (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, Mar. 12-14, 1994.

Analysis of variance (ANOVA) is a standard statistical approach to test for statistically significant differences between sets of measurements.

The Fisher exact test is a standard statistical approach to test for statistically significant differences between rates and proportions of characteristics measured in different groups.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition or is to be administered activated protein C, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material. Particularly, genetic material, containing the sequence or sequences of interest. Many methods are known in the art for collecting bodily samples and extracting genetic material from those samples. Genetic material can be extracted from blood, tissue and hair and other samples. There are many known methods for the separate isolation of DNA and RNA from biological material. Typically, DNA may be isolated from a biological sample when first the sample is lysed and then the DNA is isolated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR), Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of a mutation in a IFNG gene sequence, as described in TABLES 1B-E. The sequence of interest may also include other mutations, or may also contain some of the sequence surrounding the mutation of interest.

Detection or determination of a nucleotide identity, or the presence of one or more single nucleotide polymorphism(s) (SNP typing), may be accomplished by any one of a number methods or assays known in the art. Many DNA typing methodologies are useful detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

Alternatively, an invasive cleavage method requires an oligonucleotide called an Invader™ probe and sequence-specific probes to anneal to the target DNA with an overlap of one nucleotide. When the sequence-specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP system.

Mutation detection methods may include but are not limited to the following: Restriction Fragment Length Polymorphism (RFLP) strategy—An RFLP gel-based analysis can be used to indicate the presence or absence of a specific mutation at polymorphic sites within a gene. Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. Finally, if both the higher molecular weight band and the two lower molecular weight bands are visible then the DNA sample contained both polymorphisms, and therefore the DNA sample, and by extension the subject providing the DNA sample, was heterozygous for this polymorphism;

Sequencing—For example the Maxam-Gilbert technique for sequencing (MAXAM A M. and GILBERT W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et al. (Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Qβ replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4): 1760-1764). Other methods include those of Donis-Keller et al. (1977, Nucl. Acids Res. 4:2527-2538), SIMONCSITS A. et al. (Nature (1977) 269(5631):833-836), AXELROD V D. et al. (Nucl. Acids Res. (1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-terminator incorporation with fluorescent polarization-detection (TDI-FP) method is described by FREEMAN B D. et al. (J Mol Diagnostics (2002) 4(4):209-215) for large scale screening;

Oligonucleotide ligation assay (OLA) is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2):123-9; IANNONE M A. et al. Cytometry (2000) 39(2):131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8.);

Polymerase proofreading methods are used to determine SNPs identities, as described in WO 0181631;

Detection of single base pair DNA mutations by enzyme-amplified electronic transduction is described in PATOLSKY F. et al. Nat Biotech. (2001) 19(3):253-257;

Gene chip technologies are also known for single nucleotide polymorphism discrimination whereby numerous polymorphisms may be tested for simultaneously on a single array (EP 1120646 and GILLES P N. et al. Nat. Biotechnology (1999) 17(4):365-70);

Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43:1151-1158; LITTLE D P. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites.

Once the identity of a polymorphism(s) is determined or detected an indication may be obtained as to subject response to activated protein C or protein C like compound or protein C like compound administration based on the genotype (the nucleotide at the position) of the polymorphism of interest. As described herein, polymorphisms in IFNG gene sequences, may be used to predict a subject's response to activated protein C or protein C like compound treatment. Methods for predicting a subject's response to activated protein C or protein C like compound treatment may be useful in making decisions regarding the administration of activated protein C.

Methods of treatment of an inflammatory condition in a subject having an improved response polymorphism in a IFNG gene sequence are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more polymorphic sites in a IFNG gene sequence. Also, as previously described the sequence identity of one or more polymorphisms in a IFNG gene sequence of one or more subjects may then be detected or determined. Furthermore, subject response to administration of activated protein C or protein C like compound may be assessed as described above. For example, the APACHE II scoring system or the Brussels score may be used to assess a subject's response to treatment by comparing subject scores before and after treatment. Once subject response has been assessed, subject response may be correlated with the sequence identity of one or more polymorphism(s). The correlation of subject response may further include statistical analysis of subject outcome scores and polymorphism(s) for a number of subjects.

Methods of treatment of an inflammatory condition in a subject having one or more of the risk genotypes in IFNG associated with improved response to a therapeutic agent are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more single nucleotide polymorphic sites in IFNG sequences. Also, as previously described the sequence identity of one or more single nucleotide polymorphisms in the IFNG sequence of one or more subjects may then be detected or determined. Furthermore, subject outcome or prognosis may be assessed as described above, for example the APACHE II scoring system or the Brussels score may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis.

Cohorts

We prospectively studied a cohort of 1072 Caucasian patients having systematic inflammatory response syndrome (SIRS) who were admitted to the Intensive Care Unit (ICU) of St. Paul's Hospital. We analyzed the Caucasian subset because of the risks of population stratification of a mixed cohort. We also studied a cohort of severe sepsis patients who had received Activated Protein C (XIGRIS™) treatment (N=33) and untreated matched controls (N=199). This cohort, which includes all ethnicities due to its small sample size, is referred to as the Activated Protein C cohort. We also studied an independent Caucasian cohort (N=202) of patients scheduled for first time elective coronary artery bypass grafting that required cardiopulmonary bypass. We refer to this independent non-septic SIRS cohort as the Sirius Biological Plausibility cohort. Significant SNP-biomarker associations identified using this group of patients may provide useful insights into the cellular processes underlying the population-based SNIP-phenotype associations localized in the Caucasian SIRS cohort. The Institutional Review Board at Providence Health Care and the University of British Columbia approved this study.

Study Inclusion Criteria

All patients admitted to the ICU of St. Paul's Hospital were screened for inclusion. The ICU is a mixed medical-surgical ICU in a tertiary care, university-affiliated teaching hospital. Patients were included in the SIRS cohort if they met at least two out of four SIRS criteria: 1) fever (>38° C.) or hypothermia (<36° C.), 2) tachycardia (>90 beats/minute), 3) tachypnea (>20 breaths/minute), PaCO2<32 mm Hg, or need for mechanical ventilation, and 4) leukocytosis (total leukocyte count>12,000 mm3) or leukopenia (<4,000 mm3). Patients were included in the SIRS cohort on the calendar day on which the SIRS criteria were met. Patients were excluded if blood could not be obtained for genotype analysis.

For the Activated Protein C cohort, we identified XIGRIS™-treated subjects who were critically ill patients who had severe sepsis, no XIGRIS™ contraindications (e.g. platelet count>30,000, International normalization ration (INR)<3.0) and were treated with XIGRIS™. The control group for the Activated Protein C cohort were critically ill patients who had severe sepsis (at least 2 of 4 SIRS criteria, known or suspected infection, and APACHE II≧25), a platelet count>30,000, NR<3.0, bilirubin<20 mmol/L and were not treated with XIGRIS™. Accordingly, the control group (untreated with XIGRIS™) is comparable to the XIGRIS™-treated group.

In the Biological Plausibility cohort of non-septic SIRS patients, individuals were included in the analysis if they had undergone cardiopulmonary bypass surgery. Patients were not included in the study if they had undergone 1) urgent or emergency cardiopulmonary bypass surgery (inflammatory response to other triggers, i.e., shock) or 2) valve or repeat cardiac surgery. The first subgroup of patients may have had an inflammatory response due to other triggers (i.e., shock), while the second subgroup may have had different pre-operative pathophysiology or longer total surgical and cardiopulmonary bypass time.

Clinical Phenotype

Our primary outcome variable was 28-day mortality. Secondary outcome variables were organ dysfunctions (TABLE 2C). Baseline demographics recorded were age, gender, the admission APACHE II score (KNAUS W A. et al. Crit Care Med (1985) 13:818-829), and medical or surgical diagnosis on admission to the ICU (based on the APACHE III diagnostic codes (KNAUS W A. et al. Chest (1991) 100:1619-1636) (TABLE 2B). After meeting the inclusion criteria, data were recorded for each 24-hour period (8 am to 8 am) for 28-days after ICU admission or until hospital discharge to evaluate organ dysfunction and the intensity of SIRS and sepsis. Raw clinical and laboratory variables were recorded using the worst or most abnormal variable for each 24-hour period with the exception of Glasgow Coma Score, for which the best possible score for each 24-hour period was recorded. Missing data on the date of admission was assigned a normal value and missing data after day one was substituted by carrying forward the previous day's value. When data collection for each patient was complete, all patient identifiers were removed from all records and the patient file was assigned a unique random number linked with the blood samples. The completed raw data file was used to calculate descriptive and severity of illness scores using standard definitions as described below. A Biological Plausibility key is also found in TABLE 2D.

TABLE 2B
Baseline characteristics key.
Baseline Key

| | |
|---|---|
| AGE | Given In Years |
| SEX | Percentage of Male Subjects |
| APACHEII | APACHE II score |
| SURGICAL | The percentage of subjects who had a surgical ICU |
| | admitting diagnosis |
| SEVSEP.ADMIT | Severe sepsis upon admission |
| SS.ADMIT | Septic shock upon admission |

Note.
Data reported as 25%-ile/median/75%-ile.

TABLE 2C
Primary and secondary outcome variables key.
Days alive and free (DAF) of organ dysfunction Key

| | |
|---|---|
| SURV | 28-Day Survival |
| DA | Number of days alive out of the 28- |
| | day period |
| ***.DAF | Days Alive and Free of *** |
| ALI.DAF | Acute Lung Injury |
| PRESS.DAF | Any vasopressors |
| PRESS2.DAF | More than 2 ug/min of vasopressors |
| PRESS5.DAF | More than 5 ug/min of vasopressors |
| PRESS15.DAF | More than 15 ug/min of vasopressors |
| INO.DAF | Inotropes |
| MSIRS.DAF | 2 of 4 SIRS criteria |
| MSIRS3.DAF | 3 of 4 SIRS criteria |
| MSIRS4.DAF | 4 of 4 SIRS criteria |
| CVS.DAF | Cardiovascular dysfunction |
| RESP.DAF | Respiratory dysfunction |
| PFRATIO.DAF | PaO2/FiO2 less than 300 |
| CNS.DAF | Neurological Dysfunction |
| COAG.DAF | Coagulation Dysfunction |
| INR.DAF | International normalized ratio >1.5 |
| RENAL.DAF | Acute renal failure |
| ANYREN.DAF | Any type of renal dysfunction |
| RENSUP.DAF | renal support |
| LIVER.DAF | Acute hepatic dysfunction |
| ANYLIVER.DAF | Any type of hepatic dysfunction |

Note.
Data reported as 25%-ile/median/75%-ile

TABLE 2D
Biological Plausibility Key.
Biological Plausibility Key

| H.TENSE | Hypertensive |
|---|---|
| EJEC.FRAC | Ejection Fraction |
| BYPASS | Bypass Time |
| CLAMP | Clamp Time |
| APROTININ | Aprotinin Use |
| GCSF | Granulocyte Colony Stimulating Factor |
| IL10 | Interleukin 10 |
| IL1ra | Interleukin receptor 1a |
| IL6 | Interleukin 6 |
| IL8 | Interleukin 8 |
| MCP | Monocyte Chemoattractant Protein |
| med | Median |
| SD | Standard Deviation |
| F | F Statistic |
| d.f. | Degrees of Freedom |
| ***.diff | Difference between 3 hours postoperatively and |
| | preoperative *** |
| ***.0 | *** levels preoperatively |
| ***.3 | *** levels 3 hours postoperatively |

Note.
Data reported as 25%-ile/median/75%-ile

Organ dysfunction was evaluated at baseline and daily using the Brussels score (SIBBALD W J. and VINCENT J L. Chest (1995) 107(2):522-7) (TABLE 2A). If the Brussels score was moderate, severe, or extreme dysfunction then organ dysfunction was recorded as present on that day. To correct for deaths during the observation period, we calculated the days alive and free of organ dysfunction (RUSSELL J A. et al. Crit Care Med (2000) 28(10):3405-11 and BERNARD G R. et al. Chest (1997) 112(1): 164-72). For example, the severity of cardiovascular dysfunction was assessed by measuring days alive and free of cardiovascular dysfunction over a 28-day observation period. Days alive and free of cardiovascular dysfunction was calculated as the number of days after inclusion that a patient was alive and free of cardiovascular dysfunction over 28-days. Thus, a lower score for days alive and free of cardiovascular dysfunction indicates more cardiovascular dysfunction. The reason that days alive and free of cardiovascular dysfunction is preferable to simply presence or absence of cardiovascular dysfunction is that critical illness has a high acute mortality so that early death (within 28-days) precludes calculation of the presence or absence of cardiovascular dysfunction in dead patients. Organ dysfunction has been evaluated in this way in observational studies (Russell J A. et al. Crit Care Med (2000) 28(10):3405-11) and in randomized controlled trials of new therapy in sepsis, acute respiratory distress syndrome (BERNARD G R. et al. N Engl J Med (1997) 336(13):912-8) and in critical care (HEBERT P C. et al. N Engl J Med (1999) 340(6):409-17).

We scored the presence of three or four of the SIRS criteria each day over the 28-day observation period as a cumulative measure of the severity of SIRS. Severe sepsis was defined as the presence of at least two systemic inflammatory response syndrome criteria and a known or suspected source of infection plus at least one new organ dysfunction by Brussels criteria (at least moderate, severe or extreme).

Haplotype Determination and Selection of htSNPs

We used two steps to determine haplotypes and then haplotype clades of the interferon gamma gene. We inferred haplotypes using PHASE software using un-phased Caucasian genotype data (from http://pga.mbt.washington.edu/) (STEPHENS M. et al. Am J Hum Genet (2001) 68(4):978-89). We then used MEGA 2 to infer a phylogenetic tree so that we could identify major haplotype clades (KUMAR S. et al. Bioinformatics (2001) 17:1244-1245). Haplotypes were sorted according to this phylogenetic tree and this haplotype structure was inspected to choose SNPs that tagged each major haplotype clade, so-called haplotype tag SNPs (htSNPs) (not shown). Polymorphisms genotyped are listed in TABLE 1B. Polymorphisms included in the linkage analysis are listed in TABLE 1C with all flanking sequences in TABLES 1D.

Genotyping

Discarded whole blood samples, stored at 4° C., were collected from the hospital laboratory. The buffy coat was extracted and the samples were transferred to 1.5 mL cryotubes, bar coded and cross-referenced with the unique patient number and stored at −80° C. DNA was extracted from the buffy coat using a QIAamp DNA Midi kit (Qiagen, Mississauga, ON, Canada). Of the enrolled SIRS patients, 854 Caucasians were successfully genotyped for rs1861493 using the 5′ nuclease, TaqMan™ (Applied Biosystems; Foster City, Calif.) polymerase chain reaction (PCR) method. Similarly, 851 Caucasians were successfully genotyped at rs2069718 and 847 Caucasians were successfully genotyped at rs2069727.

Data Analysis

We recorded and compared baseline characteristics (age, gender, admitting APACHE II score, and medical versus surgical admitting diagnosis) across the IFNG SNP genotype groups using a chi-squared or Kruskal-Wallis test where appropriate. We used a chi-square test to assess whether the rs1861493, rs2069718, rs2069727 polymorphisms were significantly associated with 28-day survival. We used a Kruskal-Wallis test to test for differences in days alive and free of various organ dysfunctions and treatments. We used logistic regression with a Genotype*Gender interaction term to test for a significant genotype-gender interaction.

For the Activated Protein C Cohort, the 28 day survival rate (%) for patients who were treated with XIGRIS™ (activated protein C) was compared to control patients who were not treated with XIGRIS™ using a chi-squared test. We considered a by-genotype effect to be significant when two criteria were fulfilled. First, we required an increase of ≧15% in 28-day survival rate in the XIGRIS™ treated group compared to the control group. Second, we required that p<0.1 for this comparison. When both criteria were met we considered the polymorphism allele or genotype which predicted increased 28-day survival with XIGRIS™ treatment to be an “Improved Response Polymorphism” (IRP).

### 3. Results

1.1 rs2069718

1.1.1 Systematic Inflammatory Response Syndrome—Caucasian Cohort

Table 3.1 summarizes the baseline characteristics (age, sex, APACHE II score, medical versus surgical diagnosis, severe sepsis upon admittance, septic shock upon admittance) of 851 Caucasian systematic inflammatory response syndrome patients who were successfully genotyped (CC/CT vs. TT) at rs2069718. Significant differences were detected in gender and APACHEII distributions between the two genotype groups.

TABLE 3.1
Baseline characteristics of a cohort of Caucasian patients who had systematic inflammatory
response syndrome by genotype at rs2069718 (CC/CT vs. TT). Data is reported as percentage
for binary variables and as 25^{th} percentile/median/75^{th} percentile for all other variables.

| | | | | F or | | |
|---|---|---|---|---|---|---|
| | CC/CT | TT | Combined | Chi- | | |
| | (N = 668) | (N = 183) | (N = 851) | square | d.f. | P |
| AGE | 46/59/71 | 44/61/72 | 46/59/71 | 0 | 1,849 | 0.97 |
| SEX | 65.9% (440/668) | 56.3% (103/183) | 63.8% (543/851) | 5.71 | 1 | 0.017 |
| APACHEII | 16/21/27 | 17/23/29 | 16/22/28 | 4.42 | 1,849 | 0.036 |
| SURGICAL | 23.8% (159/668) | 20.8% (38/183) | 23.1% (197/851) | 0.74 | 1 | 0.49 |
| SEP.ADMIT | 76.2% (509/668) | 73.8% (135/183) | 75.7% (644/851) | 0.46 | 1 | 0.498 |
| SS.ADMIT | 56.0% (374/668) | 58.5% (107/183) | 56.5% (481/851) | 0.36 | 1 | 0.548 |

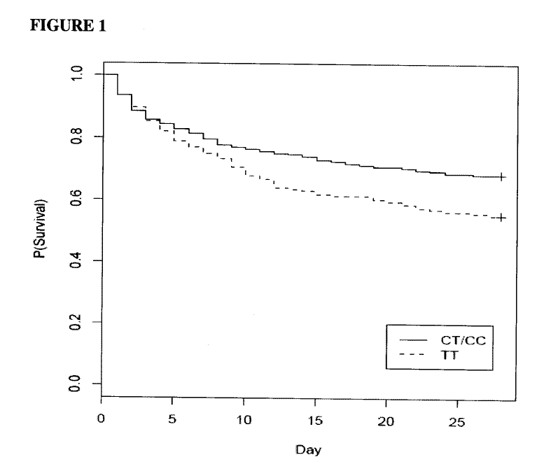

FIG. 1 and Table 3.2 summarize important SNP-phenotype associations. The TT group showed significantly decreased survival (P<0.001), significantly fewer days alive (P=0.00541) and significantly fewer days alive and free of: cardiovascular dysfunction (P=0.0353), coagulation dysfunction (P=0.0131), acute renal dysfunction (P=0.00538), acute hepatic dysfunction (P=0.00635), more than 5 ug/min of vasopressors (P=0.049), more than 15 ug/min of vasopressors (P=0.0368), inotropes (P=0.0144), INR>1.5 (P=0.00282), any renal failure (P=0.00369), renal support (P=0.00241) and any hepatic dysfunction (P=0.00335). The TT group also showed a strong trend for fewer days alive and free of any vasopressors (P=0.071), more than 2 ug/min of vasopressors (P=0.0737) and 3/4 SIRS criteria (P=0.0946). These findings suggest that Caucasians with systematic inflammatory response syndrome who carry the TT genotype at rs2069718 at greater risk of organ dysfunction (cardiovascular, coagulation, renal, hepatic) and have more vasopressor and inotrope use when admitted to the ICU.

TABLE 3.2
Days alive and free of organ dysfunction (DAF) by genotype at rs2069718 (CC/CT vs. TT) in
a cohort of Caucasian patients with systematic inflammatory response syndrome. Data is
reported as percentage for binary variables and as 25^{th} percentile/median/75^{th} percentile for all
other variables.

| | | | | F or | | |
|---|---|---|---|---|---|---|
| | CT/CC | TT | Combined | Chi- | | |
| | (N = 668) | (N = 183) | (N = 851) | square | d.f. | P |
| SURV | 68.3% (456/668) | 55.2% (101) | 65.5% (557) | 10.9 | 1 | <0.001 |
| DA | 12/28/28 | 7.5/28/28 | 10/28/28 | 7.78 | 1,849 | 0.00541 |
| CVS.DAF | 2/8/15 | 2/6/11 | 2/7/14 | 4.44 | 1,849 | 0.0353 |
| COAG.DAF | 9.75/28/28 | 5/23/28 | 8/27/28 | 6.18 | 1,849 | 0.0131 |
| RENAL.DAF | 0/12.5/27 | 0/3/26 | 0/7/26 | 7.79 | 1,849 | 0.00538 |
| LIVER.DAF | 10/28/28 | 6.5/26/28 | 8/28/28 | 7.48 | 1,849 | 0.00635 |
| PRESS.DAF | 7/25/28 | 4/22/28 | 5/24/28 | 3.27 | 1,849 | 0.071 |
| PRESS2.DAF | 7/25/28 | 4/22/28 | 5/25/28 | 3.21 | 1,849 | 0.0737 |
| PRESS5.DAF | 8/26/28 | 4/23/28 | 6/25/28 | 3.89 | 1,849 | 0.049 |
| PRESS15.DAF | 9.75/27/28 | 6.00/25.00/28 | 8/27/28 | 4.37 | 1,849 | 0.0368 |
| INO.DAF | 11/28/28 | 5/26/28 | 8/28/28 | 6.02 | 1,849 | 0.0144 |
| MSIRS3.DAF | 4/19/26 | 2/16/25 | 3/19/26 | 2.8 | 1,849 | 0.0946 |
| PFRATIO.DAF | 11.8/26/28 | 6.5/24/28 | 9/26/28 | 3.53 | 1,849 | 0.0607 |
| INR.DAF | 10/26/28 | 5/22/28 | 7/26/28 | 8.97 | 1,849 | 0.00282 |
| ANYREN.DAF | 0/6/26 | 0/0/25 | 0/1/26 | 8.48 | 1,849 | 0.00369 |
| RENSUP.DAF | 7/28/28 | 4/22/28 | 5.5/28/28 | 9.26 | 1,849 | 0.00241 |
| ANYLIVER.DAF | 6/28/28 | 3.5/22/28 | 4/28/28 | 8.65 | 1,849 | 0.00335 |

1.1.2 Severe Sepsis—Caucasian Cohort

Table 3.3 summarizes the baseline characteristics (age, sex, APACHE II score, medical versus surgical diagnosis, septic shock upon admittance) of 644 Caucasian severe sepsis patients who were successfully genotyped (CC/CT vs. TT) at rs2069718. A significant difference in gender distribution was detected between the two genotype groups.

TABLE 3.3
Baseline characteristics of a cohort of Caucasian patients who had severe sepsis by genotype
at rs2069718 (CC/CT vs. TT). Data is reported as percentage for binary variables and as 25^{th}
percentile/median/75^{th} percentile for all other variables.

| | | | | F or | | |
|---|---|---|---|---|---|---|
| | CC/CT | TT | Combined | Chi- | | |
| | (N = 509) | (N = 135) | (N = 644) | square | d.f. | P |
| AGE | 47/59/71 | 44.5/61/72 | 47/59/71 | 0.02 | 1,642 | 0.889 |
| SEX | 67.4% (343/509) | 56.3% (76/135) | 65.1% (419/644) | 5.77 | 1 | 0.0163 |
| APACHEII | 17/23/29 | 19/24/30 | 18/23/29 | 3.74 | 1,642 | 0.0534 |
| SURGICAL | 25.5% (130/509) | 19.3% (26/135) | 24.2% (156/644) | 2.29 | 1 | 0.13 |
| SS.ADMIT | 73.5% (374/509) | 79.3% (107/135) | 74.7% (481/644) | 1.89 | 1 | 0.17 |

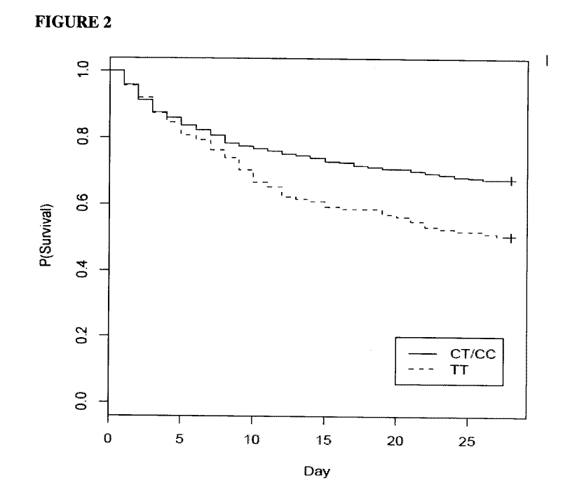

FIG. 2 and Table 3.4 summarizes important SNP-phenotype associations. The TT group showed significantly decreased survival (P<0.001), significantly fewer days alive (P=0.00189) and significantly fewer days alive and free of cardiovascular dysfunction (P=0.0463), coagulation dysfunction (P=0.00436), acute renal dysfunction (P=0.00453), acute hepatic dysfunction (P=0.0024), use of vasopressors (P=0.0359), use of more than 2 ug/min of vasopressors (P=0.0359), use of more than 5 ug/min of vasopressors (P=0.0236), use of more than lSug/min of vasopressors (P=0.0231), inotropes (P=0.00475), INR>1.5, (P<0.001), any renal dysfunction (P=0.0154), renal support (P=0.00888) and any hepatic dysfunction (P<0.001). The TT group also showed a strong trend towards fewer days alive and free of acute lung injury (P=0.053).

These findings suggest that Caucasian severe sepsis patients who carry the TT genotype at rs2069718 may be at greater risk of organ dysfunction (respiratory, cardiovascular, coagulation, renal and hepatic) and are subject to more vasopressor and inotrope use once admitted to the ICU.

TABLE 3.4
Days alive and free of organ dysfunction (DAF) by genotype at rs2069718 (CC/CT vs. TT) in
a cohort of Caucasian patients with severe sepsis. Data is reported as percentage for binary
variables and as 25^{th} percentile/median/75^{th} percentile for all other variables.

| | CC/CT | TT | Combined | F or Chi- | | |
|---|---|---|---|---|---|---|
| | (N = 509) | (N = 135) | (N = 644) | square | d.f. | P |
| SURV | 67.6% (344/509) | 50.4% (68/135) | 64.0% (412/644) | 13.7 | 1 | <0.001 |
| DA | 13/28/28 | 8/28/2028 | 10/28/28 | 9.74 | 1,642 | 0.00189 |
| CVS.DAF | 2/9/15 | 1.5/6/11 | 2/8/14 | 8.08 | 1,642 | 0.00463 |
| COAG.DAF | 10/28/28 | 6/21/28 | 8/26/28 | 8.18 | 1,642 | 0.00436 |
| RENAL.DAF | 0/10/26 | 0/1/25 | 0/4/26 | 8.12 | 1,642 | 0.00453 |
| LIVER.DAF | 11/28/28 | 7/23/28 | 9/28/28 | 9.29 | 1,642 | 0.0024 |
| ALI.DAF | 8/24/28 | 5/20/28 | 7.5/22/28 | 3.76 | 1,642 | 0.053 |
| PRESS.DAF | 7/24/28 | 4/18/27 | 5/23/27 | 4.42 | 1,642 | 0.0359 |
| PRESS2.DAF | 7/24/28 | 4/18/27 | 5/23/28 | 4.42 | 1,642 | 0.0359 |
| PRESS5.DAF | 8/25/28 | 4/18/27 | 6.5/24/28 | 5.15 | 1,642 | 0.0236 |
| PRESS15.DAF | 11/27/28 | 6/23/28 | 8/27/28 | 5.19 | 1,642 | 0.0231 |
| INO.DAF | 12/28/28 | 5/22/28 | 8/28/28 | 8.03 | 1,642 | 0.00475 |
| PFRATIO.DAF | 12/26/28 | 7/23/28 | 9/26/28 | 3.24 | 1,642 | 0.0724 |
| INR.DAF | 10/26/28 | 6/21/27 | 8/25/28 | 11.9 | 1,642 | <0.001 |
| ANYREN.DAF | 0/3/26 | 0/0/24 | 0/0/26 | 5.9 | 1,642 | 0.0154 |
| RENSUP.DAF | 6/28/28 | 4.5/19/28 | 6/27/28 | 6.89 | 1,642 | 0.00888 |
| ANYLIVER.DAF | 6/28/28 | 3/14/28 | 5/26/28 | 12.2 | 1,642 | <0.001 |

1.1.3 Septic Shock—Caucasian Cohort

Table 3.5 summarizes the baseline characteristics (age, sex, APACHE II score and medical/surgical diagnosis) of 481 Caucasian septic shock patients who were successfully genotyped (CC/CT vs. TT) at rs2069718. A significant difference in gender distribution was detected between the two genotype groups.

TABLE 3.5
Baseline characteristics of a cohort of Caucasian patients who had septic shock by genotype
of rs2069718 (CC/CT vs. TT). Data is reported as percentage for binary variables and as 25^{th}
percentile/median/75^{th} percentile for all other variables.

| | | | | F or | | |
|---|---|---|---|---|---|---|
| | CC/CT | TT | Combined | Chi- | | |
| | (N = 374) | (N = 107) | (N = 481) | square | d.f. | P |
| AGE | 48/60.5/72 | 48.5/62/73 | 48/60/72 | 0.51 | 1,479 | 0.474 |
| SEX | 67.4% (252/374) | 55.1% (59/107) | 64.7% (311/481) | 5.45 | 1 | 0.0195 |
| APACHEII | 19/25/30 | 20/26/31.5 | 20/25/31 | 1.52 | 1,479 | 0.218 |
| SURGICAL | 27.5% (103/374) | 20.6% (22/107) | 26.0% (125/481) | 2.11 | 1 | 0.147 |

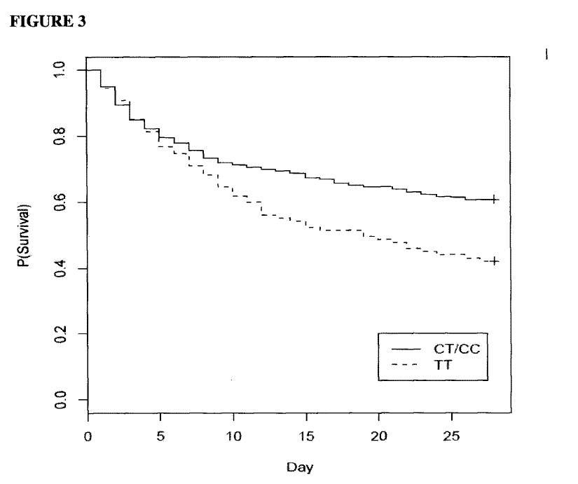

FIG. 3 and Table 3.6 summarizes important SNP-phenotype associations. The TT group showed significantly decreased survival (P<0.001), significantly fewer days alive (P=0.00758) and significantly fewer days alive and free of cardiovascular dysfunction (P=0.0427), coagulation dysfunction (P=0.0119) acute renal dysfunction (P=0.0174), use of more than 5 ug/min of vasopressors (P=0.0476), use of more than 15 ug/min of vasopressors (P=0.0461), use of inotropes (P=0.0112), INR>1.5 (P=0.00713) and any liver dysfunction (P=0.00849). The TT group also showed a strong trend towards more days alive and free of acute lung injury (P=0.0752), use of vasopressors (P=0.0768, use of more than 2 ug/min of vasopressors (P=0.0755), any renal dysfunction (P=0.08) and renal support (P=0.0508). These findings suggest that Caucasian septic shock patients who carry the TT genotype at rs2069718 may be in greater need of vasopressor and inotrope therapy and may be at greater risk of organ dysfunction (cardiovascular, coagulation, hepatic and renal) and are subject to more vasopressor and inotrope use once admitted to the ICU.

TABLE 3.6
Days alive and free of organ dysfunction (DAF) by genotype of Interferon Gamma rs2069718
(CC/CT vs. TT) in a cohort of Caucasian patients with septic shock. Data is reported as
percentage for binary variables and as 25^{th} percentile/median/75^{th} percentile for all other
variables.

| | CC/CT | TT | Combined | F or Chi- | | |
|---|---|---|---|---|---|---|
| | (N = 374) | (N = 107) | (N = 481) | square | d.f. | P |
| SURV | 60.7% (227/374) | 42.1% (45/107) | 56.5% (272/481) | 11.8 | 1 | <0.001 |
| DA | 8/28/28 | 6.5/19/28 | 7.25/28/28 | 7.19 | 1,479 | 0.00758 |
| CVS.DAF | 1/7/14 | 1/4/11 | 1/6/13 | 4.13 | 1,479 | 0.0427 |
| COAG.DAF | 5.25/25/28 | 4/15/28 | 5/24/28 | 6.37 | 1,479 | 0.0119 |
| RENAL.DAF | 0/2.5/26 | 0/0/12.5 | 0/0/25 | 5.69 | 1,479 | 0.0174 |
| ALI.DAF | 6/21/28 | 4/14/26 | 5/20/28 | 3.18 | 1,479 | 0.0752 |
| PRESS.DAF | 2/21/26 | 3/12/25 | 2/20/26 | 3.14 | 1,479 | 0.0768 |
| PRESS2.DAF | 2/21/26 | 3/12/25 | 2/20/26 | 3.17 | 1,479 | 0.0755 |
| PRESS5.DAF | 3/22/26.8 | 3/13/26 | 3/22/26 | 3.95 | 1,479 | 0.0476 |
| PRESS15.DAF | 5/26/28 | 5/15/27 | 5/25/28 | 4 | 1,479 | 0.0461 |
| INO.DAF | 7/27/28 | 4.5/17/28 | 5/25.5/28 | 6.49 | 1,479 | 0.0112 |
| INR.DAF | 6/24/28 | 4.5/15/26 | 5/22.5/28 | 7.3 | 1,479 | 0.00713 |
| ANYREN.DAF | 0/0/26 | 0/0/9.5 | 0/0/25 | 3.08 | 1,479 | 0.08 |
| RENSUP.DAF | 3/25/28 | 3.5/12/28 | 3/22/28 | 3.83 | 1,479 | 0.0508 |
| ANYLIVER.DAF | 3.25/25/28 | 3/11/28 | 3/23/28 | 6.98 | 1,479 | 0.00849 |

1.1.4 ICU Caucasians—Male and Female Cohorts

Table 3.7 summarizes the baseline characteristics (age, gender, APACHE II score and medical/surgical diagnosis, severe sepsis upon admittance, septic shock upon admittance) of: (1) Caucasian females with SIRS(N=308), (2) Caucasian males with SIRS(N=543), (3) Caucasian females with severe sepsis (N=225), (4) Caucasian males with severe sepsis (N=419), (5) Caucasian females with septic shock (N=170) and (6) Caucasian males with septic shock (N=311), who were successfully genotyped (CC/CT vs. TT) at rs2069718. For females with SIRS and severe sepsis, a significant difference in APACHE II at baseline was detected.

TABLE 3.7
Baseline characteristics (age, sex, APACHE II score, medical versus surgical diagnosis,
severe sepsis upon admittance, septic shock upon admittance) of Caucasian females and
Caucasian males by cohort (i.e. Systemic Inflammatory Response Syndrome (SIRS), severe
sepsis and septic shock) by genotype rs2069718 (CC/CT vs. TT). Data is reported as
percentage for binary variables and as 25^{th} percentile/median/75^{th} percentile for all other
variables.

| | | | | | | F or | | |
|---|---|---|---|---|---|---|---|---|
| | | Baseline | | | | Chi- | | |
| Gender | Cohort | Characteristic | CC/CT | TT | Combined | square | DF | P |
| Female | SIRS | N | 228 | 80 | 308 | | | |
| Female | SIRS | AGE | 43.8/58/71 | 44/59.5/72 | 44/58/71 | 0 | 1,306 | 0.973 |
| Female | SIRS | APACHEII | 14/20/25 | 18/23/28 | 15/22/27 | 7.87 | 1,306 | 0.00535 |
| Female | SIRS | SURGICAL | 25.9% (59/228) | 20.0% (16/80) | 24.4% (75/308) | 1.11 | 1 | 0.292 |
| Female | SIRS | SEVSEP. ADMIT | 72.8% (166/228) | 73.8% (59/80) | 73.1% (225/308) | 0.03 | 1 | 0.87 |
| Female | SIRS | SS. ADMIT | 53.5% (122/228) | 60.0% (48/80) | 55.2% (170/308) | 1.01 | 1 | 0.315 |
| Female | Severe | N | 166 | 59 | 225 | | | |
| | Sepsis | | | | | | | |
| Female | Severe | AGE | 44.2/58/70 | 46/61/72 | 45/58.5/70 | 0.11 | 1,223 | 0.743 |
| | Sepsis | | | | | | | |
| Female | Severe | APACHEII | 16/22/27 | 20/24/30 | 17/23/28 | 6.24 | 1,223 | 0.0132 |
| | Sepsis | | | | | | | |
| Female | Severe | SURGICAL | 28.9% (48/166) | 16.9% (10/59) | 25.8% (58/225) | 3.26 | 1 | 0.0711 |
| | Sepsis | | | | | | | |
| Female | Severe | SS. ADMIT | 73.5% (122/166) | 81.4% (48/59) | 75.6% (170/225) | 1.46 | 1 | 0.227 |
| | Sepsis | | | | | | | |
| Female | Septic | N | 122 | 48 | 170 | | | |
| | Shock | | | | | | | |
| Female | Septic | AGE | 45/55.5/70 | 50.5/62.5/72 | 46/59/70 | 2.54 | 1,168 | 0.113 |
| | Shock | | | | | | | |
| Female | Septic | APACHEII | 17.2/23/29 | 20.8/25.5/31.2 | 19/24/29 | 3.2 | 1,168 | 0.0754 |
| | Shock | | | | | | | |
| Female | Septic | SURGICAL | 30.3% (37/122) | 20.8% (10/48) | 27.6% (47/170) | 1.55 | 1 | 0.213 |
| | Shock | | | | | | | |
| Male | SIRS | N | 440 | 103 | 543 | | | |
| Male | SIRS | AGE | 48/59.5/71 | 45/61/73 | 47/60/71 | 0.05 | 1,541 | 0.817 |
| Male | SIRS | APACHEII | 16/22/28 | 16/23/29 | 16/22/28 | 0.45 | 1,541 | 0.502 |
| Male | SIRS | SURGICAL | 22.7% (100/440) | 21.4% | 22.5% (122/543) | 0.09 | 1 | 0.765 |
| | | | | (22/103) | | | | |
| Male | SIRS | SEVSEP. ADMIT | 78.0% (343/440) | 73.8% | 77.2% (419/543) | 0.82 | 1 | 0.364 |
| | | | | (76/103) | | | | |
| Male | SIRS | SS. ADMIT | 57.3% (252/440) | 57.3% | 57.3% (311/543) | 0 | 1 | 0.999 |
| | | | | (59/103) | | | | |
| Male | Severe | N | 343 | 76 | 419 | | | |
| | Sepsis | | | | | | | |
| Male | Severe | AGE | 48/59/71 | 44/60.5/73 | 48/60/71.5 | 0 | 1,417 | 0.956 |
| | Sepsis | | | | | | | |
| Male | Severe | APACHEII | 18/24/30 | 18.8/23.5/30.2 | 18/23/30 | 0.49 | 1,417 | 0.486 |
| | Sepsis | | | | | | | |
| Male | Severe | SURGICAL | 23.9% (82/343) | 21.1% (16/76) | 23.4% (98/419) | 0.28 | 1 | 0.595 |
| | Sepsis | | | | | | | |
| Male | Severe | SS. ADMIT | 73.5% (252/343) | 77.6% (59/76) | 74.2% (311/419) | 0.56 | 1 | 0.453 |
| | Sepsis | | | | | | | |
| Male | Septic | N | 252 | 59 | 311 | | | |
| | Shock | | | | | | | |
| Male | Septic | AGE | 49/63.5/72 | 46/62/73 | 49/62/72 | 0.02 | 1,309 | 0.876 |
| | Shock | | | | | | | |
| Male | Septic | APACHEII | 20/25/31.2 | 20/26/31.5 | 20/26/31 | 0.21 | 1,309 | 0.648 |
| | Shock | | | | | | | |
| Male | Septic | SURGICAL | 26.2% (66/252) | 20.3% (12/59) | 25.1% (78/311) | 0.87 | 1 | 0.351 |
| | Shock | | | | | | | |

Table 3.8 summarizes survival by gender in Caucasian patients with: (1) systematic inflammatory response syndrome (SIRS), (2) severe sepsis and (3) septic shock by genotype group (CC/CT vs. TT) at rs2069718. For females, the TT groups shows significantly decreased survival in the SIRS cohort (P<0.001), the severe sepsis cohort (P<0.001) and the septic shock cohort (P<0.001). For males, the TT group shows significantly decreased survival in the severe sepsis cohort (P=0.0384) and shows a strong trend for decreased survival in the septic shock cohort (P=0.08).

TABLE 3.8
Survival by genotype of rs2069718 (CC/CT vs. TT) in a cohort of Caucasian patients with
systematic inflammatory response syndrome, severe sepsis and septic shock in females and
males.

| | | | | | Chi- | | |
|---|---|---|---|---|---|---|---|
| Cohort | Gender | CC/CT | TT | Combined | Square | d.f. | P |
| SIRS | Female | 73.2% (167/228) | 52.5% | 67.9% | 11.7 | 1 | <0.001 |
| | | | (42/80) | (209/308) | | | |
| Severe Sepsis | Female | 72.3% (120/166) | 47.5% | 65.8% | 11.9 | 1 | <0.001 |
| | | | (28/59) | (148/225) | | | |
| Septic Shock | Female | 65.6% (80/122) | 37.5% | 57.6% | 11.1 | 1 | <0.001 |
| | | | (18/48) | (98/170) | | | |
| SIRS | Male | 65.7% (289/440) | 57.3% | 64.1% | 2.56 | 1 | 0.11 |
| | | | (59/103) | (348/543) | | | |
| Severe Sepsis | Male | 65.3% (224/343) | 52.6% | 63.0% | 4.29 | 1 | 0.0384 |
| | | | (40/76) | (264/419) | | | |
| Septic Shock | Male | 58.3% (147/252) | 45.8% | 55.9% | 3.07 | 1 | 0.08 |
| | | | (27/59) | (174/311) | | | |

1.1.6 Biological Plausibility Cohort

Table 3.11 summarizes the baseline characteristics (age, sex, smoker, diabetes, hypertension, preoperative ejection fraction, bypass time, cross-clamp time, and aprotinin use) of 25 non-septic SIRS patients who were successfully genotyped (CC/CT vs. TT) at rs2069718. No significant differences between the two genotype groups were detected on admission to the CSICU.

TABLE 3.11
Baseline characteristics of a cohort of non-septic CSICU patients diagnosed
with systematic inflammatory response syndrome by genotype of Interferon Gamma
rs2069718 (CC/CT vs. TT).

| | CC/CT.Mean | CC/CT.Med | CC/CT.SD | TT.Mean | TT.Med | TT.SD |
|---|---|---|---|---|---|---|
| AGE | 69 | 70 | 8.3 | 62 | 63 | 6 |
| SEX | 0.52 | 1 | 0.51 | 0.75 | 1 | 0.5 |
| SMOKER | 0.14 | 0 | 0.36 | 0 | 0 | 0 |
| DIABETES | 0.38 | 0 | 0.5 | 0 | 0 | 0 |
| H.TENSE | 0.52 | 1 | 0.51 | 0.75 | 1 | 0.5 |
| EJEC.FRAC | 0.54 | 0.59 | 0.14 | 0.52 | 0.5 | 0.15 |
| BYPASS | 1.8 | 1.7 | 0.65 | 1.3 | 1.2 | 0.29 |
| CLAMP | 1.4 | 1.28 | 0.5 | 1 | 0.97 | 0.3 |
| APROTININ | 0 | 0 | 0 | 0 | 0 | 0 |

Table 3.12 summarizes important SNP-biomarker associations. The CC/CT genotype group had significantly higher serum interleukin receptor-1a (IL1ra) levels post-cardiopulmonary bypass (P=0.0058), serum interleukin-8 (IL8) levels post-cardiopulmonary bypass (P=0.011) and serum monocyte chemoattractant protein (MCP) levels post-cardiopulmonary bypass (P=0.0348). CC/CT individuals also had a strong trend for higher serum interleukin-10 (IL10) levels post-cardiopulmonary bypass (P=0.0705). These findings suggest that non-septic SIRS patients who carry either the CC or CT genotype rs2069718 are more likely to experience a pro-inflammatory cytokine (MCP, IL1ra, IL8 and IL10) response after cardiopulmonary bypass surgery.

TABLE 3.12
Biological plausibility of Interferon Gamma association using biomarkers in a cohort
of non-septic CSICU patients diagnosed with systematic inflammatory response
syndrome by genotype at rs2069718 (CC/CT vs. TT). Data is reported as 25^{th} percentile/
median/75^{th} percentile.

| | CC/CT | TT | Combined | Test |
|---|---|---|---|---|
| | (N = 21) | (N = 4) | (N = 25) | Statistic |
| IL10.3 | 0.0/8.7/12.4 | 0.0/0.0/1.6 | 0.0/0.0/8.7 | F = 3.6 d.f. = 1,23 P = 0.070 |
| IL1ra.0 | 1203/1465/2603 | 613/745/905 | 832/1224/1873 | F = 9.7 d.f. = 1,19 |
| | | | | P = 0.0058 |
| IL8.3 | 37/69/122 | 27/27/29 | 28/45/78 | F = 7.6 d.f. = 1,23 P = 0.011 |
| MCP.0 | 152/199/262 | 65/91/128 | 135/182/245 | F = 5 d.f. = 1,23 P = 0.035 |

1.1.7 Activated Protein C (Xigris™) Cohort

Table 3.13 summarizes survival by allele of Caucasian sepsis patients treated with Xigris™ who were successfully genotyped at rs2069718. Patients treated with Xigris™ who carry the C allele have significantly increased survival compared to all other groups. Xigris™ treated C allele individuals show a greater survival response than Xigris™ treated T allele individuals when compared with an untreated control.

TABLE 3.13
28-day survival of XIGRIS ™-treated patients and matched controls (patients
not treated with XIGRIS ™) by rs2069718 in a cohort of critically ill patients who had severe
sepsis and no XIGRIS ™ contraindications. Data is presented for both IRP and non-IRP
patients. The chisquare tests and the reported P-values correspond to the comparison of IRP
Matched Controls to IRP XIGRIS ™-treated patients only (Column A versus Column B). 28-
day survival is given as % survival (N survived/N total). D.F., degrees of freedom.

| 28-Day Survival | | | | | | |
|---|---|---|---|---|---|---|
| | B | | | | | |
| A | IRP (C) | | D | | | |
| IRP (C) | XIGRIS ™- | C | non-IRP (T) | A vs B | | |
| Matched | Treated | non-IRP (T) | XIGRIS ™-Treated | Chi- | | |
| Controls | Patients | Matched Controls | Patients | square | D.F. | P-VALUE |
| 58% (119/205) | 74.2% (23/31) | 48.2% (93/193) | 48.6% (17/35) | 2.93 | 1 | 0.087 |

1.2 rs1861493

1.2.1 Systematic Inflammatory Response Syndrome—Caucasian Cohort

Table 3.14 summarizes the baseline characteristics (age, sex, APACHE II score, medical versus surgical diagnosis, severe sepsis upon admittance, septic shock upon admittance) of 854 Caucasian systematic inflammatory response syndrome patients who were successfully genotyped (GG vs. AA/GA) at rs1861493.

TABLE 3.14
Baseline characteristics of a cohort of Caucasian patients who had systematic inflammatory
response syndrome by genotype at rs1861493 (GG vs. AA/GA). Data is reported as
percentage for binary variables and as 25^{th} percentile/median/75^{th} percentile for all other
variables.

| | GG | AA/GA | Combined | F or Chi- | | |
|---|---|---|---|---|---|---|
| | (N = 87) | (N = 767) | (N = 854) | square | d.f. | P |
| AGE | 49.5/65/72.5 | 45.5/59/71 | 46/59/71 | 3.67 | 1,852 | 0.0556 |
| SEX | 56.3% (49/87) | 64.7% (496/767) | 63.8% (545/854) | 2.36 | 1 | 0.125 |
| APACHEII | 18/23/28.5 | 16/21/27.5 | 16/22/28 | 2.43 | 1,852 | 0.119 |
| SURGICAL | 24.1% (21/87) | 23.1% (177/767) | 23.2% (198/854) | 0.05 | 1 | 0.824 |
| SEVSEP.ADMIT | 71.3% (62/87) | 76.1% (584/767) | 75.6% (646/854) | 1.01 | 1 | 0.315 |
| SS.ADMIT | 58.6% (51/87) | 56.1% (430/767) | 56.3% (481/854) | 0.21 | 1 | 0.648 |

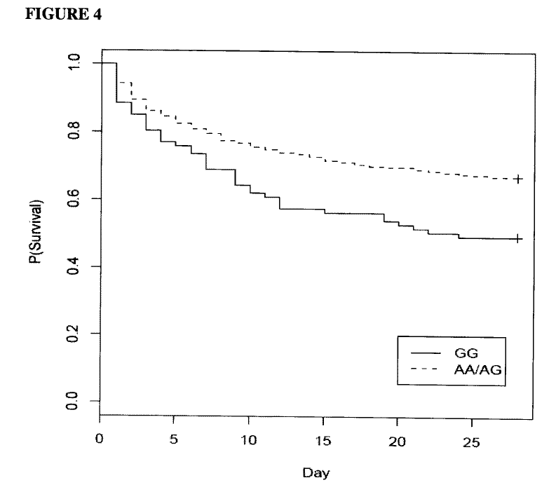

FIG. 4 and Table 3.15 summarizes important SNP-phenotype associations. The GG group showed significantly decreased survival (P=0.0011), significantly fewer days alive (P=0.00167) and significantly fewer days alive and free of cardiovascular dysfunction (P=0.0283), respiratory dysfunction (P=0.0412), coagulation dysfunction (P=0.00566), acute hepatic dysfunction (P=0.00159), acute lung injury (P=0.0352), use of more than 15 ug/min of vasopressors (P=0.0254), inotropes (P=0.00367), 4/4 SIRS criteria (P=0.0287), INR>1.5 (P=0.00243), any renal dysfunction (P=0.0415), renal support (P<0.001) and any hepatic dysfunction (P=0.00485). GG individuals also showed a strong trend for fewer days alive of neurological dysfunction (P=0.0785), vasopressors (P=0.0621), more than 2 ug/min of vasopressors (P=0.0633) and more than 5 ug/min of vasopressors (P=0.0502). These findings suggest that Caucasian systematic inflammatory response patients who carry the GG genotype at IFNG rs1861493 may be at greater risk of organ dysfunction (cardiovascular, respiratory, neurological, coagulation and hepatic) and are subject to more vasopressor and inotrope use once admitted to the ICU.

TABLE 3.15
Days alive and free of organ dysfunction (DAF) by genotype at rs1861493 (GG vs. AA/GA)
in a cohort of Caucasian patients with systematic inflammatory response syndrome. Data is
reported as percentage for binary variables and as 25^{th} percentile/median/75^{th} percentile for all
other variables.

| | GG | AA/GA | Combined | F or Chi- | | |
|---|---|---|---|---|---|---|
| | (N = 87) | (N = 767) | (N = 854) | square | d.f. | P |
| SURV | 49.4% (43/87) | 67% (514/767) | 65.2% (557/854) | 10.7 | 1 | 0.0011 |
| DA | 6/24/28 | 11/28/28 | 10/28/28 | 9.95 | 1,852 | 0.00167 |
| CVS.DAF | 1/6/10.5 | 2/7/14 | 2/7/14 | 4.83 | 1,852 | 0.0283 |
| RESP.DAF | 1/12/26 | 2/21/26 | 2/20/26 | 4.18 | 1,852 | 0.0412 |
| CNS.DAF | 1/15/28 | 4/24/28 | 3/24/28 | 3.1 | 1,852 | 0.0785 |
| COAG.DAF | 3.5/18/28 | 9/28/28 | 8/27/28 | 7.69 | 1,852 | 0.00566 |
| LIVER.DAF | 5/19/28 | 10/28/28 | 8/28/28 | 10 | 1,852 | 0.00159 |
| ALI.DAF | 3/20/28 | 8/25/28 | 7/24/28 | 4.45 | 1,852 | 0.0352 |
| PRESS.DAF | 3.5/14/28 | 6/25/28 | 5/24/28 | 3.49 | 1,852 | 0.0621 |
| PRESS2.DAF | 3.5/14/28 | 6/25/28 | 5/25/28 | 3.46 | 1,852 | 0.0633 |
| PRESS5.DAF | 4/14/28 | 7/26/28 | 6/25/28 | 3.85 | 1,852 | 0.0502 |
| PRESS15.DAF | 5.5/18/28 | 8/27/28 | 8/27/28 | 5.01 | 1,852 | 0.0254 |
| INO.DAF | 4/21/28 | 10/28/28 | 8/28/28 | 8.49 | 1,852 | 0.00367 |
| MSIRS4.DAF | 4/24/27 | 8/26/28 | 8/26/28 | 4.8 | 1,852 | 0.0287 |
| PFRATIO.DAF | 5/22/28 | 10/26/28 | 9/26/28 | 5.64 | 1,852 | 0.0177 |
| INR.DAF | 4.5/16/28 | 8.5/26/28 | 7/26/28 | 9.25 | 1,852 | 0.00243 |
| ANYREN.DAF | 0/0/25 | 0/5/26 | 0/1/26 | 4.17 | 1,852 | 0.0415 |
| RENSUP.DAF | 2/12/28 | 7/28/28 | 5.5/28/28 | 12.9 | 1,852 | <0.001 |
| ANYLIVER.DAF | 3/12/28 | 5/28/28 | 4/28/28 | 7.98 | 1,852 | 0.00485 |

1.2.2 Severe Sepsis—Caucasian Cohort

Table 3.16 summarizes the baseline characteristics (age, sex, APACHE II score, medical versus surgical diagnosis, septic shock upon admittance) of 646 Caucasian severe sepsis patients who were successfully genotyped (GG vs. AA/GA) at rs1861493.

TABLE 3.16
Baseline characteristics of a cohort of Caucasian patients who had severe sepsis by genotype
of rs1861493 (GG vs. AA/GA). Data is reported as percentage for binary variables and as
25^{th} percentile/median/75^{th} percentile for all other variables.

| | GG | AA/GA | Combined | F or Chi- | | |
|---|---|---|---|---|---|---|
| | (N = 62) | (N = 584) | (N = 646) | square | d.f. | P |
| AGE | 49.2/66/72.8 | 46/59/71 | 47/59/71 | 3.6 | 1,644 | 0.0582 |
| SEX | 54.8% (34) | 66.3% (387/584) | 65.2% (421/646) | 3.23 | 1 | 0.0725 |
| APACHEII | 19.2/25/30.8 | 17/23/29 | 18/23/29 | 2.58 | 1,644 | 0.109 |
| SURGICAL | 22.6% (14/62) | 24.3% (142/584) | 24.1% (156/646) | 0.09 | 1 | 0.762 |
| SS.ADMIT | 82.3% (51/62) | 73.6% (430/584) | 74.5% (481/646) | 2.19 | 1 | 0.139 |

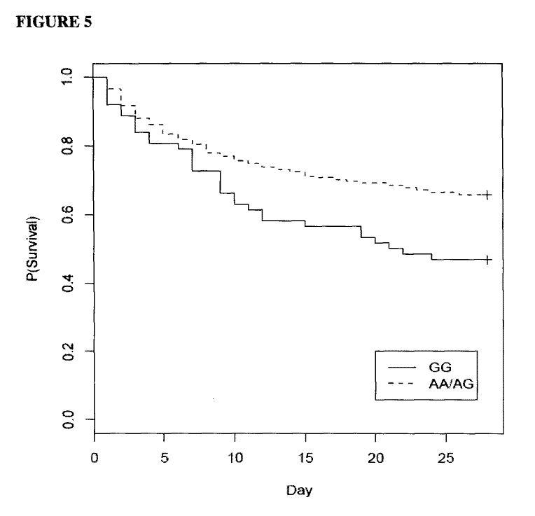

FIG. 5 and Table 3.17 summarizes important SNP-phenotype associations. The GG group showed significantly decreased survival (P=0.00339), significantly fewer days alive (P=0.00744) and significantly fewer days alive and free of: cardiovascular dysfunction (P=0.0296), respiratory dysfunction (P=0.0754), coagulation dysfunction (P=0.032), acute hepatic dysfunction (P=0.00986), inotrope (P=0.0101), INR>1.5 (P=0.0149), renal support (P=0.00837) and any hepatic dysfunction (P−0.0125). The GG group also showed a strong trend towards fewer days alive and free of acute lung injury (P=0.0696), use of vasopressors (P=0.0885), use of more than 2 ug/min of vasopressors (P=0.0942), use of more than 5 ug/min of vasopressors (P=0.0932) and use of more than 15 ug/min of vasopressors (P=0.0693). These findings suggest that Caucasian severe sepsis patients who carry the GG genotype at rs1861493 may be at greater risk of organ dysfunction (respiratory, cardiovascular, respiratory, coagulation and hepatic) and subject to more vasopressor and inotrope use once admitted to the ICU.

TABLE 3.17
Days alive and free of organ dysfunction (DAF) by genotype at rs1861493 (GG
vs. AA/GA) in a cohort of Caucasian patients with severe sepsis. Data is reported as
percentage for binary variables and as 25^{th} percentile/median/75^{th} percentile
for all other variables.

| | | | | F or | | |
|---|---|---|---|---|---|---|
| | GG | AA/GA | Combined | Chi- | | |
| | (N = 62) | (N = 584) | (N = 646) | square | d.f. | P |
| SURV | 46.8% (29/62) | 65.6% (383) | 63.8% (412) | 8.58 | 1 | 0.00339 |
| DA | 7/21.5/28 | 11/28/28 | 10/28/28 | 7.21 | 1,644 | 0.00744 |
| CVS.DAF | 1/6/11 | 2/8/15 | 2/8/14 | 4.76 | 1,644 | 0.0296 |
| RESP.DAF | 0/8.5/24.8 | 2/18/25 | 2/18/25 | 3.17 | 1,644 | 0.0754 |
| COAG.DAF | 4.5/18/28 | 8.75/27/28 | 8/26/28 | 4.62 | 1,644 | 0.032 |
| LIVER.DAF | 7/17/28 | 9.75/28/28 | 9/28/28 | 6.7 | 1,644 | 0.00986 |
| ALI.DAF | 4.25/18.5/28 | 8/23/28 | 7.5/22/28 | 3.3 | 1,644 | 0.0696 |
| PRESS.DAF | 4/13/27 | 6/23/28 | 5/23/27 | 2.91 | 1,644 | 0.0885 |
| PRESS2.DAF | 4/13/27 | 6/24/28 | 5/23/28 | 2.81 | 1,644 | 0.0942 |
| PRESS5.DAF | 4.25/14/27 | 7/25/28 | 6.5/24/28 | 2.83 | 1,644 | 0.0932 |
| PRESS15.DAF | 6/17/28 | 8/27/28 | 8/27/28 | 3.31 | 1,644 | 0.0693 |
| INO.DAF | 5/20.5/28 | 10/28/28 | 8/28/28 | 6.65 | 1,644 | 0.0101 |
| PFRATIO.DAF | 7/21/27.8 | 10/26/28 | 9/26/28 | 3.73 | 1,644 | 0.0538 |
| INR.DAF | 6.25/15.5/27 | 8/26/28 | 8/25/28 | 5.96 | 1,644 | 0.0149 |
| RENSUP.DAF | 3.25/13.5/28 | 6/28/28 | 6/27/28 | 7 | 1,644 | 0.00837 |
| ANYLIVER.DAF | 3.25/11.5/28 | 5/28/28 | 5/26/28 | 6.28 | 1,644 | 0.0125 |

Septic Shock—Caucasian Cohort

Table 3.18 summarizes the baseline characteristics (age, gender, APACHE II score and medical/surgical diagnosis) of 481 Caucasian septic shock patients who were successfully genotyped (GG vs. AA/GA) at rs1861493. A significant difference in age was detected between the two genotype groups on admission to the ICU.

TABLE 3.18
Baseline characteristics of a cohort of Caucasian patients who had septic shock by genotype at
rs1861493 (GG vs. AA/GA). Data is reported as percentage for binary variables and as 25^{th}
percentile/median/75^{th} percentile for all other variables.

| | GG | AA/GA | Combined | F or Chi- | | |
|---|---|---|---|---|---|---|
| | (N = 51) | (N = 430) | (N = 481) | square | d.f. | P |
| AGE | 56.5/67/73 | 48/60/72 | 48/60/72 | 4.28 | 1,479 | 0.0392 |
| SEX | 54.9% (28/51) | 66.0% (284/430) | 64.9% (312/481) | 2.48 | 1 | 0.115 |
| APACHEII | 20/26/32 | 20/25/31 | 20/25/31 | 0.5 | 1,479 | 0.48 |
| SURGICAL | 23.5% (12/51) | 26.3% (113/430) | 26.0% (125/481) | 0.18 | 1 | 0.672 |

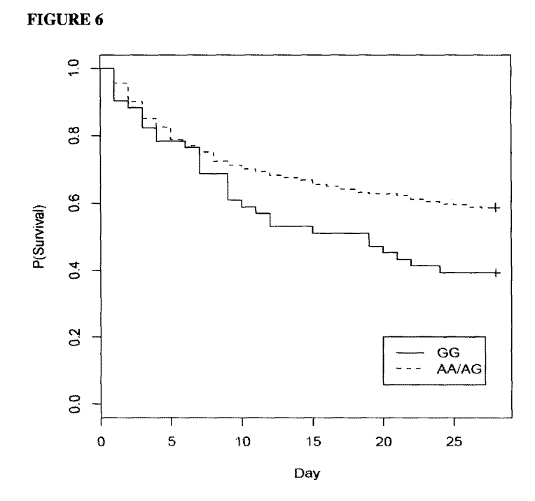

FIG. 6 and Table 3.19 summarizes important SNP-phenotype associations. The GG group showed significantly decreased survival (P=0.00826), significantly fewer days alive (P=0.0278) and significantly fewer days alive and free of: acute hepatic dysfunction (P=0.0221), inotropes (P=0.037) and renal support (P=0.04). GG individuals also showed a strong trend for fewer days alive and free of: cardiovascular dysfunction (P=0.0624), coagulation dysfunction (P=0.0748) and INR>1.5 (P=0.0664). These findings suggest that Caucasian septic shock patients who carry the GG genotype at rs1861493 may be in greater need of steroid, inotrope and vasopressor therapy and may be at greater risk of organ dysfunction (cardiovascular, coagulation and hepatic) and are subject to more inotrope use once admitted to the ICU.

TABLE 3.19
Days alive and free of organ dysfunction (DAF) by genotype at rs1861493 (GG vs. AA/GA)
in a cohort of Caucasian patients with septic shock. Data is reported as percentage for binary
variables and as 25^{th} percentile/median/75^{th} percentile for all other variables.

| | GG | AA/GA | Combined | F or Chi- | | |
|---|---|---|---|---|---|---|
| | (N = 51) | (N = 430) | (N = 481) | square | d.f. | P |
| SURV | 39.2% (20/51) | 58.6% (252/430) | 56.5% (272/481) | 6.98 | 1 | 0.00826 |
| DA | 7/19/28 | 8/28/28 | 7.25/28/28 | 4.87 | 1,479 | 0.0278 |
| CVS.DAF | 1/5/9.5 | 1/7/14 | 1/6/13 | 3.49 | 1,479 | 0.0624 |
| COAG.DAF | 3.5/15/28 | 5/24/28 | 5/24/28 | 3.19 | 1,479 | 0.0748 |
| LIVER.DAF | 5.5/12/28 | 6/26/28 | 6/26/28 | 5.27 | 1,479 | 0.0221 |
| INO.DAF | 4.5/13/28 | 6/26/28 | 5/25.5/28 | 4.38 | 1,479 | 0.037 |
| INR.DAF | 4.5/14/26 | 5/23/28 | 5/22.5/28 | 3.39 | 1,479 | 0.0664 |
| RENSUP.DAF | 2/11/28 | 3/24/28 | 3/22/28 | 4.24 | 1,479 | 0.04 |

Table 3.20 summarizes the baseline characteristics (age, gender, APACHE II score and medical/surgical diagnosis, severe sepsis upon admittance, septic shock upon admittance) of: (1) Caucasian females with SIRS(N=309), (2) Caucasian males with SIRS(N=545), (3) Caucasian females with severe sepsis (N=225), (4) Caucasian males with severe sepsis (N=421), (5) Caucasian females with septic shock (N=169) and (6) Caucasian males with septic shock (N=312), who were successfully genotyped (GG vs. AA/GA) at rs1861493. For females with severe sepsis and septic shock, a significant difference in age at baseline was detected. For females with SIRS a significant difference in APACHEII score was detected.

TABLE 3.20
Baseline characteristics (age, sex, APACHE II score, medical versus surgical diagnosis,
sepsis upon admittance, septic shock upon admittance) of Caucasian females and Caucasian
males by cohort (i.e. Systemic Inflammatory Response Syndrome (SIRS), sepsis and septic
shock) by genotype rs1861493 (GG vs. AA/GA). Data is reported as percentage for binary
variables and as 25^{th} percentile/median/75^{th} percentile for all other variables.

| | | | | | | F or | | |
|---|---|---|---|---|---|---|---|---|
| | | Baseline | | | | Chi- | | |
| Gender | Cohort | Characteristic | GG | NA/GA | Combined | square | DF | P |
| Female | SIRS | N | 38 | 271 | 309 | | | |
| Female | SIRS | AGE | 55.5/64.5/72 | 43/55/71 | 44.0/58.0/71.0 | 3.21 | 1,307 | 0.0742 |
| Female | SIRS | APACHEII | 19.2/23.5/28 | 14/21/26 | 15.0/22.0/27.0 | 4.22 | 1,307 | 0.0407 |
| Female | SIRS | SURGICAL | 23.7% (9/38) | 24.4% (66/271) | 24.3% (75) | 0.01 | 1 | 0.928 |
| Female | SIRS | SEVSEP. ADMIT | 73.7% (28/38) | 72.7% (197/271) | 72.8% (225) | 0.02 | 1 | 0.898 |
| Female | SIRS | SS. ADMIT | 60.5% (23/38) | 53.9% (146/271) | 54.7% (169) | 0.6 | 1 | 0.44 |
| Female | Severe | N | 28 | 197 | 225 | | | |
| | Sepsis | | | | | | | |
| Female | Severe | AGE | 58.8/65.5/72 | 43/55/70 | 45.0/58.5/70.0 | 5.27 | 1,223 | 0.0227 |
| | Sepsis | | | | | | | |
| Female | Severe | APACHEII | 20.8/25/28 | 16/23/280 | 17.0/23.0/28.0 | 2.55 | 1,223 | 0.112 |
| | Sepsis | | | | | | | |
| Female | Severe | SURGICAL | 21.4% (6/28) | 25.9% (51/197) | 25.3% (57) | 0.26 | 1 | 0.612 |
| | Sepsis | | | | | | | |
| Female | Severe | SS. ADMIT | 82.1% (23/28) | 74.1% (146/197) | 75.1% (169) | 0.85 | 1 | 0.358 |
| | Sepsis | | | | | | | |
| Female | Septic | N | 23 | 146 | 169 | | | |
| | Shock | | | | | | | |
| Female | Septic | AGE | 61.5/67/72 | 45/55/70 | 46.0/59.0/70.0 | 9.8 | 1,167 | 0.00206 |
| | Shock | | | | | | | |
| Female | Septic | APACHEII | 19.5/25/29 | 18.2/23/29 | 19.0/24.0/29.0 | 0.36 | 1,167 | 0.549 |
| | Shock | | | | | | | |
| Female | Septic | SURGICAL | 26.1% (6/23) | 27.4% (40/146) | 27.2% (46) | 0.02 | 1 | 0.896 |
| | Shock | | | | | | | |
| Male | SIRS | N | 49 | 496 | 545 | | | |
| Male | SIRS | AGE | 44/65/73 | 48/59/71 | 47/60/71 | 1.23 | 1,543 | 0.269 |
| Male | SIRS | APACHEII | 16/23/31 | 16/22/28 | 16/22/28 | 0.25 | 1,543 | 0.62 |
| Male | SIRS | SURGICAL | 24.5% (12/49) | 22.4% (111/496) | 22.6% (123) | 0.11 | 1 | 0.736 |
| Male | SIRS | SEVSEP. ADMIT | 69.4% (34/49) | 78.0% (387/496) | 77.2% (421) | 1.89 | 1 | 0.169 |
| Male | SIRS | SS. ADMIT | 57.1% (28/49) | 57.3% (284/496) | 57.2% (312) | 0 | 1 | 0.988 |
| Male | Severe | N | 34 | 387 | 421 | | | |
| | Sepsis | | | | | | | |
| Male | Severe | AGE | 43.2/68/73 | 48/59/71 | 48.0/60.0/71.5 | 0.43 | 1,419 | 0.512 |
| | Sepsis | | | | | | | |
| Male | Severe | APACHEII | 18.2/24.0/31.8 | 18/24/30 | 18.0/23.0/30.0 | 0.85 | 1,419 | 0.356 |
| | Sepsis | | | | | | | |
| Male | Severe | SURGICAL | 23.5% (8) | 23.5% (91/387) | 23.5% (99) | 0 | 1 | 0.998 |
| | Sepsis | | | | | | | |
| Male | Severe | SS.ADMIT | 82.4% (28) | 73.4% (284/387) | 74.1% (312) | 1.31 | 1 | 0.252 |
| | Sepsis | | | | | | | |
| Male | Septic | N | 28 | 284 | 312 | | | |
| | Shock | | | | | | | |
| Male | Septic | AGE | 43.8/69.5/73.5 | 49/63/72 | 49.0/62.0/72.0 | 0.15 | 1,310 | 0.702 |
| | Shock | | | | | | | |
| Male | Septic | APACHEII | 20.0/27.0/33.2 | 20/26/31 | 20.0/26.0/31.0 | 0.49 | 1,310 | 0.483 |
| | Shock | | | | | | | |
| Male | Septic | SURGICAL | 21.4% (6) | 25.7% (73/284) | 25.3 (79) | 0.25 | 1 | 0.62 |
| | Shock | | | | | | | |

Table 3.21 summarizes survival by gender in Caucasian patients with: (1) systematic inflammatory response syndrome (SIRS), (2) severe sepsis and (3) septic shock by genotype group (GG vs. AA/GA) at rs1861493. For females, the GG groups shows significantly decreased survival in the SIRS cohort (P=0.0131), the severe sepsis cohort (P=0.0063) and the septic shock cohort (P=0.00397). For males, the GG group shows significantly decreased survival in the SIRS cohort (P=0.0231).

TABLE 3.21
Survival by genotype at rs1861493 (GG vs. AA/GA) in a cohort of Caucasian patients with
systematic inflammatory response syndrome, sepsis and septic shock in females and males.

| | | | | | Chi- | | |
|---|---|---|---|---|---|---|---|
| Cohort | Gender | GG | AA/GA | Combined | Square | d.f. | P |
| SIRS | Female | 50.0% (19/38) | 70.1% (190/271) | 67.6% (209/309) | 6.16 | 1 | 0.0131 |
| Severe Sepsis | Female | 42.9% (12/28) | 69.0% (136/197) | 65.8% (148/225) | 7.46 | 1 | 0.0063 |
| Septic Shock | Female | 30.4% (7/23) | 62.3% (91/146) | 58.0% (98/169) | 8.3 | 1 | 0.00397 |
| SIRS | Male | 49.0% (24/49) | 65.3% (324/496) | 63.9% (348/545) | 5.16 | 1 | 0.0231 |
| Severe Sepsis | Male | 50.0% (17/34) | 63.8% (247/387) | 62.7% (264/421) | 2.55 | 1 | 0.11 |
| Septic Shock | Male | 46.4% (13/28) | 56.7% (161/284) | 55.8% (174/312) | 1.09 | 1 | 0.297 |

1.2.5 Biological Plausibility Cohort

Table 3.24 summarizes the baseline characteristics (age, gender, smoker, diabetes, hypertension, preoperative ejection fraction, bypass time, cross-clamp time, and aprotinin use) of 24 non-septic SIRS patients who were successfully genotyped (GG vs. AA/GA) at rs1861493. No significant differences between the two genotype groups were detected on admission to the CSICU.

TABLE 3.24
Baseline characteristics of a cohort of non-septic CSICU patients
diagnosed with systematic inflammatory response syndrome by
genotype at rs1861493 (GG vs. AA/GA).

| | GG. | GG. | | AA/GA. | AA/GA. | AA/ |
|---|---|---|---|---|---|---|
| | Mean | Med | GG.SD | Mean | Med | GA.SD |
| AGE | 64 | 63 | 2.3 | 69 | 70 | 8.4 |
| GENDER | 0.67 | 1 | 0.58 | 0.57 | 1 | 0.51 |
| SMOKER | 0 | 0 | 0 | 0.14 | 0 | 0.36 |
| DIABETES | 0 | 0 | 0 | 0.33 | 0 | 0.48 |
| H. TENSE | 0.67 | 1 | 0.58 | 0.52 | 1 | 0.51 |
| EJEC. FRAC | 0.46 | 0.4 | 0.11 | 0.55 | 0.59 | 0.15 |
| BYPASS | 1.1 | 1.1 | 0.18 | 1.8 | 1.7 | 0.63 |
| CLAMP | 0.9 | 0.8 | 0.2 | 1.4 | 1.3 | 0.48 |
| APROTININ | 0 | 0 | 0 | 0 | 0 | 0 |

Table 3.25 summarizes important SNP-biomarker associations. The AA/GA genotype group had significantly higher serum interleukin receptor-1a (IL1ra) levels post-cardiopulmonary bypass (P=0.026), serum interleukin-8 (IL8) levels post-cardiopulmonary bypass (P=0.047), bypass time (P=0.042) and clamp time (P=0.052). These findings suggest that non-septic SIRS patients who carry either the AA or GA genotype rs1861493 are more likely to experience a pro-inflammatory cytokine (IL1ra and IL8) response after cardiopulmonary bypass surgery.

TABLE 3.25
Biological plausibility Interferon Gamma association using biomarkers in a
cohort of non-septic CSICU patients diagnosed with systematic inflammatory
response syndrome by genotype at rs1861493 (GG vs. AA/GA). Data is
reported as 25^{th} percentile/median/75^{th} percentile.

| | | GG | AA/GA | Combined | Test |
|---|---|---|---|---|---|
| | N | (N = 3) | (N = 21) | (N = 24) | Statistic |
| BYPASS | 101 | 1.0/1.1/1.2 | 1.5/1.7/2.1 | 1.3/1.6/ 2.0 | F = 4.7 d.f. = 1,22 P = 0.042 |
| CLAMP | 92 | 0.78/0.80/0.97 | 1.02/1.30/1.67 | 0.92/1.29/1.70 | F = 4.2 d.f. = 1,22 P = 0.052 |
| IL1ra.0 | 96 | 566/659/893 | 1180/1462/2101 | 832/1224/1873 | F = 5.9 d.f. = 1,18 P = 0.026 |
| IL8.3 | 102 | 26/28/31 | 35/52/115 | 28/45/78 | F = 4.4 d.f. = 1,22 P = 0.047 |

1.3 rs2069727

1.3.1 Systematic Inflammatory Response Syndrome—Caucasian Cohort

Table 3.26 summarizes the baseline characteristics (age, gender, APACHE II score, severe sepsis upon admittance, septic shock upon admittance, medical/surgical diagnosis) of 847 Caucasian systematic inflammatory response syndrome patients who were successfully genotyped (AA vs. AG/GG) at rs2069727. A significant difference in APACHEII score was detected between the two genotype groups on admission to the ICU.

TABLE 3.26
Baseline characteristics of a cohort of Caucasian patients who had systematic
inflammatory response syndrome by genotype at rs2069727 (AA vs. AG/GG).
Data is reported as percentage for binary variables and as 25^{th} percentile/
median/75^{th} percentile for all other variables.

| | | | | F or | | |
|---|---|---|---|---|---|---|
| | AA | AG/GG | Combined | Chi- | | |
| | (N = 273) | (N = 574) | (N = 847) | square | d.f. | P |
| AGE | 44/59/71 | 47/59/71 | 46/59/71 | 0.55 | 1,845 | 0.459 |
| SEX | 61.2% (167/265) | 64.8% (372/552) | 63.6% (539/817) | 1.06 | 1 | 0.304 |
| APACHEII | 17/23/28 | 15/21/27 | 16/22/28 | 4.9 | 1,845 | 0.0271 |
| SURGICAL | 20.9% (57/265) | 23.9% (137/552) | 22.9% (194/817) | 0.94 | 1 | 0.333 |
| SEVSEP. AD | 74.7% (204/265) | 76.3% (438/552) | 75.8% (642/817) | 0.25 | 1 | 0.616 |
| MIT | | | | | | |
| SS. ADMIT | 56.0% (153/265) | 56.6% (325/552) | 56.4% (478/817) | 0.02 | 1 | 0.874 |

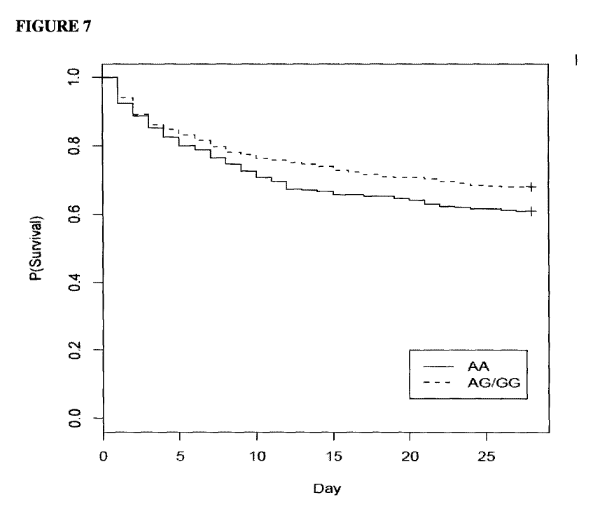

FIG. 7 and Table 3.27 summarizes important SNP-phenotype associations. The AA group showed significantly decreased survival (P=0.0409) and significantly fewer days alive and free of renal dysfunction (P=0.0213), INR>1.5 (P=0.0135), any renal failure (P=0.00142) and renal support (P=0.0046). The AA group also showed a strong trend by fewer days alive and free of SIRS (P=0.088) and 3/4 SIRS criteria (P=0.0954). These findings suggest that Caucasian systematic inflammatory response patients who carry the AA genotype at rs2069727 may be at greater risk of organ dysfunction (renal, coagulation) once admitted to the ICU.

TABLE 3.27
Days alive and free of organ dysfunction (DAF) by genotype of rs2069727
(AA vs. AG/GG) in a cohort of Caucasian patients with systematic inflammatory
response syndrome. Data is reported as percentage for binary variables
and as 25^{th} percentile/median/75^{th} percentile for all other variables.

| | AA | AG/GG | Combined | F or Chi- | | |
|---|---|---|---|---|---|---|
| | (N = 273) | (N = 574) | N = 847) | square | d.f. | P |
| SURV | 60.8% (166/273) | 67.9% (390/574) | 65.6% (556/847) | 4.18 | 1 | 0.0409 |
| DA | 8/28/28 | 13/28/28 | 10/28/28 | 3.68 | 1,845 | 0.0555 |
| RENAL.DAF | 0/5/26 | 0/14.5/27 | 0/7/26 | 5.32 | 1,845 | 0.0213 |
| MSIRS.DAF | 0/8/21 | 1/12/22.8 | 0/11/22 | 2.92 | 1,845 | 0.088 |
| MSIRS3.DAF | 2/17/25 | 4/19/26 | 3/19/26 | 2.79 | 1,845 | 0.0954 |
| INR.DAF | 6/24/28 | 10/26/28 | 7/26/28 | 6.12 | 1,845 | 0.0135 |
| ANYREN.DAF | 0/0/25 | 0/8/26 | 0/1/26 | 10.2 | 1,845 | 0.00142 |
| RENSUP.DAF | 4/24/28 | 7/28/28 | 5.5/28/28 | 8.07 | 1,845 | 0.0046 |

1.3.2 Severe Sepsis—Caucasian Cohort

Table 3.29 summarizes the baseline characteristics (age, gender, APACHE II score, severe septic shock upon admittance and medical/surgical diagnosis) of 642 Caucasian sepsis patients who were successfully genotyped (AA vs. AG/GG) at rs2069727. A significant difference in APACHEII score was detected between the two genotype groups on admission to the ICU.

TABLE 3.29
Baseline characteristics of a cohort of Caucasian patients who had sepsis by genotype of
rs2069727 (AA vs. AG/GG). Data is reported as percentage for binary variables and as 25^{th}
percentile/median/75^{th} percentile for all other variables.

| | | | | F or | | |
|---|---|---|---|---|---|---|
| | AA | AG/GG | Combined | Chi- | | |
| | (N = 204) | (N = 438) | (N = 642) | square | d.f. | P |
| AGE | 44/59/72 | 48/59/71 | 47/59/71 | 0.46 | 1,640 | 0.496 |
| SEX | 61.3% (125/204) | 66.7% (292/438) | 65.0% (417/642) | 1.78 | 1 | 0.182 |
| APACHEII | 18.8/24/30 | 17/23/29 | 18/23/29 | 4.65 | 1,640 | 0.0314 |
| SURGICAL | 20.1% (41/204) | 25.6% (112/438) | 23.8% (153/642) | 2.3 | 1 | 0.13 |
| SS. ADMIT | 75.0% (153/204) | 74.2% (325/438) | 74.5% (478/642) | 0.05 | 1 | 0.829 |

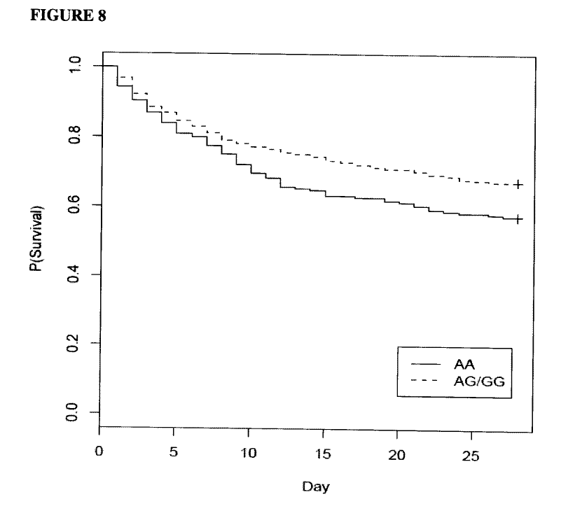

FIG. 8 and Table 3.30 summarizes important SNP-phenotype associations. The AA group showed significantly decreased survival (P=0.0139), significantly fewer days alive (P=0.0187) and significantly fewer days alive and free of: coagulation dysfunction (P=0.0379), acute renal dysfunction (P=0.0307), acute hepatic dysfunction (P=0.0427), 3/4 SIRS criteria (P=0.0455), INR>1.5 (P=0.00424), any renal failure (P=0.00844), renal support (P=0.0037) and any hepatic dysfunction (P=0.0337). AA individuals also showed a strong trend for fewer days alive and free of neurological dysfunction (P=0.0593) and inotropes (P=0.0737), SIRS (P 0.0562). These findings suggest that Caucasian severe sepsis patients who carry the AA genotype at rs2069727 may be at greater risk of organ dysfunction (neurological, coagulation, renal and hepatic) and subject to more use of inotropes once admitted to the ICU.

TABLE 3.30
Days alive and free of organ dysfunction (DAF) by genotype of rs2069727 (AA vs. AG/GG)
in a cohort of Caucasian patients with severe sepsis. Data is reported as percentage for binary
variables and as 25^{th} percentile/median/75^{th} percentile for all other variables.

| | | | | F or | | |
|---|---|---|---|---|---|---|
| | AA | AG/GG | Combined | Chi- | | |
| | (N = 204) | (N = 438) | (N = 642) | square | d.f. | P |
| SURV | 57.4% (117/204) | 67.4% (295/438) | 64.2% (412/642) | 6.05 | 1 | 0.0139 |
| DA | 8.75/28/28 | 14/28/28 | 10/28/28 | 5.56 | 1,640 | 0.0187 |
| CNS.DAF | 2/19/27 | 5/22/28 | 4/22/28 | 3.57 | 1,640 | 0.0593 |
| COAG.DAF | 7/25/28 | 10.2/28/28 | 8/26/28 | 4.33 | 1,640 | 0.0379 |
| RENAL.DAF | 0/3/26 | 0/10.5/26 | 0/4/26 | 4.69 | 1,640 | 0.0307 |
| LIVER.DAF | 7/26/28 | 11/28/28 | 9/28/28 | 4.12 | 1,640 | 0.0427 |
| INO.DAF | 7/28/28 | 12/28/28 | 8/28/28 | 3.21 | 1,640 | 0.0737 |
| MSIRS.DAF | 0/4/19 | 0/9/19 | 0/8/20 | 3.66 | 1,640 | 0.0562 |
| MSIRS3.DAF | 2/15/23 | 4/18/25 | 3/17/24 | 4.01 | 1,640 | 0.0455 |
| INR.DAF | 6.75/22.5/28 | 10/26/28 | 8/25/28 | 8.24 | 1,640 | 0.00424 |
| ANYREN.DAF | 0/0/25 | 0/5/26 | 0/0/26 | 6.98 | 1,640 | 0.00844 |
| RENSUP.DAF | 4/21/28 | 7/28/28 | 6/27/28 | 8.49 | 1,640 | 0.0037 |
| ANYLIVER.DAF | 4/22.5/28 | 6/28/28 | 5/26/28 | 4.53 | 1,640 | 0.0337 |

1.3.3 Septic Shock—Caucasian Cohort

Table 3.31 summarizes the baseline characteristics (age, gender, APACHE II score and medicausurgical diagnosis) of 478 Caucasian septic shock patients who were successfully genotyped (AA vs. AG/GG) at rs2069727. No Significant differences were detected between the two genotype groups on admission to the ICU.

TABLE 3.31
Baseline characteristics of a cohort of Caucasian patients who had septic shock by
genotype of rs2069727 (AA vs. AG/GG). Data is reported as percentage for binary
variables and as 25^{th} percentile/median/75^{th} percentile for all other
variables.

| | | | | F or | | |
|---|---|---|---|---|---|---|
| | AA | AG/GG | Combined | Chi- | | |
| | (N = 153) | (N = 325) | (N = 478) | square | d.f. | P |
| AGE | 48/62/72 | 48/60/72 | 48/60/72 | 0.05 | 1,476 | 0.83 |
| SEX | 59.5% (91/153) | 67.1% (218/325) | 64.6% (309/478) | 2.63 | 1 | 0.105 |
| APACHEII | 20/26/31 | 19/25/30 | 20/25/31 | 3.52 | 1,476 | 0.0611 |
| SURGICAL | 20.9% (32/153) | 27.7% (90/325) | 25.5% (122/478) | 2.51 | 1 | 0.113 |

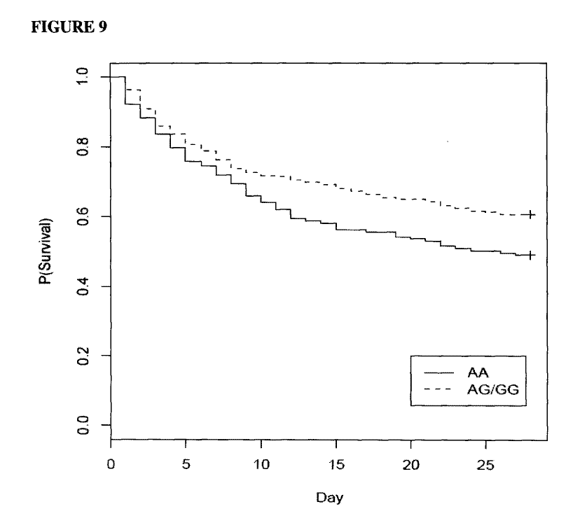

FIG. 9 and Table 3.32 summarizes important SNP-phenotype associations. The AA group showed significantly decreased survival (P=0.0169), significantly fewer days alive (P=0.0246) and significantly fewer days alive and free of coagulation dysfunction (P=0.0251), acute renal dysfunction (P=0.0293), INR>1.5 (P=0.0118), any renal dysfunction (P=0.0156) and renal support (P=0.0122). AA individuals also showed a strong trend for fewer days alive and free of: neurological dysfunction (P=0.0812) and acute hepatic dysfunction (P=0.0625), acute lung injury (P=0.068), use of vasopressors (P=0.0891), use of more than 2 ug/min of vasopressors (P=0.09), use of more than 5 ug/min of vasopressors (P=0.0718), inotropes (P=0.0554), 3/4 SIRS criteria (P=0.0791) and any hepatic dysfunction (P=0.0885). These findings suggest that Caucasian septic shock patients who carry the AA genotype at rs2069727 may be in greater need of vasopressor and steroid therapy and may be at greater risk of organ dysfunction (neurological, coagulation, respiratory, renal and cardiovascular) and are subject to more use of vasopressors and inotropes once admitted to the ICU.

TABLE 3.32
Days alive and free of organ dysfunction (DAF) by genotype at rs2069727 (AA vs. AG/GG)
in a cohort of Caucasian patients with septic shock. Data is reported as percentage for binary
variables and as 25^{th} percentile/median/75^{th} percentile for all other variables.

| | | | | F or | | |
|---|---|---|---|---|---|---|
| | AA | AG/GG | Combined | Chi- | | |
| | (N = 153) | (N = 325) | (N = 478) | square | d.f. | P |
| SURV | 49.0% (75/153) | 60.6% (197/325) | 56.9% (272/478) | 5.7 | 1 | 0.0169 |
| DA | 6/26/28 | 8/28/28 | 7.25/28/28 | 5.08 | 1,476 | 0.0246 |
| CNS.DAF | 1/14/26 | 2/19/26 | 2/18/26 | 3.05 | 1,476 | 0.0812 |
| COAG.DAF | 4/18/28 | 6/25/28 | 5/24/28 | 5.05 | 1,476 | 0.0251 |
| RENAL.DAF | 0/0/23 | 0/3/26 | 0/0/25 | 4.78 | 1,476 | 0.0293 |
| LIVER.DAF | 5/22/28 | 7/26/28 | 6/26/28 | 3.48 | 1,476 | 0.0625 |
| ALI.DAF | 4/15/26 | 6/21/28 | 5/20/28 | 3.35 | 1,476 | 0.068 |
| PRESS.DAF | 3/14/25 | 2/21/26 | 2/20/26 | 2.9 | 1,476 | 0.0891 |
| PRESS2.DAF | 3/15/26 | 2/21/26 | 2/20/26 | 2.89 | 1,476 | 0.09 |
| PRESS5.DAF | 3/16/26 | 3/22/27 | 3/22/26 | 3.26 | 1,476 | 0.0718 |
| INO.DAF | 5/22/28 | 7/27/28 | 5/25.5/28 | 3.69 | 1,476 | 0.0554 |
| MSIRS3.DAF | 1/10/21 | 2/13/23 | 2/12/23 | 3.1 | 1,476 | 0.0791 |
| INR.DAF | 4/19/27 | 6/25/28 | 5/22.5/28 | 6.39 | 1,476 | 0.0118 |
| ANYREN.DAF | 0/0/12 | 0/0/26 | 0/0/25 | 5.89 | 1,476 | 0.0156 |
| RENSUP.DAF | 3/13/28 | 4/26/28 | 3/22/28 | 6.33 | 1,476 | 0.0122 |
| ANYLIVER.DAF | 3/15/28 | 4/24/28 | 3/23/28 | 2.91 | 1,476 | 0.0885 |

Table 3.33 summarizes the baseline characteristics (age, gender, APACHE II score and medical/surgical diagnosis, severe sepsis upon admittance, septic shock upon admittance) of: (1) Caucasian females with SIRS(N=308), (2) Caucasian males with SIRS(N=539), (3) Caucasian females with severe sepsis (N=225), (4) Caucasian males with severe sepsis (N=417), (5) Caucasian females with septic shock (N=169) and (6) Caucasian males with septic shock (N=309), who were successfully genotyped (GG vs. AA/GT) at rs1861493. A significant difference in APACHEII score was detected at baseline for females with SIRS.

TABLE 3.33
Baseline characteristics (age, sex, APACHE II score, medical versus surgical diagnosis,
sepsis upon admittance, septic shock upon admittance) of Caucasian females and Caucasian
males by cohort (i.e. Systemic Inflammatory Response Syndrome (SIRS), severe sepsis and
septic shock) by genotype rs2069727 (AA vs. AG/GG). Data is reported as percentage for
binary variables and as 25^{th} percentile/median/75^{th} percentile for all other variables.

| | | | | | | F or | | |
|---|---|---|---|---|---|---|---|---|
| | | Baseline | | | | Chi- | | |
| Gender | Cohort | Characteristic | AA | AG/GG | Combined | square | DF | P |
| Female | SIRS | N | 106 | 202 | 308 | Statistic | | |
| Female | SIRS | AGE | 43/59/72 | 44.2/57.5/71 | 44/58/71 | 0.15 | 1,306 | 0.698 |
| Female | SIRS | APACHEII | 17.2/22.5/27.8 | 14/20.5/25 | 15/22/27 | 4.45 | 1,306 | 0.0358 |
| Female | SIRS | SURGICAL | 20.8% (22/106) | 25.2% (51/202) | 23.7% (73/308) | 0.78 | 1 | 0.378 |
| Female | SIRS | SEVSEP.ADMIT | 74.5% (79/106) | 72.3% (146/202) | 73.1% (225/308) | 0.18 | 1 | 0.672 |
| Female | SIRS | SS.ADMIT | 58.5% (62/106) | 53.0% (107202) | 54.9% (169/308) | 0.86 | 1 | 0.355 |
| Female | Severe | N | 79 | 146 | 225 | | | |
| | Sepsis | | | | | | | |
| Female | Severe | AGE | 43.5/61/72 | 45/55.5/70 | 45/58.5/70 | 0.07 | 1,223 | 0.79 |
| | Sepsis | | | | | | | |
| Female | Severe | APACHEII | 18.5/23/28 | 15.2/22/27.8 | 17/23/28 | 2.72 | 1,223 | 0.101 |
| | Sepsis | | | | | | | |
| Female | Severe | SURGICAL | 19% (15/79) | 28.1% (41/146) | 24.9% (56/225) | 2.27 | 1 | 0.132 |
| | Sepsis | | | | | | | |
| Female | Severe | SS.ADMIT | 78.5% (62/79) | 73.3% (107/146) | 75.1% (169/225) | 0.74 | 1 | 0.39 |
| | Sepsis | | | | | | | |
| Female | Septic | N | 62 | 107 | 169 | | | |
| | Shock | | | | | | | |
| Female | Septic | AGE | 49/62/72 | 45/55/70 | 46/59/70 | 1.74 | 1,167 | 0.189 |
| | Shock | | | | | | | |
| Female | Septic | APACHEII | 20/24/30 | 17/23/29 | 19/24/29 | 1.45 | 1,167 | 0.23 |
| | Shock | | | | | | | |
| Female | Septic | SURGICAL | 21.0% (13/62) | 29.9% (32/107) | 26.6% (45/169) | 1.61 | 1 | 0.205 |
| | Shock | | | | | | | |
| Male | SIRS | N | 167 | 372 | 539 | | | |
| Male | SIRS | AGE | 46/59/71 | 48/60/71 | 47/60/71 | 0.39 | 1,537 | 0.531 |
| Male | SIRS | APACHEII | 17/23/29 | 16/21/28 | 16/22/28 | 1.4 | 1,537 | 0.237 |
| Male | SIRS | SURGICAL | 21.0% (35/167) | 23.1% (86/372) | 22.4% (121/539) | 0.31 | 1 | 0.578 |
| Male | SIRS | SEVSEP.ADMIT | 74.9% (125/167) | 78.5% (292/372) | 77.4% (417/539) | 0.87 | 1 | 0.35 |
| Male | SIRS | SS.ADMIT | 54.5% (91/167) | 58.6% (218/372) | 57.3% (309/539) | 0.8 | 1 | 0.372 |
| Male | Severe | N | 125 | 292 | 417 | | | |
| | Sepsis | | | | | | | |
| Male | Severe | AGE | 45/58/71 | 48.8/60.5/72 | 48/60/71.5 | 1.04 | 1,415 | 0.309 |
| | Sepsis | | | | | | | |
| Male | Severe | APACHEII | 19/24/31 | 18/23/29.2 | 18/23/30 | 2.23 | 1,415 | 0.136 |
| | Sepsis | | | | | | | |
| Male | Severe | SURGICAL | 20.8% (26/125) | 24.3% (71/292) | 23.3% (97/417) | 0.61 | 1 | 0.436 |
| | Sepsis | | | | | | | |
| Male | Severe | SS.ADMIT | 72.8% (91/125) | 74.7% (218)/292 | 74.1% (309/417) | 0.16 | 1 | 0.692 |
| | Sepsis | | | | | | | |
| Male | Septic | N | 91 | 218 | 309 | | | |
| | Shock | | | | | | | |
| Male | Septic | AGE | 46.5/62/72 | 49/63.5/72 | 49/62/72 | 0.39 | 1,307 | 0.534 |
| | Shock | | | | | | | |
| Male | Septic | APACHEII | 21/27/32 | 19/25/31 | 20/26/31 | 2.61 | 1,307 | 0.107 |
| | Shock | | | | | | | |
| Male | Septic | SURGICAL | 20.9% (19/91) | 26.6% (58/218) | 24.9% (77/309) | 1.13 | 1 | 0.289 |
| Male | Shock | | | | | | | |

Table 3.34 summarizes survival by gender in Caucasian patients with: (1) systematic inflammatory response syndrome (SIRS), (2) severe sepsis and (3) septic shock by genotype group (AA vs. AG/GG) at rs2069727. For females, the AA groups shows significantly decreased survival in the SIRS cohort (P=0.00501), the severe sepsis cohort (P=0.00832) and the septic shock cohort (P=0.0101). In contrast, there were no significant differences in survival between genotype groups for males. Using logistic regression with genotype, gender and genotype*gender interaction terms, there is a strong trend towards a significant genotype*gender interaction at rs2069727 (P=0.0556).

TABLE 3.34
Survival by genotype at rs2069727 (AA vs. AG/GG) in a cohort of Caucasian patients with
systematic inflammatory response syndrome, severe sepsis and septic shock in females and
males.

| | | | | | Chi- | | |
|---|---|---|---|---|---|---|---|
| Cohort | Gender | AA | AG/GG | Combined | square | d.f. | P |
| SIRS | Female | 57.5% (61/106) | 73.3% (148/202) | 67.9% (209/308) | 7.88 | 1 | 0.00501 |
| Severe Sepsis | Female | 54.4% (43/79) | 71.9% (105/146) | 65.8% (148/225) | 6.96 | 1 | 0.00832 |
| Septic Shock | Female | 45.2% (28/62) | 65.4% (70/107) | 58.0% (98/169) | 6.61 | 1 | 0.0101 |
| SIRS | Male | 62.9% (105/167) | 65.1% (242/372) | 64.4% (347/539) | 0.24 | 1 | 0.625 |
| Severe Sepsis | Male | 59.2% (74/125) | 65.1% (190/292) | 63.3% (264/417) | 1.3 | 1 | 0.255 |
| Septic Shock | Male | 51.6% (47/91) | 58.3% (127/218) | 56.3% (174/309) | 1.14 | 1 | 0.286 |

1.3.5 Biological Plausibility Cohort

Table 3.37 summarizes the baseline characteristics (age, gender, smoker, diabetes, hypertension, preoperative ejection fraction, bypass time, cross-clamp time, and aprotinin use) of 61 non-septic SIRS patients who were successfully genotyped (AA vs. AG/GG) at rs2069727. No significant differences between the two genotype groups were detected on admission to the CSICU.

TABLE 3.37
Baseline characteristics of a cohort of non-septic CSICU patients
diagnosed with systematic inflammatory response syndrome by
genotype at rs2069727 (AA vs. AG/GG).

| | AA.Mean | AA.Med | AA.SD | AG/GG.Mean | AG/GG.Med | AG/GG.SD |
|---|---|---|---|---|---|---|
| AGE | 67 | 69 | 8.2 | 65 | 65 | 8.2 |
| GENDER | 0.63 | 1 | 0.5 | 0.67 | 1 | 0.48 |
| SMOKER | 0.26 | 0 | 0.45 | 0.17 | 0 | 0.38 |
| DIABETES | 0.21 | 0 | 0.42 | 0.33 | 0 | 0.48 |
| H. TENSE | 0.58 | 1 | 0.51 | 0.57 | 1 | 0.5 |
| EJEC. FRAC | 0.48 | 0.5 | 0.13 | 0.53 | 0.55 | 0.11 |
| BYPASS | 1.7 | 1.6 | 0.65 | 1.7 | 1.7 | 0.58 |
| CLAMP | 1.3 | 1.1 | 0.57 | 1.3 | 1.3 | 0.48 |
| APROTININ | 0.105 | 0 | 0.32 | 0.048 | 0 | 0.22 |

Table 3.38 summarizes important SNP-biomarker associations. The AG/GG genotype group had significantly higher serum interleukin receptor-1a (IL1ra) levels post-cardiopulmonary bypass (P=0.0084), serum interleukin-8 (IL8) levels post-cardiopulmonary bypass (P=0.028), and a strong trend for higher serum monocyte chemoattractant protein (MCP) levels post-cardiopulmonary bypass (P=0.073). These findings suggest that non-septic SIRS patients who carry either the AG or GG genotype rs2069727 are more likely to experience a pro-inflammatory cytokine (IL1ra IL8 and MCP) response after cardiopulmonary bypass surgery.

TABLE 3.38
Biological plausibility Interferon Gamma association using biomarkers in a cohort of non-
septic CSICU patients diagnosed with systematic inflammatory response syndrome by
genotype at rs2069727 (AA vs. AG/GG). Data is reported as 25^{th} percentile/median/75^{th}
percentile

| | AA | AG/GG | Combined | Test |
|---|---|---|---|---|
| | (N = 19) | (N = 42) | (N = 61) | Statistic |
| IL1ra.0 | 682/1125/1315 | 1176/1463/2028 | 832/1224/1873 | F = 7.5 d.f. = 1, 55 P = 0.0084 |
| IL8.3 | 26/34/51 | 34/64/120 | 28/45/78 | F = 5.1 d.f. = 1, 59 P = 0.028 |
| IL8.DIF | 20/28/42 | 27/48/93 | 22/36/67 | F = 4.1 d.f = 1, 59 P = 0.047 |
| MCP.3 | 335/485/761 | 418/733/1627 | 364/597/1215 | F = 3.3 d.f. = 1, 59 P = 0.073 |

### REFERENCES

1. Billiau A, Heremans H, Vermeire K, Matthys P. Immunomodulatory properties of interferon-gamma. An update. Ann N Y Acad Sci. 1998; 856:22-32.
2. Dinarello C A. Proinflammatory cytokines. Chest. 2000; 118(2):503-8.
3. Lainee P, Efron P, Tschoeke S K, Elies L, De Winter H, LOrre K, Moldawer L L. Delayed neutralization of interferon-gamma prevents lethality in primate Gram-negative bacteremic shock. Crit Care Med. 2005; 33(4):797-805.
4. Rigato O, Salomao R. Impaired production of interferon-gamma and tumor necrosis factor-alpha but not of interleukin 10 in whole blood of patients with sepsis. Shock. 2003; 19(2):113-6.
5. Kox W J, Bone R C, Krausch D, Docke W D, Kox S N, Wauer H, Egerer K, Querner S, Asadullah K, von Baehr R, Volk H D. Interferon gamma-1b in the treatment of compensatory anti-inflammatory response syndrome. A new approach: proof of principle. Arch Intern Med. 1997; 157(4):389-93.
6. Docke W D, Randow F, Syrbe U, Krausch D, Asadullah K, Reinke P, Volk H D, Kox W. Monocyte deactivation in septic patients: restoration by IFN-gamma treatment. Nat Med. 1997; 3(6):678-81.
7. Hotchkiss R S, Chang K C, Grayson M H, Tinsley K W, Dunne B S, Davis C G, Osborne D F, Karl I E. Adoptive transfer of apoptotic splenocytes worsens survival, whereas adoptive transfer of necrotic splenocytes improves survival in sepsis. Proc Natl Acad Sci USA. 2003; 100(11):6724-9.
8. Yin K, Hock C E, Lai P S, Ross J T, Yue G. Role of interferon-gamma in lung inflammation following cecal ligation and puncture in rats. Shock. 1999; 12(3):215-21.
9. Zisman D A, Kunkel S L, Strieter R M, Gauldie J, Tsai W C, Bramson J, Wilkowski J M, Bucknell K A, Standiford T J. Anti-interleukin-12 therapy protects mice in lethal endotoxemia but impairs bacterial clearance in murine Escherichia coli peritoneal sepsis. Shock. 1997; 8(5):349-56.
10. Redmond H P, Chavin K D, Bromberg J S, Daly J M. Inhibition of macrophage-activating cytokines is beneficial in the acute septic response. Ann Surg. 1991; 214(4):502-8; discussion 508-9.
11. Cavet J, Dickinson A M, Norden J, Taylor P R, Jackson G H, Middleton P G. Interferon-gamma and interleukin-6 gene polymorphisms associate with graft-versus-host disease in HLA-matched sibling bone marrow transplantation. Blood. 2001 98(5): 1594-600.
12. Awad M, Pravica V, Perrey C, et al. CA repeat allele polymorphism in the first intron of the human interferon-gamma gene is associated with lung allograft fibrosis. Hum Immunol. 1999 60(4):343-6.
13. Wu J M, Bensen-Kennedy D, Miura Y, Thoburn C J, Armstrong D, Vogelsang G B, Hess A D. The effects of interleukin 10 and interferon gamma cytokine gene polymorphisms on survival after autologous bone marrow transplantation for patients with breast cancer. Biol Blood Marrow Transplant. 2005 11(6):455-64.
14. Kamali-Sarvestani E, Merat A, Talei A R. Polymorphism in the genes of alpha and beta tumor necrosis factors (TNF-alpha and TNF-beta) and gamma interferon (IFN-gamma) among Iranian women with breast cancer. Cancer Lett. 2005 223(1):113-9.
15. Lu K C, Jaramillo A, Lecha R L, Schuessler R B, Aloush A, Trulock E P, Mendeloff E N, Huddleston C B, Alexander Patterson G, Mohanakumar T. Interleukin-6 and interferon gamma gene polymorphisms in the development of bronchiolitis obliterans syndrome after lung transplantation. Transplantation. 2002 74(9):1297-302.
16. Bravo M J, de Dios Colmenero J, Alonso A, Caballero A. Polymorphisms of the interferon gamma and interleukin 10 genes in human brucellosis. Eur J Immunogenet. 2003 (6):433-5.
17. Cabantous S, Poudiougou B, Traore A, Keita M, Cisse M B, Doumbo O, Dessein A J, Marquet S. Evidence that interferon-gamma plays a protective role during cerebral malaria. J Infect Dis. 2005 192(5):854-60.
18. Lio D, Scola L, Forte G I, Accomando S, Giacalone A, Crivello A, Cataldo F. TNFalpha, IFNgamma and IL-10 gene polymorphisms in a sample of Sicilian patients with coeliac disease. Dig Liver Dis. 2005 37(10):756-60.
19. Tinckam K, Rush D, Hutchinson I, Dembinski I, Pravica V, Jeffery J, Nickerson P. The relative importance of cytokine gene polymorphisms in the development of early and late acute rejection and six-month renal allograft pathology. Transplantation. 2005 79(7):836-41.
20. Kitawaki J, Koshiba H, Kitaoka Y, Teramoto M, Hasegawa G, Nakamura N, Yoshikawa T, Ohta M, Obayashi H, Honjo H. Interferon-gamma gene dinucleotide (CA) repeat and interleukin-4 promoter region (−590C/T) polymorphisms in Japanese patients with endometriosis. Hum Reprod. 2004 19(8):1765-9.
21. Stanford M R, Vaughan R W, Kondeatis E, Chen Y, Edelsten C E, Graham E M, Wallace G R. Are cytokine gene polymorphisms associated with outcome in patients with idiopathic intermediate uveitis in the United Kingdom? Br J Opthalmol. 2005 89(8): 1013-6.
22. Masutani K, Miyake K, Nakashima H, Hirano T, Kubo M, Hirakawa M, Tsuruya K, Fukuda K, Kanai H, Otsuka T, Hirakata H, Iida M. Impact of interferon-gamma and interleukin-4 gene polymorphisms on development and progression of IgA nephropathy in Japanese patients. Am J Kidney Dis. 2003 41(2):371-9.
23. Yu H, Zhu Q R, Gu S Q, Fei L E, Pu D B. Association of interferon-gamma+874 gene single nucleotide polymorphism with susceptibility to intrauterine HBV infection Zhonghua Er Ke Za Zhi. 2004 42(6):421-3.
24. Liu D, O'Day S J, Yang D, Boasberg P, Milford R, Kristedja T, Groshen S, Weber J. Impact of gene polymorphisms on clinical outcome for stage IV melanoma patients treated with biochemotherapy: an exploratory study. Clin Cancer Res. 2005 11(3): 1237-46.
25. Kantarci O H, Goris A, Hebrink D D, Heggarty S, Cunningham S, Alloza I, Atkinson E J, de Andrade M, McMurray C T, Graham C A, Hawkins S A, Billiau A, Dubois B, Weinshenker B G, Vandenbroeck K. IFNG polymorphisms are associated with gender differences in susceptibility to multiple sclerosis. Genes Immun. 2005 6(2):153-61.
26. Carrozzo M, Uboldi de Capei M, Dametto E, Fasano M E, Arduino P, Broccoletti R, Vezza D, Rendine S, Curtoni E S, Gandolfo S. Tumor necrosis factor-alpha and interferon-gamma polymorphisms contribute to susceptibility to oral lichen planus. J Invest Dermatol. 2004 122(1):87-94. Erratum in: J Invest Dermatol. 2004 123(4):805.
27. Halma M A, Wheelhouse N M, Barber M D, Powell J J, Fearon K C, Ross J A. Interferon-gamma polymorphisms correlate with duration of survival in pancreatic cancer. Hum Immunol. 2004 65(11):1405-8.
28. Lopez-Maderuelo D, Arnalich F, Serantes R, Gonzalez A, Codoceo R, Madero R, Vazquez J J, Montiel C. Interferon-gamma and interleukin-10 gene polymorphisms in pulmonary tuberculosis. Am J Respir Crit Care Med. 2003 167(7):970-5.
29. Stassen N A, Leslie-Norfleet L A, Robertson A M, Eichenberger M R, Polk H C Jr. Interferon-gamma gene polymorphisms and the development of sepsis in patients with trauma. Surgery. 2002 132(2):289-92.
30. Chevillard C, Moukoko C E, Elwali N E, Bream J H, Kouriba B, Argiro L, Rahoud S, Mergani A, Henri S, Gaudart J, Mohamed-Ali Q, Young H A, Dessein A J. IFN-gamma polymorphisms (IFN-gamma+2109 and IFN-gamma+3810) are associated with severe hepatic fibrosis in human hepatic schistosomiasis (Schistosoma mansoni). J Immunol. 2003 171(10):5596-601.
31. Khani-Hanjani A, Lacaille D, Hoar D, Chalmers A, Horsman D, Anderson M, Balshaw R, Keown P A. Association between dinucleotide repeat in non-coding region of interferon-gamma gene and susceptibility to, and severity of, rheumatoid arthritis. Lancet. 2000 356(9232):820-5.
32. Natividad A, Wilson J, Koch O, Holland M J, Rockett K, Faal N, Jallow O, Joof H M, Burton M J, Alexander N D, Kwiatkowski D P, Mabey D C, Bailey R L. Risk of trachomatous scarring and trichiasis in Gambians varies with SNP haplotypes at the interferon-gamma and interleukin-10 loci. Genes Immun. 2005 6(4):332-40.
33. Tso H W, Ip W K, Chong W P, Tam C M, Chiang A K, Lau Y L. Association of interferon gamma and interleukin 10 genes with tuberculosis in Hong Kong Chinese. Genes Immun. 2005 6(4):358-63.
34. Jahromi M, Millward A, Demaine A. A CA repeat polymorphism of the IFN-gamma gene is associated with susceptibility to type 1 diabetes. J Interferon Cytokine Res. 2000 20(2): 187-90.
35. Spriewald B M, Witzke O, Wassmuth R, Wenzel R R, Arnold M L, Philipp T, Kalden J R. Distinct tumour necrosis factor alpha, interferon gamma, interleukin 10, and cytotoxic T cell antigen 4 gene polymorphisms in disease occurrence and end stage renal disease in Wegener's granulomatosis. Ann Rheum Dis. 2005 64(3):457-61.
36. Gray P W and Goeddel, D V. Structure of the human immune interferon gene. Nature. 1982 298, 859-863.
37. Pravica V, Asderakis A, Perrey C, Hajeer A, Sinnott P J, Hutchinson I V. In vitro production of IFN-gamma correlates with CA repeat polymorphism in the human IFN-gamma gene. Eur J Immunogenet. 1999 26, 1-3.
38. Pravica V, Perrey C, Stevens A, Lee J H, Hutchinson I V. A single nucleotide polymorphism in the first intron of the human IFN-gamma gene: absolute correlation with a polymorphic CA microsatellite marker of high IFN-gamma production. Hum Immunol. 2000 61, 863-866.
39. Koch O, Rockett K, Jallow M, Pinder M, Sisay-Joff F, Kwiakowski D. Investigation of malaria susceptibility determinants in the IFNG/IL26/IL22 genomic region. Genes Immun. 2005 6, 312-318.
40. R Development Core Team (www.R-project.org). R: A language and environment for statistical computing. Vienna, Austria. 2005.
41. Gordon A C, Waheed U, Hansen T K, Hitman G A, Garrard C S, Turner M W, Klein N J, Brett S J, Hinds C J. Mannose-binding lectin polymorphisms in severe sepsis; relationship to levels, incidence and outcome Shock 2006; 25 (1) 88-93.
42. Westendorp R G J, Hottenga J, Slagboom P E. Variation in plasminogen-activator-inhibitor-1 gene and risk of meningococcal septic shock. Lancet 1999; 354: 561-63
43. Manocha S, Walley K R, Holmes C L, Russell J A. TNFα +252 A: TNFα −308 G haplotype has a different effect on outcome in patients with SIRS, sepsis and septic shock. Critical Care Medicine 2003; 31(12 Supplement):A3.
44. Bone R C. Sir Isaac Newton, sepsis, SIRS, and CARS. Critical Care Medicine 1996; 24:1125-1128

1. A method for obtaining a prognosis for a subject having, or at risk of developing, an inflammatory condition, the method comprising
determining a genotype of said subject which includes one or more polymorphic sites in the subject's interferon gamma (IFNG) gene sequence selected from one or more of the following: rs1861493; rs2069718; and rs2069727 or one or more polymorphic sites in linkage disequilibrium thereto, selected from one or more of the following: rs2069705; rs2069733; rs10467155; rs7973244; rs7137993; rs12315837; rs4913277; rs2080414; rs7956817; rs2069718; rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763; rs2193046; rs1861493; rs10878774; rs10878786; rs10878784; rs971545; rs12301088; rs7969024; rs1177081; rs12317232; rs1177083; rs10878766; rs7969592; rs10878781; rs2870950; and rs10492197,
which genotype is indicative of the subject's ability to recover from the inflammatory condition.

2. (canceled)

3. The method of claim 1, further comprising comparing the genotype so determined with known genotypes which are known to be indicative of a prognosis for recovery from:
(i) the subject's type of inflammatory condition; or
(ii) another inflammatory condition.

4. The method of claim 1, further comprising obtaining IFNG gene sequence information for the subject.

5. The method of claim 1, wherein the genotype is determined using a nucleic acid sample from the subject.

6. The method of claim 5, further comprising the step of obtaining the nucleic acid sample from the subject.

7. The method of claim 1, wherein said genotype is determined using one or more of the following techniques:
(a) restriction fragment length analysis;
(b) sequencing;
(c) a micro-sequencing assay;
(d) hybridization;
(e) invader assay;
(f) a gene chip hybridization assay;
(g) oligonucleotide ligation assay;
(h) ligation rolling circle amplification;
(i) 5′ nuclease assay;
(j) a polymerase proofreading method;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

8. The method of claim 1, wherein the prognosis is increased risk of death or organ dysfunction from the inflammatory condition, or severe cardiovascular or respiratory dysfunction.

9. (canceled)

10. The method of claim 8, wherein the genotype comprises at least one of the following risk genotypes: rs1861493G; rs2069718T; and rs2069727A.

11. The method of claim 1, wherein the prognosis is a prognosis of decreased risk of death or organ dysfunction from the inflammatory condition, or of mild cardiovascular or respiratory dysfunction.

12. (canceled)

13. The method of claim 11, wherein the genotype comprises at least one of the following reduced risk genotypes: rs1861493A; rs2069718C; and rs2069727G.

14. The method of claim 1, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, a reaction to an ingested, inhaled, infused, injected, or delivered substance, glomerulonephritis, bowel infection, an opportunistic infections, an inflammatory response due to major surgery transplant or dialysis leading to an immunocompromised state treatment with an immunosuppressive agent, HIV/AIDS, endocarditis, fever, cystic fibrosis, diabetes mellitus, chronic renal failure, bronchiectasis, chronic obstructive lung disease, chronic bronchitis, emphysema, asthma, febrile neutropenia, meningitis, septic arthritis, urinary tract infection, necrotizing fasciitis, Group A streptococcus infection, splenectomy, recurrent or suspected enterococcus infection, other medical and surgical conditions associated with increased risk of infection: Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism and venous thrombosis, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella infection, Lyme disease, Influenza A infection, Epstein-Barr virus infection, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, graft-versus-host disease, transplant rejection, sickle cell anemia, nephrotic syndrome, toxicity of OKT3 therapy or cytokine therapy, and cirrhosis.

15. The method of claim 1, wherein the inflammatory condition is SIRS, sepsis, or septic shock.

16-17. (canceled)

18. A method for identifying a subject having an improved response genotype or an adverse response genotype in an interferon gamma (IFNG) gene sequence, the method comprising determining a genotype of said subject at one or more polymorphic sites in the subject's IFNG gene sequence, wherein said genotype is indicative of the subject's response to administration of activated protein C or a protein C-like compound wherein
(a) the improved response genotype is rs2069718C or one or more polymorphic sites in linkage disequilibrium thereto,
(b) the adverse response genotype is rs2069718T or one or more polymorphic sites in linkage disequilibrium thereto,
(c) the one or more polymorphic sites in linkage disequilibrium thereto is one or more of: rs2069705; rs2069733; rs2193046; rs741344; rs4913405; rs759488; rs4913418; rs10748099; rs10784688; rs2193050; rs7959933; rs7302226; rs4913415; rs10784684; rs1861493; rs7302488; rs759487; rs4913278; rs2216163; rs7132697; rs7133554; rs2111059; rs10878763; rs10784683; rs6581795; rs6581794; rs7138107; rs1118866; rs2098394; rs10878779; rs2193049; rs9888400; rs2870952; rs2193048; rs2870953; rs3181034; rs10467155; rs1861494; rs2193045; rs7973244; rs2870951; rs2193047; rs7137993; rs12315837; rs1076025; rs12312186; rs7137814; rs2080414; rs7956817; rs9888319; rs7298410; rs4913277; rs2058739; rs2216164; and rs2041864.

19-24. (canceled)

25. The method of claim 18, wherein the genotype is determined using a nucleic acid sample obtained from the subject.

26. The method of claim 25, further comprising a step of obtaining the nucleic acid sample from the subject.

27. The method of claim 18, wherein said genotype is determined using one or more of the following techniques:
(a) restriction fragment length analysis;
(b) sequencing;
(c) a micro-sequencing assay;
(d) hybridization;
(e) invader assay;
(f) a gene chip hybridization assay;
(g) oligonucleotide ligation assay;
(h) ligation rolling circle amplification;
(i) 5′ nuclease assay;
(j) a polymerase proofreading method;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

28. (canceled)

29. The method of claim 18, wherein the subject is critically ill with an inflammatory condition.

30. The method of claim 18, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, a reaction to an ingested, inhaled, infused, injected, or delivered substance, glomerulonephritis, bowel infection, an opportunistic infections, an inflammatory response due to major surgery transplant or dialysis leading to an immunocompromised state, treatment with an immunosuppressive agent, HIV/AIDS, endocarditis, fever, cystic fibrosis, diabetes mellitus, chronic renal failure, bronchiectasis, chronic obstructive lung disease, chronic bronchitis, emphysema, asthma, febrile neutropenia, meningitis, septic arthritis, urinary tract infection, necrotizing fasciitis, Group A streptococcus infection, splenectomy, recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism and venous thrombosis, mycobacterial tuberculosis, Pneumocystis carinii, pneumonia, Leishmaniasis, hemolytic uremic syndrome/thrombotic thrombocytopenic purpura, Dengue hemorrhagic fever, pelvic inflammatory disease, Legionella infection, Lyme disease, Influenza A infection, Epstein-Barr virus infection, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, graft-versus-host disease, transplant rejection, sickle cell anemia, nephrotic syndrome, toxicity of OKT3 therapy or cytokine therapy, and cirrhosis.

31. (canceled)

32. The method of claim 18, wherein a subject having one or more improved response genotype(s) in his IFNG gene sequences is selectively administered activated protein C or a protein C-like compound.

33. The method of claim 18, wherein a subject having one or more adverse response genotype(s) in their IFNG gene sequences selectively is not administered activated protein C or a protein C-like compound.

34. A kit for determining a genotype at a defined nucleotide position within a polymorphic site in a IFNG gene sequence in a subject to predict a subject's response to activated protein C or protein C-like compound administration, the kit comprising:
(a) a restriction enzyme capable of distinguishing alternate nucleotides at the polymorphic site; or
(b) a labeled oligonucleotide having sufficient complementary to the polymorphic site so as to be capable of hybridizing distinctively to said alternate nucleotide, wherein the polymorphic site is one or more of the following: rs1861493: rs2069718; rs2069727; rs2069705; rs2069733; rs10467155; rs7973244, rs7137993; rs12315837; rs4913277; rs2080414; rs7956817; rs2069718, rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763: rs2193046; rs1861493; rs10878774; rs10878786; rs10878784; rs971545; rs12301088; rs7969024; rs1177081; rs12317232; rs1177083; rs10878766; rs7969592; rs10878781; rs2870950; and rs10492197.

35. (canceled)

36. The kit of claim 34, further comprising an oligonucleotide or a set of oligonucleotides operable to amplify a region including the polymorphic site.

37. The kit of claim 34, further comprising a polymerization agent.

38. The kit of claim 34, further comprising instructions for using the kit to determine genotype.

39. A method for selecting a group of subjects for determining the efficacy of a candidate drug known or suspected of being useful for the treatment of an inflammatory condition, the method comprising
(a) determining a genotype at one or more polymorphic sites in a IFNG gene sequence for each subject, wherein said genotype is indicative of the subject's response to the candidate drug, and
(b) sorting subjects based on their genotype.

40. The method of claim 39 further comprising, administering the candidate drug to the subjects or a subset of subjects and determining each subject's ability to recover from the inflammatory condition.

41. The method of claim 40, further comprising comparing the subject's response to the candidate drug based on genotype of the subject.

42. (canceled)

43. A method of treating an inflammatory condition in a subject in need thereof:
(a) selecting a subject having an improved response genotype in his IFNG gene sequence; and
(b) administering to said subject activated protein C or protein C-like compound.

44.-50. (canceled)

51. The method of claim 43, further comprising determining the subject's APACHE II score as an assessment of subject risk.

52. The method of claim 43, further comprising determining the number of organ system failures for the subject as an assessment of subject risk.

53. The method of claim 51, wherein an APACHE II score≧25 is indicative of an increased risk.

54. The method of claim 52, wherein two or more organ system failures are indicative of increased risk.

55. The method of claim 43, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, a reaction to an ingested, inhaled, infused, injected, or delivered substance, glomerulonephritis, bowel infection, an opportunistic infections, an inflammatory response due to major surgery transplant or dialysis leading to an immunocompromised state treatment with an immunosuppressive agent, HIV/AIDS, endocarditis, fever, cystic fibrosis, diabetes mellitus, chronic renal failure, bronchiectasis, chronic obstructive lung disease, chronic bronchitis, emphysema, asthma, febrile neutropenia, meningitis, septic arthritis, urinary tract infection, necrotizing fasciitis, Group A streptococcus infection, splenectomy, recurrent or suspected enterococcus infection, other medical and surgical conditions associated with increased risk of infection: Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism and venous thrombosis, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella infection, Lyme disease, Influenza A infection, Epstein-Barr virus infection, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, graft-versus-host disease, transplant rejection, sickle cell anemia, nephrotic syndrome, toxicity of OKT3 therapy or cytokine therapy, and cirrhosis.

56. The method of claim 43, wherein the inflammatory condition is selected from systemic inflammatory response syndrome (SIRS), sepsis, and septic shock.

57.-58. (canceled)

59. The method of claim 43 of claim, wherein the polymorphic site is one or more of the following: rs2069727; rs2069718; and rs1861493.

60. The method of claim 43, wherein the improved response polymorphism is one or more of the following: rs2069727G; rs2069718C; and rs1861493A.

61. The method of claim 43, wherein the activated protein C or protein C— like compound is drotecogin alfa activated.

62. Two or more oligonucleotides or peptide nucleic acids of about 10 to about 400 nucleotides that hybridize specifically to a nucleotide sequence contained in a human target sequence consisting of a subject's IFNG gene sequence, a complementary sequence of the target sequence or an RNA equivalent of the target sequence, and
wherein the oligonucleotides or peptide nucleic acids are operable in determining the presence or absence of two or more improved response polymorphism(s) in said IFNG gene sequence selected from of the following polymorphic sites:
rs1861493; rs2069718; rs2069727; rs2069705; rs2069733; rs10467155; rs7973244; rs7137993; rs12315837; rs4913277; rs2080414; rs7956817; rs2069718; rs1076025; rs12312186; rs7137814; rs2098395; rs9888319; rs7298410; rs2058739; rs2216164; rs2041864; rs2870951; rs2193047; rs741344; rs4913405; rs6581794; rs10784683; rs1118866; rs10784684; rs9888400; rs7138107; rs1861494; rs2098394; rs10878779; rs2193045; rs2193049; rs2870952; rs2193048; rs2870953; rs3181034; rs759488; rs2193050; rs4913418; rs10784688; rs10748099; rs6581795; rs7302488; rs759487; rs7959933; rs4913278; rs4913415; rs2216163; rs7132697; rs7302226; rs7133554; rs2111059; rs10878763; rs2193046; rs1861493; rs10878774; rs10878786; rs10878784; rs971545; rs12301088; rs7969024; rs1177081; rs12317232; rs1177083; rs10878766; rs7969592; rs10878781; rs2870950; and rs10492197.

63. The oligonucleotides or peptide nucleic acid of claim 62, wherein the improved response polymorphism is:
rs2069727G; rs2069718C; and rs1861493A or a polymorphism in linkage disequilibrium thereto.

64. Two or more oligonucleotides or peptide nucleic acids according to claim 62, selected from the group consisting of:
(a) an oligonucleotide or peptide nucleic acid that hybridizes under high stringency conditions to a nucleic acid molecule comprising SEQ ID NO:1 having a G at position 260 but not to a nucleic acid molecule comprising SEQ ID NO:1 having an A at position 260;
(b) an oligonucleotide or peptide nucleic acid that hybridizes under high stringency conditions to a nucleic acid molecule comprising SEQ ID NO:1 having an A at position 260 but not to a nucleic acid molecule comprising SEQ ID NO:1 having a G at position 260;
(c) an oligonucleotide or peptide nucleic acid that hybridizes under high stringency conditions to a nucleic acid molecule comprising SEQ ID NO:2 having a T at position 201 but not to a nucleic acid molecule comprising SEQ ID NO:2 having a C at position 201;
(d) an oligonucleotide or peptide nucleic acid that hybridizes under high stringency conditions to a nucleic acid molecule comprising SEQ ID NO:2 having an C at position 201 but not to a nucleic acid molecule comprising SEQ ID NO:2 having a T at position 201;
(e) an oligonucleotide or peptide nucleic acid that hybridizes under high stringency conditions to a nucleic acid molecule comprising SEQ ID NO:3 having an A at position 201 but not to a nucleic acid molecule comprising SEQ ID NO:3 having a G at position 201;
(f) an oligonucleotide or peptide nucleic acid that hybridizes under high stringency conditions to a nucleic acid molecule comprising SEQ ID NO:3 having a G at position 201 but not to a nucleic acid molecule comprising SEQ ID NO:3 having an A at position 201;
(g) an oligonucleotide or peptide nucleic acid that hybridizes under high stringency conditions to a nucleic acid molecule comprising SEQ ID NO:4 having a T at position 473 but not to a nucleic acid molecule comprising SEQ ID NO:4 having a C at position 473;
(h) an oligonucleotide or peptide nucleic acid that hybridizes under high stringency conditions to a nucleic acid molecule comprising SEQ ID NO:4 having a C at position 473 but not to a nucleic acid molecule comprising SEQ ID NO:4 having a T at position 473;
(i) an oligonucleotide or peptide nucleic acid that hybridizes under high stringency conditions to a nucleic acid molecule comprising SEQ ID NO:5 having a T at position 709 but not to a nucleic acid molecule comprising SEQ ID NO:5 having a C at position 709;
(j) an oligonucleotide or peptide nucleic acid that hybridizes under high stringency conditions to a nucleic acid molecule comprising SEQ ID NO:5 having a C at position 709 but not to a nucleic acid molecule comprising SEQ ID NO:5 having a T at position 709;
(k) an oligonucleotide or peptide nucleic acid that hybridizes under high stringency conditions to a nucleic acid molecule comprising SEQ ID NO:6 having a G at position 402 but not to a nucleic acid molecule comprising SEQ ID NO:6 having a T at position 402;
(l) an oligonucleotide or peptide nucleic acid that hybridizes under high stringency conditions to a nucleic acid molecule comprising SEQ ID NO:6 having a T at position 402 but not to a nucleic acid molecule comprising SEQ ID NO:6 having a G at position 402;
(m) an oligonucleotide or peptide nucleic acid that hybridizes under high stringency conditions to a nucleic acid molecule comprising SEQ ID NO:7 having a C at position 734 but not to a nucleic acid molecule comprising SEQ ID NO:7 having a T at position 734;
(n) an oligonucleotide or peptide nucleic acid that hybridizes under high stringency conditions to a nucleic acid molecule comprising SEQ ID NO:7 having a T at position 734 but not to a nucleic acid molecule comprising SEQ ID NO:7 having a C at position 734;
(o) an oligonucleotide or peptide nucleic acid that hybridizes under high stringency conditions to a nucleic acid molecule comprising SEQ ID NO:8 having a C at position 201 but not to a nucleic acid molecule comprising SEQ ID NO:8 having a T at position 201;
(p) an oligonucleotide or peptide nucleic acid that hybridizes under high stringency conditions to a nucleic acid molecule comprising SEQ ID NO:8 having a T at position 201 but not to a nucleic acid molecule comprising SEQ ID NO:8 having a C at position 201;
(q) an oligonucleotide or peptide nucleic acid that hybridizes under high stringency conditions to a nucleic acid molecule comprising SEQ ID NO:9 having a C at position 278 but not to a nucleic acid molecule comprising SEQ ID NO:9 having a T at position 278;
(r) an oligonucleotide or peptide nucleic acid that hybridizes under high stringency conditions to a nucleic acid molecule comprising SEQ ID NO:9 having a T at position 278 but not to a nucleic acid molecule comprising SEQ ID NO:9 having a C at position 278;
(s) an oligonucleotide or peptide nucleic acid that hybridizes under high stringency conditions to a nucleic acid molecule comprising SEQ ID NO:10 having a G at position 501 but not to a nucleic acid molecule comprising SEQ ID NO:10 having an A at position 501;
(t) an oligonucleotide or peptide nucleic acid that hybridizes under high stringency conditions to a nucleic acid molecule comprising SEQ ID NO:10 having an A at position 501 but not to a nucleic acid molecule comprising SEQ ID NO:10 having a G at position 501;
(u) an oligonucleotide or peptide nucleic acid that hybridizes under high stringency conditions to a nucleic acid molecule comprising SEQ ID NO:1 having a G at position 201 but not to a nucleic acid molecule comprising SEQ ID NO:11 having an A at position 201;
(v) an oligonucleotide or peptide nucleic acid that hybridizes under high stringency conditions to a nucleic acid molecule comprising SEQ ID NO:11 having an A at position 201 but not to a nucleic acid molecule comprising SEQ ID NO:11 having a G at position 201;
(w) an oligonucleotide or peptide nucleic acid that hybridizes under high stringency conditions to a nucleic acid molecule comprising SEQ ID NO:12 having a C at position 1303 but not to a nucleic acid molecule comprising SEQ ID NO:12 having a T at position 1303;
(x) an oligonucleotide or peptide nucleic acid that hybridizes under high stringency conditions to a nucleic acid molecule comprising SEQ ID NO:12 having a T at position 1303 but not to a nucleic acid molecule comprising SEQ ID NO:12 having a C at position 1303;
(y) an oligonucleotide or peptide nucleic acid that hybridizes under high stringency conditions to a nucleic acid molecule comprising SEQ ID NO:13 having a C at position 304 but not to a nucleic acid molecule comprising SEQ ID NO:13 having a T at position 304;
(z) an oligonucleotide or peptide nucleic acid that hybridizes under high stringency conditions to a nucleic acid molecule comprising SEQ ID NO:13 having a T at position 304 but not to a nucleic acid molecule comprising SEQ ID NO:13 having a C at position 304;
(aa) an oligonucleotide or peptide nucleic acid that hybridizes under high stringency conditions to a nucleic acid molecule comprising SEQ ID NO:14 having a G at position 1958 but not to a nucleic acid molecule comprising SEQ ID NO:14 having a T at position 1958;
(bb) an oligonucleotide or peptide nucleic acid that hybridizes under high stringency conditions to a nucleic acid molecule comprising SEQ ID NO:14 having a T at position 1958 but not to a nucleic acid molecule comprising SEQ ID NO:14 having a G at position 1958;
(cc) an oligonucleotide or peptide nucleic acid that hybridizes under high stringency conditions to a nucleic acid molecule comprising SEQ ID NO:15 having a G at position 272 but not to a nucleic acid molecule comprising SEQ ID NO:15 having a T at position 272;
(dd) an oligonucleotide or peptide nucleic acid that hybridizes under high stringency conditions to a nucleic acid molecule comprising SEQ ID NO:15 having a T at position 272 but not to a nucleic acid molecule comprising SEQ ID NO:15 having a G at position 272;
(ee) an oligonucleotide or peptide nucleic acid that hybridizes under high stringency conditions to a nucleic acid molecule comprising SEQ ID NO:16 having a G at position 201 but not to a nucleic acid molecule comprising SEQ ID NO:16 having an A at position 201;
(ff) an oligonucleotide or peptide nucleic acid that hybridizes under high stringency conditions to a nucleic acid molecule comprising SEQ ID NO:16 having an A at position 201 but not to a nucleic acid molecule comprising SEQ ID NO:16 having a G at position 201;
(gg) an oligonucleotide or peptide nucleic acid that hybridizes under high stringency conditions to a nucleic acid molecule comprising SEQ ID NO:17 having a C at position 501 but not to a nucleic acid molecule comprising SEQ ID NO:17 having a T at position 501;
(hh) an oligonucleotide or peptide nucleic acid that hybridizes under high stringency conditions to a nucleic acid molecule comprising SEQ ID NO:17 having a T at position 501 but not to a nucleic acid molecule comprising SEQ ID NO:17 having a C at position 501;
(ii) an oligonucleotide or peptide nucleic acid that hybridizes under high stringency conditions to a nucleic acid molecule comprising SEQ ID NO:18 having a G at position 301 but not to a nucleic acid molecule comprising SEQ ID NO:18 having an A at position 301;
(jj) an oligonucleotide or peptide nucleic acid that hybridizes under high stringency conditions to a nucleic acid molecule comprising SEQ ID NO:18 having an A at position 301 but not to a nucleic acid molecule comprising SEQ ID NO:18 having a G at position 301;
(kk) an oligonucleotide or peptide nucleic acid that hybridizes under high stringency conditions to a nucleic acid molecule comprising SEQ ID NO:19 having a G at position 368 but not to a nucleic acid molecule comprising SEQ ID NO:19 having a T at position 368;
(ll) an oligonucleotide or peptide nucleic acid that hybridizes under high stringency conditions to a nucleic acid molecule comprising SEQ ID NO:19 having a T at position 368 but not to a nucleic acid molecule comprising SEQ ID NO:19 having a G at position 368;
(mm) an oligonucleotide or peptide nucleic acid that hybridizes under high stringency conditions to a nucleic acid molecule comprising SEQ ID NO:20 having a G at position 284 but not to a nucleic acid molecule comprising SEQ ID NO:20 having an A at position 284;
(nn) an oligonucleotide or peptide nucleic acid that hybridizes under high stringency conditions to a nucleic acid molecule comprising SEQ ID NO:20 having an A at position 284 but not to a nucleic acid molecule comprising SEQ ID NO:20 having a G at position 284;
(oo) an oligonucleotide or peptide nucleic acid that hybridizes under high stringency conditions to a nucleic acid molecule comprising SEQ ID NO:21 having a G at position 301 but not to a nucleic acid molecule comprising SEQ ID NO:21 having a T at position 301;
(pp) an oligonucleotide or peptide nucleic acid that hybridizes under high stringency conditions to a nucleic acid molecule comprising SEQ ID NO:21 having a T at position 301 but not to a nucleic acid molecule comprising SEQ ID NO:21 having a G at position 301;
(qq) an oligonucleotide or peptide nucleic acid that hybridizes under high stringency conditions to a nucleic acid molecule comprising SEQ ID NO:22 having a C at position 272 but not to a nucleic acid molecule comprising SEQ ID NO:22 having a T at position 272;
(rr) an oligonucleotide or peptide nucleic acid that hybridizes under high stringency conditions to a nucleic acid molecule comprising SEQ ID NO:22 having a T at position 272 but not to a nucleic acid molecule comprising SEQ ID NO:22 having a C at position 272;
(ss) an oligonucleotide or peptide nucleic acid that hybridizes under high stringency conditions to a nucleic acid molecule comprising SEQ ID NO:23 having a C at position 256 but not to a nucleic acid molecule comprising SEQ ID NO:23 having a T at position 256;
(tt) an oligonucleotide or peptide nucleic acid that hybridizes under high stringency conditions to a nucleic acid molecule comprising SEQ ID NO:23 having a T at position 256 but not to a nucleic acid molecule comprising SEQ ID NO:23 having a C at position 256;
(uu) an oligonucleotide or peptide nucleic acid that hybridizes under high stringency conditions to a nucleic acid molecule comprising SEQ ID NO:24 having a C at position 301 but not to a nucleic acid molecule comprising SEQ ID NO:24 having a T at position 301;
(vv) an oligonucleotide or peptide nucleic acid that hybridizes under high stringency conditions to a nucleic acid molecule comprising SEQ ID NO:24 having a T at position 301 but not to a nucleic acid molecule comprising SEQ ID NO:24 having a C at position 301;
(ww) an oligonucleotide or peptide nucleic acid that hybridizes under high stringency conditions to a nucleic acid molecule comprising SEQ ID NO:25 having a G at position 501 but not to a nucleic acid molecule comprising SEQ ID NO:25 having an A at position 501;
(xx) an oligonucleotide or peptide nucleic acid that hybridizes under high stringency conditions to a nucleic acid molecule comprising SEQ ID NO:25 having an A at position 501 but not to a nucleic acid molecule comprising SEQ ID NO:25 having a G at position 501;
(yy) an oligonucleotide or peptide nucleic acid that hybridizes under high stringency conditions to a nucleic acid molecule comprising SEQ ID NO:26 having an A at position 501 but not to a nucleic acid molecule comprising SEQ ID NO:26 having a C at position 501;
(zz) an oligonucleotide or peptide nucleic acid that hybridizes under high stringency conditions to a nucleic acid molecule comprising SEQ ID NO:26 having a C at position 501 but not to a nucleic acid molecule comprising SEQ ID NO:26 having an A at position 501;
(aaa) an oligonucleotide or peptide nucleic acid that hybridizes under high stringency conditions to a nucleic acid molecule comprising SEQ ID NO:27 having an A at position 501 but not to a nucleic acid molecule comprising SEQ ID NO:27 having a C at position 501;
(bbb) an oligonucleotide or peptide nucleic acid that hybridizes under high stringency conditions to a nucleic acid molecule comprising SEQ ID NO:27 having a C at position 501 but not to a nucleic acid molecule comprising SEQ ID NO:27 having an A at position 501;
(ccc) an oligonucleotide or peptide nucleic acid that hybridizes under high stringency conditions to a nucleic acid molecule comprising SEQ ID NO:28 having a C at position 1083 but not to a nucleic acid molecule comprising SEQ ID NO:28 having a T at position 1083;
(ddd) an oligonucleotide or peptide nucleic acid that hybridizes under high stringency conditions to a nucleic acid molecule comprising SEQ ID NO:28 having a T at position 1083 but not to a nucleic acid molecule comprising SEQ ID NO:28 having a C at position 1083;
(eee) an oligonucleotide or peptide nucleic acid that hybridizes under high stringency conditions to a nucleic acid molecule comprising SEQ ID NO:29 having a C at position 349 but not to a nucleic acid molecule comprising SEQ ID NO:29 having a T at position 349;
(fff) an oligonucleotide or peptide nucleic acid that hybridizes under high stringency conditions to a nucleic acid molecule comprising SEQ ID NO:29 having a T at position 349 but not to a nucleic acid molecule comprising SEQ ID NO:29 having a C at position 349;
(ggg) an oligonucleotide or peptide nucleic acid that hybridizes under high stringency conditions to a nucleic acid molecule comprising SEQ ID NO:30 having a G at position 201 but not to a nucleic acid molecule comprising SEQ ID NO:30 having an A at position 201;
(hhh) an oligonucleotide or peptide nucleic acid that hybridizes under high stringency conditions to a nucleic acid molecule comprising SEQ ID NO:30 having an A at position 201 but not to a nucleic acid molecule comprising SEQ ID NO:30 having a G at position 201;
(iii) an oligonucleotide or peptide nucleic acid that hybridizes under high stringency conditions to a nucleic acid molecule comprising SEQ ID NO:31 having an A at position 295 but not to a nucleic acid molecule comprising SEQ ID NO:31 having a T at position 295;
(jjj) an oligonucleotide or peptide nucleic acid that hybridizes under high stringency conditions to a nucleic acid molecule comprising SEQ ID NO:31 having a T at position 295 but not to a nucleic acid molecule comprising SEQ ID NO:31 having an A at position 295;
(kkk) an oligonucleotide or peptide nucleic acid that hybridizes under high stringency conditions to a nucleic acid molecule comprising SEQ ID NO:32 having an A at position 259 but not to a nucleic acid molecule comprising SEQ ID NO:32 having a C at position 259;
(lll) an oligonucleotide or peptide nucleic acid that hybridizes under high stringency conditions to a nucleic acid molecule comprising SEQ ID NO:32 having a C at position 259 but not to a nucleic acid molecule comprising SEQ ID NO:32 having an A at position 259;
(mmm) an oligonucleotide or peptide nucleic acid that hybridizes under high stringency conditions to a nucleic acid molecule comprising SEQ ID NO:33 having a G at position 1060 but not to a nucleic acid molecule comprising SEQ ID NO:33 having an A at position 1060;
(nnn) an oligonucleotide or peptide nucleic acid that hybridizes under high stringency conditions to a nucleic acid molecule comprising SEQ ID NO:33 having an A at position 1060 but not to a nucleic acid molecule comprising SEQ ID NO:33 having a G at position 1060;
(ooo) an oligonucleotide or peptide nucleic acid that hybridizes under high stringency conditions to a nucleic acid molecule comprising SEQ ID NO:34 having a C at position 256 but not to a nucleic acid molecule comprising SEQ ID NO:34 having a T at position 256;
(ppp) an oligonucleotide or peptide nucleic acid that hybridizes under high stringency conditions to a nucleic acid molecule comprising SEQ ID NO:34 having a T at position 256 but not to a nucleic acid molecule comprising SEQ ID NO:34 having a C at position 256;
(qqq) an oligonucleotide or peptide nucleic acid that hybridizes under high stringency conditions to a nucleic acid molecule comprising SEQ ID NO:35 having a G at position 265 but not to a nucleic acid molecule comprising SEQ ID NO:35 having an A at position 265;
(rrr) an oligonucleotide or peptide nucleic acid that hybridizes under high stringency conditions to a nucleic acid molecule comprising SEQ ID NO:35 having an A at position 265 but not to a nucleic acid molecule comprising SEQ ID NO:35 having a G at position 265;
(sss) an oligonucleotide or peptide nucleic acid that hybridizes under high stringency conditions to a nucleic acid molecule comprising SEQ ID NO:36 having a C at position 530 but not to a nucleic acid molecule comprising SEQ ID NO:36 having a T at position 530;
(ttt) an oligonucleotide or peptide nucleic acid that hybridizes under high stringency conditions to a nucleic acid molecule comprising SEQ ID NO:36 having a T at position 530 but not to a nucleic acid molecule comprising SEQ ID NO:36 having a C at position 530;
(uuu) an oligonucleotide or peptide nucleic acid that hybridizes under high stringency conditions to a nucleic acid molecule comprising SEQ ID NO:37 having a C at position 297 but not to a nucleic acid molecule comprising SEQ ID NO:37 having a T at position 297;
(vvv) an oligonucleotide or peptide nucleic acid that hybridizes under high stringency conditions to a nucleic acid molecule comprising SEQ ID NO:37 having a T at position 297 but not to a nucleic acid molecule comprising SEQ ID NO:37 having a C at position 297;
(www) an oligonucleotide or peptide nucleic acid that hybridizes under high stringency conditions to a nucleic acid molecule comprising SEQ ID NO:38 having a C at position 543 but not to a nucleic acid molecule comprising SEQ ID NO:38 having a T at position 543;
(xxx) an oligonucleotide or peptide nucleic acid that hybridizes under high stringency conditions to a nucleic acid molecule comprising SEQ ID NO:38 having a T at position 543 but not to a nucleic acid molecule comprising SEQ ID NO:38 having a C at position 543;
(yyy) an oligonucleotide or peptide nucleic acid that hybridizes under high stringency conditions to a nucleic acid molecule comprising SEQ ID NO:39 having a G at position 223 but not to a nucleic acid molecule comprising SEQ ID NO:39 having a C at position 223;
(zzz) an oligonucleotide or peptide nucleic acid that hybridizes under high stringency conditions to a nucleic acid molecule comprising SEQ ID NO:39 having a C at position 223 but not to a nucleic acid molecule comprising SEQ ID NO:39 having a G at position 223;
(aaaa) an oligonucleotide or peptide nucleic acid that hybridizes under high stringency conditions to a nucleic acid molecule comprising SEQ ID NO:40 having a G at position 201 but not to a nucleic acid molecule comprising SEQ ID NO:40 having a T at position 201;
(bbbb) an oligonucleotide or peptide nucleic acid that hybridizes under high stringency conditions to a nucleic acid molecule comprising SEQ ID NO:40 having a T at position 201 but not to a nucleic acid molecule comprising SEQ ID NO:40 having a G at position 201;
(cccc) an oligonucleotide or peptide nucleic acid that hybridizes under high stringency conditions to a nucleic acid molecule comprising SEQ ID NO:41 having a C at position 112 but not to a nucleic acid molecule comprising SEQ ID NO:41 having a T at position 112;
(dddd) an oligonucleotide or peptide nucleic acid that hybridizes under high stringency conditions to a nucleic acid molecule comprising SEQ ID NO:41 having a T at position 112 but not to a nucleic acid molecule comprising SEQ ID NO:41 having a C at position 112;
(eeee) an oligonucleotide or peptide nucleic acid that hybridizes under high stringency conditions to a nucleic acid molecule comprising SEQ ID NO:42 having a G at position 85 but not to a nucleic acid molecule comprising SEQ ID NO:42 having an A at position 85;
(ffff) an oligonucleotide or peptide nucleic acid that hybridizes under high stringency conditions to a nucleic acid molecule comprising SEQ ID NO:42 having an A at position 85 but not to a nucleic acid molecule comprising SEQ ID NO:42 having a G at position 85;
(gggg) an oligonucleotide or peptide nucleic acid that hybridizes under high stringency conditions to a nucleic acid molecule comprising SEQ ID NO:43 having a C at position 422 but not to a nucleic acid molecule comprising SEQ ID NO:43 having a T at position 422;
(hhhh) an oligonucleotide or peptide nucleic acid that hybridizes under high stringency conditions to a nucleic acid molecule comprising SEQ ID NO:43 having a T at position 422 but not to a nucleic acid molecule comprising SEQ ID NO:43 having a C at position 422;
(iiii) an oligonucleotide or peptide nucleic acid that hybridizes under high stringency conditions to a nucleic acid molecule comprising SEQ ID NO:44 having a C at position 497 but not to a nucleic acid molecule comprising SEQ ID NO:44 having a T at position 497;
(jjjj) an oligonucleotide or peptide nucleic acid that hybridizes under high stringency conditions to a nucleic acid molecule comprising SEQ ID NO:44 having a T at position 497 but not to a nucleic acid molecule comprising SEQ ID NO:44 having a C at position 497;
(kkkk) an oligonucleotide or peptide nucleic acid that hybridizes under high stringency conditions to a nucleic acid molecule comprising SEQ ID NO:45 having a C at position 500 but not to a nucleic acid molecule comprising SEQ ID NO:45 having a T at position 500;
(llll) an oligonucleotide or peptide nucleic acid that hybridizes under high stringency conditions to a nucleic acid molecule comprising SEQ ID NO:45 having a T at position 500 but not to a nucleic acid molecule comprising SEQ ID NO:45 having a C at position 500;
(mmmm) an oligonucleotide or peptide nucleic acid that hybridizes under high stringency conditions to a nucleic acid molecule comprising SEQ ID NO:46 having an A at position 939 but not to a nucleic acid molecule comprising SEQ ID NO:46 having a T at position 939;
(nnnn) an oligonucleotide or peptide nucleic acid that hybridizes under high stringency conditions to a nucleic acid molecule comprising SEQ ID NO:46 having a T at position 939 but not to a nucleic acid molecule comprising SEQ ID NO:46 having an A at position 939;
(oooo) an oligonucleotide or peptide nucleic acid that hybridizes under high stringency conditions to a nucleic acid molecule comprising SEQ ID NO:47 having a G at position 301 but not to a nucleic acid molecule comprising SEQ ID NO:47 having an A at position 301;
(pppp) an oligonucleotide or peptide nucleic acid that hybridizes under high stringency conditions to a nucleic acid molecule comprising SEQ ID NO:47 having an A at position 301 but not to a nucleic acid molecule comprising SEQ ID NO:47 having a G at position 301;
(qqqq) an oligonucleotide or peptide nucleic acid that hybridizes under high stringency conditions to a nucleic acid molecule comprising SEQ ID NO:48 having a C at position 501 but not to a nucleic acid molecule comprising SEQ ID NO:48 having a T at position 501;
(rrrr) an oligonucleotide or peptide nucleic acid that hybridizes under high stringency conditions to a nucleic acid molecule comprising SEQ ID NO:48 having a T at position 501 but not to a nucleic acid molecule comprising SEQ ID NO:48 having a C at position 501;
(ssss) an oligonucleotide or peptide nucleic acid that hybridizes under high stringency conditions to a nucleic acid molecule comprising SEQ ID NO:49 having a C at position 1311 but not to a nucleic acid molecule comprising SEQ ID NO:49 having a T at position 1311;
(tttt) an oligonucleotide or peptide nucleic acid that hybridizes under high stringency conditions to a nucleic acid molecule comprising SEQ ID NO:49 having a T at position 1311 but not to a nucleic acid molecule comprising SEQ ID NO:49 having a C at position 1311;
(uuuu) an oligonucleotide or peptide nucleic acid that hybridizes under high stringency conditions to a nucleic acid molecule comprising SEQ ID NO:50 having a G at position 1307 but not to a nucleic acid molecule comprising SEQ ID NO:50 having an A at position 1307;
(vvvv) an oligonucleotide or peptide nucleic acid that hybridizes under high stringency conditions to a nucleic acid molecule comprising SEQ ID NO:50 having an A at position 1307 but not to a nucleic acid molecule comprising SEQ ID NO:50 having a G at position 1307;
(wwww) an oligonucleotide or peptide nucleic acid that hybridizes under high stringency conditions to a nucleic acid molecule comprising SEQ ID NO:51 having a G at position 288 but not to a nucleic acid molecule comprising SEQ ID NO:51 having an A at position 288;
(xxxx) an oligonucleotide or peptide nucleic acid that hybridizes under high stringency conditions to a nucleic acid molecule comprising SEQ ID NO:51 having an A at position 288 but not to a nucleic acid molecule comprising SEQ ID NO:51 having a G at position 288;
(yyyy) an oligonucleotide or peptide nucleic acid that hybridizes under high stringency conditions to a nucleic acid molecule comprising SEQ ID NO:52 having a G at position 301 but not to a nucleic acid molecule comprising SEQ ID NO:52 having an A at position 301;
(zzzz) an oligonucleotide or peptide nucleic acid that hybridizes under high stringency conditions to a nucleic acid molecule comprising SEQ ID NO:52 having an A at position 301 but not to a nucleic acid molecule comprising SEQ ID NO:52 having a G at position 301;
(aaaaa) an oligonucleotide or peptide nucleic acid that hybridizes under high stringency conditions to a nucleic acid molecule comprising SEQ ID NO:53 having a C at position 354 but not to a nucleic acid molecule comprising SEQ ID NO:53 having a T at position 354;
(bbbbb) an oligonucleotide or peptide nucleic acid that hybridizes under high stringency conditions to a nucleic acid molecule comprising SEQ ID NO:53 having a T at position 354 but not to a nucleic acid molecule comprising SEQ ID NO:53 having a C at position 354;
(ccccc) an oligonucleotide or peptide nucleic acid that hybridizes under high stringency conditions to a nucleic acid molecule comprising SEQ ID NO:54 having a G at position 201 but not to a nucleic acid molecule comprising SEQ ID NO:54 having an A at position 201;
(ddddd) an oligonucleotide or peptide nucleic acid that hybridizes under high stringency conditions to a nucleic acid molecule comprising SEQ ID NO:54 having an A at position 201 but not to a nucleic acid molecule comprising SEQ ID NO:54 having a G at position 201;
(eeeee) an oligonucleotide or peptide nucleic acid that hybridizes under high stringency conditions to a nucleic acid molecule comprising SEQ ID NO:55 having an A at position 301 but not to a nucleic acid molecule comprising SEQ ID NO:55 having a T at position 301;
(fffff) an oligonucleotide or peptide nucleic acid that hybridizes under high stringency conditions to a nucleic acid molecule comprising SEQ ID NO:55 having a T at position 301 but not to a nucleic acid molecule comprising SEQ ID NO:55 having an A at position 301;
(ggggg) an oligonucleotide or peptide nucleic acid that hybridizes under high stringency conditions to a nucleic acid molecule comprising SEQ ID NO:56 having a C at position 301 but not to a nucleic acid molecule comprising SEQ ID NO:56 having a T at position 301;
(hhhhh) an oligonucleotide or peptide nucleic acid that hybridizes under high stringency conditions to a nucleic acid molecule comprising SEQ ID NO:56 having a T at position 301 but not to a nucleic acid molecule comprising SEQ ID NO:56 having a C at position 301;
(iiiii) an oligonucleotide or peptide nucleic acid that hybridizes under high stringency conditions to a nucleic acid molecule comprising SEQ ID NO:57 having a C at position 501 but not to a nucleic acid molecule comprising SEQ ID NO:57 having a T at position 501;
(jjjj) an oligonucleotide or peptide nucleic acid that hybridizes under high stringency conditions to a nucleic acid molecule comprising SEQ ID NO:57 having a T at position 501 but not to a nucleic acid molecule comprising SEQ ID NO:57 having a C at position 501;
(kkkkk) an oligonucleotide or peptide nucleic acid that hybridizes under high stringency conditions to a nucleic acid molecule comprising SEQ ID NO:58 having a G at position 501 but not to a nucleic acid molecule comprising SEQ ID NO:58 having an A at position 501;
(lllll) an oligonucleotide or peptide nucleic acid that hybridizes under high stringency conditions to a nucleic acid molecule comprising SEQ ID NO:58 having an A at position 501 but not to a nucleic acid molecule comprising SEQ ID NO:58 having a G at position 501;
(mmmmm) an oligonucleotide or peptide nucleic acid that hybridizes under high stringency conditions to a nucleic acid molecule comprising SEQ ID NO:59 having a C at position 1216 but not to a nucleic acid molecule comprising SEQ ID NO:59 having a T at position 1216;
(nnnnn) an oligonucleotide or peptide nucleic acid that hybridizes under high stringency conditions to a nucleic acid molecule comprising SEQ ID NO:59 having a T at position 1216 but not to a nucleic acid molecule comprising SEQ ID NO:59 having a C at position 1216;
(ooooo) an oligonucleotide or peptide nucleic acid that hybridizes under high stringency conditions to a nucleic acid molecule comprising SEQ ID NO:60 having a C at position 488 but not to a nucleic acid molecule comprising SEQ ID NO:60 having a T at position 488;
(ppppp) an oligonucleotide or peptide nucleic acid that hybridizes under high stringency conditions to a nucleic acid molecule comprising SEQ ID NO:60 having a T at position 488 but not to a nucleic acid molecule comprising SEQ ID NO:60 having a C at position 488;
(qqqqq) an oligonucleotide or peptide nucleic acid that hybridizes under high stringency conditions to a nucleic acid molecule comprising SEQ ID NO:61 having a G at position 301 but not to a nucleic acid molecule comprising SEQ ID NO:61 having an A at position 301;
(rrrrr) an oligonucleotide or peptide nucleic acid that hybridizes under high stringency conditions to a nucleic acid molecule comprising SEQ ID NO:61 having an A at position 301 but not to a nucleic acid molecule comprising SEQ ID NO:61 having a G at position 301;
(sssss) an oligonucleotide or peptide nucleic acid that hybridizes under high stringency conditions to a nucleic acid molecule comprising SEQ ID NO:62 having a G at position 294 but not to a nucleic acid molecule comprising SEQ ID NO:62 having a T at position 294;
(ttttt) an oligonucleotide or peptide nucleic acid that hybridizes under high stringency conditions to a nucleic acid molecule comprising SEQ ID NO:62 having a T at position 294 but not to a nucleic acid molecule comprising SEQ ID NO:62 having a G at position 294;
(uuuuu) an oligonucleotide or peptide nucleic acid that hybridizes under high stringency conditions to a nucleic acid molecule comprising SEQ ID NO:63 having a G at position 154 but not to a nucleic acid molecule comprising SEQ ID NO:63 having an A at position 154;
(vvvvv) an oligonucleotide or peptide nucleic acid that hybridizes under high stringency conditions to a nucleic acid molecule comprising SEQ ID NO:63 having an A at position 154 but not to a nucleic acid molecule comprising SEQ ID NO:63 having a G at position 154;
(wwwww) an oligonucleotide or peptide nucleic acid that hybridizes under high stringency conditions to a nucleic acid molecule comprising SEQ ID NO:64 having a C at position 201 but not to a nucleic acid molecule comprising SEQ ID NO:64 having a T at position 201;
(xxxxx) an oligonucleotide or peptide nucleic acid that hybridizes under high stringency conditions to a nucleic acid molecule comprising SEQ ID NO:64 having a T at position 201 but not to a nucleic acid molecule comprising SEQ ID NO:64 having a C at position 201;
(yyyyy) an oligonucleotide or peptide nucleic acid that hybridizes under high stringency conditions to a nucleic acid molecule comprising SEQ ID NO:65 having a C at position 201 but not to a nucleic acid molecule comprising SEQ ID NO:65 having a T at position 201;
(zzzzz) an oligonucleotide or peptide nucleic acid that hybridizes under high stringency conditions to a nucleic acid molecule comprising SEQ ID NO:65 having a T at position 201 but not to a nucleic acid molecule comprising SEQ ID NO:65 having a C at position 201;
(aaaaaa) an oligonucleotide or peptide nucleic acid that hybridizes under high stringency conditions to a nucleic acid molecule comprising SEQ ID NO:66 having an A at position 201 but not to a nucleic acid molecule comprising SEQ ID NO:66 having a T at position 201;
(bbbbbb) an oligonucleotide or peptide nucleic acid that hybridizes under high stringency conditions to a nucleic acid molecule comprising SEQ ID NO:66 having a T at position 201 but not to a nucleic acid molecule comprising SEQ ID NO:66 having an A at position 201;
(cccccc) an oligonucleotide or peptide nucleic acid that hybridizes under high stringency conditions to a nucleic acid molecule comprising SEQ ID NO:67 having a C at position 201 but not to a nucleic acid molecule comprising SEQ ID NO:67 having a T at position 201;
(dddddd) an oligonucleotide or peptide nucleic acid that hybridizes under high stringency conditions to a nucleic acid molecule comprising SEQ ID NO:67 having a T at position 201 but not to a nucleic acid molecule comprising SEQ ID NO:67 having a C at position 201;
(eeeeee) an oligonucleotide or peptide nucleic acid that hybridizes under high stringency conditions to a nucleic acid molecule comprising SEQ ID NO:68 having a G at position 527 but not to a nucleic acid molecule comprising SEQ ID NO:68 having a T at position 527;
(ffffff) an oligonucleotide or peptide nucleic acid that hybridizes under high stringency conditions to a nucleic acid molecule comprising SEQ ID NO:68 having a T at position 527 but not to a nucleic acid molecule comprising SEQ ID NO:68 having a G at position 527;
(gggggg) an oligonucleotide or peptide nucleic acid that hybridizes under high stringency conditions to a nucleic acid molecule comprising SEQ ID NO:69 having a G at position 301 but not to a nucleic acid molecule comprising SEQ ID NO:69 having an A at position 301;
(hhhhhh) an oligonucleotide or peptide nucleic acid that hybridizes under high stringency conditions to a nucleic acid molecule comprising SEQ ID NO:69 having an A at position 301 but not to a nucleic acid molecule comprising SEQ ID NO:69 having a G at position 301; and
(iiiiii) an oligonucleotide or peptide nucleic acid that hybridizes under high stringency conditions to a nucleic acid molecule comprising SEQ ID NO:70 having an A at position 357 but not to a nucleic acid molecule comprising SEQ ID NO:70 having a T at position 357; and
(jjjjj) an oligonucleotide or peptide nucleic acid that hybridizes under high stringency conditions to a nucleic acid molecule comprising SEQ ID NO:70 having a T at position 357 but not to a nucleic acid molecule comprising SEQ ID NO:70 having an A at position 357.

65. An array of oligonucleotides or peptide nucleic acids attached to a solid support, the array comprising said two or more of the oligonucleotides or peptide nucleic acids of claim 62.

66. A composition comprising:
(i) an addressable collection of the two or more oligonucleotides or peptide nucleic acids according to claim 62,
(ii) an addressable collection of two or more oligonucleotides or peptide nucleic acids consisting essentially of two or more nucleic acid molecules set out in SEQ ID NO:1-70 or complements, fragments, variants, or analogs thereof, or
(iii) an addressable collection of two or more oligonucleotides or peptide nucleic acids, consisting essentially of two or more nucleic acid molecules set out in TABLES 1D and 1E or complements, fragments, variants, or analogs thereof.

67.-68. (canceled)

69. The oligonucleotides or peptide nucleic acids of claim 62, further comprising one or more of the following:
(a) a detectable label;
(b) a quencher;
(c) a mobility modifier; and
(c) a contiguous non-target sequence that is situated
(i) 5′ or 3′ to the target sequence, or
(ii) 5′ and 3′ to the target sequence.
